SEQUENCE LISTING (170 sequences)

SEQ ID NO: 1 - DNA, 2441 bp, Oryza sativa cv. Azucena
ccatgtcgaggtgcttcccctacccgccgccggggtacgtgcgaaacccagtggtggccgtggccgcggccgaagcgcaggcgaccactaaggtttgttgaaccatcggatttacacacgcacgtgccggatcatttgctcttgcctgttggttttgatcggatctgttggttgtgcgtgtgtgatttggggatcgcacgtgcggggaagctaacctttgcatggataacttgagatttgtgaggccgcgcttcgaccagatcggtcgccaatcttttagtggctgaccgtggaaagaggatattactgaccttcggtttgctaattttggttgtgccgttgaatctgaaataaccagaatagtcatggggaaaaaagtctgatctggaaggttcgaattacatttctatatattgttgtgctcccagacgatggttgcaagaaatcactcatgctggataaaattgtggatgtaagagtctgcagtcgttaaaatctggaaacagcacattttgccgtagtaaatttgaatccatgttgctgtctcgttattggtgtgttacgagtaacctgtgtgttgttatctccgcttggactagattccaagtaatccagtgccttcatgacctgcaaattctatgcctatgaagtaacatgaacagtttgtatgtatgtattctgttgatgcatacttgcattatttgtgagatgtacatgttgtggtaaaattttgcattcaccatatagaaatagtaactgactatccttgtttagttcgaaaactactgcaggtttagttattctctgttgccaagagtgcttgttatgattgtaagggttacagttctgtgactaaccatgtaacaaatatattaaggattatcaaattattctatgtgaagtgtccgtgccctaattgtgttatcttctgtaactgatagcacaacatttgtttcctgctgtgtgcttgtgtaaattggtacttcatcattactatatatttcaaagaaaattctgcattgcattcccgtcgtccgttctaaatcagaactgacgattgctctggtggctgaagctccagaaagaaagggaaaaggctgaaaagaagaaagagaaaaggagtgacaggaaagctcttccacatggtgagatatccaagcattcaaagcgaacccaccacaagaagagaaaacatgaagacatcaataatgctgatcagaagtcccggaaggtttcctccatggaacctggtgagcaattggagaagagtggactctcagaagagcatggagctccttgctttactcagacagagcatggctctccagagagttcacaggacagcagcaagagaagaaaggttgtgttacccagtcctagccaagctaagaatggtgaggccctttcttgcatttgtcttcttttagctggtgatgttgaattggtttgacttatcctgaattatcatcttgcaggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtaccgaaacaaggtatgccaaccccagcaaaagtcatgccaagagtcgatgttcctccatctatgagggcatcaaaggaaaggattggccttcgtcctgcagagatgttggccaatgttggtccttcaccctccaaggcaaaacagattgtcaatcctgcagctgctaaggttacacaaagagttgatcctccacctgccaaggcatctcagagaattgatcctctgttgccatccaaggttcatatagatgctactcgatcttttacgaaggtctcccagacagagatcaagccggaagtacagcccccaattctgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatctcggggaccaggattggctgcttgatagtacgaggaaacctgatgctggcattggcaactgcagagaaattgttgatccacttacttctcaatcagcagagcagttctcattgcagcctagggcgattcatttaccagaccttcatgtctatcagttgccatatgtggttccattctaggtttgtgtagtgagatggagtaggtgagaag

SEQ ID NO: 2 - DNA, 1344 bp, Oryza sativa cv. Azucena
  CDS (1)..(1344)
atgtcgaggtgcttcccctacccgccgccggggtacgtgcgaaacccagtggtggccgtggccgcggccgaagcgcaggcgaccactaagctccagaaagaaagggaaaaggctgaaaagaagaaagagaaaaggagtgacaggaaagctcttccacatggtgagatatccaagcattcaaagcgaacccaccacaagaagagaaaacatgaagacatcaataatgctgatcagaagtcccggaaggtttcctccatggaacctggtgagcaattggagaagagtggactctcagaagagcatggagctccttgctttactcagacagagcatggctctccagagagttcacaggacagcagcaagagaagaaaggttgtgttacccagtcctagccaagctaagaatggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtaccgaaacaaggtatgccaaccccagcaaaagtcatgccaagagtcgatgttcctccatctatgagggcatcaaaggaaaggattggccttcgtcctgcagagatgttggccaatgttggtccttcaccctccaaggcaaaacagattgtcaatcctgcagctgctaaggttacacaaagagttgatcctccacctgccaaggcatctcagagaattgatcctctgttgccatccaaggttcatatagatgctactcgatcttttacgaaggtctcccagacagagatcaagccggaagtacagcccccaattctgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatctcggggaccaggattggctgcttgatagtacgaggaaacctgatgctggcattggcaactgcagagaaattgttgatccacttacttctcaatcagcagagcagttctcattgcagcctagggcgattcatttaccagaccttcatgtctatcagttgccatatgtggttccattctag

SEQ ID NO: 3 - protein, 447 aa, Oryza sativa cv. Azucena
MSRCFPYPPPGYVRNPVVAVAAAEAQATTKLQKEREKAEKKKEKRSDRKALPHGEISKHSKRTHHKKRKHEDINNADQKSRKVSSMEPGEQLEKSGLSEEHGAPCFTQTEHGSPESSQDSSKRRKVVLPSPSQAKNGNILRIKIRRDQDSSASLSEKSNVVQTPVHQMGSVSSLPSKKNSMQPHNTEMMVRTASTQQQSIKGDFQAVPKQGMPTPAKVMPRVDVPPSMRASKERIGLRPAEMLANVGPSPSKAKQIVNPAAAKVTQRVDPPPAKASQRIDPLLPSKVHIDATRSFTKVSQTEIKPEVQPPILKVPVAMPTINRQQIDTSQPKEEPCSSGRNAEAASVSVEKQSKSDRKKSRKAEKKEKKFKDLFVTWDPPSMEMDDMDLGDQDWLLDSTRKPDAGIGNCREIVDPLTSQSAEQFSLQPRAIHLPDLHVYQLPYVVPF

SEQ ID NO: 4 - DNA, 126 bp, Oryza sativa cv. Nipponbare
gggggtgagcttaggccggacgccggggcatcagccatgtcgaggtgcttcccctacccgccgccggggtacgtgcgaaacccagtggtggccgtggccgcggccgaagcgcaggcgaccactaag

SEQ ID NO: 5 - DNA, 1344 bp, Oryza sativa cv. Nipponbare; identical to SEQ ID NO: 2
  CDS (1)..(1344)

SEQ ID NO: 6 - protein, 447 aa, Oryza sativa cv. Nipponbare; identical to SEQ ID NO: 3

SEQ ID NO: 7 - DNA, 2461 bp, Oryza sativa cv. Teqing
gcggacgcgggacatcagccatgtcgaggtgcttcccctacccgccgccggggtacgtgcgaaacccagtggtggccgtggccgcggccgaagcgcaggcgaccactaaggtttgttgaaccatcggatttacacacgcacgtgccggatcatttgctcttgcctgttggttttgatcggatctgttggttgtgcgtgtgtgatttggggatcgcacgtgcggggaagctaacctttgcatggataacttgagatttgtgaggccgcgcttcgaccagatcggtcgccaatcttttagtggctgaccgtggaaagaggatattactgaccttcggtttgctaattttggttgtgccgttgaatctgaaataaccagaatagtcatggggaaaaaagtctgatctggaaggttcgaattacatttctatatattgttgtgctcccagacgatggttgcaagaaattactcatgctggataaaattgtggatgtaagagtctgcagttgttaaaatctggaaacagcacattttgccgtagtaaatttgaatccatgttgctgtctcgttattggtgtgttacgagtaacctgtgtgttgttatctccgcttggactagattccaagtaatccagtgccttcatgacctgcaaattctatgcctatgaagtaacatgaacagtttgtatgtattctgttgatgcatacttgcattatttgtgagatgtacatgttgtggtaaaattttgcattcaccatatagaaatagtaactgactatccttgtttagttcgaaaactactgcaggtttagttattctctgttgccaagagtgcttgttatgattgtaagggttacagttctgtgactaaccatgtaacaaatatattaaggattatcaaattattctatgtgaagtgtccgtgccctaattgtgttatcttctgtaactgatagcacaacatttgtttcctgctgtgtgcttgtgtaaattggtacttcatcattactatatatttcaaagaaaattctgcattgcattcccgtcgtccgttctaaatcagaactgacgattgctctggtggctgaagctccagaaagaaagggaaaaggccgaaaagaagaaagagaaaaagagtgacaggaaagctcttccacatggtgagatatccaagcattcaaagcgaacccacaagaagagaaaacatgaagacatcaataatgctgatcagaagtcccggaaggtttcctccatggaacctggtgagcaattggagaagagtggactctcagaagagcatggagctccttgctttactcagacagtgcatggctctccagagagttcacaggacagcagcaagagaagaaaggttgtgttacccagtcctagccaagctaagaatggtgaggccctttcttgcatttgtcttcttttagctggtgatgttgaattggtttgacttatcctgaattatcatcttgcaggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtactgaaacaaggtatgccaaccccagcaaaagtcatgccaagagtcgatgttcctccatctatgagggcatcaaaggaaagggttggccttcgtcctgcagagatgttggccaatgttggtccttcaccatccaaggcaaaacagattgtcaatcctgcagctgctaaggttacacaaagagttgatcctccacctgccaaggcatctcagagaattgatcctctgttgccatccaaggttcatatagatgctactcgatcttttacgaaggtctcccagacagagatcaagccggaagtacagcccccaattccgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatcttggggaccaggattggctgcttggtagtacgaggaaacctgatgctggcattggcaactgcagagaaattgttgatccacttacttctcaatcagcagagcagttctcattgcagcctagggcgattcatttaccagaccttcatgtctatcagttgccatatgtggttccattctaggtttgtgtagtgagatggagtaggtgagaagtagagagat

SEQ ID NO: 8 - DNA, 1341 bp, Oryza sativa cv. Teqing
  CDS (1)..(1341)
atgtcgaggtgcttcccctacccgccgccggggtacgtgcgaaacccagtggtggccgtggccgcggccgaagcgcaggcgaccactaagctccagaaagaaagggaaaaggccgaaaagaagaaagagaaaaagagtgacaggaaagctcttccacatggtgagatatccaagcattcaaagcgaacccacaagaagagaaaacatgaagacatcaataatgctgatcagaagtcccggaaggtttcctccatggaacctggtgagcaattggagaagagtggactctcagaagagcatggagctccttgctttactcagacagtgcatggctctccagagagttcacaggacagcagcaagagaagaaaggttgtgttacccagtcctagccaagctaagaatggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtactgaaacaaggtatgccaaccccagcaaaagtcatgccaagagtcgatgttcctccatctatgagggcatcaaaggaaagggttggccttcgtcctgcagagatgttggccaatgttggtccttcaccatccaaggcaaaacagattgtcaatcctgcagctgctaaggttacacaaagagttgatcctccacctgccaaggcatctcagagaattgatcctctgttgccatccaaggttcatatagatgctactcgatcttttacgaaggtctcccagacagagatcaagccggaagtacagcccccaattccgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatcttggggaccaggattggctgcttggtagtacgaggaaacctgatgctggcattggcaactgcagagaaattgttgatccacttacttctcaatcagcagagcagttctcattgcagcctagggcgattcatttaccagaccttcatgtctatcagttgccatatgtggttccattctag

SEQ ID NO: 9 - protein, 446 aa, Oryza sativa cv. Teqing
MSRCFPYPPPGYVRNPVVAVAAAEAQATTKLQKEREKAEKKKEKKSDRKALPHGEISKHSKRTHKKRKHEDINNADQKSRKVSSMEPGEQLEKSGLSEEHGAPCFTQTVHGSPESSQDSSKRRKVVLPSPSQAKNGNILRIKIRRDQDSSASLSEKSNVVQTPVHQMGSVSSLPSKKNSMQPHNTEMMVRTASTQQQSIKGDFQAVLKQGMPTPAKVMPRVDVPPSMRASKERVGLRPAEMLANVGPSPSKAKQIVNPAAAKVTQRVDPPPAKASQRIDPLLPSKVHIDATRSFTKVSQTEIKPEVQPPIPKVPVAMPTINRQQIDTSQPKEEPCSSGRNAEAASVSVEKQSKSDRKKSRKAEKKEKKFKDLFVTWDPPSMEMDDMDLGDQDWLLGSTRKPDAGIGNCREIVDPLTSQSAEQFSLQPRAIHLPDLHVYQLPYVVPF

SEQ ID NO: 10 - DNA, 451 bp, Oryza sativa cv. Lemont
cgccacgcgaaaccaaatcccgccgcgcgggatccttttccgccggattccacccgcgaatcggggttccccttacgattcgcgggcggattagcgcgaggcgcgcctccccctacctctgtgtgatccgggggtgaggttaggccggacgccggggcatcagccatgtcgaggtgcttcccctacccgccgccggggtacgtgcgaaacccagtggtggccgtggccgcggccgaagcgcaggcgaccactaaggtttgttgaaccatcggatttacacacgcacgtgccggatcatttgctcttgcctgttggttttgatcggatctgttggttgtgcgtgtgtgatttggggatcgcacgtgcggggaagctaacctttgcatggataacttgagatttgtgaggccgcgcttcgaccagatcggtcgccaatcttttagtggctgac

SEQ ID NO: 11 - DNA, 1616 bp, Oryza sativa cv. Lemont
acaaatatattaaggattatcaaattattctatgtgaagtgtccgtgccctaattgtgttatcttctgtaactgatagcacaacatttgtttcctgctgtgtgcttgtgtaaattggtacttcatcattactatatatttcaaagaaaattctgcattgcattcccgtcgtccgttctaaatcagaactgacgattgctctggtggctgaagctccagaaagaaagggaaaaggctgaaaagaagaaagagaaaaggagtgacaggaaagctcttccacatggtgagatatccaagcattcaaagcgaacccaccacaagaagagaaaacatgaagacatcaataatgctgatcagaagtcccggaaggtttcctccatggaacctggtgagcaattggagaagagtggactctcagaagagcatggagctccttgctttactcagacagagcatggctctccagagagttcacaggacagcagcaagagaagaaaggttgtgttacccagtcctagccaagctaagaatggtgaggccctttcttgcatttgtcttcttttagctggtgatgttgaattggtttgacttatcctgaattatcatcttgcaggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtaccgaaacaaggtatgccaaccccagcaaaagtcatgccaagagtcgatgttcctccatctatgagggcatcaaaggaaaggattggccttcgtcctgcagagatgttggccaatgttggtccttcaccctccaaggcaaaacagattgtcaatcctgcagctgctaaggttacacaaagagttgatcctccacctgccaaggcatctcagagaattgatcctctgttgccatccaaggttcatatagatgctactcgatcttttacgaaggtctcccagacagagatcaagccggaagtacagcccccaattctgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatctcggggaccaggattggctgcttgatagtacgaggaaacctgatgctggcattggcaactgcagagaaattgttgatccacttacttctcaatcagcagagcagttctcattgcagcctagggcgattcatttaccagaccttcatgtctatcagttgccatatgtggttccattctaggtttgtgtagtgagatggagtaggtgagaagtagagagatgttgggagagagctgtgtgggtctgggag

SEQ ID NO: 12 - DNA, 1344 bp, Oryza sativa cv. Lemont; identical to SEQ ID NO: 2
  CDS (1)..(1344)

SEQ ID NO: 13 - protein, 447 aa, Oryza sativa cv. Lemont; identical to SEQ ID NO: 3

SEQ ID NO: 14 - DNA, 2459 bp, Oryza sativa strain IR64
atgtcgaggtgcttcccctacccgccgccggggtacgtgcgaaacccagtggtggccgtggccgcggccgaagcgcaggcgaccactaaggtttgttgaaccatcggatttacacacgcacgtgccggatcatttgctcttgcctgttggttttgatcggatctgttggttgtgcgtgtgtgatttggggatcgcacgtgcggggaagctaacctttgcatggataacttgagatttgtgaggccgcgcttcgaccagatcggtcgccaatcttttagtggctgaccgtggaaagaggatattactgaccttcggtttgctaattttggttgtgccgttgaatctgaaataaccagaatagtcatggggaaaaagtctgatctggaaggttcgaattacatttctatatattgttgtgctcccagacgatggttgcaagaaattactcatgctggataaaattgtggatgtaagagtctgcagttgttaaaatctggaaacagcacattttgccgtagtaaatttgaatccatgttgctgtctcgttattggtgtgttacgagtaacctgtgtgttgttatctccgcttggactagattccaagtaatccagtgccttcatgacctgcaaattctatgcctatgaagtaacatgaacagtttgtatgtattctgttgatgcatacttgcattatttgtgagatgtacatgttgtggtaaaattttgcattcaccatatagaaatagtaattgactatccttgtttagttcgaaaactactgcaggtttagttattctctgttgccaagagtgcttgttatgattgtaagggttacagttctgtgactaaccatgtaacaaatatattaaggattatcaaattattctatgtgaagtgtccgtgccctaattgtgttatcttctgtaactgatagcacaacatttgtttcctgctgtgtgcttgtgtaaattggtacttcatcattactatatatttcaaagaaaattctgcattgcattcccgtcgtccgttctaaatcagaactgacgattgctctggtggctgaagctccagaaagaaagggaaaaggccgaaaagaagaaagagaaaaggagtgacaggaaagctcttccacatggtgagatatccaagcattcaaagcgaacccacaagaagagaaaacatgaagacatcaataatgctgatcagaagtcccggaaggtttcctccatggaacctggtgagcaattggagaagagtggactctcagaagagcatggagctccttgctttactcagacagtgcatggctctccagagagttcacaggacagcagcaagagaagaaaggttgtgttacccagtcctagccaagctaagaatggtgaggccctttcttgcatttgtcttcttttagctggtgatgttgaattggtttgacttatcctgaattatcatcttgcaggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtactgaaacaaggtatgccaaccccagcaaaagtcatgccaagagtcgatgttcctccatctatgagggcatcaaaggaaagggttggccttcgtcctgcagagatgttggccaatgttggtccttcaccctccaaggcaaaacagattgtcaatcctgcagctgctaaggttacacaaagagttgatcctccacctgccaaggcatctcagagaattgatcctctgttgccatccaaggttcatatagatgctactcgatcttttacgaagctctcccagacagagatcaagccggaagtacagcccccaattccgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatcttggggaccaggattggctgcttggtagtacgaggaaacctgatgctggcattggcaactgcagagaaattgttgatccacttacttctcaatcagcggagcagttctcattgcagcctagggcgattcatttaccagaccttcatgtctatcagttgccatatgtggttccattctaggtttgtgtagtgagatggagtaggtgagaagtagagagatgttgggagagagctgtgtg

SEQ ID NO: 15 - DNA, 1341 bp, Oryza sativa strain IR64
  CDS (1)..(1341)
atgtcgaggtgcttcccctacccgccgccggggtacgtgcgaaacccagtggtggccgtggccgcggccgaagcgcaggcgaccactaagctccagaaagaaagggaaaaggccgaaaagaagaaagagaaaaggagtgacaggaaagctcttccacatggtgagatatccaagcattcaaagcgaacccacaagaagagaaaacatgaagacatcaataatgctgatcagaagtcccggaaggtttcctccatggaacctggtgagcaattggagaagagtggactctcagaagagcatggagctccttgctttactcagacagtgcatggctctccagagagttcacaggacagcagcaagagaagaaaggttgtgttacccagtcctagccaagctaagaatggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtactgaaacaaggtatgccaaccccagcaaaagtcatgccaagagtcgatgttcctccatctatgagggcatcaaaggaaagggttggccttcgtcctgcagagatgttggccaatgttggtccttcaccctccaaggcaaaacagattgtcaatcctgcagctgctaaggttacacaaagagttgatcctccacctgccaaggcatctcagagaattgatcctctgttgccatccaaggttcatatagatgctactcgatcttttacgaagctctcccagacagagatcaagccggaagtacagcccccaattccgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatcttggggaccaggattggctgcttggtagtacgaggaaacctgatgctggcattggcaactgcagagaaattgttgatccacttacttctcaatcagcggagcagttctcattgcagcctagggcgattcatttaccagaccttcatgtctatcagttgccatatgtggttccattctag

SEQ ID NO: 16 - protein, 446 aa, Oryza sativa strain IR64
MSRCFPYPPPGYVRNPVVAVAAAEAQATTKLQKEREKAEKKKEKRSDRKALPHGEISKHSKRTHKKRKHEDINNADQKSRKVSSMEPGEQLEKSGLSEEHGAPCFTQTVHGSPESSQDSSKRRKVVLPSPSQAKNGNILRIKIRRDQDSSASLSEKSNVVQTPVHQMGSVSSLPSKKNSMQPHNTEMMVRTASTQQQSIKGDFQAVLKQGMPTPAKVMPRVDVPPSMRASKERVGLRPAEMLANVGPSPSKAKQIVNPAAAKVTQRVDPPPAKASQRIDPLLPSKVHIDATRSFTKLSQTEIKPEVQPPIPKVPVAMPTINRQQIDTSQPKEEPCSSGRNAEAASVSVEKQSKSDRKKSRKAEKKEKKFKDLFVTWDPPSMEMDDMDLGDQDWLLGSTRKPDAGIGNCREIVDPLTSQSAEQFSLQPRAIHLPDLHVYQLPYVVPF

SEQ ID NO: 17 - DNA, 2432 bp, Oryza sativa cv. Kasalath
  misc_feature (1950)..(1950): N = G or C
catgtcgaggtgcttcccctacccgccgccggggtacgtgcgaaacccagtggtggccgtggccgcggccgaagcgcaggcgaccactaaggtttgttgaaccatcggatttacacacgcacgtgccggatcatttgctcttgcctgttggttttgatcggatctgttggttgtgcgtgtgtgatttggggatcgcacgtgcggggaagctaacctttgcatggataacttgagatttgtgaggccgcgcttcgaccagatcggtcgccaatcttttagtggctgaccgtggaaagaggatattactgaccttcggtttgctaattttggttgtgccgttgaatctgaaataaccagaatagtcatggggaaaaaagtctgatctggaaggttcgaattacatttctatatattgttgtgctcccagacgatggttgcaagaaattactcatgctggataaaattgtggatgtaagagtctgcagttgttaaaatctggaaacagcacattttgccgtagtaaatttgaatccatgttgctgtctcgttattggtgtgttacgagtaacctgtgtgttgttatctccgcttggactagattccaagtaatccagtgccttcatgacctgcaaattctatgcctatgaagtaacatgaacagtttgtatgtattctgttgatgcatacttgcattatttgtgagatgtacatgttgtggtaaaattttgcattcaccatatagaaatagtaactgactatccttgtttagttcgaaaactactgcaggtttagttattctctgttgccaagagtgcttgttatgattgtaagggttacagttctgtgactaaccatgtaacaaatatattaaggattatcaaattattctatgtgaagtgtccgtgccctaattgtgttatcttctgtaactgatagcacaacatttgtttcctgctgtgtgcttgtgtaaattggtacttcatcattactatatatttcaaagaaaattctgcattgcattcccgtcgtccgttctaaatcagaactgacgattgctctggtggctgaagctccagaaagaaagggaaaaggccgaaaagaagaaagagaaaaggagtgacaggaaagctcttccacatggtgagatatccaagcattcaaagcgaacccacaagaagagaaaacatgaagacatcaataatgctgatcagaagtcccggaaggtttcctccatggaacctggtgagcaattggagaagagtggactctcagaagagcatggagctccttgctttactcagacagtgcatggctctccagagagttcacaggacagcagcaagagaagaaaggttgtgttacccagtcctagccaagctaagaatggtgaggccctttcttgcatttgtcttcttttagctggtgatgttgaattggtttgacttatcctgaattatcatcttgcaggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtactgaaacaaggtatgccaaccccagcaaaagtcatgccaagagtcgatgttcctccatctatgagggcatcaaaggaaagggttggccttcgtcctgcagagatgttggccaatgttggtccttcaccctccaaggcaaaacagattgtcaatcctgcagctgctaaggttacacaaagagttgatcctccacctgccaaggcatctcagagaattgatcctctgttgccatccaaggttcatatagatgctactcgatcttttacgaagntctcccagacagagatcaagccggaagtacagcccccaattccgaaggtgcctgtggctatgcctaccatcaatcgtcagcngattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatcttggggaccaggattggctgcttggtagtacgaggaaacctgatgctggcattggcaactgcagagaaattgttgatccacttacttctcaatcagcagagcagttctcattgcagcctagggcgattcatttaccagaccttcatgtctatcagttgccatatgtggttccattctaggtttgtgtagtgagatggagtaggtgagaa

SEQ ID NO: 18 - DNA, 1341 bp, Oryza sativa cv. Kasalath
  CDS (1)..(1341)
  misc_feature (889)..(889): n = G, C
atgtcgaggtgcttcccctacccgccgccggggtacgtgcgaaacccagtggtggccgtggccgcggccgaagcgcaggcgaccactaagctccagaaagaaagggaaaaggccgaaaagaagaaagagaaaaggagtgacaggaaagctcttccacatggtgagatatccaagcattcaaagcgaacccacaagaagagaaaacatgaagacatcaataatgctgatcagaagtcccggaaggtttcctccatggaacctggtgagcaattggagaagagtggactctcagaagagcatggagctccttgctttactcagacagtgcatggctctccagagagttcacaggacagcagcaagagaagaaaggttgtgttacccagtcctagccaagctaagaatggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtactgaaacaaggtatgccaaccccagcaaaagtcatgccaagagtcgatgttcctccatctatgagggcatcaaaggaaagggttggccttcgtcctgcagagatgttggccaatgttggtccttcaccctccaaggcaaaacagattgtcaatcctgcagctgctaaggttacacaaagagttgatcctccacctgccaaggcatctcagagaattgatcctctgttgccatccaaggttcatatagatgctactcgatcttttacgaagntctcccagacagagatcaagccggaagtacagcccccaattccgaaggtgcctgtggctatgcctaccatcaatcgtcagcngattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatcttggggaccaggattggctgcttggtagtacgaggaaacctgatgctggcattggcaactgcagagaaattgttgatccacttacttctcaatcagcagagcagttctcattgcagcctagggcgattcatttaccagaccttcatgtctatcagttgccatatgtggttccattctag

SEQ ID NO: 19 - protein, 446 aa, Oryza sativa cv. Kasalath
  misc_feature (297)..(297): The ′Xaa′ at location 297 stands for Ile, Val, Leu, or Phe.
MSRCFPYPPPGYVRNPVVAVAAAEAQATTKLQKEREKAEKKKEKRSDRKALPHGEISKHSKRTHKKRKHEDINNADQKSRKVSSMEPGEQLEKSGLSEEHGAPCFTQTVHGSPESSQDSSKRRKVVLPSPSQAKNGNILRIKIRRDQDSSASLSEKSNVVQTPVHQMGSVSSLPSKKNSMQPHNTEMMVRTASTQQQSIKGDFQAVLKQGMPTPAKVMPRVDVPPSMRASKERVGLRPAEMLANVGPSPSKAKQIVNPAAAKVTQRVDPPPAKASQRIDPLLPSKVHIDATRSFTKXSQTEIKPEVQPPIPKVPVAMPTINRQXIDTSQPKEEPCSSGRNAEAASVSVEKQSKSDRKKSRKAEKKEKKFKDLFVTWDPPSMEMDDMDLGDQDWLLGSTRKPDAGIGNCREIVDPLTSQSAEQFSLQPRAIHLPDLHVYQLPYVVPF

SEQ ID NO: 20 - DNA, 2447 bp, Oryza rufipogon strain 5948
ccctacccgccgccggggtacgtgcgaaacccagtggtggccgtggccgcggccgaagcgcaggcgaccactaaggtttgttgaaccatcggatttacacacgcacgtgccggatcatttgctcttgcctgttggttttgatcggatctgttggttgtgcgtgtgtgatttggggatcgcacgtgcggggaagctaacctttgcatggataacttgagatttgtgaggccgcgcttcgaccagatcggtcgccaatcttttagtggctgaccgtggaaagaggatattactgaccttcggtttgctaattttggttgtgccgttgaatctgaaataaccagaatagtcatggggaaaaagtctgatctggaaggttcgaattacatttctatatattgttgtgctcccagacgatggttgcaagaaattactcatgctggataaaattgtggatgtaagagtctgcagttgttaaaatctggaaacagcacattttgccgtagtaaatttgaatccatgttgctgtctcgttattggtgtgttacgagtaacctgtgtgttgttatctccgcttggactagattccaagtaatccagtgccttcatgacctgcaaattctatgcctatgaagtaacatgaacagtttgtatgtattctgttgatgcatacttgcattatttgtgagatgtacatgttgtggtaaaattttgcattcaccatatagaaatagtaattgactatccttgtttagttcgaaaacttctgcaggtttagttattctctgttgccaagagtgcttgttatgattgtaagggttacagttctgtgactaaccatgtaacaaatatattaaggattatcaaattattctatgtgaagtgtccgtgccctaattgtgttatcttctgtaactgatagcacaacatttgtttcctgctgtgtgcttgtgtaaattggtacttcatcattactatatatttcaaagaaaattctgcattgcattcccgtcgtccgttctaaatcagaactgacgattgctctggtggctgaagctccagaaagaaagggaaaaggccgaaaagaagaaagagaaaaggagtgacaggaaagctcttccacatggtgagatatccaagcattcaaagcgaacccacaagaagagaaaacatgaagacatcaataatgctgatcagaagtcccggaaggtttcctccatggaacctggtgagcaattggagaagagtggactctcagaagagcatggagctccttgctttactcagacagtgcatggctctccagagagttcacaggacagcagcaagagaagaaaggttgtgttacccagtcctagccaagctaagaatggtgaggccctttcttgcatttgtcttcttttagctggtgatgttgaattggtttgacttatcctgaattatcatcttgcaggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtactgaaacaaggtatgccaaccccagcaaaagtcatgccaagagtcgatgttcctccatctatgagggcatcaaaggaaagggttggccttcgtcctgcagagatgttggccaatgttggtccttcaccctccaaggcaaaacagattgtcaatcctgcagctgctaaggttacacaaagagttgatcctccacctgccaaggcatctcagagaattgatcctctgttgccatccaaggttcatatagatgctactcgatcttttacgaagctctcccagacagagatcaagccggaagtacagcccccaattccgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatcttggggaccaggattggctgcttggtagtacgaggaaacctgatgctggcattggcaactgcagagaaattgttgatccacttacttctcaatcagcggagcagttctcattgcagcctagggcgattcatttaccagaccttcatgtctatcagttgccatatgtggttccattctaggtttgtgtagtgagatggagtaggtgagaagtagagagatgttgggagagagctgtgtgggt

SEQ ID NO: 21 - DNA, 1341 bp, Oryza rufipogon strain 5948
  CDS (1)..(1341)
  misc_feature (1)..(15): n = A, C, G, T
nnnnnnnnnnnnnnnccctacccgccgccggggtacgtgcgaaacccagtggtggccgtggccgcggccgaagcgcaggcgaccactaagctccagaaagaaagggaaaaggccgaaaagaagaaagagaaaaggagtgacaggaaagctcttccacatggtgagatatccaagcattcaaagcgaacccacaagaagagaaaacatgaagacatcaataatgctgatcagaagtcccggaaggtttcctccatggaacctggtgagcaattggagaagagtggactctcagaagagcatggagctccttgctttactcagacagtgcatggctctccagagagttcacaggacagcagcaagagaagaaaggttgtgttacccagtcctagccaagctaagaatggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtactgaaacaaggtatgccaaccccagcaaaagtcatgccaagagtcgatgttcctccatctatgagggcatcaaaggaaagggttggccttcgtcctgcagagatgttggccaatgttggtccttcaccctccaaggcaaaacagattgtcaatcctgcagctgctaaggttacacaaagagttgatcctccacctgccaaggcatctcagagaattgatcctctgttgccatccaaggttcatatagatgctactcgatcttttacgaagctctcccagacagagatcaagccggaagtacagcccccaattccgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatcttggggaccaggattggctgcttggtagtacgaggaaacctgatgctggcattggcaactgcagagaaattgttgatccacttacttctcaatcagcggagcagttctcattgcagcctagggcgattcatttaccagaccttcatgtctatcagttgccatatgtggttccattctag

SEQ ID NO: 22 - protein, 446 aa, Oryza rufipogon strain 5948
  misc_feature (1)..(1): The ′Xaa′ at location 1 stands for Lys, Asn, Arg, Ser, Thr, Ile, Met, Glu, Asp, Gly, Ala, Val, Gln, His, Pro, Leu, Tyr, Trp, Cys, or Phe.
XXXXXPYPPPGYVRNPVVAVAAAEAQATTKLQKEREKAEKKKEKRSDRKALPHGEISKHSKRTHKKRKHEDINNADQKSRKVSSMEPGEQLEKSGLSEEHGAPCFTQTVHGSPESSQDSSKRRKVVLPSPSQAKNGNILRIKIRRDQDSSASLSEKSNVVQTPVHQMGSVSSLPSKKNSMQPHNTEMMVRTASTQQQSIKGDFQAVLKQGMPTPAKVMPRVDVPPSMRASKERVGLRPAEMLANVGPSPSKAKQIVNPAAAKVTQRVDPPPAKASQRIDPLLPSKVHIDATRSFTKLSQTEIKPEVQPPIPKVPVAMPTINRQQIDTSQPKEEPCSSGRNAEAASVSVEKQSKSDRKKSRKAEKKEKKFKDLFVTWDPPSMEMDDMDLGDQDWLLGSTRKPDAGIGNCREIVDPLTSQSAEQFSLQPRAIHLPDLHVYQLPYVVPF

SEQ ID NO: 23 - DNA, 146 bp, Oryza rufipogon strain 5949
cccctacctctgtgtgatccgggggtgagcttaggccggacgccggggcatcagccatgtcgaggtgcttcccctacccgccgccggggtacgtgcgaaacccagtggtggccgtggccgcggccgaagcgcaggcgaccactaag

SEQ ID NO: 24 - DNA, 1615 bp, Oryza rufipogon strain 5949
tctgtgactaaccatgtaacaaatatattaaggattatcaaattattctatgtgaagtgtccgtgccctaattgtgttatcttctgtaactgatagcacaacatttgtttcctgctgtgtgcttgtgtaaattggtacttcatcattactatatatttcaaagaaaattctgcattgcattcccgtcgtccgttctaaatcagaactgacgattgctctggtggctgaagctccagaaagaaagggaaaaggccgaaaagaagaaagagaaaaagagtgacaggaaagctcttccacatggtgagatatccaagcattcaaagcgaacccacaagaagagaaaacatgaagacatcaataatgctgatcagaagtcccggaaggtttcctccatggaacctggtgagcaattggagaagagtggactctcagaagagcatggagctccttgctttactcagacagtgcatggctctccagagagttcacaggacagcagcaagagaagaaaggttgtgttacccagtcctagccaagctaagaatggtgaggccctttcttgcatttgtcttctcttagctggtgatgttgaattggtttgacttatcctgaattatcatcttgcaggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtactgaaacaaggtatgccaaccccagcaaaagtcatgccaagagtcgatgttcctccatctatgagggcatcaaaggaaagggttggccttcgtcctgcagagatgttggccaatgttggtccttcaccatccaaggcaaaacagattgtcaatcctgcagctgctaaggttacacaaagagttgatcctccacctgccaaggcatctcagagaattgatcctctgttgccatccaaggttcatatagatgctactcgatcttttacgaaggtctcccagacagagatcaagccggaagtacagcccccaattccgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatcttggggaccaggattggctgcttggtagtacgaggaaacctgatgctggcattggcaactgcagagaaattgttgatccacttacttctcaatcagcagagcagttctcattgcagcctagggcgattcatttaccagaccttcatgtctatcagttgccatatgtggttccattctaggtttgtgtagtgagatggagtaggtgagaagtagagagatgttgggagagagc

SEQ ID NO: 25 - DNA, 1341 bp, Oryza rufipogon strain 5949; identical to SEQ ID NO: 8
  CDS (1)..(1341)

SEQ ID NO: 26 - protein, 446 aa, Oryza rufipogon strain 5949; identical to SEQ ID NO: 9

SEQ ID NO: 27 - DNA, 107 bp, Oryza rufipogon strain 5953
acgccggggcatcagccatgtcgaggtgcttcccctacccgccgccggggtacgtgcgaaacccagtggtggccgtggccgcggccgaagcgcaggcgaccactaag

SEQ ID NO: 28 - DNA, 1332 bp, Oryza rufipogon strain 5953
ctccagaaagaaagggaaaaggccgaaaagaagaaagagaaaaagagtgacaggaaagctcttccacatggtgagatatccaagcattcaaagcgaacccacaagaagagaaaacatgaagacatcaataatgctgatcagaagtcccggaaggtttcctccatggaacctggtgagcaattggagaagagtggactctcagaagagcatggagctccttgctttactcagacagtgcatggctctccagagagttcacaggacagcagcaagagaagaaaggttgtgttacccagtcctagccaagctaagaatggtgaggccctttcttgcatttttcttcttttagctggtgatgttgaattggtttgacttatcctgaattatcatcttgcaggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtactgaaacaaggtatgccaaccccagcaaaagtcatgccaagagtcgatgttcctccatctatgagggcatcaaaggaaagggttggccttcgtcctgcagagatgttggccaatgttggtccttcaccctccaaggcaaaacagattgtcaatcctgcagctgctaaggttacacaaagagttgatcctccacctgccaaggcatctcagagaattgatcctctgttgccatccaaggttcatatagatgctactcgatcttttacgaagctctcccagacagagatcaagccggaagtacagcccccaattccgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatcttggggaccaggattggctgcttggtagtacgaggaaacctgatgctggcattggcaactgcagagaaattgttgatccacttacttctcaatcagcggagcagttctcattgcagcctagggcgattcatttaccagaccttcatgtctatcagttgccatatgtggttccattctag

SEQ ID NO: 29 - DNA, 1341 bp, Oryza rufipogon strain 5953
  CDS (1)..(1341)
atgtcgaggtgcttcccctacccgccgccggggtacgtgcgaaacccagtggtggccgtggccgcggccgaagcgcaggcgaccactaagctccagaaagaaagggaaaaggccgaaaagaagaaagagaaaaagagtgacaggaaagctcttccacatggtgagatatccaagcattcaaagcgaacccacaagaagagaaaacatgaagacatcaataatgctgatcagaagtcccggaaggtttcctccatggaacctggtgagcaattggagaagagtggactctcagaagagcatggagctccttgctttactcagacagtgcatggctctccagagagttcacaggacagcagcaagagaagaaaggttgtgttacccagtcctagccaagctaagaatggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtactgaaacaaggtatgccaaccccagcaaaagtcatgccaagagtcgatgttcctccatctatgagggcatcaaaggaaagggttggccttcgtcctgcagagatgttggccaatgttggtccttcaccctccaaggcaaaacagattgtcaatcctgcagctgctaaggttacacaaagagttgatcctccacctgccaaggcatctcagagaattgatcctctgttgccatccaaggttcatatagatgctactcgatcttttacgaagctctcccagacagagatcaagccggaagtacagcccccaattccgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatcttggggaccaggattggctgcttggtagtacgaggaaacctgatgctggcattggcaactgcagagaaattgttgatccacttacttctcaatcagcggagcagttctcattgcagcctagggcgattcatttaccagaccttcatgtctatcagttgccatatgtggttccattctag

SEQ ID NO: 30 - protein, 446 aa, Oryza rufipogon strain 5953
MSRCFPYPPPGYVRNPVVAVAAAEAQATTKLQKEREKAEKKKEKKSDRKALPHGEISKHSKRTHKKRKHEDINNADQKSRKVSSMEPGEQLEKSGLSEEHGAPCFTQTVHGSPESSQDSSKRRKVVLPSPSQAKNGNILRIKIRRDQDSSASLSEKSNVVQTPVHQMGSVSSLPSKKNSMQPHNTEMMVRTASTQQQSIKGDFQAVLKQGMPTPAKVMPRVDVPPSMRASKERVGLRPAEMLANVGPSPSKAKQIVNPAAAKVTQRVDPPPAKASQRIDPLLPSKVHIDATRSFTKLSQTEIKPEVQPPIPKVPVAMPTINRQQIDTSQPKEEPCSSGRNAEAASVSVEKQSKSDRKKSRKAEKKEKKFKDLFVTWDPPSMEMDDMDLGDQDWLLGSTRKPDAGIGNCREIVDPLTSQSAEQFSLQPRAIHLPDLHVYQLPYVVPF

SEQ ID NO: 31 - DNA, 1341 bp, Oryza rufipogon strain IRCG105491; identical to SEQ ID NO: 8
  CDS (1)..(1341)

SEQ ID NO: 32 - protein, 446 aa, Oryza rufipogon strain IRCG105491; identical to SEQ ID NO: 9

SEQ ID NO: 33 - DNA, 180 bp, Zea mays mays strain BS7
gcatgtcgaggtgcttcccctacccgccaccggggtacgtgcggaacccagtggccgtggccgagccggagtcgaccgctaaggtttgttgaaccttcggatttacacacgcacgtgccagatcgtttgttcaatctgtaggttttgcgcggatctgtgtgtttgcgcgtgcgtgatgtg

SEQ ID NO: 34 - DNA, 1447 bp, Zea mays mays strain BS7
tcagaactgacgattgctctggtggctgaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggtgagactattctcttgtttttgctattctgattgattttttattatagaagaaatcaatcgcttgttcaggattttattcatcccaacttgattttacaggaaacattcttcgcttcaagattaaaagtagtcaagayccccaatcagctgttctggagaaaccaagggttcttgagcaaccattggtccaacaaatgggatcaggttcatcccygtcgggcaagcaaaattcaatccatcataagatgaatgtgagatctacctctggtcagcggagggtcgatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaaacatctggcgaggtagatgagaattagatgagatgttgggagagagctg

SEQ ID NO: 35 - DNA, 1347 bp, Zea mays mays strain BS7
  CDS (1)..(1347)
  misc_feature (1)..(1347): The Xaa at position 170 stands for Leu or Pro
atgtcgaggtgcttcccctacccgccaccggggtacgtgcggaacccagtggccgtggccgagccggagtcgaccgctaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagayccccaatcagctgttctggagaaaccaagggttcttgagcaaccattggtccaacaaatgggatcaggttcatcccygtcgggcaagcaaaattcaatccatcataagatgaatgtgagatctacctctggtcagcggagggtcgatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 36 - protein, 448 aa, Zea mays mays strain BS7
  misc_feature (170)..(170): The ′Xaa′ at location 170 stands for Pro, or Leu.
MSRCFPYPPPGYVRNPVAVAEPESTAKLLKEKEKAEKKKEKRSDRKAPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSXSGKQNSIHHKMNVRSTSGQRRVDGDSQAVQKCLITESPAKTMQRLVPQPAAKVTHPVDPQSAVKVPVGRSGLPLKSSGSVDPSPARVMRRFDPPPVKMMSQRVHHPASMVSQKVDPPFPKVLHKETGSVVRLPEATRPTVLQKPKDLPAIKQQDIRTSSSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPYVVPF

SEQ ID NO: 37 - DNA, 2646 bp, Zea mays mays strain HuoBai
gcggggtagagcgcggtcgacgtcggcatgtcgaggtgcttcccctacccgccaccggggtacgtgcggaacccagtggccgtggccgagccggagtcgaccgctaaggtttgttgaaccttcggatttacacacgcacgtgccagatcgtttgttcaatctgtaggttttgcgcggatctgtggtttgcgcgtgcgtgatgtgggtattgcccgtgccttgaaagctaaccgagctgaggaagtgtatggatcttgtgtagctgcacgaggtcctccaaatcgattgtaaaatttaagttgtatggccggtaggccaagattgggttattccggttttcgaaaactggtagcatggttatcggggacattgaaagaatggtagaacatcaaattcgattcaaaactgtgctagatttgcatatttagtcgccctaaaattacgtggacgtgggtgatccgaattggttgttgtatgatggttggaagtgactggccaaatttttttgtttctcaaagttttctttgaaaaactgtttgtcgagcgtcaattcgtatttacctgaatttactaattcttaatacagtatgtcgttattttgggctaagcttgtgtaagaagggtcgtttgacattttgtactgtattgatgctgttttgtgtttctttgttcggagcagcattcaatgctccttttgttgtttgagagaatctgatatttgccatcgtaccgaaagtccgaaaccaactattcaaattgggatttcatttctttttttttctactgtttttagagttctctttttcgctgctgtgctcttgtgggtcagtacgtgcatttctctttttttctttttttttctgatgttactcttctgttgaccaaaggagttcagaattattttggccctgtatatcaatagcaaccaacaccatttattgagcccatttttagttttcttgttctgtagagtatgcattgttgcaggtcttaactgttgtcagggaagtaacgtgttcaacatgattgtaaacgaatacaattctgttgctaactgtgtaatgatgagaaggataattgaataatctttgtgaagtattactgtctgaactgtacgcaaatgctacatttattctttgtgttcgtgtaaatatcattatacataaaaatgctgcattgcattcccgtcgtccgttctaaatcagaactgacgattgctctggtggctgaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggtgagactattctcttgtttttgctattctgattgattttttattatagaagaaatcaatatcttgttcaggattttattcatcccaacttgattttacaggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaaacatctggcgaggtagatgagaattagatgagatgttgggagagagc

SEQ ID NO: 38 - DNA, 1347 bp, Zea mays mays strain HuoBai
  CDS (1)..(1347)
atgtcgaggtgcttcccctacccgccaccggggtacgtgcggaacccagtggccgtggccgagccggagtcgaccgctaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 39 - protein, 448 aa, Zea mays mays strain HuoBai
MSRCFPYPPPGYVRNPVAVAEPESTAKLLKEKEKAEKKKEKRSDRKAPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSLSGKQNSIHHKMNVRSTSGQRRVNGDSQAVQKCLITESPAKTMQRLVPQPAAKVTHPVDPQSAVKVPVGRSGLPLKSSGSVDPSPARVMRRFDPPPVKMMSQRVHHPASMVSQKVDPPFPKVLHKETGSVVRLPEATRPTVLQKPKDLPAIKQQDIRTSSSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPYVVPF

SEQ ID NO: 40 - DNA, 262 bp, Zea mays mays strain Makki
gaacgaatttgaatcctttgtgatctctacggcggggtagagcgcggtcgaccgtcggccatgtcgaggtgcttcccctacccgccaccggggtacgtgcggaacccagtggccgtggccgagccggagtcgaccgctaaggtttgttgaaccttcggatttacacacgcacgtgccagatcgtttgttcaatctgtaggttttgcgcggatctgtggtttgcgcgtgcgtgatgtgggtattgcccgtgccttgaaagcta

SEQ ID NO: 41 - DNA, 2311 bp, Zea mays mays strain Makki
tttcgaaaactggtagcatggttatcggggacattgaaagaatggtagaacatcaaattcgattcaaaactgtgctagatttgcatatttagtcgccctaaaattacgtggacgtgggtgatccgaattggttgttgtatgatggttggaagtgactggccaaatttttttgtttctcaaagttttctttgacaaactgtttgtcgagcgtcaattcgtatttacctgaatttactaattcttaatacagtatgtcgttattttgggctaagcttgtgtaagaagggtcgtttgacattttgtactgtattgatgctgttttgtgtttctttgttcggagcagcattcaatgctccttttgttgtttgagagaatctgatatttgccatcgtaccgaaagtccgaaaccaactattcaaattgggatttcatttctttttttttctactgtttttagagttctctttttcgctgctgtgctcttgtgggtcagtacgtgcatttctctttttttctttttttttctgatgttactcttctgttgaccaaaggagttcagaattattttggacctgtatatcaatagcaaccaacaccatttattgagcccatttttagttttcttgttctgtagagtatgcattgttgcaggtcttaactgttgtcagggaagtaacgtgttcaacatgattgtaaacgaatacaattctgttgctaactgtgtaatgatgagaaggataattgaataatctttgtgaagtattactgtctgaactgtacgcaaatgctacattcattctttgtgttcgtgtaaatatcattatacataaaaatgctgcattgcattcccgtcgtccgttctaaatcagaactgacgattgctctggtggctgaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggtgagactattctcttgtttttgctattctgattgattttttattatagaagaaatcaatcgcttgttcaggattttattcatcccaacttgattttacaggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcrggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaaacatctggcgaggtagatgagaattagatgagatgttgggagagagct

SEQ ID NO: 42 - DNA, 1347 bp, Zea mays mays strain Makki
  CDS (1)..(1347)
  misc_feature (1)..(1347): The Xaa at position 247 stands for Ser
atgtcgaggtgcttcccctacccgccaccggggtacgtgcggaacccagtggccgtggccgagccggagtcgaccgctaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcrggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 43 - protein, 448 aa, Zea mays mays strain Makki
  misc_feature (247)..(247): The ′Xaa′ at location 247 stands for Ser.
MSRCFPYPPPGYVRNPVAVAEPESTAKLLKEKEKAEKKKEKRSDRKAPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSLSGKQNSIHHKMNVRSTSGQRRVNGDSQAVQKCLITESPAKTMQRLVPQPAAKVTHPVDPQSAVKVPVGRSGLPLKSXGSVDPSPARVMRRFDPPPVKMMSQRVHHPASMVSQKVDPPFPKVLHKETGSVVRLPEATRPTVLQKPKDLPAIKQQDIRTSSSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPYVVPF

SEQ ID NO: 44 - DNA, 125 bp, Zea mays mays strain Min13
ctttgtgatctctcggcggggtagagcgcggtcgaccgtcggccatgtcgaggtgcttcccctacccgccaccggggtacgtgcggaacccagtggccgtggccgagccggagtcgaccgctaag

SEQ ID NO: 45 - DNA, 198 bp, Zea mays mays strain Min13
cttaatacagtatgtcgttattttgggctaagcttgtgtaagaagggtcgtttgacattttgtactgtattgatgctgttttgtgtttctttgttcggagcagcattcaatgctccttttgttgtttgagagaatctgatatttgccatcgtaccgaaagtccgaaaccaactattcaaattgggatttcatttcttt

SEQ ID NO: 46 - DNA, 1787 bp, Zea mays mays strain Min13
ttctgatgttactcttctgttgaccaaaggagttcagaattattttggccctgtatatcaatagcaaccaacaccatttattgagcccatttttagttttcttgttctgtagagtatgcattgttgcaggtcttaactgttgtcagggaagtaacgtgttcaacatgattgtaaacgaatacaattctgttgctaactgtgtaatgatgagaaggataattgaataatctttgtgaagtattactgtctgaactgtacgcaaatgctacattcattctttgtgttcgtgtaaatatcattatacataaaaatgctgcattgcattcccgtcgtccgttctaaatcagaactgacgattgctctggtggctgaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggtgagactattctcttgtttttgctattctgattgattttttattatagaagaaatcaatcgcttgttcaggattttattcatcccaacttgattttacaggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaggatactaagctctcccggtcagayatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaaacatctggcgaggtagatgagaattagatgagatgttgggagagag

SEQ ID NO: 47 - DNA, 1347 bp, Zea mays mays strain Min13
  CDS (1)..(1347)
atgtcgaggtgcttcccctacccgccaccggggtacgtgcggaacccagtggccgtggccgagccggagtcgaccgctaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaggatactaagctctcccggtcagayatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 48 - protein, 448 aa, Zea mays mays strain Min13; identical to SEQ ID NO: 39

SEQ ID NO: 49 - DNA, 495 bp, Zea mays mays strain Pira
ctcggcgggtagagcgcggtcgacgtcggcatgtcgaggtgcttcccctacccgccaccggggtacgtgcggaacccagtggccgtggccgagccggagtcgaccgctaaggttgttgaaccttcggatttacacacgcacgtgccagatcgttgttcaatctgtaggttttgcgcggatctgtggtttgcgcgtgcgtgatgtgggtattgsccgtgccttgaaagctaaccgagctgaggaagtgtatggatcttgtgtagctgcacgaggtcctccaaatcgattgtaaaatttaagttgtatggscggtaggscaagattgggttagtccggttttcgaaaactggtagcatggttatcggggacattgaaagaatggtagaacatcaaattcgattcaaaactgtgctagatttgcatatttagtcgccctaaaattacgtggacgtgggtgatccgaattggttattgtatgatggttggaatatgagc

SEQ ID NO: 50 - DNA, 1768 bp, Zea mays mays strain Pira
ctgttgaccaatggagttcagaattattttggccctgtatatcaatagcaaccaacaccatttattgagcccatttttagttttcttgttctgtagagtatgcattgttgcaggtcttaactgttgtcagggaagtaacgtgttcaacatgattgtaaacgaatacattctgttgctaactgtgtaatgatgagaaggataattgaataatctttgtgaagtattactgtctgaactgtacgcaatgctacattcattctttgtgttcgtgtaaatatcattatacataaaaatgctgcttgcattcccgtcgtccgttctaaatcagaactgacgattgctctggtggctgaagctcctgaaagaaaaggaaaaagccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggtgagactattctcttgtttttgctattctgattgatttattattatagaagaaatcaatcacttgttcaggattttattcatcccaacttgattttacaggaaacattcttcgcttcaagattaaaagtagtcaagatccccaatcagctgttctggagaaaccaagggttcttgagcaaccattggtccaacaaatgggatcaggttcatccctgtctggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggagatcaggacctcttyctcaaaagaagagccctgcttctctggtaggaatgcagaagcagttcaagtgcaggatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaaacatctggcgaggtagatgagaattagatgagatgttgggagagag

SEQ ID NO: 51 - DNA, 1347 bp, Zea mays mays strain Pira
  CDS (1)..(1347)
  misc_feature (1)..(1347): The Xaa at location 329 stands for Ser or Phe
atgtcgaggtgcttcccctacccgccaccggggtacgtgcggaacccagtggccgtggccgagccggagtcgaccgctaagctcctgaaagaaaaggaaaaagccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagatccccaatcagctgttctggagaaaccaagggttcttgagcaaccattggtccaacaaatgggatcaggttcatccctgtctggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggagatcaggacctcttyctcaaaagaagagccctgcttctctggtaggaatgcagaagcagttcaagtgcaggatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 52 - protein, 448 aa, Zea mays mays strain Pira
  misc_feature (329)..(329): The ′Xaa′ at location 329 stands for Ser, or Phe.
MSRCFPYPPPGYVRNPVAVAEPESTAKLLKEKEKAEKKKEKRSDRKAPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSLSGKQNSIHHKMNVRSTSGQRRVNGDSQAVQKCLITESPAKTMQRLVPQPAAKVTHPVDPQSAVKVPVGRSGLPLKSSGSVDPSPARVMRRFDPPPVKMMSQRVHHPASMVSQKVDPPFPKVLHKETGSVVRLPEATRPTVLQKPKDLPAIKQQEIRTSXSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPYVVPF

SEQ ID NO: 53 - DNA, 212 bp, Zea mays mays strain Sari
gcgcggtcgaccgtcggcatgtcgaggtgcttcccctacccgccaccggggtacgtgcggaacccagtggccgtggccgagccggagtcgaccgctaaggtttgttgaaccttcggatttacacacgcacgtgccagatcgtttgttcaatctgtaggttttgcgcggatctgtggtttgcgcgtgcgtgatgtgggtattgcccgtgcctt

SEQ ID NO: 54 - DNA, 1803 bp, Zea mays mays strain Sari
ttttttcctttttttttctgatgttactcttctgttgaccaaaggagttcagaattattttggccctgtatatcaatagcaaccaacaccatttattgagcccatttttagttttcttgttctgtagagtatgcattgttgcaggtcttaactgttgtcagggaagtaacgtgttcaacatgattgtaaacgaatacaattctgttgctaactgtgtaatgatgagaaggataattgaataatctttgtgaagtattactgtctgaactgtacgcaaatgctacattcattctttgtgttcgtgtaaatatcattatacataaaaatgctgcattgcattcccgtcgtccgttctaaatcagaactgacgattgctctggtggctgaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggtgagactattctcttgtttttgctattctgattgattttttattatagaagaaatcaatcgcttgttcaggattttattcatcccaacttgattttacaggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttawggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcargatactaagctctcccggtcagayatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaaacatctggcgaggtagatgagaattagatgagatgttgggagagagc

SEQ ID NO: 55 - DNA, 1347 bp, Zea mays mays strain Sari
  CDS (1)..(1347)
  misc_feature (1)..(1347): The Xaa at position 234 stands for Lys or Met
atgtcgaggtgcttcccctacccgccaccggggtacgtgcggaacccagtggccgtggccgagccggagtcgaccgctaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttawggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcargatactaagctctcccggtcagayatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 56 - protein, 448 aa, Zea mays mays strain Sari
  misc_feature (234)..(234): The ′Xaa′ at location 234 stands for Lys, or Met.
MSRCFPYPPPGYVRNPVAVAEPESTAKLLKEKEKAEKKKEKRSDRKAPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSLSGKQNSIHHKMNVRSTSGQRRVNGDSQAVQKCLITESPAKTMQRLVPQPAAKVTHPVDPQSAVXVPVGRSGLPLKSSGSVDPSPARVMRRFDPPPVKMMSQRVHHPASMVSQKVDPPFPKVLHKETGSVVRLPEATRPTVLQKPKDLPAIKQQDIRTSSSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPYVVPF

SEQ ID NO: 57 - DNA, 305 bp, Zea mays mays strain Smena
gattgatttcgagcgattcgattccttgtgatctctcggcggggtagagcgcggtcgaccgtcggccatgtcgaggtgcttcccctacccgccaccggggtacgtgcggaacccagtggccgtggccgagccggagtcgaccgctaaggtttgttgaaccttcggatttacacacgcacgtgccagatcgtttgttcaatatgtaggttttgcgcggatctgtggtttgcgcgtgcgtgatgtgggtattgcccgtgcctaagctaaccgagctgaggaagtgtatggatcttgtgtagctgcac

SEQ ID NO: 58 - DNA, 2208 bp, Zea mays mays strain Smena
tttagtcgccctaaaaatacgtggacgtgggtgatccgaattggttgttgtatgatggttggaatatgagccatctagtgcttccgtgactggccaaatttttttgtttctcaaagttttctttgaaaaactgtttgtcgagcgtcaattcgtatttacctgaatttactaattcttaatacagtatgtcgttattttgggctaagcttgtgtaagaagggtcgtttgacattttgtactgtattaatgctgttttgtgtttctttgttcggagcagcattcaatgctccttttgttgtttgagagaatctgatatttgccatcgtaccgaaagtccgaaaccaactattcaaattgggatttcatttctttttttttctactgtttttagagttctctttttcgctgctgtgctcttgtgggtcagtacgtgcatttctctctttttttctttttttttctgatgttactcttctgttgaccaaaggagttcagaattattttggccctgtatatcaatttgcaaccaacaccatttattgagcccatttttagttttcttgttctgtagagttatgcattgtttcaggtcttaactgttgtcagggaagtaacgtgttcaacatgattgtaaacgaatacaattctgttgctaactgtgtaatgatgagaaggataattgaatagtctttgtgaagtattactgtctgaactgtacgcaaatgctacattcattctgtgttcatgtaaatatcattatacataaaaatgctgcattgcattcccgtcgtccgttctaaatcagaactgacgattgctctggtggctgaagctcctgaaagaaaaggaaaaggccgaaaagaagaaagagaaaaggagtgacaggaaagatcccaagcagtgtgagacgtccaaacactcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgggactctcctgagagctcacaggacagcggcaagagacgaaaggttgtcctgtccagtcctagccaacctaagaatggtgagactattctcttgtttttgctattctgattgatttattattatagaagaaatcaatcacttgttcaggattttattcatcccaacttgattttacaggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcaggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgccttctatcaagcagcaggagatcaggacctcttcctcaaaagaagagccctgcttctctggtaggaatgcagaagctgttcaagtgcaggatactaagctctcccggtcagatatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaaacatctggc

SEQ ID NO: 59 - DNA, 1640 bp, Zea mays parviglumis strain Wilkes
tcagggaagtaacgtgttcaacatgattgtaaacgaataccattctgttgctaactgtgtaatgatgagaaggataattgaataatctttgtgaagtattactgtctgaactgtacgcctaatgctacattcattctttgtgttcgtgtaaatatcattatacataaatgctgcattgcattcccgtcgtccgttctaaatcagaactgacgattgctctggtggctgaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggtgagactattctcttgtttttgctattctgattgattttttattatagaagaaatcaatcgcttgttcaggattttattcatcccaacttgattttacaggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaaacatctggcgaggtagatgagaattagatgagatgttgggag

SEQ ID NO: 60 - DNA, 1347 bp, Zea mays mays strain Smena
  CDS (1)..(1347)
atgtcgaggtgcttcccctacccgccaccggggtacgtgcggaacccagtggccgtggccgagccggagtcgaccgctaagctcctgaaagaaaaggaaaaggccgaaaagaagaaagagaaaaggagtgacaggaaagatcccaagcagtgtgagacgtccaaacactcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgggactctcctgagagctcacaggacagcggcaagagacgaaaggttgtcctgtccagtcctagccaacctaagaatggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcaggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgccttctatcaagcagcaggagatcaggacctcttcctcaaaagaagagccctgcttctctggtaggaatgcagaagctgttcaagtgcaggatactaagctctcccggtcagatatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 61 - protein, 448 aa, Zea mays mays strain Smena
MSRCFPYPPPGYVRNPVAVAEPESTAKLLKEKEKAEKKKEKRSDRKDPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSLSGKQNSIHHKMNVRSTSGQRRVNGDSQAVQKCLITESPAKTMQRLVPQPAAKVTHPVDPQSAVKVPVGRSGLPLKSSGSVDPSPARVMRRFDPPPVKMMSQRVHHPASMVSQKVDPPFPKVLHKETGSVVRLPEATRPTVLQKPKDLPSIKQQEIRTSSSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPYVVPF

SEQ ID NO: 62 - DNA, 893 bp, Zea mays mays strain W22
atgtcgaggtgcttcccctacccgccaccggggtacgtgcggaacccagtggccgtggccgagccggagtcgaccgctaaggtttgttgaaccttcggatttacacacgcacgtgccagatcgtttgttcaatctgtaggttttgcgcggatctgtggtttgcgcgtgcgtgatgtggccctgtgccttgaaagctaaccgagctgaggaagtgtatggatcttgtgtagctgcacgaggtcctccaaatcgattgtaaaatttaagttgtatggccggtaggccaagattgggttagtccggttttcgaaaactggtagcatggttatcggggacattgaaagaatggtagaacatcaaattcgattcaaaactgtgctagatttgcatatttagtcgccctaaaattacgtggacgtgggtgatccgaattggttgttgtatgatggttggaagtgactggccaaatttttttgtttctcaaagttttctttgaaaaactgtttgtcgagcgtcaattcgtatttacctgaatttactaattcttaatacagtatttcgttattttcggctaagcttgtgtaagaagggtcgtttgacattttgtactgtattaatgctgttttgtgtttctttgttcggagcagcattcaatgctccttttgttgtttgagagaatctgatatttgccatcgtaccgaaagtccgaaaccaactattcaaattgggatttcatttcttttttctactgtttttagagttctctttttcgctgctgtgctcttgtgggtcagtacgtgcatttctctttttttttctgatgttactcttctgttgaccaaaggagttcagaattattttggccctgtatatcaatagcaaccaacaccatt

SEQ ID NO: 63 - DNA, 1411 bp, Zea mays mays strain W22
ctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggtgagactattctcttgtttttgctattctgattgattttttattatagaagaaatcaatcgcttgttcaggattttattcatcccaacttgattttacaggaaacattcttcgcttcaagattaaaagtagtcaagacccccaatcagctgttctggagaaaccaagggttcttgagcaaccattggtccaacaaatgggatcaggttcatccccgtcgggcaagcaaaattcaatccatcataagatgaatgtgagatctacctctggtcagcggagggtcgatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaaacatctggcgaggtagatagaattagatagatgttgggagagag

SEQ ID NO: 64 - DNA, 1347 bp, Zea mays mays strain W22
  CDS (1)..(1347)
atgtcgaggtgcttcccctacccgccaccggggtacgtgcggaacccagtggccgtggccgagccggagtcgaccgctaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagacccccaatcagctgttctggagaaaccaagggttcttgagcaaccattggtccaacaaatgggatcaggttcatccccgtcgggcaagcaaaattcaatccatcataagatgaatgtgagatctacctctggtcagcggagggtcgatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 65 - protein, 448 aa, Zea mays mays strain W22
MSRCFPYPPPGYVRNPVAVAEPESTAKLLKEKEKAEKKKEKRSDRKAPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSPSGKQNSIHHKMNVRSTSGQRRVDGDSQAVQKCLITESPAKTMQRLVPQPAAKVTHPVDPQSAVKVPVGRSGLPLKSSGSVDPSPARVMRRFDPPPVKMMSQRVHHPASMVSQKVDPPFPKVLHKETGSVVRLPEATRPTVLQKPKDLPAIKQQDIRTSSSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPYVVPF

SEQ ID NO: 66 - DNA, 2644 bp, Zea mays parviglumis strain Benz
atgtcgaggtgcttcccctacccgccaccggggtacgtgcggaacccagtggccgtggccgagccggagtcgaccgctaaggtttgttgaaccttcggatttacacacgcacgtgccagatcgtttgttcaatctgtaggttttgcgcggatctgtggtttgcgcgtgcgtgatgtgggtattgcccgtgccttgaaagctaaccgagctgaggaagtgtatggatcttgtgtagctgcacgaggtcctccaaatcgattgtaaaatttaagttgtatggccggtaggccaagattgggttattccggttttcgaaaactggtagcatggttatcggggacattgaaagaatggtagaacatcaaattcgattcaaaactgtgctagatttgcatatttagtcgccctaaaattacgtggacgtgggtgatccgaattggttgttgtatgatggttggaagtgactggccaaatttttttgtttctcaaagttttctttgacaaactgtttgtcgagcgtcaattcgtatttacctgaatttactaattcttaatacagtatgtcgttattttgggctaagcttgtgtaagaagggtcgtttgacattttgtactgtattgatgctgttttgtgtttctttgttcggagcagcattcaatgctccttttgttgtttgagagaatctgatatttgccatcgtaccgaaagtccgaaaccaactattcaaattgggatttcatttcttttttttctactgtttttagagttctctttttcgctgctgtgctcttgtgggtcagtacgtgcatttctctttttttctttttttttctgatgttactcttctgttgaccaaaggagttcagaattattttggccctgtatatcaatagcaaccaacaccatttattgagcccatttttagttttcttgttctgtagagtatgcattgttgcaggtcttaactgttgtcagggaagtaacgtgttcaacatgattgtaaacgaatacaattctgttgctaactgtgtaatgatgagaaggataattgaataatctttgtgaagtattactgtctgaactgtacgcaaatgctacattcattctttgtgttcgtgtaaatatcattatacataaaaatgctgcattgcattcccgtcgtccgttctaaatcagaactgacgattgctctggtggctgaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggtgagactattctcttgtttttgctattctgattgattttttattatagaagaaatcaatcgcttgttcaggattttattcatcccaacttgattttacaggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaaacatctggcgaggtagatgagaattagatgagatgttgggagagagctgtgtgaacagtaggccgggtagctt

SEQ ID NO: 67 - DNA, 1347 bp, Zea mays parviglumis strain Benz; identical to SEQ ID NO: 38
  CDS (1)..(1347)

SEQ ID NO: 68 - protein, 448 aa, Zea mays parviglumis strain Benz; identical to SEQ ID NO: 39

SEQ ID NO: 69 - DNA, 586 bp, Zea mays parviglumis strain BK4
acgtcggccatgtcgaggtgcttcccctacccgccaccggggtacgtgcggaacccagtggccgtggccgagccggagtcgaccgctaaggtttgttgaaccttcggatttacacacgcacgtgccagatcgtttgttcaatctgtaggttttgcgcggatctgtggtttgcgcgtgcgtgatgtggcccgtgccttgaaagctaaccgagctgaggaagtgtatggatcttgtgtagctgcacgaggtcctccaaatcgattgtaaaatttaagttgtatggccggtaggccaagattgggttagtccggttttcgaaaactggtagcatggttatcggggacattgaaagaatggtagaacatcaaattcgattcaaaactgtgctagatttgcatatttagtcgccctaaaattacgtggacgtgggtgatccgaattggttgttgtatgatggttggaagtgactggccaaattttttgtttctcaaagttttctttgaaaaactgtttgtcgagcgtcaattcgtatttacctgaatttactaattcttaatacagtatttcgttattttcggctaagctt

SEQ ID NO: 70 - DNA, 1775 bp, Zea mays parviglumis strain BK4
tcttctgttgaccaaaggagttcagaattattttggccctgtatatcaatagcaaccaacaccatttattgatcccatttttagttttcttgttctgtagagtatgcattgttgcaggtcttaactgttgtcagggaagtaacgtgttcaacatgattgtaaacgaatacaattctgttgctaactgtgtaatgatgagaaggataattgaataatctttgtgaagtattactgtctgaactgtacgcaaatgctacattcattctttgtgttcgtgtaaatatcattatacataaaaatgctgcattgcattcccgtcgtccgttctaatcagaactgacgattgctctggtggctgaagctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggtgagactattctcttgtttttgctattctgattgattttttattatagaagaaatcaatcgcttgttcaggattttattcatcccaacttgattttacaggaaacattcttcgcttcaagattaaaagtagtcaagacccccaatcagctgttctggagaaaccaagggttcttgagcaaccattggtccaacaaatgggatcaggttcatccccgtcgggcaagcaaaattcaatccatcataagatgaatgtgagatctacctctggtcagcggagggtcgatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaaacatctggcgaggtagatgagaattagatgagatgttgggagagagc

SEQ ID NO: 71 - DNA, 1347 bp, Zea mays parviglumis strain BK4; identical to SEQ ID NO: 64
  CDS (1)..(1347)

SEQ ID NO: 72 - protein, 448 aa, Zea mays parviglumis strain BK4; identical to SEQ ID NO: 65

SEQ ID NO: 73 - DNA, 305 bp, Zea mays parviglumis strain IA19
gattgatttcgagcgattcgactccttgtgatctctacggcggggtagagcgcggtcgaccgtcggccatgtcgaggtgcttcccctacccgccaccggggtacgtgcggaacccagtggccgtggccgagccggagtcgaccgctaaggtttgttgaaccttcggatttacacacgcacgtgccagatcgtttgttcaatctgtaggttttgcgcggatctgtggtttgcgcgtgcgtgatgtgggtattgcccgtgccttgaaagctaaccgagctgaggaagtgtatggatcttgtgtagct

SEQ ID NO: 74 - DNA, 1309 bp, Zea mays parviglumis strain IA19
tcaaagcacagccataagaagagaaagcttgaagatgtcatcaaagctgagcaggttcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggtgagactattctcttgtttttgctattctgattgattttttattatagaagaaatcaatcgcttgttcaggattttattcatcccaacttgattttacaggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggakatcaggacctcttcctcaaaagaagagccctgcttctctggtaggaatgcagaagcagttcaagtgcaggatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaaacatctgtcgaggtagatgagaattagat

SEQ ID NO: 75 - DNA, 1347 bp, Zea mays parviglumis strain IA19
  CDS (1)..(1332)
  misc_feature (82)..(168): n = A, C, T, or G
atgtcgaggtgcttcccctacccgccaccggggtacgtgcggaacccagtggccgtggccgagccggagtcgaccgctaagnnnnnnnnnnnnnnnnnnnnnnnnnnnnnnnnnnnnnnnnnnnnnnnnnnnnnnnnnnnnnnnnnnnnnnnnnnnnnnnnnnnnnnntcaaagcacagccataagaagagaaagcttgaagatgtcatcaaagctgagcaggttcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggakatcaggacctcttcctcaaaagaagagccctgcttctctggtaggaatgcagaagcagttcaagtgcaggatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 76 - protein, 444 aa, Zea mays parviglumis strain IA19
  misc_feature (28)..(28): The ′Xaa′ at location 28 stands for Lys, Asn, Arg, Ser, Thr, Ile, Met, Glu, Asp, Gly, Ala, Val, Gln, His, Pro, Leu, Tyr, Trp, Cys, or Phe.
MSRCFPYPPPGYVRNPVAVAEPESTAKXXXXXXXXXXXXXXXXXXXXXXXXXXXXXSKHSHKKRKLEDVIKAEQVPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSLSGKQNSIHHKMNVRSTSGQRRVNGDSQAVQKCLITESPAKTMQRLVPQPAAKVTHPVDPQSAVKVPVGRSGLPLKSSGSVDPSPARVMRRFDPPPVKMMSQRVHHPASMVSQKVDPPFPKVLHKETGSVVRLPEATRPTVLQKPKDLPAIKQQXIRTSSSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPY

SEQ ID NO: 77 - DNA, 86 bp, Zea mays parviglumis strain Wilkes
ctctcggcggggtagagcgcggtcgaccgtcggccatgtcgaggtgcttcccctacccgccaccggggtacgtgcggaacccagtg

SEQ ID NO: 78 - DNA, 1347 bp, Zea mays parviglumis strain Wilkes
  CDS (1)..(1347)
  misc_feature (52)..(81): N = A, C, G, or T
atgtcgaggtgcttcccctacccgccaccggggtacgtgcggaacccagtgnnnnnnnnnnnnnnnnnnnnnnnnnnnnnnctcctgaaagaaaaggaaaaggccgaaaagaagaaagagaaaaggagtgacaggaaagctcccaagcagtgtgagacgtccaaacattcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagtcttcgggaagtgtggacccttcgcctgctagagttatgagaagatttgatcctccacctgttaagatgatgtcacagagagttcaccatccagcttccatggtgtcgcagaaagttgatcctccgtttccgaaggtattacataaggaaaccggatctgttgttcgcctaccagaagctacccggcctactgttcttcaaaaacccaaggacttgcctgctatcaagcagcaggatatcaggacctcttcctcaaaagaagagccctgcttctctggtaggaatgcagaagcagttcaagtgcaagatactaagctctcccggtcagacatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatggtgcagcagaagccttctttacaacccagggcaacatttttgccggaccttaatatgtaccagctgccatatgtcgtaccattttaa

SEQ ID NO: 79 - protein, 448 aa, Zea mays parviglumis strain Wilkes
  misc_feature (18)..(18): The ′Xaa′ at location 18 stands for Lys, Asn, Arg, Ser, Thr, Ile, Met, Glu, Asp, Gly, Ala, Val, Gln, His, Pro, Leu, Tyr, Trp, Cys, or Phe.
MSRCFPYPPPGYVRNPVXXXXXXXXXXLLKEKEKAEKKKEKRSDRKAPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSLSGKQNSIHHKMNVRSTSGQRRVNGDSQAVQKCLITESPAKTMQRLVPQPAAKVTHPVDPQSAVKVPVGRSGLPLKSSGSVDPSPARVMRRFDPPPVKMMSQRVHHPASMVSQKVDPPFPKVLHKETGSVVRLPEATRPTVLQKPKDLPAIKQQDIRTSSSKEEPCFSGRNAEAVQVQDTKLSRSDMKKIRKAEKKDKKFRDLFVTWNPVLIENEGSDLGDEDWLFSSKRNSDAIMVQSRATDSSVPIHPMVQQKPSLQPRATFLPDLNMYQLPYVVPF

SEQ ID NO: 80 - DNA, 225 bp, Zea diploperennis
agcgcggtcgaccgtcggccatgtcgaggtgcttcccctacccgccaccggggtacgtgcggaacccagtggccgtggccgagccggagtcgaccgctaaggtttgttgaaccttcggatttacacacgcacgtgccagatcgtttgttcaatctgtaggttttgcgcggatctgtggtttgcgcgtgcgtgatgtgggtattgcccgtgccttgaaagctaacc

SEQ ID NO: 81 - DNA, 1672 bp, Zea diploperennis
agcccatttttagttttattgttctgtagagtatgcattgttgcaggtcttaactgttgtcagggaagtaacgtgttcaacatgattgtaaacgaatacaattctgttgctaactgtgtaatgatgagaaggataattgaataatctttgtgaagtattactgtctgaactgtacgcaaatgctacattcattctttgtgttcgtgtaaatatcattatacataaaaatgctgcattgcattcccgtcgtccgttctaaatcagaactgacgattgctctggtggctgaagctcctgaaagaaaaggaaaaggccgaaaagaagaaagagaaaaggagtgacaggaaagctcccaagcagtgtgagacgtccaaacactcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggtgagactattctcttgtttttgctattctgattgattttttattatagaagaaatcaatcacttgttcaggattttattcatcccaacttgattttacaggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcgcaagcagtacaaaaatgtttgattacagaatccccggcaaagaccatgcagagacttgtcccccagcctgcagctaaggtcacacatcctgttgatccccagtcagctgttaaggtgccagttggaaggtcgggcctacctctcaagttttcgggaagtatggacccttcgcctgctagagttatgggaagatttgatcctccacctgttaagatgatgtcacagagagttcaccatccagcttccatggtgtcgcagaaagttgatcctccgttaccgaaggtattacataaggaaaccggatctgttgttcgcctaccagaagctacccggcctactgttcttcaaaaacccaaggacttgcctgctatcaagcagcagcagatcaggacctcttcctcaaaagaagagccctgcttctctggtaggaatgcagaagcagttcaagtgcatgatactaagctctcccggtcagatatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatkgtgcagcagaaaccttctttacaacccagggcaacatttttgccggaccttaatatgtaccagctgccatatgtcgtaccattttaaacatctgtcgaggt

SEQ ID NO: 82 - DNA, 1347 bp, Zea diploperennis
  CDS (1)..(1347)
  misc_feature (1)..(1347): The Xaa at  position 420 stands for Met or Leu
atgtcgaggtgcttcccctacccgccaccggggtacgtgcggaacccagtggccgtggccgagccggagtcgaccgctaagctcctgaaagaaaaggaaaaggccgaaaagaagaaagagaaaaggagtgacaggaaagctcccaagcagtgtgagacgtccaaacactcaaagcacagccataagaagagaaagcttgaagatgtcatcaaagctgagcagggtcccaaaagagtacccaaagaatcagttgagcagttggagaagagtggactctcagaagagcatggagctccttcttttgtacatacgatacgtgactctcctgagagctcacaggacagcggcaagagacgaaaggttgtcctgtccagtcctagccaacctaagaatggaaacattcttcgcttcaagattaaaagtagtcaagatccccaatcagctgttctggagaaaccaagggttcttgagcaaccattggtccaacaaatgggatcaggttcatccctgtcgggcaagcaaaattcaatccatcataagatgaatgtgagatctacctctggtcagcggagggtcaatggtgactcgcaagcagtacaaaaatgtttgattacagaatccccggcaaagaccatgcagagacttgtcccccagcctgcagctaaggtcacacatcctgttgatccccagtcagctgttaaggtgccagttggaaggtcgggcctacctctcaagttttcgggaagtatggacccttcgcctgctagagttatgggaagatttgatcctccacctgttaagatgatgtcacagagagttcaccatccagcttccatggtgtcgcagaaagttgatcctccgttaccgaaggtattacataaggaaaccggatctgttgttcgcctaccagaagctacccggcctactgttcttcaaaaacccaaggacttgcctgctatcaagcagcagcagatcaggacctcttcctcaaaagaagagccctgcttctctggtaggaatgcagaagcagttcaagtgcatgatactaagctctcccggtcagatatgaagaaaatccgcaaagctgagaaaaaagataagaagttcagagatctgtttgttacctggaatccggtattgatagagaatgaaggttcagatcttggtgatgaagactggctgttcagcagtaaaaggaactccgatgctatcatggttcaaagcagagctactgatagttcagtgccgatccatccaatkgtgcagcagaaaccttctttacaacccagggcaacatttttgccggaccttaatatgtaccagctgccatatgtcgtaccattttaa

SEQ ID NO: 83 - protein, 448 aa, Zea diploperennis
  misc_feature (420)..(420): The ′Xaa′ at location 420 stands for Met, or Ile.
MSRCFPYPPPGYVRNPVAVAEPESTAKLLKEKEKAEKKKEKRSDRKAPKQCETSKHSKHSHKKRKLEDVIKAEQGPKRVPKESVEQLEKSGLSEEHGAPSFVHTIRDSPESSQDSGKRRKVVLSSPSQPKNGNILRFKIKSSQDPQSAVLEKPRVLEQPLVQQMGSGSSLSGKQNSIHHKMNVRSTSGQRRVNGDSQAVQKCLITESPAKTMQRLVPQPAAKVTHPVDPQSAVKVPVGRSGLPLKFSGSMDPSPARVMGRFDPPPVKMMSQRVHHPASMVSQKVDPPLPKVLHKETGSVVRLPEATRPTVLQKPKDLPAIKQQQIRTSSSKEEPCFSGRNAEAVQVHDTKLSRSDMKKIRKAEKKDKKFRDLFVTWNPVLIENEGSDLGDEDWLFSSKRNSDAIMVQSRATDSSVPIHPXVQQKPSLQPRATFLPDLNMYQLPYVVPF

SEQ ID NO: 84 - DNA, 2423 bp, Zea luxurians
ggccatgtcgaggtgcttcccctacccgccaccggggtacgtgcggaacccagtggccgtggccgagccggagtcgaccgctaaggtttgttgaaccttcggatttacacacgcacgtgccagatcgtttggtcaatctgttggttttgcgcggatctgtggtttgcgcgtgcgtgatgtgggtattgcccgtgccttgaaagctaaccgagatgaggaagtgtatggatcttgtttagctgcacgaggtcctccaaatcgattgaaaaatttaagttggatggccggtaggccaagattgggttagtccggtttttgataactggtaccatggttatcggggacattgaacagaacggtagaacatcaaattcgattcaaaactgtgctagatttgcacatttagtcgccctaagattacgtggacgtgggtggtccgaattggttgttgttgtatgatggttggaatatgagccatttagtgcttccgtgactggccaaatatttttgtttctcaaatttttctttgaaaaactgtttgtcgagcgtcaattcttaatacagtatgtcgttattttgggctaagcttgtgaaacaagggtcgtttgacatttgtactgtattaacctgatgttactcttctggttgaccaaaggagttttagaattattttggtcctgtaaatcaatagcaactaacaccatctattgtgcccatttttagttttgtatagttttgtatgcagtgttgcaggtcttaactgttgtcaggaaagtaacgtgttcacatgattgtaaacgaatacaattctgttgctaactgtgtaatgatgagaacgataattgaataatctttgtgaagtattactgtctgaactgtacacaaatgctacattcattctttgtgttcgtgtaaatgtcattatacataaaaaatgctgcattgcattcccgtcgtccgttctaaatcagaactgacgattgctctggtggctgaagctcccgaaagaaaaggaaaaggccgaaaagaagaaagagaaacggagtgacaggaaagctcccaagcagtgtgagacgtccaaacattcaaagcacatccataagaagagaaagcttgaagatgtcatcaaagctgggcagggtcccaaaagagtacccaaagaatcagttgagcagttggagaagagtggactctcagaagagcatggagctccttcttttgtacataagatacgcgactctcctgagagctcacaggacagcggcaagagacgaaaggttgtcctgtccagtcctagccaacctaagaatggtgagactattctcttgtttttgctattctgattgattttttattatagaagaaatcaatcacttgttccggattttattcatcccaacttgacattttacaggaaacattcttcgcttcaagattaaaagtaatcaagatccccaatcagctgttctggagaaaccaagggttcttgaccaaccattggtccaacaaatgggatcaggttcatccctgtcgggcaagcaaaattcaatccatcataagatgaatgtgagatctacctctggtcagcggagggtcaatggtgaatcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagttttcgggaagtgtggacccttcgcctgctagagttatgggaagatttgatcctccacctgttaagatgatgtcacagagagttcaccatccagcttccatggtgtcgcagaaagttgatcctccgttaccgaaggtattacataaggaaaccggatctgttgttcgcctaccagaagctacccggcctactgttcttcaaaaacccaaggacttgcctgctatcaagcagcaggagatcaggacctcttcctcaaaagaagagccctgcttctctggtaggaatgcagaagcagttcaagtgcaggatactaagctctcccggtcagatgtgaagaaaatccgcaaagctgagaaaaaagataagaagttcagagatctgtttgttacctggaatccggtgttgatagagaatgaaggttcagatcttggtgatgaagactggctgttcagcagtaaaaggaactccgatgctatcatggctcaaagcagagctactgatagttcagtgccgatccatccaatggtgcagcagaagccttctttgcaacccagggcaacgtttttgccggaccttaatatctaccagctgccatatgtcgtaccattttaaacatctgtcgaggtagatgagaattagatgagatgttgggagagagctgtgtgaac

SEQ ID NO: 85 - DNA, 1347 bp, Zea luxurians
  CDS (1)..(1347)
atgtcgaggtgcttcccctacccgccaccggggtacgtgcggaacccagtggccgtggccgagccggagtcgaccgctaagctcccgaaagaaaaggaaaaggccgaaaagaagaaagagaaacggagtgacaggaaagctcccaagcagtgtgagacgtccaaacattcaaagcacatccataagaagagaaagcttgaagatgtcatcaaagctgggcagggtcccaaaagagtacccaaagaatcagttgagcagttggagaagagtggactctcagaagagcatggagctccttcttttgtacataagatacgcgactctcctgagagctcacaggacagcggcaagagacgaaaggttgtcctgtccagtcctagccaacctaagaatggaaacattcttcgcttcaagattaaaagtaatcaagatccccaatcagctgttctggagaaaccaagggttcttgaccaaccattggtccaacaaatgggatcaggttcatccctgtcgggcaagcaaaattcaatccatcataagatgaatgtgagatctacctctggtcagcggagggtcaatggtgaatcccaagcagtacaaaaatgtttgattacagaatccccggcaaagaccatgcagagacttgtcccccagcctgcagctaaggtcacacatcctgttgatccccagtcagctgttaaggtgccagttggaagatcgggcctacctctgaagttttcgggaagtgtggacccttcgcctgctagagttatgggaagatttgatcctccacctgttaagatgatgtcacagagagttcaccatccagcttccatggtgtcgcagaaagttgatcctccgttaccgaaggtattacataaggaaaccggatctgttgttcgcctaccagaagctacccggcctactgttcttcaaaaacccaaggacttgcctgctatcaagcagcaggagatcaggacctcttcctcaaaagaagagccctgcttctctggtaggaatgcagaagcagttcaagtgcaggatactaagctctcccggtcagatgtgaagaaaatccgcaaagctgagaaaaaagataagaagttcagagatctgtttgttacctggaatccggtgttgatagagaatgaaggttcagatcttggtgatgaagactggctgttcagcagtaaaaggaactccgatgctatcatggctcaaagcagagctactgatagttcagtgccgatccatccaatggtgcagcagaagccttctttgcaacccagggcaacgtttttgccggaccttaatatctaccagctgccatatgtcgtaccattttaa

SEQ ID NO: 86 - protein, 448 aa, Zea luxurians
MSRCFPYPPPGYVRNPVAVAEPESTAKLPKEKEKAEKKKEKRSDRKAPKQCETSKHSKHIHKKRKLEDVIKAGQGPKRVPKESVEQLEKSGLSEEHGAPSFVHKIRDSPESSQDSGKRRKVVLSSPSQPKNGNILRFKIKSNQDPQSAVLEKPRVLDQPLVQQMGSGSSLSGKQNSIHHKMNVRSTSGQRRVNGESQAVQKCLITESPAKTMQRLVPQPAAKVTHPVDPQSAVKVPVGRSGLPLKFSGSVDPSPARVMGRFDPPPVKMMSQRVHHPASMVSQKVDPPLPKVLHKETGSVVRLPEATRPTVLQKPKDLPAIKQQEIRTSSSKEEPCFSGRNAEAVQVQDTKLSRSDVKKIRKAEKKDKKFRDLFVTWNPVLIENEGSDLGDEDWLFSSKRNSDAIMAQSRATDSSVPIHPMVQQKPSLQPRATFLPDLNIYQLPYVVPF

SEQ ID NO: 87 - DNA, 21 bp, Artificial Sequence primer
  primer
caattctctgagatgccttgg

SEQ ID NO: 88 - DNA, 21 bp, Artificial Sequence primer
  primer
caattctctgagatgccttgg

SEQ ID NO: 89 - DNA, 1402 bp, Oryza rufipogon
  misc_feature (1)..(1402): n= a, c, t, or g
gatgagctcacgcggggcggcgcggctcgagctcgagccgcctatgagggcatcaaaggaaagggttggccttcgtcctgcagagatgttggccaatgttggtccttcaccctccaaggcaaaacagattgtcaatcctgcagctgctaaggttacacaaagagttgatcctccacctgccaaggcatctcagagaattgatcctctgttgccatccaaggttcatatagatgctactcaatcttttacgaaggtctcccagacagagatcaagccggaagtacagcccccaattccgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatcttggggaccaggattggctgcttggtagtacgaggaaacctgatgctggcattggcaactgcagagaaattgttgatccactttacttctcaatcagcagagcagttctcattgcagcctanggcgattcatttacccagaccttcatgtctatcagttgccatatgtggttccattctaggtttgtgtagtgagatggagtagtgagaagtaagagatgttgggaagagagctgtgtgggtctgggagattatggttccctggcacagtttcccagctttgttcccagcgttcttgtttcacggttgctactgtccaacttcctgtgtnggttttttggcgccgctattgnggcttggactccccattgatncctcacacaaggaaattcgagtagttcaagcgctatttgattaccggcgaaccacccaaagggggggggccggtaccccacgacctttggttccccctcaactagaaggggtnatattgtcgcgccgggggtaacaatgngcacanaaccagtcacggtgnngaaagnttttatccggtccccaaaatatntcccncccancaaatntnaatacccgggggcactacagttnttataaaccngtggggcnctacaanngtggacgatctcacaaattataatcatatttgtagtatntgccgangttcgcaaccgtcanacacnatcagttgtcgacgcnacgattatttttcnacagccgngctacacaancgaccgccgaaangnatgtataggatgangtacatacnatacctgactcaanacgtaccanacatcagcatcntgcgcgnntgatgantactcaggaagnagcgtccctacntccgattgaaatngtgac

SEQ ID NO: 90 - DNA, 1341 bp, Oryza rufipogon strain IRCG105491; identical to SEQ ID NO: 8

SEQ ID NO: 91 - DNA, 2157 bp, Oryza sativa cv. Nipponbare
tcgaccagatcggtcgccaatcttttagtggctgaccgtggaaagaggatattactgacttcggtttgctaattttggttgtgccgttgaatctgaaataaccagaatagtcatggggaaaaaagtctgatctggaaggttcgaattacatttctatatattgttgtgctcccagacgatggttgcaagaaatcactcatgctggataaaattgtggatgtaagagtctgcagtcgttaaaatctggaaacagcacattttgccgtagtaaatttgaatccatgttgctgtctcgttattggtgtgttacgagtaacctgtgtgttgttatctccgcttggactagattccaagtaatccagtgccttcatgacctgcaaattctatgcctatgaagtaacatgaacagtttgtatgtatgtattctgttgatgcatacttgcattatttgtgagatgtacatgttgtggtaaaattttgcattcaccatatagaaatagtaactgactatccttgtttagttcgaaaactactgcaggtttagttattctctgttgccaagagtgcttgttatgattgtaagggttacagttctgtgactaaccatgtaacaaatatattaaggattatcaaattattctatgtgaagtgtccgtgccctaattgtgttatcttctgtaactgatagcacaacatttgtttcctgctgtgtgcttgtgtaaattggtacttcatcattactatatatttcaaagaaaattctgcattgcattcccgtcgtccgttctaaatcagaactgacgattgctctggtggctgaagctccagaaagaaagggaaaaggctgaaaagaagaaagagaaaaggagtgacaggaaagctcttccacatggtgagatatccaagcattcaaagcgaacccaccacaagaagagaaaacatgaagacatcaataatgctgatcagaagtcccggaaggtttcctccatggaacctggtgagcaattggagaagagtggactctcagaagagcatggagctccttgctttactcagacagagcatggctctccagagagttcacaggacagcagcaagagaagaaaggttgtgttacccagtcctagccaagctaagaatggtgaggccctttcttgcatttgtcttcttttagctggtgatgttgaattggtttgacttatcctgaattatcatcttgcaggtaacatccttcgaataaagataagaagagatcaagattcttcagcttccctttcggagaaatctaatgttgtacaaacaccagttcatcaaatgggatcagtttcatctctgccaagtaagaaaaactcaatgcaaccacacaacaccgaaatgatggtgagaacagcatcaacccagcagcaaagcatcaaaggtgattttcaagcagtaccgaaacaaggtatgccaaccccagcaaaagtcatgccaagagtcgatgttcctccatctatgagggcatcaaaggaaaggattggccttcgtcctgcagagatgttggccaatgttggtccttcaccctccaaggcaaaacagattgtcaatcctgcagctgctaaggttacacaaagagttgatcctccacctgccaaggcatctcagagaattgatcctctgttgccatccaaggttcatatagatgctactcgatcttttacgaaggtctcccagacagagatcaagccggaagtacagcccccaattctgaaggtgcctgtggctatgcctaccatcaatcgtcagcagattgacacctcgcagcccaaagaagagccttgctcctctggcaggaatgctgaagctgcttcagtatcagtagagaagcagtccaagtcagatcgcaaaaagagccgcaaggctgagaagaaagagaagaagttcaaagatttatttgttacctgggatcctccgtctatggaaatggatgatatggatctcggggaccaggattggctgcttgatagtacgaggaaacctgatgctggcattggcaactgcagagaaattgttgatccacttacttctcaatcagcagagcagttctcattgcagcctagggcgattcatttaccagaccttcatgtctatcagttgccatatgtggttccattctag

SEQ ID NO: 92 - DNA, 1259 bp, Oryza rufipogon strain 5948
atcaaagggcgccctgcattaaagcatgccactggaaattggcgtgcatgttttttcatcctaggtaatttgttaaagatgcatagcatatcccataaaactttggcacagtacttgaaggaattccttgtttcttcgcactagtgagtaatgcttctatatggttttgaatcgtacaatcctgtatttaccttttgccaaaatattttggtgacatacaacacaaaagaaatgctggtcgaagtaccagatagcatactttacagatcaattgaaaaatgctgtgcacatttttatctgttctgcaatgagtagctttgaagtttcagaaatgctagtttggtgacaggggatgaatgctgtgagagactggcctattatggtattgcaaagaacctagttacttatctgaaaacaaatcttcatcaaggcaaccttgaagctgcaagaaatgttacaacttggcaggggacatgctacctaacacccctcattggtgccctcctagcagattcttactggggaaagtactggactattgctgctttctcagcaatttattttattgtaagtacaagcctattgctatagaagatattagatattacctacttcggtgcacttgcaccatgtgctgaactgatcttttcaaaataatttcatatctgaaacatggataatttctgaacttttttactgaagggtctggttgctttgacgctgtcagcatcagttccagctctgcagccgcctaaatgttcaggatctatttgtccagaagcaagcttactccagtatggtgtatttttctctggcctctatatgatagccctcgggactggaggcatcaaaccttgtgtatcatcctttggagctgatcaatttgatgacagtgatccagcagacagagtaaagaagggctccttcttcaattggttttacttctgtataaatatcggtgcatttgtatcaggcaccgttatagtttggatacaagataactcaggttgggggataggatttgccattcctactatatttatggcattagcgattgcaagtttctttgttgcctcaaatatgtacagatttcagaaacctggtggaagccctcttacaagagtgtgtcaggttgttgttgcagcattccgtaagtggcacactgaagtgccacatgatacatctcttttatatgaggttgatggccagacttcagcgattgagggaagccggaagctggagcacacaagtgaacttga

SEQ ID NO: 93 - DNA, 868 bp, Oryza rufipogon strain 5948
attctttgacaaggctgccatcatctcatctgatgatgccaagagtgactcctttacaaatccgtggaggctatgcactgtcacccaggtggaagaactgaaaattctaatcagaatgtttcccatttgggccactactattatattcaacgcggtgtatgctcagaactcttctatgttcatagagcagggaatggttcttgacaagcgagttggatctttcattgtccctcctgcatccctctcaacttttgatgtcatcagtgtcatcatctggattccgttttatgaccgtgtgcttgtgccaatagctagaaagttcactggaagggagaagggtttctctgagttacagcggattggaatcggattagccctctccatccttgcaatgctatctgcagctcttgttgagttgaggcgtttagagatcgccagatctgaaggtcttattcatgaggatgttgctgttccgatgagcattctttggcaaataccgcagtatttcttggttggcgctgctgaggtctttgctgccataggtcaggttgagttcttctacaatgaagcccctgatgccatgaggagtttgtgtagtgcatttgcgcttgtaacagtctcactggggagctatttaagctcaatcatattaaccttggtgtcatattttacaactcaaggaggggatcctggatggatcccagataacctgaatgaaggccacctagatcggttcttttcattgattgctgggatcaactttgtgaatttactggttttcactggttgtgcaatgagatacagatacaagaaagcatgatgactgtactcatggtaaggtcagtttgtgtaagtaataacagatttt

SEQ ID NO: 94 - DNA, 1659 bp, Oryza rufipogon strain 5948
  CDS (1)..(1659)
atcaaagggcgccctgcattaaagcatgccactggaaattggcgtgcatgttttttcatcctaggggatgaatgctgtgagagactggcctattatggtattgcaaagaacctagttacttatctgaaaacaaatcttcatcaaggcaaccttgaagctgcaagaaatgttacaacttggcaggggacatgctacctaacacccctcattggtgccctcctagcagattcttactggggaaagtactggactattgctgctttctcagcaatttattttattggtctggttgctttgacgctgtcagcatcagttccagctctgcagccgcctaaatgttcaggatctatttgtccagaagcaagcttactccagtatggtgtatttttctctggcctctatatgatagccctcgggactggaggcatcaaaccttgtgtatcatcctttggagctgatcaatttgatgacagtgatccagcagacagagtaaagaagggctccttcttcaattggttttacttctgtataaatatcggtgcatttgtatcaggcaccgttatagtttggatacaagataactcaggttgggggataggatttgccattcctactatatttatggcattagcgattgcaagtttctttgttgcctcaaatatgtacagatttcagaaacctggtggaagccctcttacaagagtgtgtcaggttgttgttgcagcattccgtaagtggcacactgaagtgccacatgatacatctcttttatatgaggttgatggccagacttcagcgattgagggaagccggaagctggagcacacaagtgaacttgaattctttgacaaggctgccatcatctcatctgatgatgccaagagtgactcctttacaaatccgtggaggctatgcactgtcacccaggtggaagaactgaaaattctaatcagaatgtttcccatttgggccactactattatattcaacgcggtgtatgctcagaactcttctatgttcatagagcagggaatggttcttgacaagcgagttggatctttcattgtccctcctgcatccctctcaacttttgatgtcatcagtgtcatcatctggattccgttttatgaccgtgtgcttgtgccaatagctagaaagttcactggaagggagaagggtttctctgagttacagcggattggaatcggattagccctctccatccttgcaatgctatctgcagctcttgttgagttgaggcgtttagagatcgccagatctgaaggtcttattcatgaggatgttgctgttccgatgagcattctttggcaaataccgcagtatttcttggttggcgctgctgaggtctttgctgccataggtcaggttgagttcttctacaatgaagcccctgatgccatgaggagtttgtgtagtgcatttgcgcttgtaacagtctcactggggagctatttaagctcaatcatattaaccttggtgtcatattttacaactcaaggaggggatcctggatggatcccagataacctgaatgaaggccacctagatcggttcttttcattgattgctgggatcaactttgtgaatttactggttttcactggttgtgcaatgagatacagatacaagaaagcatga

SEQ ID NO: 95 - protein, 552 aa, Oryza rufipogon strain 5948
IKGRPALKHATGNWRACFFILGDECCERLAYYGIAKNLVTYLKTNLHQGNLEAARNVTTWQGTCYLTPLIGALLADSYWGKYWTIAAFSAIYFIGLVALTLSASVPALQPPKCSGSICPEASLLQYGVFFSGLYMIALGTGGIKPCVSSFGADQFDDSDPADRVKKGSFFNWFYFCINIGAFVSGTVIVWIQDNSGWGIGFAIPTIFMALAIASFFVASNMYRFQKPGGSPLTRVCQVVVAAFRKWHTEVPHDTSLLYEVDGQTSAIEGSRKLEHTSELEFFDKAAIISSDDAKSDSFTNPWRLCTVTQVEELKILIRMFPIWATTIIFNAVYAQNSSMFIEQGMVLDKRVGSFIVPPASLSTFDVISVIIWIPFYDRVLVPIARKFTGREKGFSELQRIGIGLALSILAMLSAALVELRRLEIARSEGLIHEDVAVPMSILWQIPQYFLVGAAEVFAAIGQVEFFYNEAPDAMRSLCSAFALVTVSLGSYLSSIILTLVSYFTTQGGDPGWIPDNLNEGHLDRFFSLIAGINFVNLLVFTGCAMRYRYKKA

SEQ ID NO: 96 - DNA, 1230 bp, Oryza rufipogon strain 5949
cactggaaattggcgtgcatgttttttcatcctaggtaatttgttaaagatgcatagcatatcccataaaactttggcacagtacttgaaggaattccttgtttcttcgcactagtgagtaatgcttctatatggttttgaatcgtacaatcctgtatttaccttttgccaaaatattttggtgacatacaacacaaaagaaatgctggtcgaagtaccagatagcatactttacagatcaattgaaaaatgctgtgcacatttttatctgttctgcaatgagtagctttgaagtttcagaaatgctagtttggtgacaggggatgaatgctgtgagagactggcctattatggtattgcaaagaacctagttacttatctgaaaacaaatcttcatcaaggcaaccttgaagctgcaagaaatgttacaacttggcaggggacatgctacctaacacccctcattggtgccctcctagcagattcttactggggaaagtactggactattgctgctttctcagcaatttattttattgtaagtacaagcctattgctatagaagatattagatattacctacttcggtgcacttgcaccatgtgctgaactgatcttttcaaaataatttcatatctgaaacatggataatttctgaactttttaactgaagggtctggttgctttgacgctgtcagcatcagttccagctctgcagccgcctaaatgttcaggatctatttgtccagaagcaagcttactccagtatggtgtatttttctctggcctctatatgatagccctcgggactggaggcatcaaaccttgtgtatcatcctttggagctgatcaatttgatgacagtgatccagcagacagagtaaagaagggctccttcttcaattggttttacttctgtataaatatcggtgcatttgtatcaggcaccgttatagtttggatacaagataactcaggttgggggataggatttgccattcctactatatttatggcattagcgattgcaagtttctttgttgcctcaaatatgtacagatttcagaaacctggtggaagccctcttacaagagtgtgtcaggttgttgttgcagcattccgtaagtggcacactgaagtgccacatgatacatctcttttatatgaggttgatggccagacttcagcgattgagggaagccggaagctggagcacacaagtgaacttga

SEQ ID NO: 97 - DNA, 1225 bp, Oryza rufipogon strain 5949
ataaaactgatacactaccttcttgtactgttccattttgggattggtggaaattaaatactaaatgcaacaaaaagaatatggataaggccatacagcagaacgctagtagtatattagtagtttgtccatggcatgcaattcttataagtctacttataattactattactggtgcctataattaatatgggaccattagaggtatatttgtataatgactgaaaatatcagggtagcacaagcaatatatgtcagtaggtggcttgctttacagacacatttcttttacttttttttagacaatataatatattgtgttttcttgtctgactgaaattactttttgttatacagattctttgacaaggctgccatcatctcatctgatgatgccaagagtgactcctttacaaatccgtggaggctatgcactgtcacccaggtggaagaactgaaaattctaatcagaatgtttcccatttgggccactactattatattcaacgcggtgtatgctcagaactcttctatgttcatagagcagggaatggttcttgacaagcgagttggatctttcattgtccctcctgcatccctctcaacttttgatgtcatcagtgtcatcatctggattccgttttatgaccgtgtgcttgtgccaatagctagaaagttcactggaagggagaagggtttctctgagttacagcggattggaatcggattagccctctccatccttgcaatgctatctgcagctcttgttgagttgaggcgtttagagatcgccagatctgaaggtcttattcatgaggatgttgctgttccgatgagcattctttggcaaataccgcagtatttcttggttggcgctgctgaggtctttgctgccataggtcaggttgagttcttctacaatgaagcccctgatgccatgaggagtttgtgtagtgcatttgcgcttgtaacagtctcactggggagctatttaagctcaatcatattaaccttggtgtcatattttacaactcaaggaggggatcctggatggatcccagataacctgaatgaaggccacctagatcggttcttttcattgattgctgggatcaactttgtgaatttactggttttcactggttgtgcaatgagatacagatacaagaaagcatgatgactgtactcatggtaaggtcagtttgtgtaagtaataacagatttt

SEQ ID NO: 98 - DNA, 1632 bp, Oryza rufipogon strain 5949
  CDS (1)..(1632)
  misc_feature (1)..(2): N = A, T, C, or G
nncactggaaattggcgtgcatgttttttcatcctaggggatgaatgctgtgagagactggcctattatggtattgcaaagaacctagttacttatctgaaaacaaatcttcatcaaggcaaccttgaagctgcaagaaatgttacaacttggcaggggacatgctacctaacacccctcattggtgccctcctagcagattcttactggggaaagtactggactattgctgctttctcagcaatttattttattggtctggttgctttgacgctgtcagcatcagttccagctctgcagccgcctaaatgttcaggatctatttgtccagaagcaagcttactccagtatggtgtatttttctctggcctctatatgatagccctcgggactggaggcatcaaaccttgtgtatcatcctttggagctgatcaatttgatgacagtgatccagcagacagagtaaagaagggctccttcttcaattggttttacttctgtataaatatcggtgcatttgtatcaggcaccgttatagtttggatacaagataactcaggttgggggataggatttgccattcctactatatttatggcattagcgattgcaagtttctttgttgcctcaaatatgtacagatttcagaaacctggtggaagccctcttacaagagtgtgtcaggttgttgttgcagcattccgtaagtggcacactgaagtgccacatgatacatctcttttatatgaggttgatggccagacttcagcgattgagggaagccggaagctggagcacacaagtgaacttgaattctttgacaaggctgccatcatctcatctgatgatgccaagagtgactcctttacaaatccgtggaggctatgcactgtcacccaggtggaagaactgaaaattctaatcagaatgtttcccatttgggccactactattatattcaacgcggtgtatgctcagaactcttctatgttcatagagcagggaatggttcttgacaagcgagttggatctttcattgtccctcctgcatccctctcaacttttgatgtcatcagtgtcatcatctggattccgttttatgaccgtgtgcttgtgccaatagctagaaagttcactggaagggagaagggtttctctgagttacagcggattggaatcggattagccctctccatccttgcaatgctatctgcagctcttgttgagttgaggcgtttagagatcgccagatctgaaggtcttattcatgaggatgttgctgttccgatgagcattctttggcaaataccgcagtatttcttggttggcgctgctgaggtctttgctgccataggtcaggttgagttcttctacaatgaagcccctgatgccatgaggagtttgtgtagtgcatttgcgcttgtaacagtctcactggggagctatttaagctcaatcatattaaccttggtgtcatattttacaactcaaggaggggatcctggatggatcccagataacctgaatgaaggccacctagatcggttcttttcattgattgctgggatcaactttgtgaatttactggttttcactggttgtgcaatgagatacagatacaagaaagcatga

SEQ ID NO: 99 - protein, 543 aa, Oryza rufipogon strain 5949
  misc_feature (1)..(1): The ′Xaa′ at location 1 stands for Asn, Ser, Thr, Ile, Asp, Gly, Ala, Val, His, Arg, Pro, Leu, Tyr, Cys, or Phe.
XTGNWRACFFILGDECCERLAYYGIAKNLVTYLKTNLHQGNLEAARNVTTWQGTCYLTPLIGALLADSYWGKYWTIAAFSAIYFIGLVALTLSASVPALQPPKCSGSICPEASLLQYGVFFSGLYMIALGTGGIKPCVSSFGADQFDDSDPADRVKKGSFFNWFYFCINIGAFVSGTVIVWIQDNSGWGIGFAIPTIFMALAIASFFVASNMYRFQKPGGSPLTRVCQVVVAAFRKWHTEVPHDTSLLYEVDGQTSAIEGSRKLEHTSELEFFDKAAIISSDDAKSDSFTNPWRLCTVTQVEELKILIRMFPIWATTIIFNAVYAQNSSMFIEQGMVLDKRVGSFIVPPASLSTFDVISVIIWIPFYDRVLVPIARKFTGREKGFSELQRIGIGLALSILAMLSAALVELRRLEIARSEGLIHEDVAVPMSILWQIPQYFLVGAAEVFAAIGQVEFFYNEAPDAMRSLCSAFALVTVSLGSYLSSIILTLVSYFTTQGGDPGWIPDNLNEGHLDRFFSLIAGINFVNLLVFTGCAMRYRYKKA

SEQ ID NO: 100 - DNA, 2599 bp, Oryza sativa strain Azucena
atcaaagggcgccctgcattaaagcatgccactggaaattggcgtgcatgttttttcatcctaggtaatttgttaaagatgcatagcatatcccataaaactttggcacagtacttgaaggaattccttgtttcttcgcactagtgagtaatgcttctatatggttttgaatcgtacaatcctgtatttaccttttgccaaaatattttggtgatatacaacacaaaagaaatgctggtcgaagtaccagatagcatactttacagatcaattgaaaaatgctgtgcacatttttatctgttctgcaatgagtagctttgaagtttcagaaatgctagtttggtgacaggggatgaatgctgtgagagactggcctattatggtattgcaaagaacctagttacttatctgaaaacaaatcttcatcaaggcaaccttgaagctgcaagaaatgttacaacttggcaggggacatgctacctaacacccctcattggtgccctcctagcagattcttactggggaaagtactggactattgctgctttctcagcaatttattttattgtaagtacaagcctattgctatagaagatattagatattacctacttcggtgcacttgcaccatgtgctgaactgatcttttcaaaataatttcatatctgaaacatggataatttctgaacttttttactgaagggtctggttgctttgacgctgtcagcatcagttccagctctgcagccgcctaaatgttcaggatctatttgtccagaagcaagcttactccagtatggtgtatttttctctggcctctatatgatagccctcgggactggaggcatcaaaccttgtgtatcatcctttggagctgatcaatttgatgacagtgatccagcagacagagtaaagaagggctccttcttcaattggttttacttctgtataaatatcggtgcatttgtatcaggcaccgttatagtttggatacaagataactcaggttgggggataggatttgccattcctactatatttatggcattagcgattgcaagtttctttgttgcctcaaatatgtacagatttcagaaacctggtggaagccctcttacaagagtgtgtcaggttgttgttgcagcattccgtaagtggcacactgaagtgccacatgatacatctcttttatatgaggttgatggccagacttcagcgattgagggaagccggaagctggagcacacaagtgaacttgagtaattcctggatttttgcaatgcatcattgtctcacttttattcattctgttacaaagaaaaaaggaggaaagtctggatggggacaacaccagccatttgcagttggatgtatacataaaactgatacactaccttcttgtactgttccattttgggattggtggaaattaaatactaaatgcaacaaaaagaatatggataaggccatacagcagaacgctagtagtatattagtagtttgtccatggcatgcaattcttataagtctacttataattactattactggtgcctataattaatatgggaccattagaggtatatttgtataatgactgaaaatatcagggtagcacaagcaatatatgtcagtaggtggcttgctttacagacacatttcttttacttttttagacaatataatatattgtgttttcttgtctgactgaaattactttttgttatacagattctttgacaaggctgccatcatctcatctgatgatgccaagagtgactcctttacaaatccgtggaggctatgcactgtcacccaggtggaagaactgaaaattctaatcagaatgtttcccatttgggccactactattatattcaacgcggtgtatgctcacaactcttctatgttcatagagcagggaatggttcttgacaagcgagttggatctttcattgtccctcctgcatccctctcaacttttgatgtcatcagtgtcatcatctggattccgttttatggccgtgtgcttgtgccaatagctagaaagttcactggaagggagaagggtttctctgagttacagcggattggaatcggattagccctctccatccttgcaatgctatctgcagctcttgttgagttgaggcgtttagggatcgccagatctgaaggtcttattcatgaggatgttgctgttccgatgagcattctttggcaaataccgcagtatttcttggttggcgctgctgaggtctttgctgccataggtcaggttgagttcttctacaatgaagcccctgatgccatgaggagtttgtgtagtgcatttgcgcttgtaacagtctcactggggagctatttaagctcaatcatattaaccttggtgtcatattttacaactcaaggaggggatcctggatggatcccagataacctgaatgaaggccacctagatcggttcttttcattgattgctgggatcaactttgtgaatttactggttttcactggttgtgcaatgagatacagatacaagaaagcatgatgactgtactcatggtaaggtcagtttgtgttagtaataacagatttt

SEQ ID NO: 101 - DNA, 1659 bp, Oryza sativa strain Azucena
  CDS (1)..(1659)
atcaaagggcgccctgcattaaagcatgccactggaaattggcgtgcatgttttttcatcctaggggatgaatgctgtgagagactggcctattatggtattgcaaagaacctagttacttatctgaaaacaaatcttcatcaaggcaaccttgaagctgcaagaaatgttacaacttggcaggggacatgctacctaacacccctcattggtgccctcctagcagattcttactggggaaagtactggactattgctgctttctcagcaatttattttattggtctggttgctttgacgctgtcagcatcagttccagctctgcagccgcctaaatgttcaggatctatttgtccagaagcaagcttactccagtatggtgtatttttctctggcctctatatgatagccctcgggactggaggcatcaaaccttgtgtatcatcctttggagctgatcaatttgatgacagtgatccagcagacagagtaaagaagggctccttcttcaattggttttacttctgtataaatatcggtgcatttgtatcaggcaccgttatagtttggatacaagataactcaggttgggggataggatttgccattcctactatatttatggcattagcgattgcaagtttctttgttgcctcaaatatgtacagatttcagaaacctggtggaagccctcttacaagagtgtgtcaggttgttgttgcagcattccgtaagtggcacactgaagtgccacatgatacatctcttttatatgaggttgatggccagacttcagcgattgagggaagccggaagctggagcacacaagtgaacttgaattctttgacaaggctgccatcatctcatctgatgatgccaagagtgactcctttacaaatccgtggaggctatgcactgtcacccaggtggaagaactgaaaattctaatcagaatgtttcccatttgggccactactattatattcaacgcggtgtatgctcacaactcttctatgttcatagagcagggaatggttcttgacaagcgagttggatctttcattgtccctcctgcatccctctcaacttttgatgtcatcagtgtcatcatctggattccgttttatggccgtgtgcttgtgccaatagctagaaagttcactggaagggagaagggtttctctgagttacagcggattggaatcggattagccctctccatccttgcaatgctatctgcagctcttgttgagttgaggcgtttagggatcgccagatctgaaggtcttattcatgaggatgttgctgttccgatgagcattctttggcaaataccgcagtatttcttggttggcgctgctgaggtctttgctgccataggtcaggttgagttcttctacaatgaagcccctgatgccatgaggagtttgtgtagtgcatttgcgcttgtaacagtctcactggggagctatttaagctcaatcatattaaccttggtgtcatattttacaactcaaggaggggatcctggatggatcccagataacctgaatgaaggccacctagatcggttcttttcattgattgctgggatcaactttgtgaatttactggttttcactggttgtgcaatgagatacagatacaagaaagcatga

SEQ ID NO: 102 - protein, 552 aa, Oryza sativa strain Azucena
IKGRPALKHATGNWRACFFILGDECCERLAYYGIAKNLVTYLKTNLHQGNLEAARNVTTWQGTCYLTPLIGALLADSYWGKYWTIAAFSAIYFIGLVALTLSASVPALQPPKCSGSICPEASLLQYGVFFSGLYMIALGTGGIKPCVSSFGADQFDDSDPADRVKKGSFFNWFYFCINIGAFVSGTVIVWIQDNSGWGIGFAIPTIFMALAIASFFVASNMYRFQKPGGSPLTRVCQVVVAAFRKWHTEVPHDTSLLYEVDGQTSAIEGSRKLEHTSELEFFDKAAIISSDDAKSDSFTNPWRLCTVTQVEELKILIRMFPIWATTIIFNAVYAHNSSMFIEQGMVLDKRVGSFIVPPASLSTFDVISVIIWIPFYGRVLVPIARKFTGREKGFSELQRIGIGLALSILAMLSAALVELRRLGIARSEGLIHEDVAVPMSILWQIPQYFLVGAAEVFAAIGQVEFFYNEAPDAMRSLCSAFALVTVSLGSYLSSIILTLVSYFTTQGGDPGWIPDNLNEGHLDRFFSLIAGINFVNLLVFTGCAMRYRYKKA

SEQ ID NO: 103 - DNA, 2601 bp, Oryza sativa strain IR64
atcaaagggcgccctgcattaaagcatgccactggaaattggcgtgcatgttttttcatcctaggtaatttgttaaagatgcatagcatatcccataaaactttggcacagtacttgaaggaattccttgtttcttcgcactagtgagtaatgcttctatatggttttgaatcgtacaatcctgtatttaccttttgccaaaatattttggtgacatacaacacaaaagaaatgctggtcgaagtaccagatagcatactttacagatcaattgaaaaatgctgtgcacatttttatctgttctgcaatgagtagctttgaagtttcagaaatgctagtttggtgacaggggatgaatgctgtgagagactggcctattatggtattgcaaagaacctagttacttatctgaaaacaaatcttcatcaaggcaaccttgaagctgcaagaaatgttacaacttggcaggggacatgctacctaacacccctcattggtgccctcctagcagattcttactggggaaagtactggactattgctgctttctcagcaatttattttattgtaagtacaagcctattgctatagaagatattagatattacctacttcggtgcacttgcaccatgtgctgaactgatcttttcaaaataatttcatatctgaaacatggataatttctgaacttttttactgaagggtctggttgctttgacgctgtcagcatcagttccagctctgcagccgcctaaatgttcaggatctatttgtccagaagcaagcttactccagtatggtgtatttttctctggcctctatatgatagccctcgggactggaggcatcaaaccttgtgtatcatcctttggagctgatcaatttgatgacagtgatccagcagacagagtaaagaagggctccttcttcaattggttttacttctgtataaatatcggtgcatttgtatcaggcaccgttatagtttggatacaagataactcaggttgggggataggatttgccattcctactatatttatggcattagcgattgcaagtttctttgttgcctcaaatatgtacagatttcagaaacctggtggaagccctcttacaagagtgtgtcaggttgttgttgcagcattccgtaagtggcacactgaagtgccacatgatacatctcttttatatgaggttgatggccagacttcagcgattgagggaagccggaagctggagcacacaagtgaacttgagtaattcctggatttttgcaatgcatcattgtctcacttttattcattctgttacaaagaaaaaagggggaaagtctggatggggacaacaccagccatttgcagttggatgtatacataaaactgatacactaccttcttgtactgttccattttgggattggtggaaattaaatactaaatgcaacaaaaagaatatggataaggccatacagcagaacgctagtagtatattagtagtttgtccatggcatgcaattcttataagtctacttataattactattactggtgcctataattaatatgggaccattagaggtatatttgtataatgactgaaaatatcagggtagcacaagcaatatatgtcagtaggtggcttgctttacagacacatttcttttacttttttttagacaatataatatattgtgttttcttgtctgactgaaattactttttgttatacagattctttgacaaggctgccatcatctcatctgatgatgccaagagtgactcctttacaaatccgtggaggctatgcactgtcacccaggtggaagaactgaaaattctaatcagaatgtttcccatttgggccactactattatattcaacgcggtgtatgctcagaactcttctatgttcatagagcagggaatggttcttgacaagcgagttggatctttcattgtccctcctgcatccctctcaacttttgatgtcatcagtgtcatcatctggattccgttttatgaccgtgtgcttgtgccaatagctagaaagttcactggaagggagaagggtttctctgagttacagcggattggaatcggattagccctctccatccttgcaatgctatctgcagctcttgttgagttgaggcgtttagagatcgccagatctgaaggtcttattcatgaggatgttgctgttccgatgagcattctttggcaaataccgcagtatttcttggttggcgctgctgaggtctttgctgccataggtcaggttgagttcttctacaatgaagcccctgatgccatgaggagtttgtgtagtgcatttgcgcttgtaacagtctcactggggagctatttaagctcaatcatattaaccttggtgtcatattttacaactcaaggaggggatcctggatggatcccagataacctgaatgaaggccacctagatcggttcttttcattgattgctgggatcaactttgtgaatttactggttttcactggttgtgcaatgagatacagatacaagaaagcatgatgactgtactcatggtaaggtcagtttgtgtaagtaataacagatttt

SEQ ID NO: 104 - DNA, 1659 bp, Oryza sativa strain IR64; identical to SEQ ID NO: 94
  CDS (1)..(1659)

SEQ ID NO: 105 - protein, 552 aa, Oryza sativa strain IR64; identical to SEQ ID NO: 95

SEQ ID NO: 106 - DNA, 2601 bp, Oryza sativa strain Kasalath
atcaaagggcgccctgcattaaagcatgccactggaaattggcgtgcatgttttttcatcctaggtaatttgttaaagatgcatagcatatcccataaaactttggcacagtacttgaaggaattccttgtttcttcgcactagtgagtaatgcttctatatggttttgaatcgtacaatcctgtatttaccttttgccaaaatattttggtgacatacaacacaaaagaaatgctggtcgaagtaccagatagcatactttacagatcaattgaaaaatgctgtgcacatttttatctgttctgcaatgagtagctttgaagtttcagaaatgctagtttggtgacaggggatgaatgctgtgagagactggcctattatggtattgcaaagaacctagttacttatctgaaaacaaatcttcatcaaggcaaccttgaagctgcaagaaatgttacaacttggcaggggacatgctacctaacacccctcattggtgccctcctagcagattcttactggggaaagtactggactattgctgctttctcagcaatttattttattgtaagtacaagcctattgctatagaagatattagatattacctacttcggtgcacttgcaccatgtgctgaactgatcttttcaaaataatttcatatctgaaacatggataatttctgaacttttttactgaagggtctggttgctttgacgctgtcagcatcagttccagctctgcagccgcctaaatgttcaggatctatttgtccagaagcaagcttactccagtatggtgtatttttctctggcctctatatgatagccctcgggactggaggcatcaaaccttgtgtatcatcctttggagctgatcaatttgatgacagtgatccagcagacagagtaaagaagggctccttcttcaattggttttacttctgtataaatatcggtgcatttgtatcaggcactgttatagtttggatacaagataactcaggttgggggataggatttgccattcctactatatttatggcattagcgattgcaagtttctttgttgcctcaaatatgtacagatttcagaaacctggtggaagccctcttacaagagtgtgtcaggttgttgttgcagcattccgtaagtggcacactgaagtgccacatgatacatctcttttatatgaggttgatggccagacttcagcgattgagggaagccggaagctggagcacacaagtgaacttgagtaattcctggatttttgcaatgcatcattgtctcacttttattcattctgttacaaagaaaaaagggggaaagtctggatggggacaacaccagccatttgcagttggatgtatacataaaactgatacactaccttcttgtactgttccattttgggattggtggaaattaaatactaaatgcaacaaaaagaatatggataaggccatacagcagaacgctagtagtatattagtagtttgtccatggcatgcaattcttataagtctacttataattactattactggtgcctataattaatatgggaccattagaggtatatttgtataatgactgaaaatatcagggtagcacaagcaatatatgtcagtaggtggcttgctttacagacacatttcttttacttttttttagacaatataatatattgtgttttcttgtctgactgaaattactttttgttatacagattctttgacaaggctgccatcatctcatctgatgatgccaagagtgactcctttacaaatccgtggaggctatgcactgtcacccaggtggaagaactgaaaattctaatcagaatgtttcccatttgggccactactattatattcaacgcggtgtatgctcagaactcttctatgttcatagagcagggaatggttcttgacaagcgagttggatctttcattgtccctcctgcatccctctcaacttttgatgtcatcagtgtcatcatctggattccgtttaatgaccgtgtgcttgtgccaatagctagaaagttcactggaagggagaagggtttctctgagttacagcggattggaatcggattagccctctccatccttgcaatgctatctgcagctcttgttgagttgaggcgtttagagatcgccagatctgaaggtcttattcatgaggatgttgctgttccgatgagcattctttggcaaataccgcagtatttcttggttggcgctgctgaggtctttgctgccataggtcaggttgagttcttctacaatgaagcccctgatgccatgaggagtttgtgtagtgcatttgcgcttgtaacagtctcactggggagctatttaagctcaatcatattaaccttggtgtcatattttacaactcaaggaggggatcctggatggatcccagataacctgaatgaaggccacctagatcggttcttttcattgattgctgggatcaactttgtgaatttactggttttcactggttgtgcaatgagatacagatacaagaaagcatgatgactgtactcatggtaaggtcagtttgtgtaagtaataacagatttt

SEQ ID NO: 107 - DNA, 1659 bp, Oryza sativa strain Kasalath
  CDS (1)..(1659)
atcaaagggcgccctgcattaaagcatgccactggaaattggcgtgcatgttttttcatcctaggggatgaatgctgtgagagactggcctattatggtattgcaaagaacctagttacttatctgaaaacaaatcttcatcaaggcaaccttgaagctgcaagaaatgttacaacttggcaggggacatgctacctaacacccctcattggtgccctcctagcagattcttactggggaaagtactggactattgctgctttctcagcaatttattttattggtctggttgctttgacgctgtcagcatcagttccagctctgcagccgcctaaatgttcaggatctatttgtccagaagcaagcttactccagtatggtgtatttttctctggcctctatatgatagccctcgggactggaggcatcaaaccttgtgtatcatcctttggagctgatcaatttgatgacagtgatccagcagacagagtaaagaagggctccttcttcaattggttttacttctgtataaatatcggtgcatttgtatcaggcactgttatagtttggatacaagataactcaggttgggggataggatttgccattcctactatatttatggcattagcgattgcaagtttctttgttgcctcaaatatgtacagatttcagaaacctggtggaagccctcttacaagagtgtgtcaggttgttgttgcagcattccgtaagtggcacactgaagtgccacatgatacatctcttttatatgaggttgatggccagacttcagcgattgagggaagccggaagctggagcacacaagtgaacttgaattctttgacaaggctgccatcatctcatctgatgatgccaagagtgactcctttacaaatccgtggaggctatgcactgtcacccaggtggaagaactgaaaattctaatcagaatgtttcccatttgggccactactattatattcaacgcggtgtatgctcagaactcttctatgttcatagagcagggaatggttcttgacaagcgagttggatctttcattgtccctcctgcatccctctcaacttttgatgtcatcagtgtcatcatctggattccgtttaatgaccgtgtgcttgtgccaatagctagaaagttcactggaagggagaagggtttctctgagttacagcggattggaatcggattagccctctccatccttgcaatgctatctgcagctcttgttgagttgaggcgtttagagatcgccagatctgaaggtcttattcatgaggatgttgctgttccgatgagcattctttggcaaataccgcagtatttcttggttggcgctgctgaggtctttgctgccataggtcaggttgagttcttctacaatgaagcccctgatgccatgaggagtttgtgtagtgcatttgcgcttgtaacagtctcactggggagctatttaagctcaatcatattaaccttggtgtcatattttacaactcaaggaggggatcctggatggatcccagataacctgaatgaaggccacctagatcggttcttttcattgattgctgggatcaactttgtgaatttactggttttcactggttgtgcaatgagatacagatacaagaaagcatga

SEQ ID NO: 108 - protein, 552 aa, Oryza sativa strain Kasalath
IKGRPALKHATGNWRACFFILGDECCERLAYYGIAKNLVTYLKTNLHQGNLEAARNVTTWQGTCYLTPLIGALLADSYWGKYWTIAAFSAIYFIGLVALTLSASVPALQPPKCSGSICPEASLLQYGVFFSGLYMIALGTGGIKPCVSSFGADQFDDSDPADRVKKGSFFNWFYFCINIGAFVSGTVIVWIQDNSGWGIGFAIPTIFMALAIASFFVASNMYRFQKPGGSPLTRVCQVVVAAFRKWHTEVPHDTSLLYEVDGQTSAIEGSRKLEHTSELEFFDKAAIISSDDAKSDSFTNPWRLCTVTQVEELKILIRMFPIWATTIIFNAVYAQNSSMFIEQGMVLDKRVGSFIVPPASLSTFDVISVIIWIPFNDRVLVPIARKFTGREKGFSELQRIGIGLALSILAMLSAALVELRRLEIARSEGLIHEDVAVPMSILWQIPQYFLVGAAEVFAAIGQVEFFYNEAPDAMRSLCSAFALVTVSLGSYLSSIILTLVSYFTTQGGDPGWIPDNLNEGHLDRFFSLIAGINFVNLLVFTGCAMRYRYKKA

SEQ ID NO: 109 - DNA, 2599 bp, Oryza sativa strain Lemont; identical to SEQ ID NO: 100

SEQ ID NO: 110 - DNA, 1659 bp, Oryza sativa strain Lemont; identical to SEQ ID NO: 101
  CDS (1)..(1659)

SEQ ID NO: 111 - protein, 552 aa, Oryza sativa strain Lemont; identical to SEQ ID NO: 102

SEQ ID NO: 112 - DNA, 1259 bp, Oryza sativa strain Nipponbare
atcaaagggcgccctgcattaaagcatgccactggaaattggcgtgcatgttttttcatcctaggtaatttgttaaagatgcatagcatatcccataaaactttggcacagtacttgaaggaattccttgtttcttcgcactagtgagtaatgcttctatatggttttgaatcgtacaatcctgtatttaccttttgccaaaatattttggtgatatacaacacaaaagaaatgctggtcgaagtaccagatagcatactttacagatcaattgaaaaatgctgtgcacatttttatctgttctgcaatgagtagctttgaagtttcagaaatgctagtttggtgacaggggatgaatgctgtgagagactggcctattatggtattgcaaagaacctagttacttatctgaaaacaaatcttcatcaaggcaaccttgaagctgcaagaaatgttacaacttggcaggggacatgctacctaacacccctcattggtgccctcctagcagattcttactggggaaagtactggactattgctgctttctcagcaatttattttattgtaagtacaagcctattgctatagaagatattagatattacctacttcggtgcacttgcaccatgtgctgaactgatcttttcaaaataatttcatatctgaaacatggataatttctgaacttttttactgaagggtctggttgctttgacgctgtcagcatcagttccagctctgcagccgcctaaatgttcaggatctatttgtccagaagcaagcttactccagtatggtgtatttttctctggcctctatatgatagccctcgggactggaggcatcaaaccttgtgtatcatcctttggagctgatcaatttgatgacagtgatccagcagacagagtaaagaagggctccttcttcaattggttttacttctgtataaatatcggtgcatttgtatcaggcaccgttatagtttggatacaagataactcaggttgggggataggatttgccattcctactatatttatggcattagcgattgcaagtttctttgttgcctcaaatatgtacagatttcagaaacctggtggaagccctcttacaagagtgtgtcaggttgttgttgcagcattccgtaagtggcacactgaagtgccacatgatacatctcttttatatgaggttgatggccagacttcagcgattgagggaagccggaagctggagcacacaagtgaacttga

SEQ ID NO: 113 - DNA, 864 bp, Oryza sativa strain Nipponbare
attctttgacaaggctgccatcatctcatctgatgatgccaagagtgactcctttacaaatccgtggaggctatgcactgtcacccaggtggaagaactgaaaattctaatcagaatgtttcccatttgggccactactattatattcaacgcggtgtatgctcacaactcttctatgttcatagagcagggaatggttcttgacaagcgagttggatctttcattgtccctcctgcatccctctcaacttttgatgtcatcagtgtcatcatctggattccgttttatggccgtgtgcttgtgccaatagctagaaagttcactggaagggagaagggtttctctgagttacagcggattggaatcggattagccctctccatccttgcaatgctatctgcagctcttgttgagttgaggcgtttagggatcgccagatctgaaggtcttattcatgaggatgttgctgttccgatgagcattctttggcaaataccgcagtatttcttggttggcgctgctgaggtctttgctgccataggtcaggttgagttcttctacaatgaagcccctgatgccatgaggagtttgtgtagtgcatttgcgcttgtaacagtctcactggggagctatttaagctcaatcatattaaccttggtgtcatattttacaactcaaggaggggatcctggatggatcccagataacctgaatgaaggccacctagatcggttcttttcattgattgctgggatcaactttgtgaatttactggttttcactggttgtgcaatgagatacagatacaagaaagcatgatgactgtactcatggtaaggtcagtttgtgttagtaataacaga

SEQ ID NO: 114 - DNA, 1659 bp, Oryza sativa strain Nipponbare; identical to SEQ ID NO: 101
  CDS (1)..(1659)

SEQ ID NO: 115 - protein, 552 aa, Oryza sativa strain Nipponbare; identical to SEQ ID NO: 102

SEQ ID NO: 116 - DNA, 2601 bp, Oryza sativa strain Teqing; identical to SEQ ID NO: 103

SEQ ID NO: 117 - DNA, 1659 bp, Oryza sativa strain Teqing; identical to SEQ ID NO: 94
  CDS (1)..(1659)

SEQ ID NO: 118 - protein, 552 aa, Oryza sativa strain Teqing; identical to SEQ ID NO: 95

SEQ ID NO: 119 - DNA, 531 bp, Zea mays mays strain BS7
atggttcttgacaagcgcattgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccctgacaatctgaatgaaggccat

SEQ ID NO: 120 - DNA, 531 bp, Zea mays mays strain BS7; identical to SEQ ID NO: 119
  CDS (1)..(531)

SEQ ID NO: 121 - protein, 177 aa, Zea mays mays strain BS7
MVLDKRIGSFNIPPASLSTFDVISVIMWVPLYDRILVPLARKFTGREKGFSELQRMGIGLVLSILAMVSAALVELKRLEIARSEGLIHEKAAVPMSILWQIPQYFLVGAAEVFTCIGQVEFFYDQAPDAMRSLCSALALITVSLGNYISSIILTLVSYITTQGGDPGWIPDNLNEGH

SEQ ID NO: 122 - DNA, 536 bp, Zea mays mays strain Enano
gaccaagcagagagagctaagaagggttcattcttcaattggttctacttctgtataaatataggttcattcatatcaggcactatgatagtgtggatacaagataacactggttggggaataggctttgcgattcctactatattcatggcattagctatttcattcttcttctcagcttcaaataagtacagattccaaaaacctggtgggagtccactcacaagagtgtgccaggtggttatagcagcatttcgtaagtggcacattgaagtgccacatgatacatctctcctatatgaagttgatggccaaacttcagcaattgaaggaagccggaagctggagcacacaaatgagctcgagtaattctaattttctgcaatgcaatcacttctttagtttcagtatcatgtgatgcaacatttatggaatatttgggaatgacagtacttatatgtcataaagacattgaacttcactgcaaagtttttattatttcaacttgaggatactggaaaattagtacataaata

SEQ ID NO: 123 - DNA, 550 bp, Zea mays mays strain Enano
aaacaacactggtttgtgagtttatgacaaagagtagtagtatgttgtagttctacaagacatccgacaatagaaatagtggctgccacttaattctcaatactcactactcagcagtcaatagtaatccaaattaatctgaaactccgtggaggaagcaaatgcaccgtgacacatgttactgtgtacttgtgatcttgttaaatgcttgcttgtaaatgcaacgattgtaactaagtaacataacttaaggctcaggaggaccagacacatgttacttgcataattgtatttttttcaagtatgttatgcctgtaaggagcttgatttgtggatttcatatcttttattgtgtggctactataatattttatctacctctcttgtgtgtaggttccttgatagagctgctgttatctcatctgctgatctgaagagtgaatcctttaccgacccatggaagctttgcacagttacccaggtggaagaattgaagatcctaataagaatgtttcccatttgggctactactatcatattcagtgctgtt

SEQ ID NO: 124 - DNA, 533 bp, Zea mays mays strain Enano
atggttcttgacaagcgcattgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccctgacaatctgaatgaaggccatct

SEQ ID NO: 125 - DNA, 531 bp, Zea mays mays strain Enano; identical to SEQ ID NO: 119
  CDS (1)..(531)

SEQ ID NO: 126 - protein, 177 aa, Zea mays mays strain Enano; identical to SEQ ID NO: 121

SEQ ID NO: 127 - DNA, 525 bp, Zea mays mays strain Huobai
tgatgacactgaccaagcagagagagctaagaagggttcattcttcaattggttctacttctgtataaatataggttcattcatatcaggcactatgatagtgtggatacaagataacactggttggggaataggctttgcgattcctactatattcatggcattagctatttcattcttcttctcagcttcaaataagtacagattccaaaaacctggtgggagtccactcacaagagtgtgccaggtggttatagcagcatttcgtaagtggcacattgaagtgccacatgatacatctctcctatatgaagttgatggccaaacttcagcaattgaaggaagccggaagctggagcacacaaatgagctcgagtaattctaattttctgcaatgcaatcacttctttagtttcagtatcatgtgatgcaacatttatggaatatttggaaatgacagtacttatatgtcataaagacattgaacttcactgcaaagtttttattatttcaacttgaggatac

SEQ ID NO: 128 - DNA, 334 bp, Zea mays mays strain Huobai
aggctcaggaggaccagacacatgttacttgcataattgtatttttttcaagtatgttatgcctgtaaggagcttgatttgtggatttcatatcttttattgtgtggctactataatattttatctacctctcttgtgtgtaggttccttgatagagctgctgttatctcatctgctgatctgaagagtgaattctttaccgacccatggaagctttgcacagttacccaggtggaagaattgaagatcctaataagaatgtttcccatttgggctactactatcatattcagtgctgtttatgcccaaaactcttccatgttcatagagcagg

SEQ ID NO: 129 - DNA, 529 bp, Zea mays mays strain Huobai
gggcatggttcttgacaagcgcattgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccctgacaatctgaatgaa

SEQ ID NO: 130 - DNA, 528 bp, Zea mays mays strain Huobai
  CDS (1)..(528)
ggcatggttcttgacaagcgcattgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccctgacaatctgaatgaa

SEQ ID NO: 131 - protein, 176 aa, Zea mays mays strain Huobai
GMVLDKRIGSFNIPPASLSTFDVISVIMWVPLYDRILVPLARKFTGREKGFSELQRMGIGLVLSILAMVSAALVELKRLEIARSEGLIHEKAAVPMSILWQIPQYFLVGAAEVFTCIGQVEFFYDQAPDAMRSLCSALALITVSLGNYISSIILTLVSYITTQGGDPGWIPDNLNE

SEQ ID NO: 132 - DNA, 513 bp, Zea mays mays strain Makki
ggcatggttcttgacaagcgcattgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccct

SEQ ID NO: 133 - DNA, 513 bp, Zea mays mays strain Makki; identical to SEQ ID NO: 132
  CDS (1)..(513)

SEQ ID NO: 134 - protein, 171 aa, Zea mays mays strain Makki
GMVLDKRIGSFNIPPASLSTFDVISVIMWVPLYDRILVPLARKFTGREKGFSELQRMGIGLVLSILAMVSAALVELKRLEIARSEGLIHEKAAVPMSILWQIPQYFLVGAAEVFTCIGQVEFFYDQAPDAMRSLCSALALITVSLGNYISSIILTLVSYITTQGGDPGWIP

SEQ ID NO: 135 - DNA, 545 bp, Zea mays mays strain Min13
gatgacactgaccaagcagagagagctaagaagggttcattcttcaattggttctacttctgtataaatataggttcattcatatcaggcactatgatagtgtggatacaagataacactggttggggaataggctttgcgattcctactatattcatggcattagctatttcattcttcttctcagcttcaaataagtacagattccaaaaacctggtgggagtccactcacaagagtgtgccaggtggttatagcagcatttcgtaagtggcacattgaagtgccacatgatacatctctcctatatgaagttgatggccaaacttcagcaattgaaggaagccggaagctggagcacacaaatgagctcgagtaattctaattttctgcaatgcaatcacttctttagtttcagtatcatgtgatgcaacatttatggaatatttgggaatgacagtacttatatgtcataaagacattgaacttcactgcaaagtttttattatttcaacttgaggatactggaaaattagtacataaata

SEQ ID NO: 136 - DNA, 570 bp, Zea mays mays strain Min13
caacacttgtttgtgagtttatgacaaagagtagtagtatgttgtagttctacaagacatccgacaatagaaatagtggctgccacttaattctcaatactcactactcagcagtcaatagtaatccaaattaatctgaaactccgtggaggaagcaaatgcaccgtgacacatgttactgtgtacttgtgatcttgttaaatgcttgcttgtaaatgcaacgattgtaactaagtaacataacttaaggctcaggaggaccagacacatgttacttgcataattgtatttttttcaagtatgttatgcctgtaaggagcttgatttgtggatttcatatcttttattgtgtggctactataatattttatctacctctcttgtgtgtaggttccttgatagagctgctgttatctcatctgctgatctgaagagtgaatcctttaccgacccatggaagctttgcacagttacccaggtggaagaattgaagatcctaataagaatgtttcccatttgggctactactatcatattcagtgctgtttatgcccaaaactcttccatgtt

SEQ ID NO: 137 - DNA, 525 bp, Zea mays mays strain Min13
ggcatggttcttgacaagcgcattgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccctgacaatctgaat

SEQ ID NO: 138 - DNA, 525 bp, Zea mays mays strain Min13; identical to SEQ ID NO: 137
  CDS (1)..(525)

SEQ ID NO: 139 - protein, 175 aa, Zea mays mays strain Min13
GMVLDKRIGSFNIPPASLSTFDVISVIMWVPLYDRILVPLARKFTGREKGFSELQRMGIGLVLSILAMVSAALVELKRLEIARSEGLIHEKAAVPMSILWQIPQYFLVGAAEVFTCIGQVEFFYDQAPDAMRSLCSALALITVSLGNYISSIILTLVSYITTQGGDPGWIPDNLN

SEQ ID NO: 140 - DNA, 526 bp, Zea mays mays strain Pira
gacactgaccaagcagagagagctaagaagggttcattcttcaattggttctacttctgtataaatataggttcattcatatcaggcactatgatagtgtggatacaagataacactggttggggaataggctttgcgattcctactatattcatggcattagctatttcattcttcttctcagcttcaaataagtacagattccaaaaacctggtgggagtccactcacaagagtgtgccaggtggttatagcagcatttcgtaagtggcacattgaagtgccacatgatacatctctcctatatgaagttgatggccaaacttcagcaattgaaggaagccggaagctggagcacacaaatgagctcgagtaattctaattttctgcaatgcaatcacttctttagtttcagtatcatgtgatgcaacatttatggaatatttgggaatgacagtacttatatgtcataaagacattgaacttcactgcaaagtttttattatttcaacttgaggatactggaa

SEQ ID NO: 141 - DNA, 525 bp, Zea mays mays strain Pira; identical to SEQ ID NO: 137

SEQ ID NO: 142 - DNA, 525 bp, Zea mays mays strain Pira; identical to SEQ ID NO: 137
  CDS (1)..(525)

SEQ ID NO: 143 - protein, 175 aa, Zea mays mays strain Pira; identical to SEQ ID NO: 139

SEQ ID NO: 144 - DNA, 499 bp, Zea mays mays strain Sari
accaagcagagagagctaagaagggttcattcttcaattggttctacttctgtataaatataggttcattcatatcaggcactatgatagtgtggatacaagataacactggttggggaataggctttgcgattcctactatattcatggcattagctatttcattcttcttctcagcttcaaataagtacagattccaaaaacctggtgggagtccactcacaagagtgtgccaggtggttatagcagcatttcgtaagtggcacattgaagtgccacatgatacatctctcctatatgaagttgatggccaaacttcagcaattgaaggaagccggaagctggagcacacaaatgagctcgagtaattctaattttctgcaatgcaatcacttctttagtttcagtatcatgtgatgcaacatttatggaatatttggaaatgacagtacttatatgtcataaagacattgaacttcactgcaaagtttttattatt

SEQ ID NO: 145 - DNA, 607 bp, Zea mays parviglumis
ttaggagactttcaaattgctaatgtcctaaacaacacttgtttgtgagtttatgacaaagagtagtagtatgttgtagttctacaagacatccgacaatagaaatagtggctgccacttaattctcaatactcactactcagcagtcaatagtaatccaaattaatctgaaactccgtggaggaagcaaatgcaccgtgacacatgttactgtgtacttgtgatcttgttaaatgcttgcttgtaaatgcaacgattgtaactaagtaacataacttaaggctcaggaggaccagacacatgttacttgcataattgtatttttttcaagtatgttatgcctgtaaggagcttgatttgtggatttcatatcttttattgtgtggctactataatattttatctacctctcttgtgtgtaggttccttgatagagctgctgttatctcatctgctgatctgaagagtgaatcctttaccgacccatggaagctttgcacagttacccaggtggaagaattgaagatcctaataagaatgtttcccatttgggctactactatcatattcagtgctgtttatgcccaaaactcttccatgttcatag

SEQ ID NO: 146 - DNA, 520 bp, Zea mays mays strain Sari
catggttcttgacaagcgcattgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccctgacaatctg

SEQ ID NO: 147 - DNA, 519 bp, Zea mays parviglumis strain Sari
  CDS (1)..(519)
atggttcttgacaagcgcattgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccctgacaatctg

SEQ ID NO: 148 - protein, 173 aa, Zea mays parviglumis strain Sari
MVLDKRIGSFNIPPASLSTFDVISVIMWVPLYDRILVPLARKFTGREKGFSELQRMGIGLVLSILAMVSAALVELKRLEIARSEGLIHEKAAVPMSILWQIPQYFLVGAAEVFTCIGQVEFFYDQAPDAMRSLCSALALITVSLGNYISSIILTLVSYITTQGGDPGWIPDNL

SEQ ID NO: 149 - DNA, 543 bp, Zea mays mays strain Smena
gacactgaccaagcagagagagctaagaagggttcattcttcaattggttctacttctgtataaatataggttcattcatatcaggcactatgatagtgtggatacaagataacactggttggggaataggctttgcgattcctactatattcatggcattagctatttcattcttcttctcagcttcaaataagtacagattccaaaaacctggtgggagtccactcacaagagtgtgccaggtggttatagcagcatttcgtaagtggcacattgaagtgccacatgatacatctctcctatatgaagttgatggccaaacttcagcaattgaaggaagccggaagctggagcacacaaatgagctcgagtaattctaattttctgcaatgcaatcacttctttagtttcagtatcatgtgatgcaacatttatggaatatttgggaatgacagtacttatatgtcataaagacattgaacttcactgcaaagtttttattatttcaacttgaggatactggaaaattagtacataaatac

SEQ ID NO: 150 - DNA, 443 bp, Zea mays mays strain Smena
aattaatctgaaactccgtggaggaagcaaatgcaccgtgacacatgttactgtgtacttgtgatcttgttaaatgcttgcttgtaaatgcaacgattgtaactaagtaacataacttaaggctcaggaggaccagacacatgttacttgcataattgtatttttttcaagtatgttatgcctgtaaggagcttgatttgtggatttcatatcttttattgtgtggctactataatattttatctacctctcttgtgtgtaggttccttgatagagctgctgttatctcatctgctgatctgaagagtgaatcctttaccgacccatggaagctttgcacagttacccaggtggaagaattgaagatcctaataagaatgtttcccatttgggctactactatcatattcagtgctgtttatgcccaaaactcttccatgttc

SEQ ID NO: 151 - DNA, 523 bp, Zea mays mays strain Smena
gcatggttcttgacaagcgcattgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccctgacaatctgaa

SEQ ID NO: 152 - DNA, 519 bp, Zea mays parviglumis strain Smena; identical to SEQ ID NO: 147
  CDS (1)..(519)

SEQ ID NO: 153 - protein, 173 aa, Zea mays parviglumis strain Smena; identical to SEQ ID NO: 148

SEQ ID NO: 154 - DNA, 488 bp, Zea mays mays strain W22
gggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccc

SEQ ID NO: 155 - DNA, 486 bp, Zea mays mays strain W22
  CDS (1)..(486)
gggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatc

SEQ ID NO: 156 - protein, 162 aa, Zea mays mays strain W22
GSFNIPPASLSTFDVISVIMWVPLYDRILVPLARKFTGREKGFSELQRMGIGLVLSILAMVSAALVELKRLEIARSEGLIHEKAAVPMSILWQIPQYFLVGAAEVFTCIGQVEFFYDQAPDAMRSLCSALALITVSLGNYISSIILTLVSYITTQGGDPGWI

SEQ ID NO: 157 - DNA, 516 bp, Zea mays mays strain Benz
atggttcttgacaagcgcattgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcataagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccctgacaat

SEQ ID NO: 158 - DNA, 516 bp, Zea mays parviglumis strain Benz; identical to SEQ ID NO: 157
  CDS (1)..(516)

SEQ ID NO: 159 - protein, 172 aa, Zea mays parviglumis strain Benz
MVLDKRIGSFNIPPASLSTFDVISVIMWVPLYDRILVPLARKFTGREKGFSELQRMGIGLVLSILAMVSAALVELKRLEIARSEGLIHKKAAVPMSILWQIPQYFLVGAAEVFTCIGQVEFFYDQAPDAMRSLCSALALITVSLGNYISSIILTLVSYITTQGGDPGWIPDN

SEQ ID NO: 160 - DNA, 385 bp, Zea mays parviglumis strain BK4
tgacactgaccaagcagagagagctaagaagggttcattcttcaattggttctacttctgtataaatataggttcattcatatcaggcactatgatagtgtggatacaagataacactggttggggaataggctttgcgattcctactatattcatggcattagctatttcattcttcttctcagcttcaaataagtacagattccaaaaacctggtgggagtccactcacaagagtgtgccaggtggttatagcagcatttcgtaagtggcacattgaagtgccacatgatacatctctcctatatgaagttgatggccaaacttcagcaattgaaggaagccggaagctggagcacacaaatgagctcgagtaattctaattt

SEQ ID NO: 161 - DNA, 613 bp, Zea mays parviglumis strain BK4
aaagctgccttttaggagactttcaaattgctaatgtcctaaacaacacttgtttgtgagtttatgacaaagagtagtagtatgttgtagttctacaagacatccgacaatagaaatagtggctgccacttaattctcaatactcactactcagcagtcaatagtaatccaaattaatctgaaactccgtggaggaagcaaatgcaccgtgacacatgttactgtgtacttgtgatcttgttaaatgcttgcttgtaaatgcaacgattgtaactaagtaacataacttaaggctcaggaggaccagacacatgttacttgcataattgtatttttttcaagtatgttatgcctgtaaggagcttgatttgtggatttcatatcttttattgtgtggctactataatattttatctacctctcttgtgtgtaggttccttgatagagctgctgttatctcatctgctgatctgaagagtgaatcctttaccgacccatggaagctttgcacagttacccaggtggaagaattgaagatcctaataagaatgtttcccatttgggctactactatcatattcagtgctgtttatgcccaaaactcttccatgtt

SEQ ID NO: 162 - DNA, 462 bp, Zea mays parviglumis strain BK4
attgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattaca

SEQ ID NO: 163 - DNA, 462 bp, Zea mays parviglumis strain BK4; identical to SEQ ID NO: 162
  CDS (1)..(462)

SEQ ID NO: 164 - protein, 154 aa, Zea mays parviglumis strain BK4
IGSFNIPPASLSTFDVISVIMWVPLYDRILVPLARKFTGREKGFSELQRMGIGLVLSILAMVSAALVELKRLEIARSEGLIHEKAAVPMSILWQIPQYFLVGAAEVFTCIGQVEFFYDQAPDAMRSLCSALALITVSLGNYISSIILTLVSYIT

SEQ ID NO: 165 - DNA, 556 bp, Zea mays parviglumis strain Wilkes
agagagctaagaagggttcattcttcaattggttctacttctgtataaatataggttcattcatatcaggcactatgatagtgtggatacaagataacactggttggggaataggctttgcgattcctactatattcatggcattagctatttcattcttcttctcagcttcaaataagtacagattccaaaaacctggtgggagtccactcacaagagtgtgccaggtggttatagcagcatttcgtaagtggcacattgaagtgccacatgatacatctctcctatatgaagttgatggccaaacttcagcaattgaaggaagccggaagctggagcacacaaatgagctcgagtaattctaattttctgcaatgcaatcacttctttagtttcagtatcatgtgatgcaacatttatggaatatttggaaatgacagtacttatatgtcataaagacattgaacttcactgcaaagtttttattatttcaacttgaggatactggaaaattagtacataaataccatgaaaaagaattaggaattattgatag

SEQ ID NO: 166 - DNA, 552 bp, Zea mays parviglumis strain Wilkes
ctaaacaacacttgtttgtgagtttatgacaaagagtagtagtatgttgtagttctacaagacatccgacaatagaaatagtggctgccacttaattctcaatactcactactcagcagtcaatagtaatccaaattaatctgaaactccgtggaggaagcaaatgcaccgtgacacatgttactgtgtacttgtgatcttgttaaatgcttgcttgtaaatgcaacgattgtaactaagtaacataacttaaggctcaggaggaccagacacatgttacttgcataattgtatttttttcaagtatgttatgcctgtaaggagcttgatttgtggatttcatatcttttattgtgtggctactataatattttatctacctctcttgtgtgtaggttccttgatagagctgctgttatctcatctgctgatctgaagagtgaatcctttaccgacccatggaagctttgcacagttacccaggtggaagaattgaagatcctaataagaatgtttcccatttgggctactactatcatattcagtgctgtt

SEQ ID NO: 167 - DNA, 511 bp, Zea mays parviglumis strain Wilkes
gcattgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccctgacaatctgaatgaagg

SEQ ID NO: 168 - DNA, 507 bp, Zea mays parviglumis strain Wilkes
  CDS (1)..(507)
attgggtctttcaacattcctcctgcatctctctccacttttgatgtaatcagcgtcatcatgtgggtcccactctatgaccgcatcctggtgccactagctagaaaattcactggaagggagaagggtttttctgagctacagcggatgggaattggattagtcctgtccattctcgcgatggtatctgcagctctagttgagttgaagcgtttagagattgccaggtctgaaggtctcattcatgagaaggctgctgttccaatgagcattctttggcaaataccacaatatttcttggtgggcgctgctgaggtgtttacttgtattggtcaagttgagttcttttacgatcaggccccagatgccatgaggagtttatgtagtgcacttgcacttattacagtctcactgggaaactatataagctccatcatactgacattggtgtcgtacattacaactcagggaggagatcctggatggatccctgacaatctgaatgaa

SEQ ID NO: 169 - protein, 169 aa, Zea mays parviglumis strain Wilkes
IGSFNIPPASLSTFDVISVIMWVPLYDRILVPLARKFTGREKGFSELQRMGIGLVLSILAMVSAALVELKRLEIARSEGLIHEKAAVPMSILWQIPQYFLVGAAEVFTCIGQVEFFYDQAPDAMRSLCSALALITVSLGNYISSIILTLVSYITTQGGDPGWIPDNLNE

SEQ ID NO: 170 - protein, 550 aa, Arabidopsis thaliana
GNPPLKEKTGNWKACPFILGNECCERLAYYGIAGNLITYLTTKLHQGNVSAATNVTTWQGTCYLTPLIGAVLADAYWGRYWTIACFSGIYFIGMSALTLSASVPALKPAECIGDFCPSATPAQYAMFFGGLYLIALGTGGIKPCVSSFGADQFDDTDSRERVRKASFFNWFYFSINIGALVSSSLLVWIQENRGWGLGFGIPTVFMGLAIASFFFGTPLYRFQKPGGSPITRISQVVVASFRKSSVKVPEDATLLYETQDKNSAIAGSRKIEHTDDCQYLDKAAVISEEESKSGDYSNSWRLCTVTQVEELKILIRMFPIWASGIIFSAVYAQMSTMFVQQGRAMNCKIGSFQLPPAALGTFDTASVIIWVPLYDRFIVPLARKFTGVDKGFTEIQRMGIGLFVSVLCMAAAAIVEIIRLHMANDLGLVESGAPVPISVLWQIPQYFILGAAEVFYFIGQLEFFYDQSPDAMRSLCSALALLTNALGNYLSSLILTLVTYFTTRNGQEGWISDNLNSGHLDYFFWLLAGLSLVNMAVYFFSAARYKQKKA

(19) United States
(12) Patent Application Publication
(10) Pub. No.: US 2009/0133164 A1
(43) Pub. Date: May 21, 2009

(54) EG1117 polynucleotides and uses thereof

(75) Inventor: Walter Messier, Longmont, CO (US)

Correspondence Address: SWANSON & BRATSCHUN, L.L.C., 8210 SOUTHPARK TERRACE, LITTLETON, CO, 80120, US

(73) Assignee: EVOLUTIONARY GENOMICS, INC., Lafayette, CO (US)

(21) Appl. No.: 12/255,502

(22) Filed: Oct. 21, 2008

Related U.S. Application Data
(63) Continuation-in-part of application No. 10/345,820, filed on Jan. 16, 2003, now Pat. No. 7,439,018.
(60) Provisional application No. 60/349,088, filed on Jan. 16, 2002.
(60) Provisional application No. 60/349,661, filed on Jan. 17, 2002.
(60) Provisional application No. 60/368,541, filed on Mar. 29, 2002.

(51) Int. Cl. A01H 5/00; C07H 21/04; C07K 14/415; C12Q 1/68; C12P 19/34; C40B 40/06; C07H 1/06; C12P 21/02; C12N 15/82; C12N 5/10

(52) U.S. Cl. 800/298; 536/23.6; 530/350; 435/6; 435/468; 435/419; 435/69.1; 435/91.2; 536/25.4; 506/16

(57) ABSTRACT

The present invention provides methods for identifying polynucleotide and polypeptide sequences which may be associated with commercially or aesthetically relevant traits in domesticated plants or animals. The methods employ comparison of homologous genes from the domesticated organism and its ancestor to identify evolutionarily significant changes and evolutionarily neutral changes. Sequences thus identified may be useful in enhancing commercially or aesthetically desirable traits in domesticated organisms or their wild ancestors.

### CROSS-REFERENCE TO RELATED APPLICATIONS

This application is a continuation-in-part of U.S. application Ser. No. 10/345,820, filed Jan. 16, 2003, entitled “EG1117 Polynucleotides And Uses Thereof,” now U.S. Pat. No. 7,439,018, which claims priority under 35 U.S.C. § 119 from U.S. Application Ser. No. 60/349,088, filed Jan. 16, 2002, entitled “Methods to Identify Evolutionarily Significant Changes in Polynucleotide and Polypeptide Sequences in Domesticated Plants and Animals;” U.S. Application Ser. No. 60/349,661, filed Jan. 17, 2002, entitled “Validation of Agriculturally Important Gene Candidates Selected by an Adapted-Traits Discovery Platform;” and U.S. Application Ser. No. 60/368,541, filed Mar. 29, 2002, entitled “Methods to Identify Evolutionarily Significant Changes in Polynucleotide and Polypeptide Sequences in Domesticated Plants and Animals.” The aforementioned U.S. applications are incorporated herein in their entirety by reference.

### TECHNICAL FIELD

This invention relates to using molecular and evolutionary techniques to identify polynucleotide and polypeptide sequences corresponding to commercially or aesthetically relevant traits in domesticated plants and animals.

### BACKGROUND ART

Humans have bred plants and animals for thousands of years, selecting for certain commercially valuable and/or aesthetic traits. Domesticated plants differ from their wild ancestors in such traits as yield, short day length flowering, protein and/or oil content, ease of harvest, taste, disease resistance and drought resistance. Domesticated animals differ from their wild ancestors in such traits as fat and/or protein content, milk production, docility, fecundity and time to maturity. At the present time, most genes underlying the above differences are not known, nor, as importantly, are the specific changes that have evolved in these genes to provide these capabilities. Understanding the basis of these differences between domesticated plants and animals and their wild ancestors will provide useful information for maintaining and enhancing those traits. In the case of crop plants, identification of the specific genes that control desired traits will allow direct and rapid improvement in a manner not previously possible.

Although comparison of homologous genes or proteins between domesticated species and their wild ancestors may provide useful information with respect to conserved molecular sequences and functional features, this approach is of limited use in identifying genes whose sequences have changed due to human imposed selective pressures. With the advent of sophisticated algorithms and analytical methods, much more information can be teased out of DNA sequence changes with regard to which genes have been positively selected. The most powerful of these methods, “K_{A}/K_{S},” involves pairwise comparisons between aligned protein-coding nucleotide sequences of the ratios of

nonsynonymous nucleotide substitutions per nonsynonymous site (K_{A})/synonymous substitutions per synonymous site (K_{S})

(where nonsynonymous means substitutions that change the encoded amino acid and synonymous means substitutions that do not change the encoded amino acid). “K_{A}/K_{S}-type methods” include this and similar methods.

These methods have been used to demonstrate the occurrence of Darwinian (i.e., natural) molecular-level positive selection, resulting in amino acid differences in homologous proteins. Several groups have used such methods to document that a particular protein has evolved more rapidly than the neutral substitution rate, and thus supports the existence of Darwinian molecular-level positive selection. For example, McDonald and Kreitman (1991) Nature 351:652-654, propose a statistical test of the neutral protein evolution hypothesis based on comparison of the number of amino acid replacement substitutions to synonymous substitutions in the coding region of a locus. When they apply this test to the Adh locus of three Drosophila species, they conclude that it shows instead that the locus has undergone adaptive fixation of selectively advantageous mutations and that selective fixation of adaptive mutations may be a viable alternative to the clocklike accumulation of neutral mutations as an explanation for most protein evolution. Jenkins et al. (1995) Proc. R. Soc. Lond. B 261:203-207 use the McDonald & Kreitman test to investigate whether adaptive evolution is occurring in sequences controlling transcription (non-coding sequences).

Nakashima et al. (1995) Proc. Natl. Acad. Sci. USA 92:5606-5609, use the method of Miyata and Yasunaga to perform pairwise comparisons of the nucleotide sequences of ten PLA2 isozyme genes from two snake species; this method involves comparing the number of nucleotide substitutions per site for the noncoding regions including introns (K_{N}) and the K_{A} and K_{S}. They conclude that the protein coding regions have been evolving at much higher rates than the noncoding regions including introns. The highly accelerated substitution rate is responsible for Darwinian molecular-level evolution of PLA2 isozyme genes to produce new physiological activities that must have provided strong selective advantage for catching prey or for defense against predators. Endo et al. (1996) Mol. Biol. Evol. 13(5):685-690 use the method of Nei and Gojobori, wherein d_{N} is the number of nonsynonymous substitutions and d_{S} is the number of synonymous substitutions, for the purpose of documenting natural selection on genes. Metz and Palumbi (1996) Mol. Biol. Evol. 13(2):397-406 use the McDonald & Kreitman (supra) test as well as a method attributed to Nei and Gojobori, Nei and Jin, and Kumar, Tamura, and Nei; examining the average proportions of Pₙ, the replacement substitutions per replacement site, and Pₛ, the silent substitutions per silent site, to look for evidence of positive selection on binding genes in sea urchins to investigate whether they have rapidly evolved as a prelude to species formation. Goodwin et al. (1996) Mol. Biol. Evol. 13(2):346-358 uses similar methods to examine the evolution of a particular murine gene family and conclude that the methods provide important fundamental insights into how selection drives genetic divergence in an experimentally manipulatable system. Edwards et al. (1995) use degenerate primers to pull out MHC loci from various species of birds and an alligator species, which are then analyzed by the Nei and Gojobori methods (d_{N}:d_{S} ratios) to extend MHC studies to nonmammalian vertebrates. Whitfield et al. (1993) Nature 364:713-715 use K_{A}/K_{S} analysis to look for directional selection in the regions flanking a conserved region in the SRY gene (that determines male sex). They suggest that the rapid evolution of SRY could be a significant cause of reproductive isolation, leading to new species. Wettsetin et al. (1996) Mol. Biol. Evol. 13(1):56-66 apply the MEGA program of Kumar, Tamura and Nei and phylogenetic analysis to investigate the diversification of MHC class I genes in squirrels and related rodents. Parham and Ohta (1996) Science 272:67-74 state that a population biology approach, including tests for selection as well as for gene conversion and neutral drift are required to analyze the generation and maintenance of human MHC class I polymorphism. Hughes (1997) Mol. Biol. Evol. 14(1):1-5 compared over one hundred orthologous immunoglobulin C2 domains between human and rodent, using the method of Nei and Gojobori (d_{N}:d_{S} ratios) to test the hypothesis that proteins expressed in cells of the vertebrate immune system evolve unusually rapidly. Swanson and Vacquier (1998) Science 281:710-712 use d_{N}:d_{S} ratios to demonstrate concerted evolution between the lysin and the egg receptor for lysin and discuss the role of such concerted evolution in forming new species (speciation). Messier and Stewart (1997) Nature 385:151-154, used K_{A}/K_{S} to demonstrate positive selection in primate lysozymes.

The genetic changes associated with domestication have been most extensively investigated in maize (the preferred agricultural term for corn) (Dorweiler (1993) Science 262:232-235). For maize, (Zea mays ssp. mays), a small number of single-gene changes apparently accounts for all the differences between our present domesticated maize plant and its wild ancestor, teosinte (Zea mays ssp paruiglumis) (Dorweiler, 1993). QTL (quantitative trait locus) analysis has demonstrated (Doebley (1990) PNAS USA 87:9888-9892) that no more than fifteen genes control traits of interest in maize and explain the profound difference in morphology between maize and teosinte (Wang (1999) Nature 398:236-239).

Importantly, a similarly small number of genes may control traits of interest in other grass-derived crop plants, including rice, wheat, millet and sorghum (Paterson (1995) Science 269:1714-1718). In fact, for most of these relevant genes in maize, the homologous gene may control similar traits in other grass-derived crop plants (Paterson, 1995). Thus, identification of these genes in one grass-derived crop plant would facilitate identification of homologous genes in all of the others.

As can be seen from the papers cited above, analytical methods of molecular evolution to identify rapidly evolving genes (K_{A}/K_{S}-type methods) can be applied to achieve many different purposes, most commonly to confirm the existence of Darwinian molecular-level positive selection, but also to assess the frequency of Darwinian molecular-level positive selection, to elucidate mechanisms by which new species are formed, or to establish single or multiple origin for specific gene polymorphisms. What is clear is from the papers cited above and others in the literature is that none of the authors applied K_{A}/K_{S}-type methods to identify evolutionary changes in domesticated plants and animals brought about by artificial selective pressures. While Turcich et al. (1996) Sexual Plant Reproduction 9:65-74, describes the use of K_{S} analysis on plant genes, it is believed that no one has used K_{A}/K_{S} type analysis as a systematic tool for identifying in domesticated plants and animals those genes that contain evolutionarily significant sequence changes that can be exploited in the development, maintenance or enhancement of desirable commercial or aesthetic traits.

The identification in domesticated species of genes that have evolved to confer unique, enhanced or altered functions compared to homologous ancestral genes could be used to develop agents to modulate these functions. The identification of the underlying domesticated species genes and the specific nucleotide changes that have evolved, and the further characterization of the physical and biochemical changes in the proteins encoded by these evolved genes, could provide valuable information on the mechanisms underlying the desired trait. This valuable information could be applied to developing agents that further enhance the function of the target proteins. Alternatively, further engineering of the responsible genes could modify or augment the desired trait. Additionally, the identified genes may be found to play a role in controlling traits of interest in other domesticated plants. A similar process can identify genes for traits of interest in domestic animals.

All references cited herein are hereby incorporated by reference in their entirety.

### DISCLOSURE OF THE INVENTION

The subject invention concerns methods of identifying polynucleotides that control commercially valuable traits in domesticated plants or animals. These polynucleotides that, in accordance with the methods of the subject invention, are found to control commercially valuable traits can be used to further enhance those traits. Polynucleotides identified to control commercially valuable traits such as drought-, disease-, or stress-resistance or yield, protein content, short day length flowering, oil content, ease of harvest, taste, and the like can be used to develop compositions and methods to further enhance the commercial value of domesticated plants. While it is desired to identify polynucleotides that control valuable traits, it is challenging to identify such polynucleotides among the tens of thousands of genes in plant and animal genomes. The invention comprises narrowing the search for such polynucleotides by comparing the corresponding polynucleotide sequences of domesticated and ancestor organisms to select those sequences containing nucleotide changes that are evolutionarily significant, which is typically indicated by a Ka/Ks ratio of 1.0 or greater. For example, the subset of ancestor-modern plant polynucleotide pairs with Ka/Ks ratios of 1.0 should contain polynucleotides affected by neutral evolution, that is those for which the trait has not been under pressure, imposed by man or nature, to either be conserved or to change. Such polynucleotides can then be tested for those encoding traits such as such as drought-, disease-, or stress-resistance, because these functions have been dramatically supplemented by domestication, alleviating natural selection pressures on these polynucleotides. The subset of ancestor-modem plant polynucleotide pairs with Ka/Ks ratios greater than 1.0 should contain polynucleotides affected by selection. Such polynucleotides can then be tested for those encoding traits such as yield, protein content, short day length flowering, oil content, ease of harvest, taste, and the like, because these traits have been under intense, unidirectional, unremitting selective pressure by humans in the course of domestication of plants such as food crops.

Thus, in one embodiment, the present invention provides methods for identifying polynucleotide and polypeptide sequences having evolutionarily significant changes, which are associated with commercial or aesthetic traits in domesticated organisms including plants and animals. The invention uses comparative genomics to identify specific gene changes which may be associated with, and thus responsible for, structural, biochemical or physiological conditions, such as commercially or aesthetically relevant traits, and using the information obtained from these polynucleotide or polypeptide sequences to develop domesticated organisms with enhanced traits of interest.

In one preferred embodiment, a polynucleotide or polypeptide of a domesticated plant or animal has undergone artificial selection that resulted in an evolutionarily significant change present in the domesticated species that is not present in the wild ancestor. One example of this embodiment is that the polynucleotide or polypeptide may be associated with enhanced crop yield as compared to the ancestor. Other examples include short day length flowering (i.e., flowering only if the daily period of light is shorter than some critical length), protein content, oil content, ease of harvest, and taste. The present invention can thus be useful in gaining insight into the genes and/or molecular mechanisms that underlie functions or traits in domesticated organisms. This information can be useful in designing the polynucleotide so as to further enhance the function or trait. For example, a polynucleotide determined to be responsible for improved crop yield could be subjected to random or directed mutagenesis, followed by testing of the mutant genes to identify those which further enhance the trait.

Accordingly, in one aspect, methods are provided for identifying a polynucleotide sequence encoding a polypeptide of a domesticated organism (e.g., a plant or animal), wherein the polypeptide may be associated with a commercially or aesthetically relevant trait that is unique, enhanced or altered in the domesticated organism as compared to the ancestor of the domesticated organism, comprising the steps of: a) comparing protein-coding nucleotide sequences of said domesticated organism to protein-coding nucleotide sequences of said wild ancestor; and b) selecting a polynucleotide sequence in the domesticated organism that contains a nucleotide change as compared to a corresponding sequence in the wild ancestor, wherein said change is evolutionarily significant.

In another aspect of the invention, methods are provided for identifying an evolutionarily significant change in a protein-coding nucleotide sequence of a domesticated organism (e.g., a plant or animal), comprising the steps of: a) comparing protein-coding nucleotide sequences of the domesticated organism to corresponding sequences of a wild ancestor of the domesticated organism; and b) selecting a polynucleotide sequence in said domesticated organism that contains a nucleotide change as compared to the corresponding sequence of the wild ancestor, wherein the change is evolutionarily significant.

In some embodiments, the nucleotide change identified by any of the methods described herein is a non-synonymous substitution. In some embodiments, the evolutionary significance of the nucleotide change is determined according to the non-synonymous substitution rate (K_{A}) of the nucleotide sequence. In some embodiments, the evolutionarily significant changes are assessed by determining the K_{A}/K_{S} ratio between the domesticated organism polynucleotide and the corresponding ancestral polynucleotide. In some of these embodiments, preferably the ratio is at least about 0.75, or more preferably 1.0. With increasing preference, the ratio is at least about 1.0, 1.25, 1.50, 2.00, or greater.

In another aspect, the invention provides a method of identifying an agent which may modulate the relevant trait in the domesticated organism, said method comprising contacting at least one candidate agent with a cell, model system or transgenic plant or animal that expresses the polynucleotide sequence having the evolutionarily significant change, or a composition comprising the evolutionarily significant polypeptide wherein the agent is identified by its ability to modulate function or synthesis of the polypeptide.

Also provided is a method for large scale sequence comparison between protein-coding nucleotide sequences of a domesticated organism and protein-coding sequences from a wild ancestor, said method comprising: a) aligning the domesticated organism sequences with corresponding sequences from the wild ancestor according to sequence homology; and b) identifying any nucleotide changes within the domesticated organism's sequences as compared to the homologous sequences from the wild ancestor organism.

In another aspect, the subject invention provides a method for correlating an evolutionarily significant nucleotide change to a commercially or aesthetically relevant trait that is unique, enhanced or altered in a domesticated organism, comprising: a) identifying a nucleotide sequence having an evolutionarily significant change according to the methods described herein; and b) analyzing the functional effect of the presence or absence of the identified sequence in the domesticated organism or in a model system.

The domesticated plants used in the subject methods can be maize, rice, tomatoes, potatoes or any domesticated plant for which the wild ancestor is extant and known. For example, the ancestor of maize is teosinte (Zea mays parviglumis); ancestors of wheat are Triticum monococcum, T. speltoides and Aegilops tauschii; and an ancestor of rice is O. rufipogon. The relevant trait can be any commercially or aesthetically relevant trait such as yield, short day length flowering, protein content, oil content, drought resistance, taste, ease of harvest or disease resistance. In a preferred embodiment, the domesticated plant is rice, and the relevant trait is yield.

In another embodiment of the invention, methods for the identification of polynucleotides associated with stress-resistance in an ancestor organism are provided. In this embodiment, a polynucleotide in the domesticated organism has undergone neutral evolution relative to a polynucleotide in the ancestor which is or is suspected of being associated with stress-resistance, whereby mutations have accumulated in the domesticated organism's polynucleotide. The stress-resistance trait in the ancestor may be unique, enhanced or altered relative to the domesticated organism.

The method for identifying the polynucleotide sequence comprises a) comparing polypeptide-coding nucleotide sequences of the domesticated organism to polypeptide coding nucleotide sequences of the wild ancestor; and b) selecting a polynucleotide sequence in the ancestor organism that contains at least one nucleotide change as compared to a corresponding sequence in the domesticated organism, wherein the change is evolutionarily neutral. The stress-resistance trait may be drought resistance, disease resistance, pest resistance, high salt level resistance or other stress-resistance traits of commercial interest.

Also provided is a method for identifying an evolutionarily neutral change in a polypeptide-coding polynucleotide sequence of a wild ancestor of a domesticated organism comprising: a) comparing polypeptide-coding polynucleotide sequences of said wild ancestor to corresponding sequences of said domesticated organism; and b) selecting a polynucleotide sequence in the domesticated organism that contains a nucleotide change as compared to the corresponding sequence of the wild ancestor, wherein the change is evolutionarily neutral and the polynucleotide is associated with a stress-resistance trait in the wild ancestor.

Neutral evolution is typically indicated by a K_{A}/K_{S} ratio of between about 0.75 and 1.25, more preferably between about 0.9 and 1.1, and most preferably about 1.0. The K_{A}/K_{S} comparison may be calculated as ancestor to domestic organism, or domestic to ancestor organism.

In another aspect, the invention provides for a method of identifying an agent that may modulate a stress-resistance trait in an organism (ancestor or domesticated organism), wherein at least one candidate agent is contacted with the ancestor, domesticated organism or with a cell or transgenic organism that expresses the polynucleotide sequence associated with stress-resistance, wherein the agent is identified by its ability to modulate the function of the polypeptide encoded by the polynucleotide.

Also provided is a method for large scale sequence comparison between polypeptide-coding nucleotide sequences of a wild ancestor and those of a domesticated organism, wherein the ancestor polypeptide confers or is suspected of conferring a stress-related trait that is unique, enhanced or altered in the wild ancestor as compared to the domesticated organism, comprising: a) aligning the ancestor and domesticated sequences according to sequence homology, and b) identifying any nucleotide changes in the domesticated organism sequence as compared to the ancestor homologous sequence, wherein said changes are evolutionarily neutral.

In another aspect, the subject invention provides a method for correlating an evolutionarily neutral nucleotide change to a commercially or aesthetically relevant trait that is unique, enhanced or altered in a domesticated organism, comprising: a) identifying a nucleotide sequence having an evolutionarily neutral change according to the methods described herein; and b) analyzing the functional effect of the presence or absence of the identified sequence in the domesticated organism or in a model system.

### BRIEF DESCRIPTION OF THE FIGURES

FIG. 1 shows a nucleotide alignment of O. sativa cv. Nipponbare and O. rufipogon (NSGC5953) for EG307. This alignment includes untranslated regions (UTR) on the 5′ end and notes the start and stop codons for this gene.

FIG. 2 shows a protein alignment of O. sativa cv. Nipponbare and O. rufipogon (NSGC5953) for EG307. This alignment includes the complete coding (CDS) region.

FIG. 3 shows a nucleotide sequence of EG307 in Zea mays mays and Zea mays parviglumis (teosinte, strain Benz967) for coding region of the gene. Start and stop codons are identified.

FIG. 4 shows a protein alignment of Zea mays mays and Zea mays parviglumis EG307. This alignment includes the full-length deduced protein sequence.

FIG. 5 shows markers CDO1387 and RZ672 mapped to five different genetic rice maps, indicating that the range of these markers is consistent among the five maps. EG307 is upstream of CDO1387 (about 200 kb) and a QTL for 1000 Grain Weight is associated with marker RZ672.

FIG. 6 shows the nucleotide alignment of O. sativa (strain Nipponbare) and O. rufipogon (strain 5498) for EG1117, and indicates there are three nonsynomous changes.

FIG. 7 shows the protein alignment of O. sativa (strain Nipponbare) and O. rufipogon (strain 5498) for EG1117. This alignment includes the partial CDS region with the stop codon. The three amino acid difference between O. sativa and O. rufipogon are shown in bold.

FIG. 8 shows the protein alignment of O. sativa (strain Nipponbare) and Araidopsis PTR2-B (histidine transporting protein, NP_{—}178313).

### DETAILED DESCRIPTION OF THE INVENTION

In one embodiment, the present invention utilizes comparative genomics to identify positively selected genes and specific gene changes which are associated with, and thus may contribute to or be responsible for, commercially or aesthetically relevant traits in domesticated organisms (e.g., plants and animals).

In another embodiment, the invention identifies evolutionarily neutral genes and gene changes that are associated with stress-resistance in ancestors of domesticated organisms.

The practice of the present invention employs, unless otherwise indicated, conventional techniques of molecular biology, genetics and molecular evolution, which are within the skill of the art. Such techniques are explained fully in the literature, such as: “Molecular Cloning: A Laboratory Manual”, second edition (Sambrook et al., 1989); “Oligonucleotide Synthesis” (M. J. Gait, ed., 1984); “Current Protocols in Molecular Biology” (F. M. Ausubel et al., eds., 1987); “PCR: The Polymerase Chain Reaction”, (Mullis et al., eds., 1994); “Molecular Evolution”, (Li, 1997).

### I. DEFINITIONS

As used herein, a “polynucleotide” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified polynucleotides such as methylated and/or capped polynucleotides, polynucleotides containing modified bases, backbone modifications, and the like. The terms “polynucleotide” and “nucleotide sequence” are used interchangeably.

As used herein, a “gene” refers to a polynucleotide or portion of a polynucleotide comprising a sequence that encodes a protein. It is well understood in the art that a gene also comprises non-coding sequences, such as 5′ and 3′ flanking sequences (such as promoters, enhancers, repressors, and other regulatory sequences) as well as introns.

The terms “polypeptide,” “peptide,” and “protein” are used interchangeably herein to refer to polymers of amino acids of any length. These terms also include proteins that are post-translationally modified through reactions that include glycosylation, acetylation and phosphorylation.

The term “domesticated organism” refers to an individual living organism or population of same, a species, subspecies, variety, cultivar or strain, that has been subjected to artificial selection pressure and developed a commercially or aesthetically relevant trait. In some preferred embodiments, the domesticated organism is a plant selected from the group consisting of maize, wheat, rice, sorghum, tomato or potato, or any other domesticated plant of commercial interest, where an ancestor is known. A “plant” is any plant at any stage of development, particularly a seed plant.

In other preferred embodiments, the domesticated organism is an animal selected from the group consisting of cattle, horses, pigs, cats and dogs. A domesticated organism and its ancestor may be related as different species, subspecies, varieties, cultivars or strains or any combination thereof.

The term “wild ancestor” or “ancestor” means a forerunner or predecessor organism, species, subspecies, variety, cultivar or strain from which a domesticated organism, species, subspecies, variety, cultivar or strain has evolved. A domesticated organism can have one or more than one ancestor. Typically, domesticated plants can have one or a plurality of ancestors, while domesticated animals usually have only a single ancestor.

The term “commercially or aesthetically relevant trait” is used herein to refer to traits that exist in domesticated organisms such as plants or animals whose analysis could provide information (e.g., physical or biochemical data) relevant to the development of improved organisms or of agents that can modulate the polypeptide responsible for the trait, or the respective polynucleotide. The commercially or aesthetically relevant trait can be unique, enhanced or altered relative to the ancestor. By “altered,” it is meant that the relevant trait differs qualitatively or quantitatively from traits observed in the ancestor.

The term “K_{A}/K_{S}-type methods” means methods that evaluate differences, frequently (but not always) shown as a ratio, between the number of nonsynonymous substitutions and synonymous substitutions in homologous genes (including the more rigorous methods that determine non-synonymous and synonymous sites). These methods are designated using several systems of nomenclature, including but not limited to K_{A}/K_{S}, d_{N}/d_{S}, D_{N}/D_{S}.

The terms “evolutionarily significant change” and “adaptive evolutionary change” refer to one or more nucleotide or peptide sequence change(s) between two organisms, species, subspecies, varieties, cultivars and/or strains that may be attributed to either relaxation of selective pressure or positive selective pressure. One method for determining the presence of an evolutionarily significant change is to apply a K_{A}/K_{S}-type analytical method, such as to measure a K_{A}/K_{S} ratio. Typically, a K_{A}/K_{S} ratio of 1.0 or greater is considered to be an evolutionarily significant change.

Strictly speaking, K_{A}/K_{S} ratios of exactly 1.0 are indicative of relaxation of selective pressure (neutral evolution), and K_{A}/K_{S} ratios greater than 1.0 are indicative of positive selection. However, it is commonly accepted that the ESTs in GenBank and other public databases often suffer from some degree of sequencing error, and even a few incorrect nucleotides can influence K_{A}/K_{S} ratios. For this reason, polynucleotides with K_{A}/K_{S} ratios as low as 0.75 can be carefully resequenced and re-evaluated for relaxation of selective pressure (neutral evolutionarily significant change), positive selection pressure (positive evolutionarily significant change), or negative selective pressure (evolutionarily conservative change).

The term “positive evolutionarily significant change” means an evolutionarily significant change in a particular organism, species, subspecies, variety, cultivar or strain that results in an adaptive change that is positive as compared to other related organisms. An example of a positive evolutionarily significant change is a change that has resulted in enhanced yield in crop plants. As stated above, positive selection is indicated by a K_{A}/K_{S} ratio greater than 1.0. With increasing preference, the K_{A}/K_{S} value is greater than 1.25, 1.5 and 2.0.

The term “neutral evolutionarily significant change” refers to a polynucleotide or polypeptide change that appears in a domesticated organism relative to its ancestral organism, and which has developed under neutral conditions. A neutral evolutionary change is evidenced by a K_{A}/K_{S} value of between about 0.75-1.25, preferably between about 0.9 and 1.1, and most preferably equal to about 1.0. Also, in the case of neutral evolution, there is no “directionality” to be inferred. The gene is free to accumulate changes without constraint, so both the ancestral and domesticated versions are changing with respect to one another.

The term “resistant” means that an organism exhibits an ability to avoid, or diminish the extent of, a disease condition and/or development of the disease, preferably when compared to non-resistant organisms.

The term “susceptibility” means that an organism fails to avoid, or diminish the extent of, a disease condition and/or development of the disease condition, preferably when compared to an organism that is known to be resistant.

It is understood that resistance and susceptibility vary from individual to individual, and that, for purposes of this invention, these terms also apply to a group of individuals within a species, and comparisons of resistance and susceptibility generally refer overall to inter-specific differences, although comparisons within species may be used. Taxonomic classification of wild relatives is fairly changeable. Thus, a species difference based on a taxonomic classification may change to an intra-specific difference if taxonomic classifications are changed.

The term “stress-resistance” refers to the ability to withstand drought, disease, pests (including, but not limited to, insects, animal herbivores, and microbes), high salt levels, and other adverse stimuli, internal or external, that tend to disturb the plant's homeostasis, and may lead to disorder, disease, or death if uncorrected.

The term “homologous” or “homologue” or “ortholog” is known and well understood in the art and refers to related sequences that share a common ancestor and is determined based on degree of sequence identity. These terms describe the relationship between a gene found in one species, subspecies, variety, cultivar or strain and the corresponding or equivalent gene in another species, subspecies, variety, cultivar or strain. For purposes of this invention homologous sequences are compared. “Homologous sequences” or “homologues” or “orthologs” are thought, believed, or known to be functionally related. A functional relationship may be indicated in any one of a number of ways, including, but not limited to, (a) degree of sequence identity; (b) same or similar biological function. Preferably, both (a) and (b) are indicated. The degree of sequence identity may vary, but is preferably at least 50% (when using standard sequence alignment programs known in the art), more preferably at least 60%, more preferably at least about 75%, more preferably at least about 85%. Homology can be determined using software programs readily available in the art, such as those discussed in Current Protocols in Molecular Biology (F. M. Ausubel et al., eds., 1987) Supplement 30, section 7.718, Table 7.71. Preferred alignment programs are MacVector (Oxford Molecular Ltd, Oxford, U.K.) and ALIGN Plus (Scientific and Educational Software, Pennsylvania). Another preferred alignment program is Sequencher (Gene Codes, Ann Arbor, Mich.), using default parameters.

The term “nucleotide change” refers to nucleotide substitution, deletion, and/or insertion, as is well understood in the art.

“Housekeeping genes” is a term well understood in the art and means those genes associated with general cell function, including but not limited to growth, division, stasis, metabolism, and/or death. “Housekeeping” genes generally perform functions found in more than one cell type. In contrast, cell-specific genes generally perform functions in a particular cell type and/or class.

The term “agent”, as used herein, means a biological or chemical compound such as a simple or complex organic or inorganic molecule, a peptide, a protein or an oligonucleotide that modulates the function of a polynucleotide or polypeptide. A vast array of compounds can be synthesized, for example oligomers, such as oligopeptides and oligonucleotides, and synthetic organic and inorganic compounds based on various core structures, and these are also included in the term “agent”. In addition, various natural sources can provide compounds for screening, such as plant or animal extracts, and the like. Compounds can be tested singly or in combination with one another.

The term “to modulate function” of a polynucleotide or a polypeptide means that the function of the polynucleotide or polypeptide is altered when compared to not adding an agent. Modulation may occur on any level that affects function. A polynucleotide or polypeptide function may be direct or indirect, and measured directly or indirectly.

A “function of a polynucleotide” includes, but is not limited to, replication; translation; expression pattern(s). A polynucleotide function also includes functions associated with a polypeptide encoded within the polynucleotide. For example, an agent which acts on a polynucleotide and affects protein expression, conformation, folding (or other physical characteristics), binding to other moieties (such as ligands), activity (or other functional characteristics), regulation and/or other aspects of protein structure or function is considered to have modulated polynucleotide function.

A “function of a polypeptide” includes, but is not limited to, conformation, folding (or other physical characteristics), binding to other moieties (such as ligands), activity (or other functional characteristics), and/or other aspects of protein structure or functions. For example, an agent that acts on a polypeptide and affects its conformation, folding (or other physical characteristics), binding to other moieties (such as ligands), activity (or other functional characteristics), and/or other aspects of protein structure or functions is considered to have modulated polypeptide function. The ways that an effective agent can act to modulate the function of a polypeptide include, but are not limited to 1) changing the conformation, folding or other physical characteristics; 2) changing the binding strength to its natural ligand or changing the specificity of binding to ligands; and 3) altering the activity of the polypeptide.

The term “target site” means a location in a polypeptide which can be a single amino acid and/or is a part of, a structural and/or functional motif, e.g., a binding site, a dimerization domain, or a catalytic active site. Target sites may be useful for direct or indirect interaction with an agent, such as a therapeutic agent.

The term “molecular difference” includes any structural and/or functional difference. Methods to detect such differences, as well as examples of such differences, are described herein.

A “functional effect” is a term well known in the art, and means any effect which is exhibited on any level of activity, whether direct or indirect.

The term “ease of harvest” refers to plant characteristics or features that facilitate manual or automated collection of structures or portions (e.g., fruit, leaves, roots) for consumption or other commercial processing.

The term “yield” refers to the amount of plant or animal tissue or material that is available for use by humans for food, therapeutic, veterinary or other markets.

The term “enhanced economic productivity” refers to the ability to modulate a commercially or aesthetically relevant trait so as to improve desired features. Increased yield and enhanced stress resistance are two examples of enhanced economic productivity.

### II. GENERAL PROCEDURES KNOWN IN THE ART

For the purposes of this invention, the source of the polynucleotide from the domesticated plant or animal or its ancestor can be any suitable source, e.g., genomic sequences or cDNA sequences. Preferably, cDNA sequences are compared. Protein-coding sequences can be obtained from available private, public and/or commercial databases such as those described herein. These databases serve as repositories of the molecular sequence data generated by ongoing research efforts. Alternatively, protein-coding sequences may be obtained from, for example, sequencing of cDNA reverse transcribed from mRNA expressed in cells, or after PCR amplification, according to methods well known in the art. Alternatively, genomic sequences may be used for sequence comparison. Genomic sequences can be obtained from available public, private and/or commercial databases or from sequencing of genomic DNA libraries or from genomic DNA, after PCR.

In some embodiments, the cDNA is prepared from mRNA obtained from a tissue at a determined developmental stage, or a tissue obtained after the organism has been subjected to certain environmental conditions. cDNA libraries used for the sequence comparison of the present invention can be constructed using conventional cDNA library construction techniques that are explained fully in the literature of the art. Total mRNAs are used as templates to reverse-transcribe cDNAs. Transcribed cDNAs are subcloned into appropriate vectors to establish a cDNA library. The established cDNA library can be maximized for full-length cDNA contents, although less than full-length cDNAs may be used. Furthermore, the sequence frequency can be normalized according to, for example, Bonaldo et al. (1996) Genome Research 6:791-806. cDNA clones randomly selected from the constructed cDNA library can be sequenced using standard automated sequencing techniques. Preferably, full-length cDNA clones are used for sequencing. Either the entire or a large portion of cDNA clones from a cDNA library may be sequenced, although it is also possible to practice some embodiments of the invention by sequencing as little as a single cDNA, or several cDNA clones.

In one preferred embodiment of the present invention, cDNA clones to be sequenced can be pre-selected according to their expression specificity. In order to select cDNAs corresponding to active genes that are specifically expressed, the cDNAs can be subject to subtraction hybridization using mRNAs obtained from other organs, tissues or cells of the same animal. Under certain hybridization conditions with appropriate stringency and concentration, those cDNAs that hybridize with non-tissue specific mRNAs and thus likely represent “housekeeping” genes will be excluded from the cDNA pool. Accordingly, remaining cDNAs to be sequenced are more likely to be associated with tissue-specific functions. For the purpose of subtraction hybridization, non-tissue-specific mRNAs can be obtained from one organ, or preferably from a combination of different organs and cells. The amount of non-tissue-specific mRNAs are maximized to saturate the tissue-specific cDNAs.

Alternatively, information from online databases can be used to select or give priority to cDNAs that are more likely to be associated with specific functions. For example, the ancestral cDNA candidates for sequencing can be selected by PCR using primers designed from candidate domesticated organism cDNA sequences. Candidate domesticated organism cDNA sequences are, for example, those that are only found in a specific tissue, such as skeletal muscle, or that correspond to genes likely to be important in the specific function. Such tissue-specific cDNA sequences may be obtained by searching online sequence databases in which information with respect to the expression profile and/or biological activity for cDNA sequences may be specified.

Sequences of ancestral homologue(s) to a known domesticated organism's gene may be obtained using methods standard in the art, such as PCR methods (using, for example, GeneAmp PCR System 9700 thermocyclers (Applied Biosystems, Inc.)). For example, ancestral cDNA candidates for sequencing can be selected by PCR using primers designed from candidate domesticated organism cDNA sequences. For PCR, primers may be made from the domesticated organism's sequences using standard methods in the art, including publicly available primer design programs such as PRIMER® (Whitehead Institute). The ancestral sequence amplified may then be sequenced using standard methods and equipment in the art, such as automated sequencers (Applied Biosystems, Inc.). Likewise, ancestors gene mimics can be used to obtain corresponding genes in domesticated organisms.

### III. IDENTIFICATION OF POSITIVELY SELECTED POLYNUCLEOTIDES IN DOMESTICATED ORGANISMS

In a preferred embodiment, the methods described herein can be applied to identify the genes that control traits of interest in agriculturally important domesticated plants. Humans have bred domesticated plants for several thousand years without knowledge of the genes that control these traits. Knowledge of the specific genetic mechanisms involved would allow much more rapid and direct intervention at the molecular level to create plants with desirable or enhanced traits.

Humans, through artificial selection, have provided intense selection pressures on crop plants. This pressure is reflected in evolutionarily significant changes between homologous genes of domesticated organisms and their wild ancestors. It has been found that only a few genes, e.g., 10-15 per species, control traits of commercial interest in domesticated crop plants. These few genes have been exceedingly difficult to identify through standard methods of plant molecular biology. The K_{A}/K_{S} and related analyses described herein can identify the genes controlling traits of interest.

For any crop plant of interest, cDNA libraries can be constructed from the domesticated species or subspecies and its wild ancestor. As is described in U.S. Ser. No. 09/240,915, filed Jan. 29, 1999, the cDNA libraries of each are “BLASTed” against each other to identify homologous polynucleotides. Alternatively, the skilled artisan can access commercially and/or publicly available genomic or cDNA databases rather than constructing cDNA libraries.

Next, a K_{A}/K_{S} or related analysis is conducted to identify selected genes that have rapidly evolved under selective pressure. These genes are then evaluated using standard molecular and transgenic plant methods to determine if they play a role in the traits of commercial or aesthetic interest. The genes of interest are then manipulated by, e.g., random or site-directed mutagenesis, to develop new, improved varieties, subspecies, strains or cultivars.

The general method of the invention is as follows. Briefly, nucleotide sequences are obtained from a domesticated organism and a wild ancestor. The domesticated organism's and ancestor's nucleotide sequences are compared to one another to identify sequences that are homologous. The homologous sequences are analyzed to identify those that have nucleic acid sequence differences between the domesticated organism and ancestor. Then molecular evolution analysis is conducted to evaluate quantitatively and qualitatively the evolutionary significance of the differences. For genes that have been positively selected, outgroup analysis can be done to identify those genes that have been positively selected in the domesticated organism (or in the ancestor). Next, the sequence is characterized in terms of molecular/genetic identity and biological function. Finally, the information can be used to identify agents that can modulate the biological function of the polypeptide encoded by the gene.

The general methods of the invention entail comparing protein-coding nucleotide sequences of ancestral and domesticated organisms. Bioinformatics is applied to the comparison and sequences are selected that contain a nucleotide change or changes that is/are evolutionarily significant change(s). The invention enables the identification of genes that have evolved to confer some evolutionary advantage and the identification of the specific evolved changes. In a preferred embodiment, the domesticated organism is Oryza sativa and the wild ancestor is Oryza rufipogon. In the case of the present invention, protein-coding nucleotide sequences were obtained from O. rufipogon clones by standard sequencing techniques.

Protein-coding sequences of a domesticated organism and its ancestor are compared to identify homologous sequences. Any appropriate mechanism for completing this comparison is contemplated by this invention. Alignment may be performed manually or by software (examples of suitable alignment programs are known in the art). Preferably, protein-coding sequences from an ancestor are compared to the domesticated species sequences via database searches, e.g., BLAST searches. The high scoring “hits,” i.e., sequences that show a significant similarity after BLAST analysis, will be retrieved and analyzed. Sequences showing a significant similarity can be those having at least about 60%, at least about 75%, at least about 80%, at least about 85%, or at least about 90% sequence identity. Preferably, sequences showing greater than about 80% identity are further analyzed. The homologous sequences identified via database searching can be aligned in their entirety using sequence alignment methods and programs that are known and available in the art, such as the commonly used simple alignment program CLUSTAL V by Higgins et al. (1992) CABIOS 8:189-191.

The present invention provides a method for identifying a polynucleotide sequence encoding a polypeptide of a domesticated organism, wherein said polypeptide is or is suspected of being associated with improved yield in said domesticated organism as compared to a wild ancestor of said domesticated organism, comprising the steps of a) comparing polypeptide-coding nucleotide sequences of said domesticated organism to polypeptide-coding nucleotide sequences of said wild ancestor; and b) selecting a polynucleotide sequence in the domesticated organism that contains a nucleotide change as compared to a corresponding sequence in the wild ancestor, wherein said change is evolutionarily significant, whereby the domesticated organism's polynucleotide sequence is identified. In a preferred embodiment, the polypeptide that is associated with improved yield is an EG307 polypeptide.

In the present case, for example, nucleotide sequences obtained from O. rufipogon were used as query sequences in a search of O. sativa ESTs in GenBank to identify homologous sequences. It should be noted that a complete protein-coding nucleotide sequence is not required. Indeed, partial cDNA sequences may be compared. Once sequences of interest are identified by the methods described below, further cloning and/or bioinformatics methods can be used to obtain the entire coding sequence for the gene or protein of interest.

Alternatively, the sequencing and homology comparison of protein-coding sequences between the domesticated organism and its ancestor may be performed simultaneously by using the newly developed sequencing chip technology. See, for example, Rava et al. U.S. Pat. No. 5,545,531.

The aligned protein-coding sequences of domesticated organism and ancestor are analyzed to identify nucleotide sequence differences at particular sites. Again, any suitable method for achieving this analysis is contemplated by this invention. If there are no nucleotide sequence differences, the ancestor protein coding sequence is not usually further analyzed. The detected sequence changes are generally, and preferably, initially checked for accuracy. Preferably, the initial checking comprises performing one or more of the following steps, any and all of which are known in the art: (a) finding the points where there are changes between the ancestral and domesticated organism sequences; (b) checking the sequence fluorogram (chromatogram) to determine if the bases that appear unique to the ancestor or domesticated organism correspond to strong, clear signals specific for the called base; (c) checking the domesticated organism hits to see if there is more than one domesticated organism sequence that corresponds to a sequence change. Multiple domesticated organism sequence entries for the same gene that have the same nucleotide at a position where there is a different nucleotide in an ancestor sequence provides independent support that the domesticated sequence is accurate, and that the change is significant. Such changes are examined using database information and the genetic code to determine whether these nucleotide sequence changes result in a change in the amino acid sequence of the encoded protein. As the definition of “nucleotide change” makes clear, the present invention encompasses at least one nucleotide change, either a substitution, a deletion or an insertion, in a protein-coding polynucleotide sequence of a domesticated organism as compared to a corresponding sequence from the ancestor. Preferably, the change is a nucleotide substitution. More preferably, more than one substitution is present in the identified sequence and is subjected to molecular evolution analysis.

Any of several different molecular evolution analyses or K_{A}/K_{S}-type methods can be employed to evaluate quantitatively and qualitatively the evolutionary significance of the identified nucleotide changes between domesticated species gene sequences and those of corresponding ancestors. Kreitman and Akashi (1995) Annu. Rev. Ecol. Syst. 26:403-422; Li, Molecular Evolution, Sinauer Associates, Sunderland, Mass., 1997. For example, positive selection on proteins (i.e., molecular-level adaptive evolution) can be detected in protein-coding genes by pairwise comparisons of the ratios of nonsynonymous nucleotide substitutions per nonsynonymous site (K_{A}) to synonymous substitutions per synonymous site (K_{S}) (Li et al., 1985; Li, 1993). Any comparison of K_{A} and K_{S} may be used, although it is particularly convenient and most effective to compare these two variables as a ratio. Sequences are identified by exhibiting a statistically significant difference between K_{A} and K_{S} using standard statistical methods.

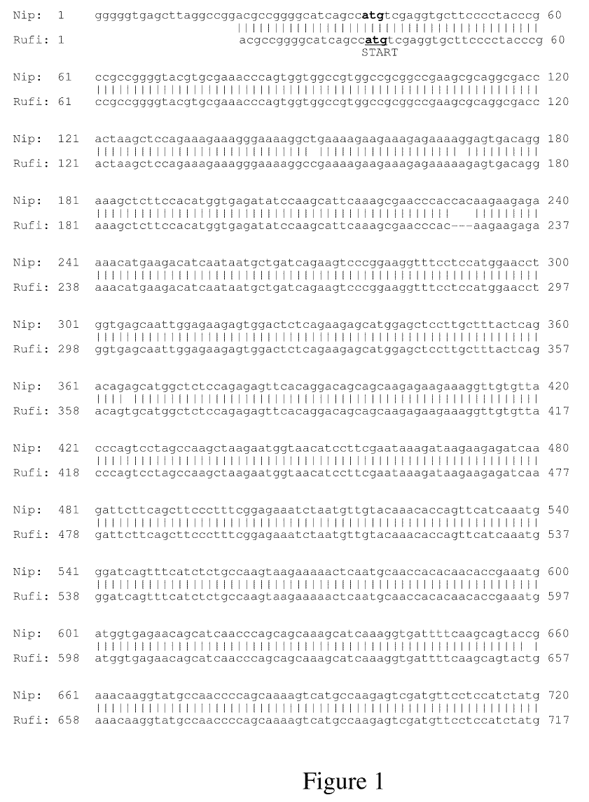

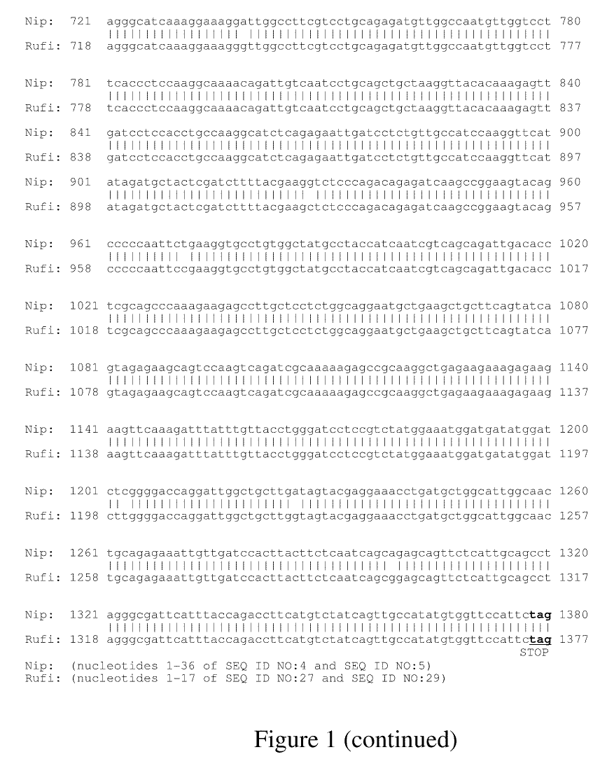

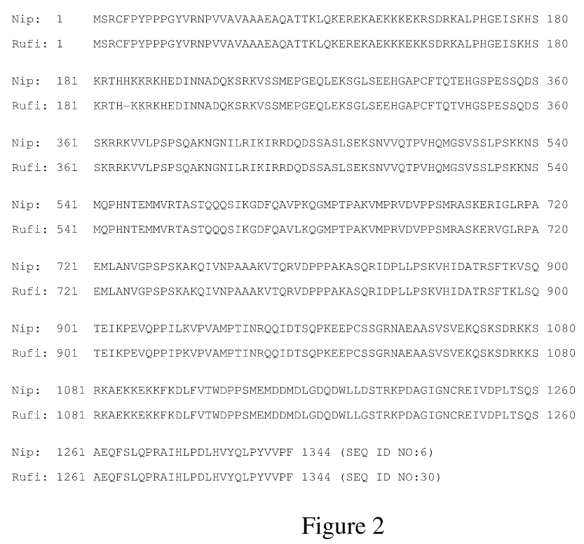

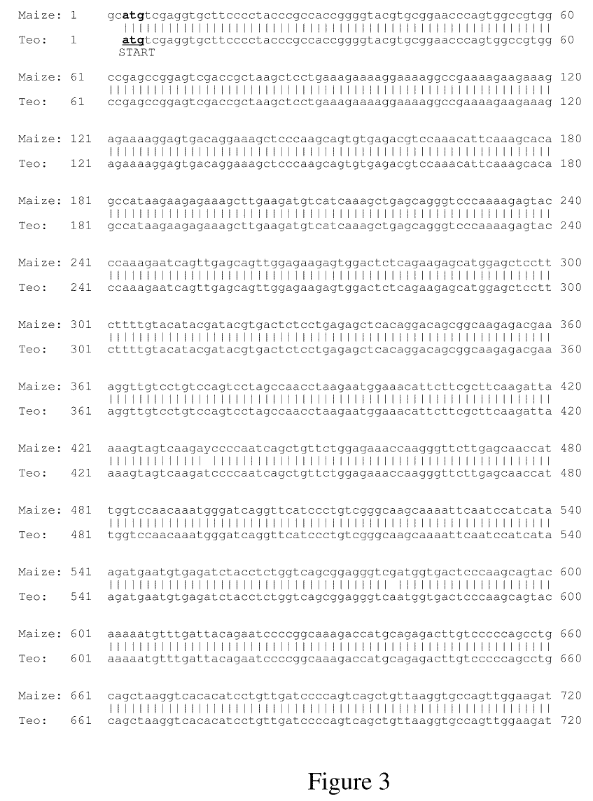

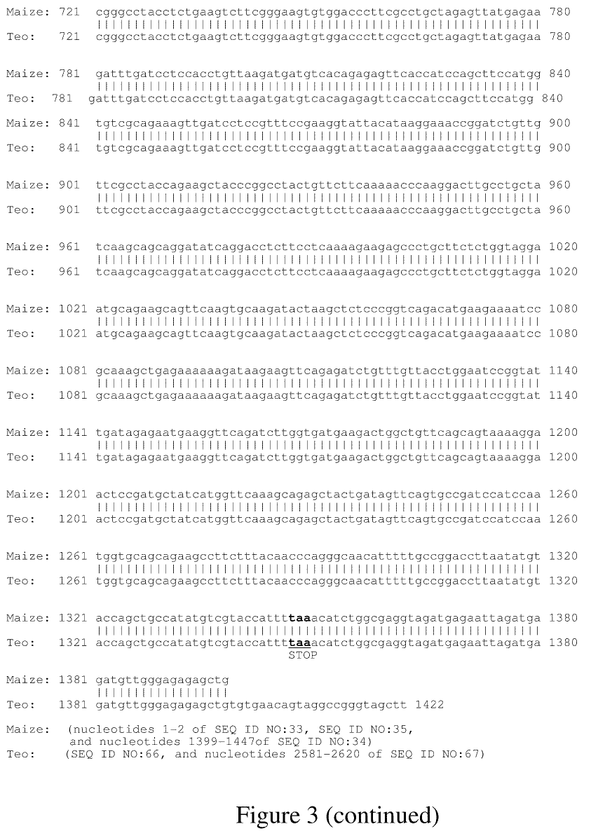

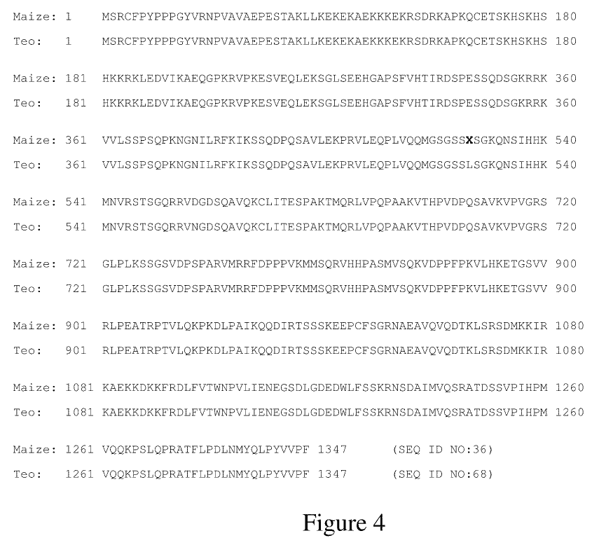

In the case of the present invention, homologous sequences from O. rufipogon and O. sativa were identified. Comparison of the sequences of one O. rufipogon clone, PBI0307H9, SEQ ID NO:31, and O. sativa in GenBank revealed a high K_{A}/K_{S} ratio. Further cloning and PCR of several different strains of O. sativa were completed in order to obtain the entire gene, named EG307, so that the entire gene sequence could be subjected to K_{A}/K_{S} analysis. These procedures are detailed in Example 10. The complete sequence of EG307 in O. rufipogon, SEQ ID NO:28, and O. sativa cv. Nipponbare 1, SEQ ID NO:25, are shown in FIG. 1. The corresponding protein sequences, SEQ ID NO:30, and SEQ ID NO:27, are shown in FIG. 2. A summary of the K_{A}/K_{S} ratios is shown in Table 1 of Example 11. Some strains were more similar to O. rufipogon due to cross-breeding between O. rufipogon and the domestic strain. High K_{A}/K_{S} ratios for some strains indicates an evolutionarily significant change.

Preferably, the K_{A}/K_{S} analysis computer program by Li et al. is used to carry out the present invention, although other analysis programs that can detect positively selected genes between species can also be used. Li et al. (1985) Mol. Biol. Evol. 2:150-174; Li (1993); see also J. Mol. Evol. 36:96-99; Messier and Stewart (1997) Nature 385:151-154; Nei (1987) Molecular Evolutionary Genetics (New York, Columbia University Press). The K_{A}/K_{S} method, which comprises a comparison of the rate of non-synonymous substitutions per non-synonymous site with the rate of synonymous substitutions per synonymous site between homologous protein-coding region of genes in terms of a ratio, is used to identify sequence substitutions that may be driven by adaptive selections or by neutral selections during evolution. A synonymous (“silent”) substitution is one that, owing to the degeneracy of the genetic code, makes no change to the amino acid sequence encoded; a non-synonymous substitution results in an amino acid replacement. The extent of each type of change can be estimated as K_{A} and K_{S}, respectively, the numbers of non-synonymous substitutions per non-synonymous site and synonymous substitutions per synonymous site. Calculations of K_{A}/K_{S} may be performed manually or by using software. An example of a suitable program is MEGA (Molecular Genetics Institute, Pennsylvania State University).

For the purpose of estimating K_{A} and K_{S}, either complete or partial protein-coding sequences are used to calculate total numbers of synonymous and non-synonymous substitutions, as well as non-synonymous and synonymous sites. The length of the polynucleotide sequence analyzed can be any appropriate length. Preferably, the entire coding sequence is compared, in order to determine any and all significant changes. Publicly available computer programs, such as Li93 (Li (1993) J. Mol. Evol. 36:96-99) or INA, can be used to calculate the K_{A} and K_{S} values for all pairwise comparisons. This analysis can be further adapted to examine sequences in a “sliding window” fashion such that small numbers of important changes are not masked by the whole sequence. “Sliding window” refers to examination of consecutive, overlapping subsections of the gene (the subsections can be of any length).

Sliding window K_{A}/K_{S} analysis of, for example, identified gene EG307 showed that there are a number of nonsynonymous changes on the 5′-end of EG307 in many of the O. sativa strains when compared to O. rufipogon. The 3′-end of the gene had a low ratio in all of the strains. These procedures and results are detailed in Example 11 and Tables 2-7.

The comparison of non-synonymous and synonymous substitution rates is represented by the K_{A}/K_{S} ratio. K_{A}/K_{S} has been shown to be a reflection of the degree to which adaptive evolution has been at work in the sequence under study. Full length or partial segments of a coding sequence can be used for the K_{A}/K_{S} analysis. The higher the K_{A}/K_{S} ratio, the more likely that a sequence has undergone adaptive evolution and the non-synonymous substitutions are evolutionarily significant. See, for example, Messier and Stewart (1997). Preferably, the K_{A}/K_{S} ratio is at least about 0.75, more preferably at least about 1.0, more preferably at least about 1.25, more preferably at least about 1.50, or more preferably at least about 2.00. Preferably, statistical analysis is performed on all elevated K_{A}/K_{S} ratios, including, but not limited to, standard methods such as Student's t-test and likelihood ratio tests described by Yang (1998) Mol. Biol. Evol. 37:441-456.

For a pairwise comparison of homologous sequences, K_{A}/K_{S} ratios significantly greater than unity strongly suggest that positive selection has fixed greater numbers of amino acid replacements than can be expected as a result of chance alone, and is in contrast to the commonly observed pattern in which the ratio is less than one. Nei (1987); Hughes and Hei (1988) Nature 335:167-170; Messier and Stewart (1994) Current Biol. 4:911-913; Kreitman and Akashi (1995) Ann. Rev. Ecol. Syst. 26:403-422; Messier and Stewart (1997). Ratios less than one generally signify the role of negative, or purifying selection: there is strong pressure on the primary structure of functional, effective proteins to remain unchanged. Ratios of about 1 indicate evolution under neutral conditions.

All methods for calculating K_{A}/K_{S} ratios are based on a pairwise comparison of the number of nonsynonymous substitutions per nonsynonymous site to the number of synonymous substitutions per synonymous site for the protein-coding regions of homologous genes from the ancestral and domesticated organisms. Each method implements different corrections for estimating “multiple hits” (i.e., more than one nucleotide substitution at the same site). Each method also uses different models for how DNA sequences change over evolutionary time. Thus, preferably, a combination of results from different algorithms is used to increase the level of sensitivity for detection of positively-selected genes and confidence in the result.

Preferably, K_{A}/K_{S} ratios should be calculated for orthologous gene pairs, as opposed to paralogous gene pairs (i.e., a gene which results from speciation, as opposed to a gene that is the result of gene duplication) Messier and Stewart (1997). This distinction may be made by performing additional comparisons with other ancestors, which allows for phylogenetic tree-building. Orthologous genes when used in tree-building will yield the known “species tree”, i.e., will produce a tree that recovers the known biological tree. In contrast, paralogous genes will yield trees which will violate the known biological tree.

It is understood that the methods described herein could lead to the identification of ancestral or domesticated organism polynucleotide sequences that are functionally related to the protein-coding sequences. Such sequences may include, but are not limited to, non-coding sequences or coding sequences that do not encode proteins. These related sequences can be, for example, physically adjacent to the protein-coding sequences in the genome, such as introns or 5′- and 3′-flanking sequences (including control elements such as promoters and enhancers). These related sequences may be obtained via searching available public, private and/or commercial genome databases or, alternatively, by screening and sequencing the organism's genomic library with a protein-coding sequence as probe. Methods and techniques for obtaining non-coding sequences using related coding sequence are well known to one skilled in the art.

The evolutionarily significant nucleotide changes, which are detected by molecular evolution analysis such as the K_{A}/K_{S} analysis, can be further assessed for their unique occurrence in the domesticated organism or the extent to which these changes are unique in the domesticated organism. For example, the identified changes in the domesticated gene can be tested for presence/absence in other sequences of related species, subspecies or other organisms having a common ancestor with the domesticated organism. This comparison (“outgroup analysis”) permits the determination of whether the positively selected gene is positively selected for in the domesticated organism at issue (as opposed to the ancestor).

For example, the identified changes in the EG307 gene were identified to various degrees in a number of O. sativa strains. See Tables 2-7. Additionally, a counterpart to EG307 was identified in maize, Zea mays mays, its wild ancestor, teosinte, Zea mays parviglumis, and also wild relatives of maize, Z. diploperennis and Z. luxurians. See Example 13 and Table 9. While EG307 in rice and maize was somewhat different at the nucleotide level, the protein sequences were more similar. Observing that rice and corn were independently domesticated from their wild ancestors, a consistent pattern emerges: the majority of the amino acid replacements in the modern crop (whether maize or rice), as compared to the ancestral plant (teosinte or ancestral rice) result in increased charge/polarity, increased solubility, and decreased hydrophobicity. This pattern is most unlikely to have occurred by chance in these two independent domestication events. This suggests that these replacements were a similar response to human imposed domestication. This is powerful evidence that EG307 has been selected as a result of human domestication of these two cereals.

The sequences with at least one evolutionarily significant change between a domesticated organism and its ancestor can be used as primers for PCR analysis of other ancestor protein-coding sequences, and resulting polynucleotides are sequenced to see whether the same change is present in other ancestors. These comparisons allow further discrimination as to whether the adaptive evolutionary changes are unique to the domesticated lineage as compared to other ancestors or whether the adaptive change is unique to the ancestor as compared to the domesticated species and other ancestors. A nucleotide change that is detected in the domesticated organism but not other ancestors more likely represents an adaptive evolutionary change in the domesticated organism. Alternatively, a nucleotide change that is detected in an ancestor that is not detected in the domesticated organism or other ancestors likely represents an ancestor adaptive evolutionary change. Other ancestors used for comparison can be selected based on their phylogenetic relationships with the domesticated organism. Statistical significance of such comparisons may be determined using established available programs, e.g., t-test as used by Messier and Stewart (1997) Nature 385:151-154. Those genes showing statistically high K_{A}/K_{S} ratios are very likely to have undergone adaptive evolution.

Sequences with significant changes can be used as probes in genomes from different domesticated populations to see whether the sequence changes are shared by more than one domesticated population. Gene sequences from different domesticated populations can be obtained from databases or, alternatively, from direct sequencing of PCR-amplified DNA from a number of unrelated, diverse domesticated populations. The presence of the identified changes in different domesticated populations would further indicate the evolutionary significance of the changes.

Sequences with significant changes between species can be further characterized in terms of their molecular/genetic identities and biological functions, using methods and techniques known to those of ordinary skill in the art. For example, the sequences can be located genetically and physically within the organism's genome using publicly available bio-informatics programs. The newly identified significant changes within the nucleotide sequence may suggest a potential role of the gene in the organism's evolution and a potential association with unique, enhanced or altered functional capabilities.

Using the techniques of the present invention, a heretofore unknown evolutionarily significant gene in rice, termed EG307, has been discovered as detailed in EXAMPLE 10. K_{A}/K_{S} analysis, performed as described in EXAMPLE 11 between O. rufipogon and certain O. sativa strains indicated an evolutionarily significant change as shown in Table 1. The gene has been positively selected. Using several different rice maps, as described in EXAMPLE 12, it was found that EG307 was within about 10 cM of marker RZ672, a marker associated with a QTL for 1000 grain weight residing on chromosome 3. (1000-grain weight is the weight (mass) of three different samples of 1000 randomly chosen fully filled grains of rice.) This is a sensitive measure of yield, which takes into account the individual variation in weight that occurs among rice grains. Thus, there only is about a 10% chance that the RZ672 marker will be separated from EG307 to crossing over in a single generation, strongly suggesting that EG307 plays an important role in controlling increased yield.

Also using the techniques of the present invention, a heretofore unknown evolutionarily significant gene in rice, termed EG1117, has been discovered as detailed in EXAMPLE 14. K_{A}/K_{S} analysis, performed as described in EXAMPLE 14 between O. rufipogon and certain O. sativa strains indicated an evolutionarily significant change as shown in Table 10. The gene has been positively selected. Using several different rice maps, as described in EXAMPLES 13 and 14, it was found that EG1117 lies on the same BAC as marker RZ672, a marker associated with a QTL for 1000 grain weight residing on chromosome 3. EG1117 lies about 2-3 cM from EG307.

From the combination of the evolutionarily significant K_{A}/K_{S} value and mapping data, one of skill in the art can reasonably conclude that EG307 and EG1117 are yield-related genes. EG307's and EG1117's yield-increasing function could be easily confirmed by making and growing a mutant or transgenic plant. Alternative methods include association analysis and pedigree analysis using the EG307 and EG117 sequence derived from rice, EG307 and EG117 genes from maize and its wild ancestor were obtained as detailed in EXAMPLE 13.

The putative gene with the identified sequences may be further characterized by, for example, homologue searching. Shared homology of the putative gene with a known gene may indicate a similar biological role or function. Another exemplary method of characterizing a putative gene sequence is on the basis of known sequence motifs. Certain sequence patterns are known to code for regions of proteins having specific biological characteristics such as signal sequences, DNA binding domains, or transmembrane domains.

The identified sequences with significant changes can also be further evaluated by looking at where the gene is expressed in terms of tissue- or cell type-specificity. For example, the identified coding sequences can be used as probes to perform in situ mRNA hybridization that will reveal the expression patterns of the sequences. Genes that are expressed in certain tissues may be better candidates as being associated with important functions associated with that tissue, for example developing endosperm tissue. The timing of the gene expression during each stage of development of a species member can also be determined.

As another exemplary method of sequence characterization, the functional roles of the identified nucleotide sequences with significant changes can be assessed by conducting functional assays for different alleles of an identified gene in the transfected domesticated organism, e.g., in the transgenic plant or animal. Current examples of plant functional assays include the use of microarrays, see Seki, et al., Monitoring the Expression Pattern of 1300 Arabidopsis Genes Under Drought and Cold Stresses Using a Full-Length cDNA Microarray. Plant Cell 13:61-72 (2001), and metabolite profiling, see Roessner, et al, Metabolic Profiling Allows Comprehensive Phenotyping of Genetically or Environmentally Modified Plant Systems. Plant Cell 13:11-29 (2001).

As another exemplary method of sequence characterization, the use of computer programs may allow modeling and visualizing the three-dimensional structure of the homologous proteins from domesticated organism and ancestor. Specific, exact knowledge of which amino acids have been replaced in the ancestor protein(s) allows detection of structural changes that may be associated with functional differences. Thus, use of modeling techniques is closely associated with identification of functional roles discussed in the previous paragraph. The use of individual or combinations of these techniques constitutes part of the present invention.

A domesticated organism's gene identified by the subject method can be used to identify homologous genes in other species that share a common ancestor. For example, maize, rice, wheat, millet, sorghum and other cereals share a common ancestor, and genes identified in rice can lead directly to homologous genes in these other grasses. Likewise, tomatoes and potatoes share a common ancestor, and genes identified in tomatoes by the subject method are expected to have homologues in potatoes, and vice versa.

The present invention also provides a method of detecting a yield-increasing gene in a plant cell comprising: a) contacting the EG307 gene or a portion thereof greater than 12 nucleotides, preferably greater than 30 nucleotides in length with a preparation of genomic DNA from the plant cell under hybridization conditions providing detection of nucleic acid molecule sequences having about 50% or greater sequence identity to the a nucleic acid molecule selected from the group consisting of SEQ ID NO:1, SEQ ID NO:91, SEQ ID. NO:2, SEQ ID NO:4, SEQ ID NO:5, SEQ ID NO:7, SEQ ID NO:10, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID. NO:23, SEQ ID NO:24, SEQ ID NO:25, SEQ ID NO:27, SEQ ID NO:28, SEQ ID NO:29, SEQ ID NO:30, SEQ ID NO:31, SEQ ID NO:33, SEQ ID NO:34, SEQ ID. NO:35, SEQ ID NO:37, SEQ ID NO:38, SEQ ID NO:40, SEQ ID NO:41, SEQ ID NO:42, SEQ ID NO:44, SEQ ID NO:45, SEQ ID NO:46, SEQ ID NO:47, SEQ ID NO:49, SEQ ID. NO:50, SEQ ID NO:51, SEQ ID NO:53, SEQ ID NO:54, SEQ ID NO:55, SEQ ID NO:57, SEQ ID NO:58, SEQ ID NO:60, SEQ ID NO:62, SEQ ID NO:63, SEQ ID NO:64, SEQ ID NO:66, SEQ ID NO:67, SEQ ID NO:69, SEQ ID. NO:70, SEQ ID NO:71, SEQ ID NO:73, SEQ ID NO:74, SEQ ID NO:75, SEQ ID NO:77, SEQ ID NO:59, SEQ ID NO:78, SEQ ID NO:80, SEQ ID NO:81, SEQ ID NO:82, SEQ ID NO:84 and SEQ ID NO:85; and b) detecting hybridization, whereby a yield-increasing gene may be identified.

The present invention also provides a method of isolating a yield-related gene from a recombinant plant cell library, comprising a) providing a preparation of plant cell DNA or a recombinant plant cell library; b) contacting the preparation or plant cell library with a detectably-labelled EG307 conserved oligonucleotide under hybridization conditions providing detection of genes having 50% or greater sequence identity; and c) isolating a yield-related gene by its association with the detectable label.

The present invention also provides a method of isolating a yield-related gene from plant cell DNA comprising a) providing a sample of plant cell DNA; b) providing a pair of oligonucleotides having sequence homology to a conserved region of an EG307 gene; c) combining the pair of oligonucleotides with the plant cell DNA sample under conditions suitable for polymerase chain reaction-mediated DNA amplification; and d) isolating the amplified yield-related gene or fragment thereof.

The sequences identified by the methods described herein can be used to identify agents that are useful in modulating domesticated organism-unique, enhanced or altered functional capabilities and/or correcting defects in these capabilities using these sequences. These methods employ, for example, screening techniques known in the art, such as in vitro systems, cell-based expression systems and transgenic animals and plants. The approach provided by the present invention not only identifies rapidly evolved genes, but indicates modulations that can be made to the protein that may not be too toxic because they exist in another species.

The present invention also provides a method of producing an EG307 polypeptide comprising: a) providing a cell transfected with a polynucleotide encoding an EG307 polypeptide positioned for expression in the cell; b) culturing the transfected cell under conditions for expressing the polynucleotide; and c) isolating the EG307 polypeptide.

The present invention also provides a method of detecting a yield-increasing gene in a plant cell comprising: a) contacting the EG1117 gene or a portion thereof greater than 12 nucleotides, preferably greater than 30 nucleotides in length with a preparation of genomic DNA from the plant cell under hybridization conditions providing detection of nucleic acid molecule sequences having about 50% or greater sequence identity to the a nucleic acid molecule selected from the group consisting of SEQ ID NO:92, SEQ ID NO:93, SEQ ID NO:94, SEQ ID NO:96, SEQ ID NO:97, SEQ ID NO:98, SEQ ID NO:100, SEQ ID NO:101, SEQ ID NO:102, SEQ ID NO:103, SEQ ID NO:104, SEQ ID NO:104, SEQ ID NO:106, SEQ ID NO:107, SEQ ID NO:109, SEQ ID NO:110, SEQ ID NO:112, SEQ ID NO:113, SEQ ID NO:114, SEQ ID NO:116, SEQ ID NO:117, SEQ ID NO:119, SEQ ID NO:120, SEQ ID NO:121, SEQ ID NO:122, SEQ ID NO:123, SEQ ID NO:124, SEQ ID NO:125, SEQ ID NO:127, SEQ ID NO:128, SEQ ID NO:129, SEQ ID NO:130, SEQ ID NO:131, SEQ ID NO:133, SEQ ID NO:135, SEQ ID NO:136, SEQ ID NO:137, SEQ ID NO:138, SEQ ID NO:140, SEQ ID NO:141, SEQ ID NO:142, SEQ ID NO:144, SEQ ID NO:145, SEQ ID NO:146, SEQ ID NO:147, SEQ ID NO:149, SEQ ID NO:150, SEQ ID NO:151, SEQ ID NO:152, SEQ ID NO:154, SEQ ID NO:155, SEQ ID NO:157, SEQ ID NO:158, SEQ ID NO:160, SEQ ID NO:161, SEQ ID NO:162, SEQ ID NO:163, SEQ ID NO:165, SEQ ID NO:166, SEQ ID NO:167, and SEQ ID NO:168; and b) detecting hybridization, whereby a yield-increasing gene may be identified.

The present invention also provides a method of isolating a yield-related gene from a recombinant plant cell library, comprising a) providing a preparation of plant cell DNA or a recombinant plant cell library; b) contacting the preparation or plant cell library with a detectably-labelled EG1117 conserved oligonucleotide under hybridization conditions providing detection of genes having 50% or greater sequence identity; and c) isolating a yield-related gene by its association with the detectable label.

The present invention also provides a method of isolating a yield-related gene from plant cell DNA comprising a) providing a sample of plant cell DNA; b) providing a pair of oligonucleotides having sequence homology to a conserved region of an EG1117 gene; c) combining the pair of oligonucleotides with the plant cell DNA sample under conditions suitable for polymerase chain reaction-mediated DNA amplification; and d) isolating the amplified yield-related gene or fragment thereof.

The sequences identified by the methods described herein can be used to identify agents that are useful in modulating domesticated organism-unique, enhanced or altered functional capabilities and/or correcting defects in these capabilities using these sequences. These methods employ, for example, screening techniques known in the art, such as in vitro systems, cell-based expression systems and transgenic animals and plants. The approach provided by the present invention not only identifies rapidly evolved genes, but indicates modulations that can be made to the protein that may not be too toxic because they exist in another species.

The present invention also provides a method of producing an EG1117 polypeptide comprising: a) providing a cell transfected with a polynucleotide encoding an EG1117 polypeptide positioned for expression in the cell; b) culturing the transfected cell under conditions for expressing the polynucleotide; and c) isolating the EG1117 polypeptide.

A. EG307 Polypeptides

One embodiment of the present invention is an isolated plant EG307 polypeptide. As used herein, an EG307 polypeptide, in one embodiment, is a polypeptide that is related to (i.e., bears structural similarity to) the O. sativa polypeptide of about 447 amino acids and having the sequence depicted in FIG. 2 (SEQ ID NO: 6). The original identification of such a polypeptide is detailed in the Examples. A preferred EG307 polypeptide is encoded by a polynucleotide that hybridizes under stringent hybridization conditions to at least one of the following genes: (a) a gene encoding an O. sativa EG307 polypeptide (i.e., an O. sativa gene); (b) a gene encoding an O. rufipogon EG307 polypeptide (i.e., an O. rufipogon gene); (c) a gene encoding a Zea mays mays EG307 gene; (d) a gene encoding a Zea mays parviglumis EG307 polypeptide (i.e., a. Z. mays parviglumis gene); (e) a gene encoding a Zea diploperennis EG307 polypeptide (i.e., a. Z. diploperennis gene); and (f) a gene encoding a Zea luxurians EG307 polypeptide (i.e., a. Z. luxurians gene). It is to be noted that the term “a” or “an” entity refers to one or more of that entity; for example, a gene refers to one or more genes or at least one gene. As such, the terms “a” (or “an”), “one or more” and “at least one” can be used interchangeably herein. It is also to be noted that the terms “comprising,” “including,” and “having” can be used interchangeably.

As used herein, stringent hybridization conditions refer to standard hybridization conditions under which polynucleotides, including oligonucleotides, are used to identify molecules having similar nucleic acid sequences. Such standard conditions are disclosed, for example, in Sambrook et al., MOLECULAR CLONING: A LABORATORY MANUAL, Cold Spring Harbor Labs Press, 1989. Examples of such conditions are provided in the Examples section of the present application.

As used herein, an O. sativa EG307 gene includes all nucleic acid sequences related to a natural O. sativa EG307 gene such as regulatory regions that control production of the O. sativa EG307 polypeptide encoded by that gene (such as, but not limited to, transcription, translation or post-translation control regions) as well as the coding region itself. In one embodiment, an O. sativa EG307 gene includes the nucleic acid sequence SEQ ID NO:4. Nucleic acid sequence SEQ ID NO:4 represents the deduced sequence of a cDNA (complementary DNA) polynucleotide, the production of which is disclosed in the Examples. It should be noted that since nucleic acid sequencing technology is not entirely error-free, SEQ ID NO:4 (as well as other sequences presented herein), at best, represents an apparent nucleic acid sequence of the polynucleotide encoding an O. sativa EG307 polypeptide of the present invention.

In another embodiment, an O. sativa EG307 gene can be an allelic variant that includes a similar but not identical sequence to SEQ ID NO:4. An allelic variant of an O. sativa EG307 gene including SEQ ID NO: 1 is a locus (or loci) in the genome whose activity is concerned with the same biochemical or developmental processes, and/or a gene that that occurs at essentially the same locus as the gene including SEQ ID NO:4, but which, due to natural variations caused by, for example, mutation or recombination, has a similar but not identical sequence. Because genomes can undergo rearrangement, the physical arrangement of alleles is not always the same. Allelic variants typically encode polypeptides having similar activity to that of the polypeptide encoded by the gene to which they are being compared. Allelic variants can also comprise alterations in the 5′ or 3′ untranslated regions of the gene (e.g., in regulatory control regions). Allelic variants are well known to those skilled in the art and would be expected to be found within a given rice cultivar or strain since the genome is diploid and/or among a population comprising two or more rice cultivars or strains. For example, it is believed that the O. sativa polynucleotide having nucleic acid sequences represented by SEQ ID NO:18, to be described in more detail below, represents allelic variants of the Kasalath strain of O. sativa.

Similarly, a Zea mays mays EG307 gene includes all nucleic acid sequences related to a natural Z. mays mays EG307 gene such as regulatory regions that control production of the Z. mays mays EG307 polypeptide encoded by that gene as well as the coding region itself. In one embodiment, a Zea mays mays EG307 gene includes the nucleic acid sequence SEQ ID NO:66. Nucleic acid sequence SEQ ID NO:66 represents the deduced sequence of a cDNA polynucleotide, the production of which is disclosed in the Examples. In another embodiment, a Zea mays mays EG307 gene can be an allelic variant that includes a similar but not identical sequence to SEQ ID NO:66.

According to the present invention, an isolated, or biologically pure, polypeptide, is a polypeptide that has been removed from its natural milieu. As such, “isolated” and “biologically pure” do not necessarily reflect the extent to which the polypeptide has been purified. An isolated EG307 polypeptide of the present invention can be obtained from its natural source, can be produced using recombinant DNA technology or can be produced by chemical synthesis. An EG307 polypeptide of the present invention may be identified by its ability to perform the function of natural EG307 in a functional assay. By “natural EG307 polypeptide,” it is meant the full length EG307 polypeptide of O. sativa, O. rufipogon, Z. mays mays, and/or Z. mays parviglumis. The phrase “capable of performing the function of a natural EG307 in a functional assay” means that the polypeptide has at least about 10% of the activity of the natural polypeptide in the functional assay. In other preferred embodiments, the EG307 polypeptide has at least about 20% of the activity of the natural polypeptide in the functional assay. In other preferred embodiments, the EG307 polypeptide has at least about 30% of the activity of the natural polypeptide in the functional assay. In other preferred embodiments, the EG307 polypeptide has at least about 40% of the activity of the natural polypeptide in the functional assay. In other preferred embodiments, the EG307 polypeptide has at least about 50% of the activity of the natural polypeptide in the functional assay. In other preferred embodiments, the polypeptide has at least about 60% of the activity of the natural polypeptide in the functional assay. In more preferred embodiments, the polypeptide has at least about 70% of the activity of the natural polypeptide in the functional assay. In more preferred embodiments, the polypeptide has at least about 80% of the activity of the natural polypeptide in the functional assay. In more preferred embodiments, the polypeptide has at least about 90% of the activity of the natural polypeptide in the functional assay. Examples of functional assays include antibody-binding assays, or yield-increasing assays, as detailed elsewhere in this specification.

As used herein, an isolated plant EG307 polypeptide can be a full-length polypeptide or any homologue of such a polypeptide. Examples of EG307 homologues include EG307 polypeptides in which amino acids have been deleted (e.g., a truncated version of the polypeptide, such as a peptide), inserted, inverted, substituted and/or derivatized (e.g., by glycosylation, phosphorylation, acetylation, myristylation, prenylation, palmitoylation, amidation and/or addition of glycerophosphatidyl inositol) such that the homolog has natural EG307 activity.

In one embodiment, when the homologue is administered to an animal as an immunogen, using techniques known to those skilled in the art, the animal will produce a humoral and/or cellular immune response against at least one epitope of a natural EG307 polypeptide. EG307 homologues can also be selected by their ability to perform the function of EG307 in a functional assay.

Plant EG307 polypeptide homologues can be the result of natural allelic variation or natural mutation. EG307 polypeptide homologues of the present invention can also be produced using techniques known in the art including, but not limited to, direct modifications to the polypeptide or modifications to the gene encoding the polypeptide using, for example, classic or recombinant DNA techniques to effect random or targeted mutagenesis.

In accordance with the present invention, a mimetope refers to any compound that is able to mimic the ability of an isolated plant EG307 polypeptide of the present invention to perform the function of an EG307 polypeptide of the present invention in a functional assay. Examples of mimetopes include, but are not limited to, anti-idiotypic antibodies or fragments thereof, that include at least one binding site that mimics one or more epitopes of an isolated polypeptide of the present invention; non-polypeptideaceous immunogenic portions of an isolated polypeptide (e.g., carbohydrate structures); and synthetic or natural organic molecules, including nucleic acids, that have a structure similar to at least one epitope of an isolated polypeptide of the present invention. Such mimetopes can be designed using computer-generated structures of polypeptides of the present invention. Mimetopes can also be obtained by generating random samples of molecules, such as oligonucleotides, peptides or other organic molecules, and screening such samples by affinity chromatography techniques using the corresponding binding partner.

The minimal size of an EG307 polypeptide homologue of the present invention is a size sufficient to be encoded by a polynucleotide capable of forming a stable hybrid with the complementary sequence of a polynucleotide encoding the corresponding natural polypeptide. As such, the size of the polynucleotide encoding such a polypeptide homologue is dependent on nucleic acid composition and percent homology between the polynucleotide and complementary sequence as well as upon hybridization conditions per se (e.g., temperature, salt concentration, and formamide concentration). It should also be noted that the extent of homology required to form a stable hybrid can vary depending on whether the homologous sequences are interspersed throughout the polynucleotides or are clustered (i.e., localized) in distinct regions on the polynucleotides. The minimal size of such polynucleotides is typically at least about 12 to about 15 nucleotides in length if the polynucleotides are GC-rich and at least about 15 to about 17 bases in length if they are AT-rich. Preferably, the polynucleotide is at least 12 bases in length.

As such, the minimal size of a polynucleotide used to encode an EG307 polypeptide homologue of the present invention is from about 12 to about 18 nucleotides in length. There is no limit, other than a practical limit, on the maximal size of such a polynucleotide in that the polynucleotide can include a portion of a gene, an entire gene, or multiple genes, or portions thereof. Similarly, the minimal size of an EG307 polypeptide homologue of the present invention is from about 4 to about 6 amino acids in length, with preferred sizes depending on whether a full-length, fusion, multivalent, or functional portions of such polypeptides are desired. Preferably, the polypeptide is at least 30 amino acids in length.

Any plant EG307 polypeptide is a suitable polypeptide of the present invention. Suitable plants from which to isolate EG307 polypeptides (including isolation of the natural polypeptide or production of the polypeptide by recombinant or synthetic techniques) include maize, wheat, barley, rye, millet, chickpea, lentil, flax, olive, fig almond, pistachio, walnut, beet, parsnip, citrus fruits, including, but not limited to, orange, lemon, lime, grapefruit, tangerine, minneola, and tangelo, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugarbeet, sunflower, rapeseed, clover, tobacco, carrot, cotton, alfalfa, rice, potato, eggplant, cucumber, Arabidopsis, and woody plants such as coniferous and deciduous trees, with rice and maize being preferred. Preferred rice plants from which to isolate EG307 polypeptides include Nipponbare1 and 2, Lemont, IR64, Teqing, Azucena, and Kasalath 1, 2, 3, and 4 strains of O. sativa.

A preferred plant EG307 polypeptide of the present invention is a compound that when expressed or modulated in a plant, is capable of increasing the yield of the plant.

One embodiment of the present invention is a fusion polypeptide that includes an EG307 polypeptide-containing domain attached to a fusion segment. Inclusion of a fusion segment as part of a EG307 polypeptide of the present invention can enhance the polypeptide's stability during production, storage and/or use. Depending on the segment's characteristics, a fusion segment can also act as an immunopotentiator to enhance the immune response mounted by an animal immunized with an EG307 polypeptide containing such a fusion segment. Furthermore, a fusion segment can function as a tool to simplify purification of an EG307 polypeptide, such as to enable purification of the resultant fusion polypeptide using affinity chromatography. A suitable fusion segment can be a domain of any size that has the desired function (e.g., imparts increased stability, imparts increased immunogenicity to a polypeptide, and/or simplifies purification of a polypeptide). It is within the scope of the present invention to use one or more fusion segments. Fusion segments can be joined to amino and/or carboxyl termini of the EG307-containing domain of the polypeptide. Linkages between fusion segments and EG307-containing domains of fusion polypeptides can be susceptible to cleavage in order to enable straightforward recovery of the EG307-containing domains of such polypeptides. Fusion polypeptides are preferably produced by culturing a recombinant cell transformed with a fusion polynucleotide that encodes a polypeptide including the fusion segment attached to either the carboxyl and/or amino terminal end of a EG307-containing domain.

Preferred fusion segments for use in the present invention include a glutathione binding domain; a metal binding domain, such as a poly-histidine segment capable of binding to a divalent metal ion; an immunoglobulin binding domain, such as Polypeptide A, Polypeptide G, T cell, B cell, Fc receptor or complement polypeptide antibody-binding domains; a sugar binding domain such as a maltose binding domain from a maltose binding polypeptide; and/or a “tag” domain (e.g., at least a portion of β-galactosidase, a strep tag peptide, other domains that can be purified using compounds that bind to the domain, such as monoclonal antibodies). More preferred fusion segments include metal binding domains, such as a poly-histidine segment; a maltose binding domain; a strep tag peptide.

Preferred plant EG307 polypeptides of the present invention are rice EG307 polypeptides and maize EG307 polypeptides. More preferred EG307 polypeptides are O. sativa, O. rufipogon, Z. mays mays, Zea mays parviglumis, Z. diploperennis and Z. luxurians EG307 polypeptides. O. sativa strains include Nipponbare, Azucena, Kasalath 1, 2, 3, and 4, Teqing, Lemont, and IR64. Z. mays parviglumis strains include Benz, BK4, IA19, and Wilkes. Z. mays mays strains include BS7, HuoBai, Makki, Min13, Pira, Sari, Smena, and W22.

One preferred O. sativa EG307 polypeptide of the present invention is a polypeptide encoded by an O. sativa polynucleotide that hybridizes under stringent hybridization conditions with complements of polynucleotides represented by SEQ ID NO:1, SEQ ID NO:91, SEQ ID. NO:2, SEQ ID NO:4, SEQ ID NO:5, SEQ ID NO:7, SEQ ID NO:10, SEQ ID NO:11, SEQ ID NO:12, SEQ ID NO:14, SEQ ID NO:15, SEQ ID NO:17, and/or SEQ ID NO:18. Such an EG307 polypeptide is encoded by a polynucleotide that hybridizes under stringent hybridization conditions with a polynucleotide having nucleic acid sequence SEQ ID NO:1, SEQ ID NO:91, SEQ ID. NO:2, SEQ ID NO:4, SEQ ID NO:5, SEQ ID NO:7, SEQ ID NO:10, SEQ ID NO:11, SEQ ID NO:12, SEQ ID NO:14, SEQ ID NO:15, SEQ ID NO:17, and/or SEQ ID NO:18.

Inspection of EG307 genomic nucleic acid sequences indicates that the genes comprise several regions, including a first exon region, a first intron region, a second exon region, a second intron region, and a third exon region.

Polynucleotides SEQ ID NO:4 and SEQ ID NO:91 represent the 5′ and 3′ ends of the EG307 gene in O. sativa (cv. Nipponbare). SEQ ID NO:4 and SEQ ID NO:91 are joined by a number of nucleotides, the exact number of which is unknown due to potential insertions/deletions in the non-coding portions of the gene, but is believed to be about 6. Translation of SEQ ID NO:4 and SEQ ID NO:91 suggests that the O. sativa EG307 polynucleotide includes an open reading frame. The reading frame encodes an O. sativa EG307 polypeptide of about 447 amino acids, the deduced amino acid sequence of which is represented herein as SEQ ID NO:6, assuming an open reading frame having an initiation (start) codon spanning from about nucleotide 37 through about nucleotide 39 of SEQ ID NO:4 and a termination (stop) codon spanning from about nucleotide 2278 through about nucleotide 2280 of SEQ ID NO:4, with the first exon spanning nucleotides 1-126 of SEQ ID NO: 4, the first intron spanning nucleotides 9-822 of SEQ ID NO:91, the second exon spanning nucleotides 823-1141 of SEQ ID NO:91, the second intron spanning nucleotides 1142-1222 of SEQ ID NO:91, and the third exon spanning nucleotides 1223-2157 of SEQ ID NO:91. The open reading frame from nucleotide 37 through about nucleotide 2280 of SEQ ID NO:4 is represented herein as SEQ ID NO:5.

Similarly, translation of O. sativa (strain Azucena) polynucleotide SEQ ID NO:1 suggests an open reading frame from about nucleotide 3 to about nucleotide 2410 of SEQ ID NO:1, with the first exon spanning nucleotides 1-92 of SEQ ID NO: 1, the first intron spanning nucleotides 93-1075 of SEQ ID NO:1, the second exon spanning nucleotides 1076-1394 of SEQ ID NO:1, the second intron spanning nucleotides 1395-1475 of SEQ ID NO:1, and the third exon spanning nucleotides 1476-2441 of SEQ ID NO:1. The open reading frame is represented herein as SEQ ID NO:2, and encodes a polypeptide represented herein as SEQ ID NO:3.

Similarly, translation of O. sativa (strain Teqing) polynucleotide SEQ ID NO:7 suggests an open reading frame from about nucleotide 21 to about nucleotide 2421, with the first exon spanning nucleotides 1-110 of SEQ ID NO:7, the first intron spanning nucleotides 111-1089 of SEQ ID NO:7, the second exon spanning nucleotides 1090-1405 of SEQ ID NO:7, the second intron spanning nucleotides 1406-1486 of SEQ ID NO:7, and the third exon spanning nucleotides 1487-2461 of SEQ ID NO:7. The open reading frame is represented herein as SEQ ID NO:8, and encodes a polypeptide represented herein as SEQ ID NO:9.

Similarly, polynucleotides SEQ ID NO:10 and SEQ ID NO:11 represent the 5′ and 3′ ends of the EG307 gene in O. sativa (strain Lemont). SEQ ID NO:10 and SEQ ID NO:11 are joined by an unknown number of nucleotides. In the genomic sequence, there may be insertions/deletions in the non-coding portions of the gene, thus the actual number of nucleotides is unknown, but is believed to be about 10. Translation of O. sativa (strain Lemont) polynucleotides SEQ ID NO:10 and SEQ ID NO:11 suggests an open reading frame from about nucleotide 166 of SEQ ID NO: 10 to about nucleotide 1547 of SEQ ID NO:11, with the first exon spanning nucleotides 1-255 of SEQ ID NO:10, the first intron spanning nucleotides 255-451 of SEQ ID NO:10 and nucleotides 1-212 of SEQ ID NO:11, the second exon spanning nucleotides 213-531 of SEQ ID NO:11, the second intron spanning nucleotides 532-612 of SEQ ID NO:11, and the third exon spanning nucleotides 613-1616 of SEQ ID NO:11. The open reading frame is represented herein as SEQ ID NO:12, and encodes a polypeptide represented herein as SEQ ID NO:13.

Similarly, translation of O. sativa (strain IR64) polynucleotide SEQ ID NO:14 suggests an open reading frame from about nucleotide 1 to about nucleotide 2400, with the first exon spanning nucleotides 1-90 of SEQ ID NO:14, the first intron spanning nucleotides 91-1068 of SEQ ID NO:14, the second exon spanning nucleotides 1069-1384 of SEQ ID NO:14, the second intron spanning nucleotides 1385-1465 of SEQ ID NO:14, and the third exon spanning nucleotides 1466-2459 of SEQ ID NO:11. The open reading frame is represented herein as SEQ ID NO:14, and encodes a polypeptide represented herein as SEQ ID NO:15.

Similarly, translation of O. sativa (strain Kasalath) polynucleotide SEQ ID NO:17 suggests an open reading frame from about nucleotide 2 to about nucleotide 2402, with the first exon spanning nucleotides 1-91 of SEQ ID NO:17, the first intron spanning nucleotides 92-1070 of SEQ ID NO:17, the second exon spanning nucleotides 1071-1386 of SEQ ID NO:17, the second intron spanning nucleotides 1387-1467 of SEQ ID NO:17, and the third exon spanning nucleotides 1468-2432 of SEQ ID NO:17.

The open reading frame is represented as SEQ ID NO:18, and encodes a polypeptide represented herein as SEQ ID NO:19. In SEQ ID NO:18, “N” at position 889 is “G”, and “N” at position 971 is “A” for strain Kasalath 1, making amino acid residue 297 in SEQ ID NO:19 a valine, and amino acid residue 324 a glutamine. In SEQ ID NO: 18, “N” at position 889 is “G”, and “N” at position 971 is “T” for strain Kasalath 2, making amino acid residue 297 in SEQ ID NO:19 a valine, and amino acid residue 324 a leucine. In SEQ ID NO: 18, “N” at position 889 is “C”, and “N” at position 971 is “A” for strain Kasalath 3, making amino acid residue 297 in SEQ ID NO:19 a leucine, and amino acid residue 324 a glutamine. In SEQ ID NO: 18, “N” at position 889 is “C”, and “N” at position 971 is “T” for strain Kasalath 4, making amino acid residue 297 in SEQ ID NO:19 a leucine, and amino acid residue 324 a leucine.

A preferred O. sativa EG307 polypeptide of the present invention is a polypeptide encoded by a polynucleotide that hybridizes under stringent hybridization conditions with polynucleotides represented by SEQ ID NO:1, SEQ ID NO:91, SEQ ID. NO:2, SEQ ID NO:4, SEQ ID NO:5, SEQ ID NO:7, SEQ ID NO:10, SEQ ID NO:11, SEQ ID NO:12, SEQ ID NO:14, SEQ ID NO:15, SEQ ID NO:17, and/or SEQ ID NO:18.

Preferred O. rufipogon EG307 polypeptides of the present invention are polypeptides encoded by an O. rufipogon polynucleotide that hybridizes under stringent hybridization conditions with complements of polynucleotides represented by SEQ ID NO:20, SEQ ID NO:21, SEQ ID. NO:23, SEQ ID NO:24, SEQ ID NO:25, SEQ ID NO:27, SEQ ID NO:28, SEQ ID NO:29, SEQ ID NO:30, and/or SEQ ID NO:31. Such an EG307 polypeptide is encoded by a polynucleotide that hybridizes under stringent hybridization conditions with a polynucleotide having nucleic acid sequence SEQ ID NO:20, SEQ ID NO:21, SEQ ID. NO:23, SEQ ID NO:24, SEQ ID NO:25, SEQ ID NO:27, SEQ ID NO:28, SEQ ID NO:29, SEQ ID NO:30, and/or SEQ ID NO:31.

Polynucleotides SEQ ID NO:27 and SEQ ID NO:28 represent the 5′ and 3′ ends of the EG307 gene in O. rufipogon (strain 5953). SEQ ID NO:27 and SEQ ID NO:28 are joined by a number of nucleotides, the exact number of which is unknown due to potential insertions/deletions in the non-coding portions of the gene, but is believed to be about 23. Translation of SEQ ID NO:27 and SEQ ID NO:28 suggests that the O. rufipogon EG307 polynucleotide includes an open reading frame. The reading frame encodes an O. rufipogon EG307 polypeptide of about 446 amino acids, the deduced amino acid sequence of which is represented herein as SEQ ID NO:30, assuming an open reading frame having an initiation (start) codon spanning from about nucleotide 18 through about nucleotide 20 of SEQ ID NO:27 and a termination (stop) codon spanning from about nucleotide 1330 through about nucleotide 1332 of SEQ ID NO:28, with the first exon spanning nucleotides 1-107 of SEQ ID NO:27, no first intron, the second exon spanning nucleotides 1-316 of SEQ ID NO:28, the second intron spanning nucleotides 317-397 of SEQ ID NO:28, and the third exon spanning nucleotides 398-1332 of SEQ ID NO:28. The open reading frame from nucleotide 18 of SEQ ID NO:27 through about nucleotide 1332 of SEQ ID NO:28 is represented herein as SEQ ID NO:29.

Similarly, translation of O. rufipogon (strain 5948) polynucleotide SEQ ID NO:20 suggests an open reading frame from about 15 nucleotides 5′ of nucleotide 1 to about nucleotide 2385, first exon not represented, the first intron spanning nucleotides 1-1053 of SEQ ID NO:20, the second exon spanning nucleotides 1054-1369 of SEQ ID NO:20, the second intron spanning nucleotides 1370-1450 of SEQ ID NO:20, and the third exon spanning nucleotides 1451-2447 of SEQ ID NO:20. The open reading frame is represented herein as SEQ ID NO:21, and encodes a polypeptide represented herein as SEQ ID NO:22.

Similarly, polynucleotides SEQ ID NO:23 and SEQ ID NO:24 represent the 5′ and 3′ ends of the EG307 gene in O. rufipogon (strain 5949). SEQ ID NO:23 and SEQ ID NO:24 are joined by a number of nucleotides, the exact number of which is unknown due to potential insertions/deletions in the non-coding portions of the gene, but is believed to be about 13. Translation of SEQ ID NO:23 and SEQ ID NO:24 suggests an open reading frame from about nucleotide 57 of SEQ ID NO:23 to about nucleotide 1562 of SEQ ID NO:24, with the first exon spanning nucleotides 1-146 of SEQ ID NO:23, the first intron spanning nucleotides 1-230 of SEQ ID NO:24, the second exon spanning nucleotides 231-546 of SEQ ID NO:24, the second intron spanning nucleotides 547-627 of SEQ ID NO:24, and the third exon spanning nucleotides 628-1615 of SEQ ID NO:24. The open reading frame is represented as SEQ ID NO:25, and encodes a polypeptide represented herein as SEQ ID NO:26.

Similarly, translation of O. rufipogon (strain IRCG 105491) polynucleotide SEQ ID NO:90 suggests an open reading frame from about nucleotide 1 to about nucleotide 1341. The open reading frame is represented herein as SEQ ID NO:31 encoding a polypeptide represented herein as SEQ ID NO:32.

A preferred O. rufipogon EG307 polypeptide of the present invention is a polypeptide encoded by a polynucleotide that hybridizes under stringent hybridization conditions with a polynucleotide represented by SEQ ID NO:20, SEQ ID NO:21, SEQ ID. NO:23, SEQ ID NO:24, SEQ ID NO:25, SEQ ID NO:27, SEQ ID NO:28, SEQ ID NO:29, SEQ ID NO:30, and/or SEQ ID NO:31.

One preferred Zea mays parviglumis EG307 polypeptide of the present invention is a polypeptide encoded by a Zea mays parviglumis polynucleotide that hybridizes under stringent hybridization conditions with complements of polynucleotides represented by SEQ ID NO:66, SEQ ID NO:67, SEQ ID NO:69, SEQ ID. NO:70, SEQ ID NO:71, SEQ ID NO:73, SEQ ID NO:74, SEQ ID NO:75, SEQ ID NO:77, SEQ ID NO:59, and/or SEQ ID NO:78. Such an EG307 polypeptide is encoded by a polynucleotide that hybridizes under stringent hybridization conditions with a polynucleotide having nucleic acid sequence SEQ ID NO:66, SEQ ID NO:67, SEQ ID NO:69, SEQ ID. NO:70, SEQ ID NO:71, SEQ ID NO:73, SEQ ID NO:74, SEQ ID NO:75, SEQ ID NO:77, SEQ ID NO:59, and/or SEQ ID NO:78.

Translation of SEQ ID NO:66 suggests that the Zea mays parviglumis EG307 polynucleotide (strain Benz) includes an open reading frame. The reading frame encodes an Zea mays parviglumis EG307 polypeptide of about 448 amino acids, the deduced amino acid sequence of which is represented herein as SEQ ID NO:68, assuming an open reading frame having an initiation (start) codon spanning from about nucleotide 1 through about nucleotide 3 of SEQ ID NO:66 and a termination (stop) codon spanning from about nucleotide 2569 through about nucleotide 2571 of SEQ ID NO:66, with the first exon spanning nucleotides 1-81 of SEQ ID NO:66, the first intron spanning nucleotides 82-1204 of SEQ ID NO:66, the second exon spanning nucleotides 1205-1517 of SEQ ID NO:66, the second intron spanning nucleotides 1518-1618 of SEQ ID NO:66, and the third exon spanning nucleotides 1619-2644 of SEQ ID NO:66. The open reading frame from nucleotide 3 through about nucleotide 2571 of SEQ ID NO:66 is represented herein as SEQ ID NO:67.

Similarly, polynucleotides SEQ ID NO:69 and SEQ ID NO:70 represent the 5′ and 3′ ends of the EG307 gene in Z. mays parviglumis (strain BK4). SEQ ID NO:69 and SEQ ID NO:70 are joined by a number of nucleotides, the exact number of which is unknown due to potential insertions/deletions in the non-coding portions of the gene, but is believed to be about 10. Translation of Z. mays parviglumis (strain BK4) polynucleotide SEQ ID NO:69 and SEQ ID NO:70 suggests an open reading frame from about nucleotide 10 of SEQ ID NO:69 to about nucleotide 1728 of SEQ ID NO:70, with the first exon spanning nucleotides 1-90 of SEQ ID NO:69, the first intron spanning nucleotides 91-586 of SEQ ID NO:69 and nucleotides 1-361 of SEQ ID NO:70, the second exon spanning nucleotides 362-674 of SEQ ID NO:70, the second intron spanning nucleotides 675-775 of SEQ ID NO:70, and the third exon spanning nucleotides 776-1775 of SEQ ID NO:11. The open reading frame is represented as SEQ ID NO:71, and encodes a polypeptide represented herein as SEQ ID NO:72.

Similarly, polynucleotides SEQ ID NO:73 and SEQ ID NO:74 represent the 5′ and 3′ ends of the EG307 gene in Z. mays parviglumis (strain IA19). SEQ ID NO:73 and SEQ ID NO:74 are joined by a number of nucleotides, the exact number of which is unknown due to potential insertions/deletions in the non-coding portions of the gene, but is believed to be about 12. Translation of Z. mays parviglumis (strain IA19) polynucleotides SEQ ID NO:73 and SEQ ID NO:74 suggests an open reading frame from about nucleotide 69 of SEQ ID NO:73 to about nucleotide 1280 of SEQ ID NO:74, with the first exon spanning nucleotides 1-149 of SEQ ID NO:73, the first intron spanning nucleotides 150-305 of SEQ ID NO:73, the second exon spanning nucleotides 1-226 of SEQ ID NO:74, the second intron spanning nucleotides 227-327 of SEQ ID NO:74, and the third exon spanning nucleotides 328-1309 of SEQ ID NO:74. The open reading frame is represented herein as SEQ ID NO:75, and encoding a polypeptide represented herein as SEQ ID NO:76.

Similarly, polynucleotides SEQ ID NO:77 and SEQ ID NO:59 represent the 5′ and 3′ ends of the EG307 gene in Z. mays parviglumis (strain Wilkes). SEQ ID NO:77 and SEQ ID NO:59 are joined by a number of nucleotides, the exact number of which is unknown due to potential insertions/deletions in the non-coding portions of the gene, but is believed to be about 14. Translation of Z. mays parviglumis (strain Wilkes) polynucleotide SEQ ID NO:77 and SEQ ID NO:59 suggests an open reading frame from about nucleotide 36 of SEQ ID NO:77 to about nucleotide 1598 of SEQ ID NO:59, with the first exon spanning nucleotides 1-86 of SEQ ID NO:77, the first intron spanning nucleotides 1-231 of SEQ ID NO:59, the second exon spanning nucleotides 232-544 of SEQ ID NO:59, the second intron spanning nucleotides 545-645 of SEQ ID NO:59, and the third exon spanning nucleotides 656-1640 of SEQ ID NO:59. The open reading frame is represented herein as SEQ ID NO:78, and encoding a polypeptide represented herein as SEQ ID NO:79.

A preferred EG307 polypeptide of the present invention is a polypeptide encoded by a polynucleotide that hybridizes under stringent hybridization conditions with a polynucleotide represented by SEQ ID NO:33, SEQ ID NO:34, SEQ ID. NO:35, SEQ ID NO:37, SEQ ID NO:38, SEQ ID NO:40, SEQ ID NO:41, SEQ ID NO:42, SEQ ID NO:44, SEQ ID NO:45, SEQ ID NO:46, SEQ ID NO:47, SEQ ID NO:49, SEQ ID. NO:50, SEQ ID NO:51, SEQ ID NO:53, SEQ ID NO:54, SEQ ID NO:55, SEQ ID NO:57, SEQ ID NO:58, SEQ ID NO:60, SEQ ID NO:62, SEQ ID NO:63, and/or SEQ ID NO:64.

One preferred Zea mays mays EG307 polypeptide of the present invention is a polypeptide encoded by an Zea mays mays polynucleotide that hybridizes under stringent hybridization conditions with complements of polynucleotides represented by SEQ ID NO:33, SEQ ID NO:34, SEQ ID. NO:35, SEQ ID NO:37, SEQ ID NO:38, SEQ ID NO:40, SEQ ID NO:41, SEQ ID NO:42, SEQ ID NO:44, SEQ ID NO:45, SEQ ID NO:46, SEQ ID NO:47, SEQ ID NO:49, SEQ ID. NO:50, SEQ ID NO:51, SEQ ID NO:53, SEQ ID NO:54, SEQ ID NO:55, SEQ ID NO:57, SEQ ID NO:58, SEQ ID NO:60, SEQ ID NO:62, SEQ ID NO:63, and/or SEQ ID NO:64. Such an EG307 polypeptide is encoded by a polynucleotide that hybridizes under stringent hybridization conditions with a polynucleotide having nucleic acid sequence SEQ ID NO:33, SEQ ID NO:34, SEQ ID. NO:35, SEQ ID NO:37, SEQ ID NO:38, SEQ ID NO:40, SEQ ID NO:41, SEQ ID NO:42, SEQ ID NO:44, SEQ ID NO:45, SEQ ID NO:46, SEQ ID NO:47, SEQ ID NO:49, SEQ ID. NO:50, SEQ ID NO:51, SEQ ID NO:53, SEQ ID NO:54, SEQ ID NO:55, SEQ ID NO:57, SEQ ID NO:58, SEQ ID NO:59, SEQ ID NO:60, SEQ ID NO:62, SEQ ID NO:63, and/or SEQ ID NO:64.

Polynucleotides SEQ ID NO:33 and SEQ ID NO:34 represent the 5′ and 3′ ends of the EG307 gene in Z. mays mays (strain BS 7). SEQ ID NO:33 and SEQ ID NO:34 are joined by a number of nucleotides, the exact number of which is unknown due to potential insertions/deletions in the non-coding portions of the gene, but is believed to be about 21.

Translation of SEQ ID NO:33 and SEQ ID NO:34 suggests that the Zea mays mays EG307 polynucleotide includes an open reading frame. The reading frame encodes an Zea mays mays EG307 polypeptide of about 448 amino acids, the deduced amino acid sequence of which is represented herein as SEQ ID NO:36, assuming an open reading frame having an initiation (start) codon spanning from about nucleotide 3 through about nucleotide 5 of SEQ ID NO:33 and a termination (stop) codon spanning from about nucleotide 1396 through about nucleotide 1398 of SEQ ID NO:34, with the first exon spanning nucleotides 1-83 of SEQ ID NO:33, the first intron spanning nucleotides 84-180 of SEQ ID NO:33 and nucleotides 1-31 of SEQ ID NO:34, the second exon spanning nucleotides 32-344 of SEQ ID NO:34, the second intron spanning nucleotides 345-445 of SEQ ID NO:34, and the third exon spanning nucleotides 446-1447 of SEQ ID NO:34. The open reading frame from nucleotide 3 of SEQ ID NO:33 through about nucleotide 1398 of SEQ ID NO:34 is represented herein as SEQ ID NO:35.

Similarly, translation of Z. mays mays (strain HuoBai) polynucleotide SEQ ID NO:37 suggests an open reading frame from about nucleotide 28 to about nucleotide 2599, with the first exon spanning nucleotides 1-108 of SEQ ID NO:37, the first intron spanning nucleotides 109-1232 of SEQ ID NO:37, the second exon spanning nucleotides 1233-1545 of SEQ ID NO:37, the second intron spanning nucleotides 1546-1646 of SEQ ID NO:37, and the third exon spanning nucleotides 1647-2646 of SEQ ID NO:37. The open reading frame is represented herein as SEQ ID NO:38, and encodes a polypeptide represented herein as SEQ ID NO:39.

Similarly, polynucleotides SEQ ID NO:40 and SEQ ID NO:41 represent 5′ end to the 3′ end of the EG307 gene in Z. mays mays (strain Makki). SEQ ID NO:40 and SEQ ID NO:41 are joined by a number of nucleotides, the exact number of which is unknown due to potential insertions/deletions in the non-coding portions of the gene, but is believed to be about 20. Translation of Z. mays mays (strain Makki) polynucleotides SEQ ID NO:40 and SEQ ID NO:41 suggests an open reading frame from about nucleotide 61 of SEQ ID NO:40 to about nucleotide 2263 of SEQ ID NO:41, with the first exon spanning nucleotides 1-141 of SEQ ID NO:40, the first intron spanning nucleotides 142-262 of SEQ ID NO:40 and nucleotides 1-896 of SEQ ID NO:41, the second exon spanning nucleotides 897-1209 of SEQ ID NO:41, the second intron spanning nucleotides 1210-1310 of SEQ ID NO:41, and the third exon spanning nucleotides 1311-2311 of SEQ ID NO:41. The open reading frame is represented as SEQ ID NO:42 encoding a polypeptide represented herein as SEQ ID NO:43.

Similarly, polynucleotides SEQ ID NO:44, SEQ ID NO:45 and SEQ ID NO:46 represent the three parts of the EG307 gene in Z. mays mays (strain Min13), from the 5′ end to the 3′ end. SEQ ID NO:44, SEQ ID NO:45 and SEQ ID NO:46 are joined by a number of nucleotides, the exact number of which is unknown due to potential insertions/deletions in the non-coding portions of the gene, but is believed to be 19 between SEQ ID NO:44 and SEQ ID NO:45, and 17 between SEQ ID NO:45 and SEQ ID NO:46. Translation of Z. mays mays (strain Min13) polynucleotides SEQ ID NO:44, SEQ ID NO:45 and SEQ ID NO:46 suggests an open reading frame from about nucleotide 45 of SEQ ID NO:44 to about nucleotide 1741 of SEQ ID NO:46, with the first exon spanning nucleotides 1-125 of SEQ ID NO:44, the first intron spanning nucleotides 1-198 of SEQ ID NO:45 and nucleotides 1-374 of SEQ ID NO:46, the second exon spanning nucleotides 375-687 of SEQ ID NO:46, the second intron spanning nucleotides 688-788 of SEQ ID NO:46, and the third exon spanning nucleotides 789-1787 of SEQ ID NO:46. The open reading frame is represented herein as SEQ ID NO:47, and encodes a polypeptide represented herein as SEQ ID NO:48.

Similarly, polynucleotides SEQ ID NO:49 and SEQ ID NO:50 represent the 5′ and 3′ ends of the EG307 gene in Z. mays mays (strain Pira). SEQ ID NO:49 and SEQ ID NO:50 are joined by a number of nucleotides, the exact number of which is unknown due to potential insertions/deletions in the non-coding portions of the gene. Translation of Z. mays mays (strain Pira) polynucleotides SEQ ID NO:49 and SEQ ID NO:50 suggests an open reading frame from about nucleotide 31 of SEQ ID NO:49 to about nucleotide 1722 of SEQ ID NO:50, with the first exon spanning nucleotides 1-111 of SEQ ID NO:49, the first intron spanning nucleotides 112-495 of SEQ ID NO:49 and nucleotides 1-355 of SEQ ID NO:50, the second exon spanning nucleotides 356-668 of SEQ ID NO:50, the second intron spanning nucleotides 669-769 of SEQ ID NO:50, and the third exon spanning nucleotides 770-1768 of SEQ ID NO:50. The open reading frame is represented herein as SEQ ID NO:51, and encodes a polypeptide represented herein as SEQ ID NO:52.

Similarly, polynucleotides SEQ ID NO:53 and SEQ ID NO:54 represent the 5′ and 3′ ends of the EG307 gene in Z. mays mays (strain Sari). SEQ ID NO:53 and SEQ ID NO:54 are joined by a number of nucleotides, the exact number of which is unknown due to potential insertions/deletions in the non-coding portions of the gene, but is believed to be about 22. Translation of Z. mays mays (strain Pira) polynucleotides SEQ ID NO:53 and SEQ ID NO:54 suggests an open reading frame from about nucleotide 19 of SEQ ID NO:53 to about nucleotide 1756 of SEQ ID NO:54, with the first exon spanning nucleotides 1-99 of SEQ ID NO:53, the first intron spanning nucleotides 100-212 of SEQ ID NO:53 and nucleotides 1-389 of SEQ ID NO:54, the second exon spanning nucleotides 390-702 of SEQ ID NO:54, the second intron spanning nucleotides 703-803 of SEQ ID NO:54, and the third exon spanning nucleotides 804-1803 of SEQ ID NO:54. The open reading frame is represented herein as SEQ ID NO:55, and encodes a polypeptide represented herein as SEQ ID NO:56.

Similarly, polynucleotides SEQ ID NO:57 and SEQ ID NO:58 represent the 5′ and 3′ ends of the EG307 gene in Z. mays mays (strain Smena). SEQ ID NO:57 and SEQ ID NO:58 are joined by a number of nucleotides, the exact number of which is unknown due to potential insertions/deletions in the non-coding portions of the gene, but is believed to be 14. Translation of Z. mays mays (strain Smena) polynucleotides SEQ ID NO:57 and SEQ ID NO:58 suggests an open reading frame from about nucleotide 68 of SEQ ID NO:57 to about nucleotide 2199 of SEQ ID NO:58, with the first exon spanning nucleotides 1-148 of SEQ ID NO:57, the first intron spanning nucleotides 149-305 of SEQ ID NO:57 and nucleotides 1-834 of SEQ ID NO:58, the second exon spanning nucleotides 835-1147 of SEQ ID NO:58, the second intron spanning nucleotides 1148-1248 of SEQ ID NO:58, and the third exon spanning nucleotides 1249-2208 of SEQ ID NO:58. Additionally, sequence SEQ ID NO:59 contains a deletion at starting after nucleotide 738 of SEQ ID NO:59. The open reading frame is represented herein as SEQ ID NO:60, and encodes a polypeptide represented herein as SEQ ID NO:61.

Similarly, polynucleotides SEQ ID NO:62 and SEQ ID NO:63 represent the 5′ and 3′ ends of the EG307 gene in Z. mays mays (strain W22). SEQ ID NO:62 and SEQ ID NO:63 are joined by a number of nucleotides, the exact number of which is unknown due to potential insertions/deletions in the non-coding portions of the gene, but is believed to be about 22. Translation of Z. mays mays (strain W22) polynucleotides SEQ ID NO:62 and SEQ ID NO:63 suggests an open reading frame from about nucleotide 1 of SEQ ID NO:62 to about nucleotide 1367 of SEQ ID NO:63, with the first exon spanning nucleotides 1-81 of SEQ ID NO:62, the first intron spanning nucleotides 82-893 of SEQ ID NO:62, the second exon spanning nucleotides 1-313 of SEQ ID NO:63, the second intron spanning nucleotides 314-414 of SEQ ID NO:63, and the third exon spanning nucleotides 415-1411 of SEQ ID NO:63. The open reading frame is represented herein as SEQ ID NO:64, and encodes a polypeptide represented herein as SEQ ID NO:65.

A preferred Z. mays mays EG307 polypeptide of the present invention is a polypeptide encoded by a polynucleotide that hybridizes under stringent hybridization conditions with a polynucleotide represented by SEQ ID NO:33, SEQ ID NO:34, SEQ ID. NO:35, SEQ ID NO:37, SEQ ID NO:38, SEQ ID NO:40, SEQ ID NO:41, SEQ ID NO:42, SEQ ID NO:44, SEQ ID NO:45, SEQ ID NO:46, SEQ ID NO:47, SEQ ID NO:49, SEQ ID. NO:50, SEQ ID NO:51, SEQ ID NO:53, SEQ ID NO:54, SEQ ID NO:55, SEQ ID NO:57, SEQ ID NO:58, SEQ ID NO:60, SEQ ID NO:62, SEQ ID NO:63, and/or SEQ ID NO:64.

A preferred O. rufipogon EG307 polypeptide of the present invention is a polypeptide encoded by a polynucleotide that hybridizes under stringent hybridization conditions with a polynucleotide represented by SEQ ID NO:20, SEQ ID NO:21, SEQ ID. NO:23, SEQ ID NO:24, SEQ ID NO:25, SEQ ID NO:27, SEQ ID NO:28, SEQ ID NO:29, SEQ ID NO:30, and/or SEQ ID NO:31.

One preferred Zea diploperennis EG307 polypeptide of the present invention is a polypeptide encoded by an Zea mays parviglumis polynucleotide that hybridizes under stringent hybridization conditions with complements of polynucleotides represented by SEQ ID NO:80, SEQ ID NO:81, and/or SEQ ID NO:82. Such an EG307 polypeptide is encoded by a polynucleotide that hybridizes under stringent hybridization conditions with a polynucleotide having nucleic acid sequence SEQ ID NO:80, SEQ ID NO:81, and/or SEQ ID NO:82.

Polynucleotides SEQ ID NO:80 and SEQ ID NO:81 represent the 5′ and 3′ ends of the EG307 gene in Z. diploperennis SEQ ID NO:80 and SEQ ID NO:81 are joined by a number of nucleotides, the exact number of which is unknown due to potential insertions/deletions in the non-coding portions of the gene, but is believed to be about 24. One preferred Zea diploperennis EG307 polypeptide of the present invention is a polypeptide encoded by an Zea diploperennis polynucleotide that hybridizes under stringent hybridization conditions with complements of polynucleotides represented by SEQ ID NO:80 and SEQ ID NO:81. Such an EG307 polypeptide is encoded by a polynucleotide that hybridizes under stringent hybridization conditions with a polynucleotide having nucleic acid sequence SEQ ID NO:80 and SEQ ID NO:81.

Translation of SEQ ID NO:80 and SEQ ID NO:81 suggests that the Zea mays diploperennis EG307 polynucleotides includes an open reading frame. The reading frame encodes an Zea diploperennis EG307 polypeptide of about 448 amino acids, the deduced amino acid sequence of which is represented herein as SEQ ID NO:83, assuming an open reading frame having an initiation (start) codon spanning from about nucleotide 21 through about nucleotide 23 of SEQ ID NO:80 and a termination (stop) codon spanning from about nucleotide 1656 through about nucleotide 1658 of SEQ ID NO: 81, with the first exon spanning nucleotides 1-101 of SEQ ID NO:80, the first intron spanning nucleotides 102-225 of SEQ ID NO:80 and nucleotides 1-291 of SEQ ID NO:81, the second exon spanning nucleotides 292-313 of SEQ ID NO:81, the second intron spanning nucleotides 314-705 of SEQ ID NO:81, and the third exon spanning nucleotides 706-1672 of SEQ ID NO:81. The open reading frame from nucleotide 21 of SEQ ID NO:80 through about nucleotide 1658 of SEQ ID NO:81 is represented herein as SEQ ID NO:82.

A preferred Z. diploperennis EG307 polypeptide of the present invention is a polypeptide encoded by a polynucleotide that hybridizes under stringent hybridization conditions with polynucleotides represented by SEQ ID NO:80, SEQ ID NO:81, and/or SEQ ID NO:82.

One preferred Zea luxurians EG307 polypeptide of the present invention is a polypeptide encoded by an Zea luxurians polynucleotide that hybridizes under stringent hybridization conditions with complements of polynucleotides represented by SEQ ID NO:84 and/or SEQ ID NO:85. Such an EG307 polypeptide is encoded by a polynucleotide that hybridizes under stringent hybridization conditions with a polynucleotide having nucleic acid sequence SEQ ID NO:84 and/or SEQ ID NO:85.

Translation of SEQ ID NO:84 suggests that the Zea luxurians EG307 polynucleotide includes an open reading frame. The reading frame encodes an Zea luxurians EG307 polypeptide of about 448 amino acids, the deduced amino acid sequence of which is represented herein as SEQ ID NO:86, assuming an open reading frame having an initiation (start) codon spanning from about nucleotide 5 through about nucleotide 7 of SEQ ID NO:84 and a termination (stop) codon spanning from about nucleotide 2365 through about nucleotide 2367 of SEQ ID NO:84, with the first exon spanning nucleotides 1-85 of SEQ ID NO:84, the first intron spanning nucleotides 86-998 of SEQ ID NO:84, the second exon spanning nucleotides 999-1311 of SEQ ID NO:84, the second intron spanning nucleotides 1312-1414 of SEQ ID NO:84, and the third exon spanning nucleotides 1415-2423 of SEQ ID NO:84. The open reading frame from nucleotide 5 through about nucleotide 2367 of SEQ ID NO:84 is represented herein as SEQ ID NO:85.

A preferred Z. luxurians EG307 polypeptide of the present invention is a polypeptide encoded by a polynucleotide that hybridizes under stringent hybridization conditions with polynucleotides represented by SEQ ID NO:84, and/or SEQ ID NO:85.

Comparison of the various O. sativa, O. rufipogon, Z. mays mays, Z. mays parviglumis, Z. diploperennis, and Z. luxurians EG307 nucleic acid sequences and amino acid sequences indicates that these species of plants possess similar EG307 genes and polypeptides. The nucleotide sequences of the coding region of EG307 from the various strains of O. sativa and O. rufipogon have 99.0% sequence identity, when compared to each other, which makes clear that they are homologous. All rice sequences, both ancestral and modern, share the same stop codon (TAG), and (for the 5′ UTR sequence that we have collected to date), the 5′ UTR sequences have 98.4% sequence identity. The protein sequences of the various strains of O. sativa and O. rufipogon have 98.2% sequence identity, again demonstrating that these are homologous sequences. The protein sequence of EG307 from rice is about 94% identical to the protein sequence of EG307 from maize, again demonstrating their homology. The protein sequences of maize EG307 and teosinte EG307 have 99.8% sequence identity.

Finding this degree of identity between O. sativa, O. rufipogon, Z. mays mays, Z. mays parviglumis, Z. diploperennis, and Z. luxurians EG307 nucleic acid sequences and amino acid sequences supports the ability to obtain any plant EG307 polypeptide and polynucleotide given the polypeptide and nucleic acid sequences disclosed herein. These plant EG307 polypeptides, and the polynucleotides that encode them, represent novel compounds with utility in increasing yield in a plant.

Preferred plant EG307 polypeptides of the present invention include polypeptides comprising amino acid sequences that are at least about 30%, preferably at least about 50%, more preferably at least about 75%, more preferably at least about 80%, more preferably at least about 85%, more preferably at least about 90%, and more preferably at least about 95%, more preferably at least about 98% identical to one or more of the amino acid sequences disclosed herein for O. sativa, O. rufipogon, Z. mays mays, Z. mays parviglumis, Z. diploperennis, and Z. luxurians EG307 polypeptides of the present invention. More preferred plant EG307 polypeptides of the present invention include: polypeptides encoded by at least a portion of SEQ ID NO. 1 and/or SEQ ID NO:2 and, as such, have amino acid sequences that include at least a portion of SEQ ID NO:3; polypeptides encoded by at least a portion of SEQ ID NO:4, SEQ ID NO:81 and/or SEQ ID NO:5 and, as such, have amino acid sequences that include at least a portion of SEQ ID NO:6; polypeptides encoded by at least a portion of SEQ ID NO:7 and/or SEQ ID NO:8 and, as such, have amino acid sequences that include at least a portion of SEQ ID NO:9; polypeptides encoded by at least a portion of SEQ ID NO:10, SEQ ID NO:11, and/or SEQ ID NO:12 and, as such, have amino acid sequences that include at least a portion of SEQ ID NO:13; polypeptides encoded by at least a portion of SEQ ID NO:14 and/or SEQ ID NO:15 and, as such, have amino acid sequences that include at least a portion of SEQ ID NO:16; polypeptides encoded by at least a portion of SEQ ID NO:17 and/or SEQ ID NO:18 and, as such, have amino acid sequences that include at least a portion of SEQ ID NO:19; polypeptides encoded by at least a portion of SEQ ID NO:20 and/or SEQ ID NO:21 and, as such, have amino acid sequences that include at least a portion of SEQ ID NO:22; polypeptides encoded by at least a portion of SEQ ID NO:23, SEQ ID NO:24, and/or SEQ ID NO:25 and, as such, have amino acid sequences that include at least a portion of SEQ ID NO:26; polypeptides encoded by at least a portion of SEQ ID NO:27, SEQ ID NO:28 and/or SEQ ID NO:29 and, as such, have amino acid sequences that include at least a portion of SEQ ID NO:30; polypeptides encoded by at least a portion of SEQ ID NO:90 and/or SEQ ID NO:31 and, as such, have amino acid sequences that include at least a portion of SEQ ID NO:32; polypeptides encoded by at least a portion of SEQ ID NO:33, SEQ ID NO:34 and/or SEQ ID NO:35 and, as such, have amino acid sequences that include at least a portion of SEQ ID NO:36; polypeptides encoded by at least a portion of SEQ ID NO:37 and/or SEQ ID NO:38 and, as such, have amino acid sequences that include at least a portion of SEQ ID NO:39; polypeptides encoded by at least a portion of SEQ ID NO:40, SEQ ID NO:41, and/or SEQ ID NO:42 and, as such, have amino acid sequences that include at least a portion of SEQ ID NO:43; polypeptides encoded by at least a portion of SEQ ID NO:44, SEQ ID NO:45, SEQ ID NO:46, and/or SEQ ID NO:47 and, as such, have amino acid sequences that include at least a portion of SEQ ID NO:48; polypeptides encoded by at least a portion of SEQ ID NO:49, SEQ ID NO:50, and/or SEQ ID NO:51 and, as such, have amino acid sequences that include at least a portion of SEQ ID NO:52; polypeptides encoded by at least a portion of SEQ ID NO:53, SEQ ID NO:54, and/or SEQ ID NO:55 and, as such, have amino acid sequences that include at least a portion of SEQ ID NO:56; polypeptides encoded by at least a portion of SEQ ID NO:57, SEQ ID NO:58, and/or SEQ ID NO:60 and, as such, have amino acid sequences that include at least a portion of SEQ ID NO:61; polypeptides encoded by at least a portion of SEQ ID NO:62, SEQ ID NO:63, and/or SEQ ID NO:64 and, as such, have amino acid sequences that include at least a portion of SEQ ID NO:65; polypeptides encoded by at least a portion of SEQ ID NO:66, and/or SEQ ID NO:67 and, as such, have amino acid sequences that include at least a portion of SEQ ID NO:68; polypeptides encoded by at least a portion of SEQ ID NO:69, SEQ ID NO:70, and/or SEQ ID NO:71 and, as such, have amino acid sequences that include at least a portion of SEQ ID NO:72; polypeptides encoded by at least a portion of SEQ ID NO:73, SEQ ID NO:74, and/or SEQ ID NO:75 and, as such, have amino acid sequences that include at least a portion of SEQ ID NO:76; polypeptides encoded by at least a portion of SEQ ID NO:77, SEQ ID NO:59, and/or SEQ ID NO:78 and, as such, have amino acid sequences that include at least a portion of SEQ ID NO:79; polypeptides encoded by at least a portion of SEQ ID NO:80, SEQ ID NO:81, and/or SEQ ID NO:82 and, as such, have amino acid sequences that include at least a portion of SEQ ID NO:83; and polypeptides encoded by at least a portion of SEQ ID NO:84, and/or SEQ ID NO:85 and, as such, have amino acid sequences that include at least a portion of SEQ ID NO:86. As used herein, “at least a portion” of a polynucleotide or polypeptide means a portion having the minimal size characteristics of such sequences, as described above, or any larger fragment of the full length molecule, up to and including the full length molecule. For example, a portion of a polynucleotide may be 12 nucleotides, 13 nucleotides, 14 nucleotides, 15 nucleotides, and so on, going up to the full length polynucleotide. Similarly, a portion of a polypeptide may be 4 amino acids, 5 amino acids, 6 amino acids, 7 amino acids, and so on, going up to the full length polypeptide. The length of the portion to be used will depend on the particular application. As discussed above, a portion of a polynucleotide useful as hybridization probe may be as short as 12 nucleotides. A portion of a polypeptide useful as an epitope may be as short as 4 amino acids. A portion of a polypeptide that performs the function of the full-length polypeptide would generally be longer than 4 amino acids.

Particularly preferred plant EG307 polypeptides of the present invention are polypeptides that include SEQ ID NO:3, SEQ ID NO:6, SEQ ID NO:9, SEQ ID NO:13, SEQ ID NO:16, SEQ ID NO:19, SEQ ID NO:22, SEQ ID NO:26, SEQ ID NO:30, SEQ ID NO:32, SEQ ID NO:36, SEQ ID NO:39, SEQ ID NO:43, SEQ ID NO:48, SEQ ID NO:52, SEQ ID NO:56, SEQ ID NO:61, SEQ ID NO:65, SEQ ID NO:68. SEQ ID NO:72, SEQ ID NO:76, SEQ ID NO:79, SEQ ID NO:83 and/or SEQ ID NO:86 (including, but not limited to the encoded polypeptides, full-length polypeptides, processed polypeptides, fusion polypeptides and multivalent polypeptides thereof) as well as polypeptides that are truncated homologues of polypeptides that include at least portions of the aforementioned SEQ ID NOs. Examples of methods to produce such polypeptides are disclosed herein, including in the Examples section.

B. EG1117 Polypeptides

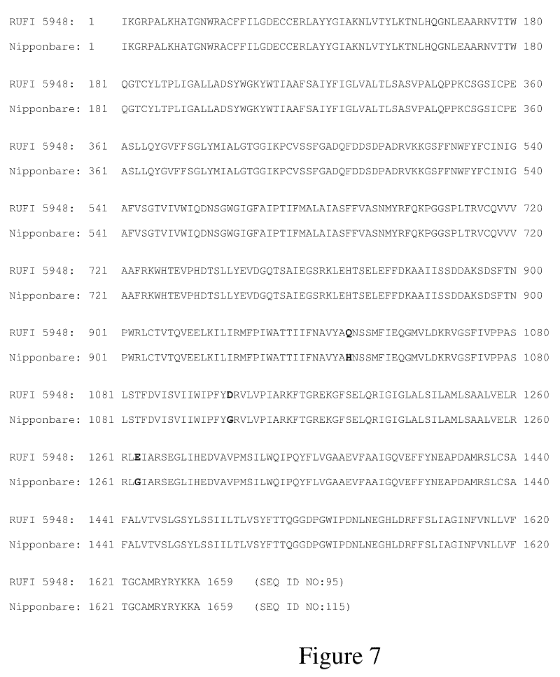

One embodiment of the present invention is an isolated plant EG1117 polypeptide. As used herein, an EG307 polypeptide, in one embodiment, is a polypeptide that is related to (i.e., bears structural similarity to) the O. rufipogon polypeptide of about 552 amino acids and having the sequence depicted in FIG. 7 (SEQ ID NO:95). The original identification of such a polypeptide is detailed in the Examples. A preferred EG1117 polypeptide is encoded by a polynucleotide that hybridizes under stringent hybridization conditions to at least one of the following genes: (a) a gene encoding an O. sativa EG1117 polypeptide (i.e., an O. sativa gene); (b) a gene encoding an O. rufipogon EG1117 polypeptide (i.e., an O. rufipogon gene); (c) a gene encoding a Zea mays mays EG1117 gene; (d) a gene encoding a Zea mays parviglumis EG1117 polypeptide (i.e., a. Z. mays parviglumis gene).

As used herein, an O. sativa EG1117 gene includes all nucleic acid sequences related to a natural O. sativa EG307 gene such as regulatory regions that control production of the O. sativa EG1117 polypeptide encoded by that gene (such as, but not limited to, transcription, translation or post-translation control regions) as well as the coding region itself. In one embodiment, an O. sativa EG1117 gene includes the nucleic acid sequence SEQ ID NO:4. Nucleic acid sequence SEQ ID NO:4 represents the deduced sequence of a cDNA (complementary DNA) polynucleotide, the production of which is disclosed in the Examples. It should be noted that since nucleic acid sequencing technology is not entirely error-free, SEQ ID NO:4 (as well as other sequences presented herein), at best, represents an apparent nucleic acid sequence of the polynucleotide encoding an O. sativa EG307 polypeptide of the present invention.

In another embodiment, an O. sativa EG1117 gene can be an allelic variant that includes a similar but not identical sequence to SEQ ID NO:92 and/or SEQ ID NO:93.

An EG1117 polypeptide of the present invention may be identified by its ability to perform the function of natural EG1117 in a functional assay. By “natural EG1117 polypeptide,” it is meant the full length EG1117 polypeptide of O. sativa, O. rufipogon, Z. mays mays, and/or Z. mays parviglumis. The phrase “capable of performing the function of a natural EG1117 in a functional assay” means that the polypeptide has at least about 10% of the activity of the natural polypeptide in the functional assay. In other preferred embodiments, the EG1117 polypeptide has at least about 20% of the activity of the natural polypeptide in the functional assay. In other preferred embodiments, the EG1117 polypeptide has at least about 30% of the activity of the natural polypeptide in the functional assay. In other preferred embodiments, the EG1117 polypeptide has at least about 40% of the activity of the natural polypeptide in the functional assay. In other preferred embodiments, the EG1117 polypeptide has at least about 50% of the activity of the natural polypeptide in the functional assay. In other preferred embodiments, the polypeptide has at least about 60% of the activity of the natural polypeptide in the functional assay. In more preferred embodiments, the polypeptide has at least about 70% of the activity of the natural polypeptide in the functional assay. In more preferred embodiments, the polypeptide has at least about 80% of the activity of the natural polypeptide in the functional assay. In more preferred embodiments, the polypeptide has at least about 90% of the activity of the natural polypeptide in the functional assay. Examples of functional assays include antibody-binding assays, or yield-increasing assays, as detailed elsewhere in this specification.

As used herein, an isolated plant EG1117 polypeptide can be a full-length polypeptide or any homologue of such a polypeptide. In one embodiment, when the homologue is administered to an animal as an immunogen, using techniques known to those skilled in the art, the animal will produce a humoral and/or cellular immune response against at least one epitope of a natural EG1117 polypeptide. EG1117 homologues can also be selected by their ability to perform the function of EG1117 in a functional assay.

Plant EG1117 polypeptide homologues can be the result of natural allelic variation or natural mutation. EG1117 polypeptide homologues of the present invention can also be produced using techniques known in the art including, but not limited to, direct modifications to the polypeptide or modifications to the gene encoding the polypeptide using, for example, classic or recombinant DNA techniques to effect random or targeted mutagenesis.

In accordance with the present invention, a mimetope refers to any compound that is able to mimic the ability of an isolated plant EG307 polypeptide of the present invention to perform the function of an EG307 polypeptide of the present invention in a functional assay. Examples of mimetopes include, but are not limited to, anti-idiotypic antibodies or fragments thereof, that include at least one binding site that mimics one or more epitopes of an isolated polypeptide of the present invention; non-polypeptideaceous immunogenic portions of an isolated polypeptide (e.g., carbohydrate structures); and synthetic or natural organic molecules, including nucleic acids, that have a structure similar to at least one epitope of an isolated polypeptide of the present invention. Such mimetopes can be designed using computer-generated structures of polypeptides of the present invention. Mimetopes can also be obtained by generating random samples of molecules, such as oligonucleotides, peptides or other organic molecules, and screening such samples by affinity chromatography techniques using the corresponding binding partner.

The minimal size of an EG307 polypeptide homologue of the present invention is a size sufficient to be encoded by a polynucleotide capable of forming a stable hybrid with the complementary sequence of a polynucleotide encoding the corresponding natural polypeptide. Minimal size characteristics are disclosed herein.

Any plant EG1117 polypeptide is a suitable polypeptide of the present invention. Suitable plants from which to isolate EG1117 polypeptides (including isolation of the natural polypeptide or production of the polypeptide by recombinant or synthetic techniques) include those described in the section entitles “EG307 Polypeptides.”

A preferred plant EG1117 polypeptide of the present invention is a compound that when expressed or modulated in a plant, is capable of increasing the yield of the plant.

One embodiment of the present invention is a fusion polypeptide that includes an EG1117 polypeptide-containing domain attached to a fusion segment.

Preferred plant EG1117 polypeptides of the present invention are rice EG1117 polypeptides and maize EG1117 polypeptides. More preferred EG1117 polypeptides are O. sativa, O. rufipogon, Z. mays mays, and Zea mays parviglumis EG1117 polypeptides. O. sativa strains include Nipponbare, Azucena, Kasalath 1, 2, 3, and 4, Teqing, Lemont, and IR64. Z. mays parviglumis strains include Benz, BK4, IA19, and Wilkes. Z. mays mays strains include BS7, HuoBai, Makki, Min13, Pira, Sari, Smena, and W22.

One preferred O. rufipogon EG1117 polypeptide of the present invention is a polypeptide encoded by an O. rufipogon polynucleotide that hybridizes under stringent hybridization conditions with complements of polynucleotides represented by SEQ ID NO:92, SEQ ID NO:93, SEQ ID NO:94, SEQ ID NO:96, SEQ ID NO:97, and/or SEQ ID NO:98.

One preferred O. sativa EG1117 polypeptide of the present invention is a polypeptide encoded by an O. sativa polynucleotide that hybridizes under stringent hybridization conditions with complements of polynucleotides represented by SEQ ID NO:100, SEQ ID NO:101, SEQ ID NO:102, SEQ ID NO:103, SEQ ID NO:104, SEQ ID NO:104, SEQ ID NO:106, SEQ ID NO:107, SEQ ID NO:109, SEQ ID NO:110, SEQ ID NO:112, SEQ ID NO:113, SEQ ID NO:114, SEQ ID NO:116, SEQ ID NO:117.

One preferred Z. mays mays EG1117 polypeptide of the present invention is a polypeptide encoded by an mays mays polynucleotide that hybridizes under stringent hybridization conditions with complements of polynucleotides represented by SEQ ID NO:119, SEQ ID NO:120, SEQ ID NO:122, SEQ ID NO:123, SEQ ID NO:124, SEQ ID NO:125, SEQ ID NO:127, SEQ ID NO:128, SEQ ID NO:129, SEQ ID NO:130, SEQ ID NO:133, SEQ ID NO:135, SEQ ID NO:136, SEQ ID NO:137, SEQ ID NO:138, SEQ ID NO:140, SEQ ID NO:141, SEQ ID NO:142, SEQ ID NO:144, SEQ ID NO:145, SEQ ID NO:146, SEQ ID NO:147, SEQ ID NO:149, SEQ ID NO:150, SEQ ID NO:151, SEQ ID NO:152, SEQ ID NO:154, SEQ ID NO:155,

One preferred Z. mays parviglumis EG1117 polypeptide of the present invention is a polypeptide encoded by an Z. mays parviglumis polynucleotide that hybridizes under stringent hybridization conditions with complements of polynucleotides represented by SEQ ID NO:157, SEQ ID NO:158, SEQ ID NO:160, SEQ ID NO:161, SEQ ID NO:162, SEQ ID NO:163, SEQ ID NO:165, SEQ ID NO:166, SEQ ID NO:167, and/or SEQ ID NO:168.

Inspection of EG1117 genomic nucleic acid sequences for rice indicates that the genes comprise several regions, including a first exon region, a first intron region, a second exon region, a second intron region, a third exon region, a third intron region, and a fourth exon region. The locations of these regions in each of the EG1117 rice and rice ancestor genomic nucleic acid sequences is summarized in the Table below:

| | SEQ ID. | | | | | | | |
|---|---|---|---|---|---|---|---|---|
| Organism | NO. | exon | intron | exon | intron | exon | intron | exon |
| O. rufipogon | 92 | 1-64 | 65-349 | 350-567 | 568-702 | 703-1259 | — | — |
| strain 5948 | | | | | | | | |
| (5′-ward end) | | | | | | | | |
| O. rufipogon | 93 | 1-868 | — | — | — | — | — | — |
| strain 5948 | | | | | | | | |
| (3′-ward end) | | | | | | | | |
| O. rufipogon | 96 | 1-35 | 36-320 | 321-538 | 539-673 | 674-1230 | — | — |
| strain 5949 | | | | | | | | |
| (5′-ward end) | | | | | | | | |
| O. rufipogon | 97 | — | 1-357 | 358-1225 | — | — | — | — |
| strain 5949 | | | | | | | | |
| (3′-ward end) | | | | | | | | |
| O. sativa | 100 | 1-64 | 65-349 | 350-567 | 568-702 | 703-1259 | 1260-1731 | 1732-2599 |
| strain | | | | | | | | |
| Azucena | | | | | | | | |
| O. sativa | 103 | 1-64 | 65-349 | 350-567 | 568-702 | 703-1259 | 1260-1733 | 1734-2601 |
| strain IR64 | | | | | | | | |
| O. sativa | 106 | 1-64 | 65-349 | 350-567 | 568-702 | 703-1259 | 1260-1733 | 1734-2601 |
| strain | | | | | | | | |
| Kasalath | | | | | | | | |
| O. sativa | 109 | 1-64 | 65-349 | 350-567 | 568-702 | 703-1259 | 1260-1731 | 1732-2599 |
| strain Lemont | | | | | | | | |
| O. sativa | 112 | 1-64 | 65-349 | 350-567 | 568-702 | 703-1259 | — | — |
| strain | | | | | | | | |
| Nipponbare | | | | | | | | |
| (5′-ward end) | | | | | | | | |
| O. sativa | 113 | 1-864 | — | — | — | — | — | — |
| strain | | | | | | | | |
| Nipponbare | | | | | | | | |
| (3′-ward end) | | | | | | | | |
| O. sativa | 116 | 1-64 | 65-349 | 350-567 | 568-702 | 703-1259 | 1260-1733 | 1734-2601 |
| strain Teqing | | | | | | | | |

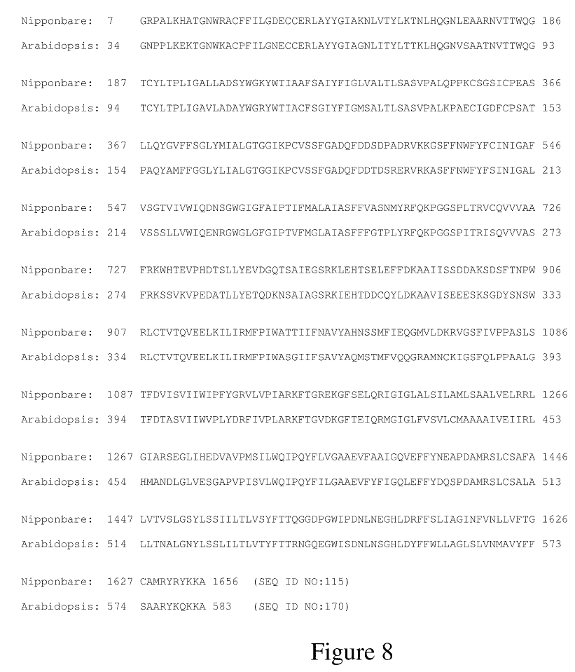

Translation of the genomic sequences suggests that the O. rufipogon and O. sativa EG1117 polynucleotide include open reading frames. The deduced protein sequence of O. sativa strain Nipponbare was used to perform a BLAST search. A very strong protein BLAST hit to Arabidopsis PTR2-B (histidine transporting protein, NP_{—}178313) suggests that only about 30 codons of coding sequence (CDS) are missing from the rice sequence (FIG. 8).

Finally, the deduced coding sequence and protein sequences are represented as follows:

| | SEQ ID NO: for | SEQ ID NO: for |
|---|---|---|
| Organism | partial CDS | partial protein |
| O. rufipogon strain 5948 | 94 | 95 |
| O. rufipogon strain 5949 | 98 | 99 |
| O. sativa strain Azucena | 101 | 102 |
| O. sativa strain IR64 | 104 | 105 |
| O. sativa strain Kasalath | 107 | 108 |
| O. sativa strain Lemont | 110 | 111 |
| O. sativa strain Nipponbare | 114 | 115 |
| O. sativa strain Teqing | 117 | 118 |

The partial sequence of EG1117 has also been determined in maize and teosinte. This information is summarized in the Table below:

| | SEQ ID | | | | |
|---|---|---|---|---|---|
| Organism | NO: | exon | intron | exon | CDS |
| Zea mays mays strain BS7 | 119 | 1-531 | — | — | 1-531 |
| Zea mays mays strain Enano | 122 | 1-365 | 366-536 | — | 1-365 |
| Zea mays mays strain Enano | 123 | — | 1-393 | 394-550 | 394-550 |
| Zea mays mays strain Enano | 124 | 1-533 | — | — | 1-533 |
| Zea mays mays strain Huobai | 127 | 1-375 | 376-525 | — | 1-375 |
| Zea mays mays strain Huobai | 128 | — | 1-143 | 144-334 | 144-334 |
| Zea mays mays strain Huobai | 129 | 1-529 | — | — | 1-529 |
| Zea mays mays strain Makki | 132 | 1-513 | — | — | 1-513 |
| Zea mays mays strain Min13 | 135 | 1-374 | 375-545 | — | 1-374 |
| Zea mays mays strain Min13 | 136 | — | 1-390 | 391-570 | 391-570 |
| Zea mays mays strain Min13 | 137 | 1-525 | — | — | 1-525 |
| Zea mays mays strain Pira | 140 | 1-371 | 372-526 | — | 1-371 |
| Zea mays mays strain Pira | 141 | 1-525 | — | — | 1-525 |
| Zea mays mays strain Sari | 144 | 1-364 | 365-499 | — | 1-364 |
| Zea mays mays strain Sari | 145 | — | 1-422 | 423-607 | 423-607 |
| Zea mays mays strain Sari | 146 | 1-520 | — | — | 1-520 |
| Zea mays mays strain Smena | 149 | 1-371 | 372-543 | — | 1-371 |
| Zea mays mays strain Smena | 150 | — | 1-262 | 263-443 | 263-443 |
| Zea mays mays strain Smena | 151 | 1-523 | — | — | 1-523 |
| Zea mays mays strain W22 | 154 | 1-488 | — | — | 1-488 |
| Zea mays parviglumis strain Benz | 157 | 1-516 | — | — | 1-516 |
| Zea mays parviglumis strain BK4 | 160 | 1-372 | 373-385 | — | 1-372 |
| Zea mays parviglumis strain BK4 | 161 | — | 1-433 | 434-613 | 434-613 |
| Zea mays parviglumis strain BK4 | 162 | 1-462 | — | — | 1-462 |
| Zea mays parviglumis strain | 165 | 1-355 | 356-556 | — | 1-355 |
| Wilkes | | | | | |
| Zea mays parviglumis strain | 166 | — | 1-395 | 396-552 | 396-552 |
| Wilkes | | | | | |
| Zea mays parviglumis strain | 167 | 1-511 | — | — | 1-511 |
| Wilkes | | | | | |

Translation of the genomic sequences suggests that the Z. mays mays and Z. mays parviglumis EG1117 polynucleotide include open reading frames.

A summary of the open reading frame information appears in the following Table:

| | | SEQ ID NO: |
|---|---|---|
| | SEQ ID NO: | for partial |
| Organism | for partial CDS | protein |
| Zea mays mays strain BS7 | 120 | 121 |
| Zea mays mays strain Enano | 125 | 126 |
| Zea mays mays strain Huobai | 130 | 131 |
| Zea mays mays strain Makki | 133 | 134 |
| Zea mays mays strain Min13 | 138 | 139 |
| Zea mays mays strain Pira | 142 | 143 |
| Zea mays mays strain Sari | 147 | 148 |
| Zea mays mays strain Smena | 152 | 153 |
| Zea mays mays strain W22 | 155 | 156 |
| Zea mays parviglumis strain Benz | 158 | 159 |
| Zea mays parviglumis strain BK4 | 163 | 164 |
| Zea mays parviglumis strain Wilkes | 168 | 169 |

Preferred plant EG1117 polypeptides of the present invention include polypeptides comprising amino acid sequences that are at least about 30%, preferably at least about 50%, more preferably at least about 75%, more preferably at least about 80%, more preferably at least about 85%, more preferably at least about 90%, and more preferably at least about 95%, more preferably at least about 98% identical to one or more of the amino acid sequences disclosed herein for O. sativa, O. rufipogon, Z. mays mays, and Z. mays parviglumis, EG1117 polypeptides of the present invention. More preferred plant EG1117 polypeptides of the present invention include: polypeptides encoded by at least a portion of SEQ ID NO. 92, SEQ ID NO. 93, and/or SEQ ID NO:94 and, as such, have amino acid sequences that include at least a portion of SEQ ID NO:95; polypeptides encoded by at least a portion of SEQ ID NO:96, SEQ ID NO:97 and/or SEQ ID NO:98 and, as such, have amino acid sequences that include at least a portion of SEQ ID NO:99; polypeptides encoded by at least a portion of SEQ ID NO:100 and/or SEQ ID NO:101 and, as such, have amino acid sequences that include at least a portion of SEQ ID NO:102; polypeptides encoded by at least a portion of SEQ ID NO:103, and/or SEQ ID NO:104 and, as such, have amino acid sequences that include at least a portion of SEQ ID NO:105; polypeptides encoded by at least a portion of SEQ ID NO:106 and/or SEQ ID NO:107 and, as such, have amino acid sequences that include at least a portion of SEQ ID NO:108; polypeptides encoded by at least a portion of SEQ ID NO:09 and/or SEQ ID NO:110 and, as such, have amino acid sequences that include at least a portion of SEQ ID NO:111; polypeptides encoded by at least a portion of SEQ ID NO:112, SEQ ID NO:113 and/or SEQ ID NO:114 and, as such, have amino acid sequences that include at least a portion of SEQ ID NO:115; polypeptides encoded by at least a portion of SEQ ID NO:116 and/or SEQ ID NO:117 and, as such, have amino acid sequences that include at least a portion of SEQ ID NO:118; polypeptides encoded by at least a portion of SEQ ID NO:119 and/or SEQ ID NO:120 and, as such, have amino acid sequences that include at least a portion of SEQ ID NO:121; polypeptides encoded by at least a portion of SEQ ID NO:122, SEQ ID NO:123 SEQ ID NO:124 and/or SEQ ID NO:125, and, as such, have amino acid sequences that include at least a portion of SEQ ID NO:126; polypeptides encoded by at least a portion of SEQ ID NO:127, SEQ ID NO:128, SEQ ID NO:129 and/or SEQ ID NO:130 and, as such, have amino acid sequences that include at least a portion of SEQ ID NO:131; polypeptides encoded by at least a portion of SEQ ID NO:132 and/or SEQ ID NO:133 and, as such, have amino acid sequences that include at least a portion of SEQ ID NO:134; polypeptides encoded by at least a portion of SEQ ID NO:135, SEQ ID NO:136, SEQ ID NO:137, and/or SEQ ID NO:138 and, as such, have amino acid sequences that include at least a portion of SEQ ID NO:139; polypeptides encoded by at least a portion of SEQ ID NO:140, SEQ ID NO:141, and/or SEQ ID NO:142 and, as such, have amino acid sequences that include at least a portion of SEQ ID NO:143; polypeptides encoded by at least a portion of SEQ ID NO:144, SEQ ID NO:145, SEQ ID NO:146 and/or SEQ ID NO:147 and, as such, have amino acid sequences that include at least a portion of SEQ ID NO:148; polypeptides encoded by at least a portion of SEQ ID NO:149, SEQ ID NO:150, SEQ ID NO:151, and/or SEQ ID NO:152 and, as such, have amino acid sequences that include at least a portion of SEQ ID NO:153; polypeptides encoded by at least a portion of SEQ ID NO:154 and/or SEQ ID NO:155 and, as such, have amino acid sequences that include at least a portion of SEQ ID NO:156; polypeptides encoded by at least a portion of SEQ ID NO:157, and/or SEQ ID NO:158 and, as such, have amino acid sequences that include at least a portion of SEQ ID NO:159; polypeptides encoded by at least a portion of SEQ ID NO:160, SEQ ID NO:161, SEQ ID NO:162, and/or SEQ ID NO:163 and, as such, have amino acid sequences that include at least a portion of SEQ ID NO:164; and polypeptides encoded by at least a portion of SEQ ID NO:165, SEQ ID NO:166, SEQ ID NO:167, and/or SEQ ID NO:168 and, as such, have amino acid sequences that include at least a portion of SEQ ID NO:169. As used herein, “at least a portion” of a polynucleotide or polypeptide means a portion having the minimal size characteristics of such sequences, as described above, or any larger fragment of the full length molecule, up to and including the full length molecule. For example, a portion of a polynucleotide may be 12 nucleotides, 13 nucleotides, 14 nucleotides, 15 nucleotides, and so on, going up to the full length polynucleotide. Similarly, a portion of a polypeptide may be 4 amino acids, 5 amino acids, 6 amino acids, 7 amino acids, and so on, going up to the full length polypeptide. The length of the portion to be used will depend on the particular application. As discussed above, a portion of a polynucleotide useful as hybridization probe may be as short as 12 nucleotides. A portion of a polypeptide useful as an epitope may be as short as 4 amino acids. A portion of a polypeptide that performs the function of the full-length polypeptide would generally be longer than 4 amino acids.

Particularly preferred plant EG 1117 polypeptides of the present invention are polypeptides that include SEQ ID NO:95, SEQ ID NO:99, SEQ ID NO:102, SEQ ID NO:105, SEQ ID NO:108, SEQ ID NO:111, SEQ ID NO:115, SEQ ID NO:118, SEQ ID NO:121, SEQ ID NO:126, SEQ ID NO:131, SEQ ID NO:134, SEQ ID NO:139, SEQ ID NO:143, SEQ ID NO:148, SEQ ID NO:153, SEQ ID NO:156, SEQ ID NO:159, SEQ ID NO:164, SEQ ID NO:169, and/or SEQ ID NO:170 (including, but not limited to the encoded polypeptides, full-length polypeptides, processed polypeptides, fusion polypeptides and multivalent polypeptides thereof) as well as polypeptides that are truncated homologues of polypeptides that include at least portions of the aforementioned SEQ ID NOs. Examples of methods to produce such polypeptides are disclosed herein, including in the Examples section.

C. EG307 Polynucleotides

One embodiment of the present invention is an isolated plant polynucleotide that hybridizes under stringent hybridization conditions with at least one of the following genes: an O. sativa EG307 gene, an O. rufipogon EG307 gene, a Z. mays mays EG307 gene, a Z. mays parviglumis EG307 gene, a Z. diploperennis EG307 gene, and a Z. luxurians gene. The identifying characteristics of such genes are heretofore described. A polynucleotide of the present invention can include an isolated natural plant EG307 gene or a homologue thereof, the latter of which is described in more detail below. A polynucleotide of the present invention can include one or more regulatory regions, full-length or partial coding regions, or combinations thereof. The minimal size of a polynucleotide of the present invention is the minimal size that can form a stable hybrid with one of the aforementioned genes under stringent hybridization conditions. Suitable and preferred plants are disclosed above.

In accordance with the present invention, an isolated polynucleotide is a polynucleotide that has been removed from its natural milieu (i.e., that has been subject to human manipulation). As such, “isolated” does not reflect the extent to which the polynucleotide has been purified. An isolated polynucleotide can include DNA, RNA, or derivatives of either DNA or RNA.

An isolated plant EG307 polynucleotide of the present invention can be obtained from its natural source either as an entire (i.e., complete) gene or a portion thereof capable of forming a stable hybrid with that gene. An isolated plant EG307 polynucleotide can also be produced using recombinant DNA technology (e.g., polymerase chain reaction (PCR) amplification, cloning) or chemical synthesis. Isolated plant EG307 polynucleotides include natural polynucleotides and homologues thereof, including, but not limited to, natural allelic variants and modified polynucleotides in which nucleotides have been inserted, deleted, substituted, and/or inverted in such a manner that such modifications do not substantially interfere with the polynucleotide's ability to encode an EG307 polypeptide of the present invention or to form stable hybrids under stringent conditions with natural gene isolates.

A plant EG307 polynucleotide homologue can be produced using a number of methods known to those skilled in the art (see, for example, Sambrook et al., ibid.). For example, polynucleotides can be modified using a variety of techniques including, but not limited to, classic mutagenesis techniques and recombinant DNA techniques, such as site-directed mutagenesis, chemical treatment of a polynucleotide to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of polynucleotides and combinations thereof. Polynucleotide homologues can be selected from a mixture of modified nucleic acids by screening for the function of the polypeptide encoded by the nucleic acid (e.g., ability to elicit an immune response against at least one epitope of an EG307 polypeptide, ability to increase yield in a transgenic plant containing an EG307 gene) and/or by hybridization with an O. sativa EG307 gene, with an O. rufipogon EG307 gene, with a Z. mays mays EG307 gene, with a Z. mays parviglumis EG307 gene, a Z. diploperennis EG307 gene and/or a Z. luxurians EG307 gene.

An isolated polynucleotide of the present invention can include a nucleic acid sequence that encodes at least one plant EG307 polypeptide of the present invention, examples of such polypeptides being disclosed herein. Although the phrase “polynucleotide” primarily refers to the physical polynucleotide and the phrase “nucleic acid sequence” primarily refers to the sequence of nucleotides on the polynucleotide, the two phrases can be used interchangeably, especially with respect to a polynucleotide, or a nucleic acid sequence, being capable of encoding an EG307 polypeptide. As heretofore disclosed, plant EG307 polypeptides of the present invention include, but are not limited to, polypeptides having full-length plant EG307 coding regions, polypeptides having partial plant EG307 coding regions, fusion polypeptides, multivalent protective polypeptides and combinations thereof.

At least certain polynucleotides of the present invention encode polypeptides that selectively bind to immune serum derived from an animal that has been immunized with an EG307 polypeptide from which the polynucleotide was isolated.

A preferred polynucleotide of the present invention, when expressed in a suitable plant, is capable of increasing the yield of the plant. As will be disclosed in more detail below, such a polynucleotide can be, or encode, an antisense RNA, a molecule capable of triple helix formation, a ribozyme, or other nucleic acid-based compound.

One embodiment of the present invention is a plant EG307 polynucleotide that hybridizes under stringent hybridization conditions to an EG307 polynucleotide of the present invention, or to a homologue of such an EG307 polynucleotide, or to the complement of such a polynucleotide. A polynucleotide complement of any nucleic acid sequence of the present invention refers to the nucleic acid sequence of the polynucleotide that is complementary to (i.e., can form a complete double helix with) the strand for which the sequence is cited. It is to be noted that a double-stranded nucleic acid molecule of the present invention for which a nucleic acid sequence has been determined for one strand, that is represented by a SEQ ID NO, also comprises a complementary strand having a sequence that is a complement of that SEQ ID NO. As such, polynucleotides of the present invention, which can be either double-stranded or single-stranded, include those polynucleotides that form stable hybrids under stringent hybridization conditions with either a given SEQ ID NO denoted herein and/or with the complement of that SEQ ID NO, which may or may not be denoted herein. Methods to deduce a complementary sequences are known to those skilled in the art. Preferred is an EG307 polynucleotide that includes a nucleic acid sequence having at least about 65 percent, preferably at least about 70 percent, more preferably at least about 75 percent, more preferably at least about 80 percent, more preferably at least about 85 percent, more preferably at least about 90 percent and even more preferably at least about 95 percent homology with the corresponding region(s) of the nucleic acid sequence encoding at least a portion of an EG307 polypeptide. Particularly preferred is an EG307 polynucleotide capable of encoding at least a portion of an EG307 polypeptide that naturally is present in plants.

Particularly preferred EG307 polynucleotides of the present invention hybridize under stringent hybridization conditions with at least one of the following polynucleotides: SEQ ID NO:1, SEQ ID NO:91, SEQ ID. NO:2, SEQ ID NO:4, SEQ ID NO:5, SEQ ID NO:7, SEQ ID NO:10, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID. NO:23, SEQ ID NO:24, SEQ ID NO:25, SEQ ID NO:27, SEQ ID NO:28, SEQ ID NO:29, SEQ ID NO:90, SEQ ID NO:31, SEQ ID NO:33, SEQ ID NO:34, SEQ ID. NO:35, SEQ ID NO:37, SEQ ID NO:38, SEQ ID NO:40, SEQ ID NO:41, SEQ ID NO:42, SEQ ID NO:44, SEQ ID NO:45, SEQ ID NO:46, SEQ ID NO:47, SEQ ID NO:49, SEQ ID. NO:50, SEQ ID NO:51, SEQ ID NO:53, SEQ ID NO:54, SEQ ID NO:55, SEQ ID NO:57, SEQ ID NO:58, SEQ ID NO:60, SEQ ID NO:62, SEQ ID NO:63, SEQ ID NO:64, SEQ ID NO:66, SEQ ID NO:67, SEQ ID NO:69, SEQ ID. NO:70, SEQ ID NO:71, SEQ ID NO:73, SEQ ID NO:74, SEQ ID NO:75, SEQ ID NO:77, SEQ ID NO:59, SEQ ID NO:78, SEQ ID NO:80, SEQ ID NO:81, SEQ ID NO:82, SEQ ID NO:84, and/or SEQ ID NO:85, or to a homologue or complement of such polynucleotide.

A preferred polynucleotide of the present invention includes at least a portion of nucleic acid sequence SEQ ID NO:1, SEQ ID NO:91, SEQ ID. NO:2, SEQ ID NO:4, SEQ ID NO:5, SEQ ID NO:7, SEQ ID NO:10, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID. NO:23, SEQ ID NO:24, SEQ ID NO:25, SEQ ID NO:27, SEQ ID NO:28, SEQ ID NO:29, SEQ ID NO:30, SEQ ID NO:31, SEQ ID NO:33, SEQ ID NO:34, SEQ ID. NO:35, SEQ ID NO:37, SEQ ID NO:38, SEQ ID NO:40, SEQ ID NO:41, SEQ ID NO:42, SEQ ID NO:44, SEQ ID NO:45, SEQ ID NO:46, SEQ ID NO:47, SEQ ID NO:49, SEQ ID. NO:50, SEQ ID NO:51, SEQ ID NO:53, SEQ ID NO:54, SEQ ID NO:55, SEQ ID NO:57, SEQ ID NO:58, SEQ ID NO:60, SEQ ID NO:62, SEQ ID NO:63, SEQ ID NO:64, SEQ ID NO:66, SEQ ID NO:67, SEQ ID NO:69, SEQ ID. NO:70, SEQ ID NO:71, SEQ ID NO:73, SEQ ID NO:74, SEQ ID NO:75, SEQ ID NO:77, SEQ ID NO:59, and/or SEQ ID NO:78 that is capable of hybridizing (i.e., that hybridizes under stringent hybridization conditions) to an O. sativa EG307 gene, to a O. rufipogon EG307 gene, to a Z. mays mays EG307 gene, to a Z. mays parviglumis EG307 gene, to a Z. diploperennis EG307 gene and/or to a Z. luxurians EG307 gene of the present invention, as well as a polynucleotide that is an allelic variant of any of those polynucleotides. Such preferred polynucleotides can include nucleotides in addition to those included in the SEQ ID NOs, such as, but not limited to, a full-length gene, a full-length coding region, a polynucleotide encoding a fusion polypeptide, and/or a polynucleotide encoding a multivalent protective compound.

The present invention also includes polynucleotides encoding a polypeptide including at least a portion of SEQ ID NO:3, polynucleotides encoding a polypeptide having at least a portion of SEQ ID NO:6, polynucleotides encoding a polypeptide having at least a portion of SEQ ID NO:9, polynucleotides encoding a polypeptide having at least a portion of SEQ ID NO:13, polynucleotides encoding a polypeptide having at least a portion of SEQ ID NO: 16, polynucleotides encoding a polypeptide having at least a portion of SEQ ID NO:19, polynucleotides encoding a polypeptide having at least a portion of SEQ ID NO:22, polynucleotides encoding a polypeptide having at least a portion of SEQ ID NO:26, polynucleotides encoding a polypeptide having at least a portion of SEQ ID NO:30, polynucleotides encoding a polypeptide having at least a portion of SEQ ID NO:36, polynucleotides encoding a polypeptide having at least a portion of SEQ ID NO:39, polynucleotides encoding a polypeptide having at least a portion of SEQ ID NO:43, polynucleotides encoding a polypeptide having at least a portion of SEQ ID NO:48, polynucleotides encoding a polypeptide having at least a portion of SEQ ID NO:52, polynucleotides encoding a polypeptide having at least a portion of SEQ ID NO:56, polynucleotides encoding a polypeptide having at least a portion of SEQ ID NO:61, polynucleotides encoding a polypeptide having at least a portion of SEQ ID NO:65, polynucleotides encoding a polypeptide having at least a portion of SEQ ID NO:68, polynucleotides encoding a polypeptide having at least a portion of SEQ ID NO:72, polynucleotides encoding a polypeptide having at least a portion of SEQ ID NO:76, polynucleotides encoding a polypeptide having at least a portion of SEQ ID NO:79, polynucleotides encoding a polypeptide having at least a portion of SEQ ID NO:83, and/or polynucleotides encoding a polypeptide having at least a portion of SEQ ID NO:86, including polynucleotides that have been modified to accommodate codon usage properties of the cells in which such polynucleotides are to be expressed.

Knowing the nucleic acid sequences of certain plant EG307 polynucleotides of the present invention allows one skilled in the art to, for example, (a) make copies of those polynucleotides, (b) obtain polynucleotides including at least a portion of such polynucleotides (e.g., polynucleotides including full-length genes, full-length coding regions, regulatory control sequences, truncated coding regions), and (c) obtain EG307 polynucleotides for other plants, particularly since, as described in detail in the Examples section, knowledge of O. sativa EG307 polynucleotides of the present invention enabled the isolation of O. rufipogon, Zea mays mays, Zea mays parviglumis, Z. diploperennis, and Z. luxurians EG307 polynucleotides of the present invention. Such polynucleotides can be obtained in a variety of ways including screening appropriate expression libraries with antibodies of the present invention; traditional cloning techniques using oligonucleotide probes of the present invention to screen appropriate libraries or DNA; and PCR amplification of appropriate libraries or DNA using oligonucleotide primers of the present invention. Preferred libraries to screen or from which to amplify polynucleotides include libraries such as genomic DNA libraries, BAC libraries, YAC libraries, cDNA libraries prepared from isolated plant tissues, including, but not limited to, stems, reproductive structures/tissues, leaves, roots, and tillers; and libraries constructed from pooled cDNAs from any or all of the tissues listed above. In the case of rice, BAC libraries, available from Clemson University, are preferred. Similarly, preferred DNA sources to screen or from which to amplify polynucleotides include plant genomic DNA. Techniques to clone and amplify genes are disclosed, for example, in Sambrook et al., ibid. and in Galun & Breiman, TRANSGENIC PLANTS, Imperial College Press, 1997.

The present invention also includes polynucleotides that are oligonucleotides capable of hybridizing, under stringent hybridization conditions, with complementary regions of other, preferably longer, polynucleotides of the present invention such as those comprising plant EG307 genes or other plant EG307 polynucleotides. Oligonucleotides of the present invention can be RNA, DNA, or derivatives of either. The minimal size of such oligonucleotides is the size required to form a stable hybrid between a given oligonucleotide and the complementary sequence on another polynucleotide of the present invention. Minimal size characteristics are disclosed herein. The size of the oligonucleotide must also be sufficient for the use of the oligonucleotide in accordance with the present invention. Oligonucleotides of the present invention can be used in a variety of applications including, but not limited to, as probes to identify additional polynucleotides, as primers to amplify or extend polynucleotides, as targets for expression analysis, as candidates for targeted mutagenesis and/or recovery, or in agricultural applications to alter EG307 polypeptide production or activity. Such agricultural applications include the use of such oligonucleotides in, for example, antisense-, triplex formation-, ribozyme- and/or RNA drug-based technologies. The present invention, therefore, includes such oligonucleotides and methods to enhance economic productivity in a plant by use of one or more of such technologies.

D. EG1117 Polynucleotides

One embodiment of the present invention is an isolated plant polynucleotide that hybridizes under stringent hybridization conditions with at least one of the following genes: an O. sativa EG1117 gene, an O. rufipogon EG1117 gene, a Z. mays mays EG1117 gene, and a Z. mays parviglumis EG1117 gene. The identifying characteristics of such genes are heretofore described. A polynucleotide of the present invention can include an isolated natural plant EG1117 gene or a homologue thereof. A polynucleotide of the present invention can include one or more regulatory regions, full-length or partial coding regions, or combinations thereof. The minimal size of a polynucleotide of the present invention is the minimal size that can form a stable hybrid with one of the aforementioned genes under stringent hybridization conditions. Suitable and preferred plants are disclosed above. Characteristics of isolated EG1117 genes and homologues thereof are described above in the section entitled “EG307 polynucleotides.”

One embodiment of the present invention is a plant EG1117 polynucleotide that hybridizes under stringent hybridization conditions to an EG1117 polynucleotide of the present invention, or to a homologue of such an EG1117 polynucleotide, or to the complement of such a polynucleotide. Preferred is an EG1117 polynucleotide that includes a nucleic acid sequence having at least about 65 percent, preferably at least about 70 percent, more preferably at least about 75 percent, more preferably at least about 80 percent, more preferably at least about 85 percent, more preferably at least about 90 percent and even more preferably at least about 95 percent homology with the corresponding region(s) of the nucleic acid sequence encoding at least a portion of an EG1117 polypeptide. Particularly preferred is an EG1117 polynucleotide capable of encoding at least a portion of an EG1117 polypeptide that naturally is present in plants.

Particularly preferred EG1117 polynucleotides of the present invention hybridize under stringent hybridization conditions with at least one of the following polynucleotides, or to a homologue or complement of such polynucleotide.

A preferred polynucleotide of the present invention includes at least a portion of nucleic acid sequence SEQ ID NO:92, that is capable of hybridizing (i.e., that hybridizes under stringent hybridization conditions) to an O. sativa EG1117 gene, to a O. rufipogon EG1117 gene, to a Z. mays mays EG1117 gene, and/or to a Z. mays parviglumis EG1117 gene of the present invention, as well as a polynucleotide that is an allelic variant of any of those polynucleotides. Such preferred polynucleotides can include nucleotides in addition to those included in the SEQ ID NOs, such as, but not limited to, a full-length gene, a full-length coding region, a polynucleotide encoding a fusion polypeptide, and/or a polynucleotide encoding a multivalent protective compound.

A preferred polynucleotide of the present invention includes at least a portion of nucleic acid sequence SEQ ID NO:92, SEQ ID NO:93, SEQ ID NO:94, SEQ ID NO:96, SEQ ID NO:97, SEQ ID NO:98, SEQ ID NO:100, SEQ ID NO:101, SEQ ID NO:102, SEQ ID NO:103, SEQ ID NO:104, SEQ ID NO:104, SEQ ID NO:106, SEQ ID NO:107, SEQ ID NO:109, SEQ ID NO:10, SEQ ID NO:112, SEQ ID NO:113, SEQ ID NO:114, SEQ ID NO:116, SEQ ID NO:117, SEQ ID NO:119, SEQ ID NO:120, SEQ ID NO:121, SEQ ID NO:122, SEQ ID NO:123, SEQ ID NO:124, SEQ ID NO:125, SEQ ID NO:127, SEQ ID NO:128, SEQ ID NO:129, SEQ ID NO:130, SEQ ID NO:131, SEQ ID NO:133, SEQ ID NO:135, SEQ ID NO:136, SEQ ID NO:137, SEQ ID NO:138, SEQ ID NO:140, SEQ ID NO:141, SEQ ID NO:142, SEQ ID NO:144, SEQ ID NO:145, SEQ ID NO:146, SEQ ID NO:147, SEQ ID NO:149, SEQ ID NO:150, SEQ ID NO:151, SEQ ID NO:152, SEQ ID NO:154, SEQ ID NO:155, SEQ ID NO:157, SEQ ID NO:158, SEQ ID NO:160, SEQ ID NO:161, SEQ ID NO:162, SEQ ID NO:163, SEQ ID NO:165, SEQ ID NO:166, SEQ ID NO:167, and/or SEQ ID NO:168, that is capable of hybridizing (i.e., that hybridizes under stringent hybridization conditions) to an O. sativa EG1117 gene, to a O. rufipogon EG1117 gene, to a Z. mays mays EG1117 gene, and/or to a Z. mays parviglumis EG1117 gene, to a gene of the present invention, as well as a polynucleotide that is an allelic variant of any of those polynucleotides. Such preferred polynucleotides can include nucleotides in addition to those included in the SEQ ID NOs, such as, but not limited to, a full-length gene, a full-length coding region, a polynucleotide encoding a fusion polypeptide, and/or a polynucleotide encoding a multivalent protective compound.

The present invention also includes polynucleotides encoding a polypeptide including at least a portion of SEQ ID NO:95, polynucleotides encoding a polypeptide having at least a portion of SEQ ID NO:99, polynucleotides encoding a polypeptide having at least a portion of SEQ ID NO:102, polynucleotides encoding a polypeptide having at least a portion of SEQ ID NO:105, polynucleotides encoding a polypeptide having at least a portion of SEQ ID NO:108, polynucleotides encoding a polypeptide having at least a portion of SEQ ID NO:111, polynucleotides encoding a polypeptide having at least a portion of SEQ ID NO:115, polynucleotides encoding a polypeptide having at least a portion of SEQ ID NO:118, polynucleotides encoding a polypeptide having at least a portion of SEQ ID NO:121, polynucleotides encoding a polypeptide having at least a portion of SEQ ID NO:126, polynucleotides encoding a polypeptide having at least a portion of SEQ ID NO:131, polynucleotides encoding a polypeptide having at least a portion of SEQ ID NO:134, polynucleotides encoding a polypeptide having at least a portion of SEQ ID NO:139, polynucleotides encoding a polypeptide having at least a portion of SEQ ID NO:143, polynucleotides encoding a polypeptide having at least a portion of SEQ ID NO:148, polynucleotides encoding a polypeptide having at least a portion of SEQ ID NO:153, polynucleotides encoding a polypeptide having at least a portion of SEQ ID NO:156, polynucleotides encoding a polypeptide having at least a portion of SEQ ID NO:159, polynucleotides encoding a polypeptide having at least a portion of SEQ ID NO:164, polynucleotides encoding a polypeptide having at least a portion of SEQ ID NO:169, including polynucleotides that have been modified to accommodate codon usage properties of the cells in which such polynucleotides are to be expressed.

Knowing the nucleic acid sequences of certain plant EG1117 polynucleotides of the present invention allows one skilled in the art to, for example, (a) make copies of those polynucleotides, (b) obtain polynucleotides including at least a portion of such polynucleotides (e.g., polynucleotides including full-length genes, full-length coding regions, regulatory control sequences, truncated coding regions), and (c) obtain EG1117 polynucleotides for other plants, particularly since, as described in detail in the Examples section, knowledge of O. rufipogon EG1117 polynucleotides of the present invention enabled the isolation of O. sativa, Zea mays mays, and Zea mays parviglumis EG1117 polynucleotides of the present invention. Such polynucleotides can be obtained in a variety of ways including screening appropriate expression libraries with antibodies of the present invention; traditional cloning techniques using oligonucleotide probes of the present invention to screen appropriate libraries or DNA; and PCR amplification of appropriate libraries or DNA using oligonucleotide primers of the present invention. Preferred libraries are described above in the section entitled “EG307 polynucleotides.”

The present invention also includes polynucleotides that are oligonucleotides capable of hybridizing, under stringent hybridization conditions, with complementary regions of other, preferably longer, polynucleotides of the present invention such as those comprising plant EG1117 genes or other plant EG1117 polynucleotides. Oligonucleotides of the present invention can be RNA, DNA, or derivatives of either. The minimal size of such oligonucleotides is the size required to form a stable hybrid between a given oligonucleotide and the complementary sequence on another polynucleotide of the present invention. Minimal size characteristics are disclosed herein. The size of the oligonucleotide must also be sufficient for the use of the oligonucleotide in accordance with the present invention. Such applications are described above in the section entitled “EG307 polynucleotides.”

E. Recombinant Molecules

The present invention also includes a recombinant vector, which includes at least one plant EG307 or EG1117 polynucleotide of the present invention, inserted into any vector capable of delivering the polynucleotide into a host cell. Such a vector contains heterologous nucleic acid sequences, that is nucleic acid sequences that are not naturally found adjacent to polynucleotides of the present invention and that are derived from a species other than the species from which the polynucleotide(s) are derived. As used herein, a derived polynucleotide is one that is identical or similar in sequence to a polynucleotide or portion of a polynucleotide, but can contain modifications, such as modified bases, backbone modifications, nucleotide changes, and the like. The vector can be either RNA or DNA, either prokaryotic or eukaryotic, and typically is a virus or a plasmid. Recombinant vectors can be used in the cloning, sequencing, and/or otherwise manipulating of plant EG307 or EG1117 polynucleotides of the present invention. One type of recombinant vector, referred to herein as a recombinant molecule and described in more detail below, can be used in the expression of polynucleotides of the present invention. Preferred recombinant vectors are capable of replicating in the transformed cell.

Suitable and preferred polynucleotides to include in recombinant vectors of the present invention are as disclosed herein for suitable and preferred plant EG307 or EG1117 polynucleotides per se. Particularly preferred polynucleotides to include in recombinant vectors, and particularly in recombinant molecules, of the present invention include SEQ ID NO:1, SEQ ID NO:91, SEQ ID. NO:2, SEQ ID NO:4, SEQ ID NO:5, SEQ ID NO:7, SEQ ID NO:10, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID. NO:23, SEQ ID NO:24, SEQ ID NO:25, SEQ ID NO:27, SEQ ID NO:28, SEQ ID NO:29, SEQ ID NO:30, SEQ ID NO:31, SEQ ID NO:33, SEQ ID NO:34, SEQ ID. NO:35, SEQ ID NO:37, SEQ ID NO:38, SEQ ID NO:40, SEQ ID NO:41, SEQ ID NO:42, SEQ ID NO:44, SEQ ID NO:45, SEQ ID NO:46, SEQ ID NO:47, SEQ ID NO:49, SEQ ID. NO:50, SEQ ID NO:51, SEQ ID NO:53, SEQ ID NO:54, SEQ ID NO:55, SEQ ID NO:57, SEQ ID NO:58, SEQ ID NO:60, SEQ ID NO:62, SEQ ID NO:63, SEQ ID NO:64, SEQ ID NO:66, SEQ ID NO:67, SEQ ID NO:69, SEQ ID. NO:70, SEQ ID NO:71, SEQ ID NO:73, SEQ ID NO:74, SEQ ID NO:75, SEQ ID NO:77, SEQ ID NO:59, and/or SEQ ID NO:78. Alternative preferred polynucleotides to include in recombinant vectors, and particularly in recombinant molecules, of the present invention include SEQ ID NO:92, SEQ ID NO:93, SEQ ID NO:94, SEQ ID NO:96, SEQ ID NO:97, SEQ ID NO:98, SEQ ID NO:100, SEQ ID NO:101, SEQ ID NO:102, SEQ ID NO:103, SEQ ID NO:104, SEQ ID NO:104, SEQ ID NO:106, SEQ ID NO:107, SEQ ID NO:109, SEQ ID NO:110, SEQ ID NO:112, SEQ ID NO:113, SEQ ID NO:114, SEQ ID NO:116, SEQ ID NO:117, SEQ ID NO:119, SEQ ID NO:120, SEQ ID NO:121, SEQ ID NO:122, SEQ ID NO:123, SEQ ID NO:124, SEQ ID NO:125, SEQ ID NO:127, SEQ ID NO:128, SEQ ID NO:129, SEQ ID NO:130, SEQ ID NO:131, SEQ ID NO:133, SEQ ID NO:135, SEQ ID NO:136, SEQ ID NO:137, SEQ ID NO:138, SEQ ID NO:140, SEQ ID NO:141, SEQ ID NO:142, SEQ ID NO:144, SEQ ID NO:145, SEQ ID NO:146, SEQ ID NO:147, SEQ ID NO:149, SEQ ID NO:150, SEQ ID NO:151, SEQ ID NO:152, SEQ ID NO:154, SEQ ID NO:155, SEQ ID NO:157, SEQ ID NO:158, SEQ ID NO:160, SEQ ID NO:161, SEQ ID NO:162, SEQ ID NO:163, SEQ ID NO:165, SEQ ID NO:166, SEQ ID NO:167, and/or SEQ ID NO:168.

Isolated plant EG307 or EG1117 polypeptides of the present invention can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. A preferred cell to culture is a recombinant cell that is capable of expressing the polypeptide, the recombinant cell being produced by transforming a host cell with one or more polynucleotides of the present invention. Transformation of a polynucleotide into a cell can be accomplished by any method by which a polynucleotide can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed polynucleotides of the present invention can remain extrachromosomal or can integrate into one or more sites within a chromosome of the transformed (i.e., recombinant) cell in such a manner that their ability to be expressed is retained. Suitable and preferred polynucleotides with which to transform a cell are as disclosed herein for suitable and preferred plant EG307 or EG1117 polynucleotides per se. Particularly preferred polynucleotides to include in recombinant cells of the present invention include SEQ ID NO:1, SEQ ID NO:91, SEQ ID. NO:2, SEQ ID NO:4, SEQ ID NO:5, SEQ ID NO:7, SEQ ID NO:10, SEQ ID NO:11, SEQ ID NO:12, SEQ ID NO:14, SEQ ID NO:15, SEQ ID NO:17, SEQ ID NO:18, SEQ ID NO:20, SEQ ID NO:21, SEQ ID. NO:23, SEQ ID NO:24, SEQ ID NO:25, SEQ ID NO:27, SEQ ID NO:28, SEQ ID NO:29, SEQ ID NO:30, SEQ ID NO:31, SEQ ID NO:33, SEQ ID NO:34, SEQ ID. NO:35, SEQ ID NO:37, SEQ ID NO:38, SEQ ID NO:40, SEQ ID NO:41, SEQ ID NO:42, SEQ ID NO:44, SEQ ID NO:45, SEQ ID NO:46, SEQ ID NO:47, SEQ ID NO:49, SEQ ID. NO:50, SEQ ID NO:51, SEQ ID NO:53, SEQ ID NO:54, SEQ ID NO:55, SEQ ID NO:57, SEQ ID NO:58, SEQ ID NO:60, SEQ ID NO:62, SEQ ID NO:63, SEQ ID NO:64, SEQ ID NO:66, SEQ ID NO:67, SEQ ID NO:69, SEQ ID. NO:70, SEQ ID NO:71, SEQ ID NO:73, SEQ ID NO:74, SEQ ID NO:75, SEQ ID NO:77, SEQ ID NO:59, SEQ ID NO:78, SEQ ID NO:80, SEQ ID NO:81, SEQ ID NO:82, SEQ ID NO:84, and/or SEQ ID NO:85. Alternative preferred polynucleotides to include in recombinant cells of the present invention include SEQ ID NO:92, SEQ ID NO:93, SEQ ID NO:94, SEQ ID NO:96, SEQ ID NO:97, SEQ ID NO:98, SEQ ID NO:100, SEQ ID NO:101, SEQ ID NO:102, SEQ ID NO:103, SEQ ID NO:104, SEQ ID NO:104, SEQ ID NO:106, SEQ ID NO:107, SEQ ID NO:109, SEQ ID NO:110, SEQ ID NO:112, SEQ ID NO:113, SEQ ID NO:114, SEQ ID NO:116, SEQ ID NO:117, SEQ ID NO:119, SEQ ID NO:120, SEQ ID NO:121, SEQ ID NO:122, SEQ ID NO:123, SEQ ID NO:124, SEQ ID NO:125, SEQ ID NO:127, SEQ ID NO:128, SEQ ID NO:129, SEQ ID NO:130, SEQ ID NO:131, SEQ ID NO:133, SEQ ID NO:135, SEQ ID NO:136, SEQ ID NO:137, SEQ ID NO:138, SEQ ID NO:140, SEQ ID NO:141, SEQ ID NO:142, SEQ ID NO:144, SEQ ID NO:145, SEQ ID NO:146, SEQ ID NO:147, SEQ ID NO:149, SEQ ID NO:150, SEQ ID NO:151, SEQ ID NO:152, SEQ ID NO:154, SEQ ID NO:155, SEQ ID NO:157, SEQ ID NO:158, SEQ ID NO:160, SEQ ID NO:161, SEQ ID NO:162, SEQ ID NO:163, SEQ ID NO:165, SEQ ID NO:166, SEQ ID NO:167, and/or SEQ ID NO:168.

Suitable host cells to transform include any cell that can be transformed with a polynucleotide of the present invention. Host cells can be either untransformed cells or cells that are already transformed with at least one polynucleotide. Host cells of the present invention either can be endogenously (i.e., naturally) capable of producing plant EG307 or EG1117 polypeptides of the present invention or can be capable of producing such polypeptides after being transformed with at least one polynucleotide of the present invention. Host cells of the present invention can be any cell capable of producing at least one polypeptide of the present invention, and include bacterial, fungal (including yeast and rice blast, Magnaporthe grisea), parasite (including nematodes, especially of the genera Xiphinema, Helicotylenchus, and Tylenchlohynchus), insect, other animal and plant cells.

Suitable host viruses to transform include any virus that can be transformed with a polynucleotide of the present invention, including, but not limited to, rice stripe virus, and echinochloa hoja blanca virus.

In a preferred embodiment, non-pathogenic symbiotic bacteria, which are able to live and replicate within plant tissues, so-called endophytes, or non-pathogenic symbiotic bacteria, which are capable of colonizing the phyllosphere or the rhizosphere, so-called epiphytes, are used. Such bacteria include bacteria of the genera Agrobacterium, Alcaligenes, Azospirillum, Azotobacter, Bacillus, Clavibacter, Enterobacter, Erwinia, Flavobacter, Klebsiella, Pseudomonas, Rhizobium, Serratia, Streptomyces and Xanthomonas. Symbiotic fungi, such as Trichoderma and Gliocladium are also possible hosts for expression of the inventive nucleotide sequences for the same purpose.

A recombinant cell is preferably produced by transforming a host cell with one or more recombinant molecules, each comprising one or more polynucleotides of the present invention operatively linked to an expression vector containing one or more transcription control sequences. The phrase “operatively linked” refers to insertion of a polynucleotide into an expression vector in a manner such that the molecule is able to be expressed in the correct reading frame when transformed into a host cell. As used herein, an expression vector is a DNA or RNA vector that is capable of transforming a host cell and of effecting expression of a specified polynucleotide. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, parasite, insect, other animal, and plant cells. Preferred expression vectors of the present invention can direct gene expression in bacterial, yeast, fungal, insect and mammalian cells and more preferably in the cell types heretofore disclosed.

Recombinant molecules of the present invention may also (a) contain secretory signals (i.e., signal segment nucleic acid sequences) to enable an expressed EG307 or EG1117 polypeptide of the present invention to be secreted from the cell that produces the polypeptide and/or (b) contain fusion sequences which lead to the expression of polynucleotides of the present invention as fusion polypeptides. Examples of suitable signal segments and fusion segments encoded by fusion segment nucleic acids are disclosed herein. Eukaryotic recombinant molecules may include intervening and/or untranslated sequences surrounding and/or within the nucleic acid sequences of polynucleotides of the present invention. Suitable signal segments include natural signal segments or any heterologous signal segment capable of directing the secretion of a polypeptide of the present invention. Preferred signal and fusion sequences employed to enhance organ and organelle specific expression include, but are not limited to, arcelin-5, see Goossens, A. et. al. The arcelin-5 Gene of Phaseolus vulgaris directs high seed-specific expression in transgenic Phaseolus acutifolius and Arabidopsis plants. Plant Physiology (1999) 120:1095-1104, phaseolin, see Sengupta-Gopalan, C. et. al. Developmentally regulated expression of the bean beta-phaseolin gene in tobacco seeds. PNAS (1985) 82:3320-3324, hydroxyproline-rich glycoprotein, serpin, see Yan, X. et. al. Gene fusions of signal sequences with a modified beta-glucuronidase gene results in retention of the beta-glucuronidase protein in the secretory pathway/plasma membrane. Plant Physiology (1997) 115:915-924, N-acetyl glucosaminyl transferase 1, see Essl, D. et. al. The N-terminal 77 amino acids from tobacco N-acetylglucosaminyltransferase I are sufficient to retain reporter protein in the Golgi apparatus of Nicotiana benthamiana cells. Febs Letters (1999) 453(1-2):169-73, albumin, see Vandekerckhove, J. et. al. Enkephalins produced in transgenic plants using modified 2S seed storage proteins. BioTechnology 7:929-932 (1989) and PR1, see Pen, J. et. al. Efficient production of active industrial enzymes in plants. Industrial Crops and Prod. (1993) 1:241-250.

Polynucleotides of the present invention can be operatively linked to expression vectors containing regulatory sequences such as transcription control sequences, translation control sequences, origins of replication, and other regulatory sequences that are compatible with the recombinant cell and that control the expression of polynucleotides of the present invention. In particular, recombinant molecules of the present invention include transcription control sequences. Transcription control sequences are sequences which control the initiation, elongation, and termination of transcription. Included are those transcription control sequences which are sufficient to render promoter-dependent gene expression controllable for cell-type specific, tissue-specific or inducible by external signals or agents; such elements may be located in the 5′ or 3′ regions of the native gene. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, fungal, insect and mammalian cells, such as, but not limited to, tac, lac, trp, trc, oxy-pro, omp/lpp, rrnB, bacteriophage lambda (λ) (such as λp_{L} and λp_{R} and fusions that include such promoters), bacteriophage T7, T7lac, bacteriophage T3, bacteriophage SP6, bacteriophage SP01, metallothionein, α-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, poxvirus, adenovirus, cytomegalovirus (such as intermediate early promoters, simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells.

Particularly preferred transcription control sequences are plant transcription control sequences. The choice of transcription control sequence will vary depending on the temporal and spatial requirements for expression, and also depending on the target species. Thus, expression of the nucleotide sequences of this invention in any plant organ (leaves, roots, seedlings, immature or mature reproductive structures, etc.) or at any stage of plant development is preferred. Although many transcription control sequences from dicotyledons have been shown to be operational in monocotyledons and vice versa, ideally dicotyledonous transcription control sequences are selected for expression in dicotyledons, and monocotyledonous promoters for expression in monocotyledons. However, there is no restriction to the provenance of selected transcription control sequences; it is sufficient that they are operational in driving the expression of the nucleotide sequences in the desired cell.

Preferred transcription control sequences that are expressed constitutively include but are not limited to promoters from genes encoding actin or ubiquitin and the CaMV 35S and 19S promoters. The nucleotide sequences of this invention can also be expressed under the regulation of promoters that are chemically regulated. This enables the EG307 or EG1117 polypeptide to be synthesized only when the crop plants are treated with the inducing chemicals. Preferred technology for chemical induction of gene expression is detailed in the published application EP 0 332 104 (to Ciba-Geigy) and U.S. Pat. No. 5,614,395. A preferred promoter for chemical induction is the tobacco PR-1a promoter.

A preferred category of promoters is that which is induced by the physiological state of the plant (i.e. wound inducible, water-stress inducible, salt-stress inducible, disease inducible, and the like). Numerous promoters have been described which are expressed at wound sites and also at the sites of phytopathogen infection. Ideally, such a promoter should only be active locally at the sites of infection, and in this way the EG307 or EG1117 polypeptides only accumulate in cells in which the accumulation is desired. Preferred promoters of this kind include those described by Stanford et al. Mol. Gen. Genet. 215: 200-208 (1989), Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), and Warner et al. Plant J. 3: 191-201 (1993).

Preferred tissue-specific expression patterns include but are not limited to green tissue specific, root specific, stem specific, and flower specific. Promoters suitable for expression in green tissue include many which regulate genes involved in photosynthesis and many of these have been cloned from both monocotyledons and dicotyledons. A preferred promoter is the maize PEPC promoter from the phosphoenol carboxylase gene (Hudspeth & Grula, Plant Molec. Biol. 12: 579-589 (1989)). A preferred promoter for root specific expression is that described by de Framond (FEBS 290: 103-106 (1991); EP 0 452 269 to Ciba-Geigy). A preferred stem specific promoter is that described in U.S. Pat. No. 5,625,136 (to Ciba-Geigy) and which drives expression of the maize trpA gene.

A recombinant molecule of the present invention is a molecule that can include at least one of any polynucleotide heretofore described operatively linked to at least one of any transcription control sequence capable of effectively regulating expression of the polynucleotide(s) in the cell to be transformed, examples of which are disclosed herein.

A recombinant cell of the present invention includes any cell transformed with at least one of any polynucleotide of the present invention. Suitable and preferred polynucleotides as well as suitable and preferred recombinant molecules with which to transfer cells are disclosed herein.

Recombinant cells of the present invention can also be co-transformed with one or more recombinant molecules including plant EG307 or EG1117 polynucleotides encoding one or more polypeptides of the present invention and one or more other polypeptides useful when expressed in plants.

It may be appreciated by one skilled in the art that use of recombinant DNA technologies can improve expression of transformed polynucleotides by manipulating, for example, the number of copies of the polynucleotides within a host cell, the efficiency with which those polynucleotides are transcribed, the efficiency with which the resultant transcripts are translated, and the efficiency of post-translational modifications. Recombinant techniques useful for increasing the expression of polynucleotides of the present invention include, but are not limited to, operatively linking polynucleotides to high-copy number plasmids, integration of the polynucleotides into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotides of the present invention to correspond to the codon usage of the host cell, deletion of sequences that destabilize transcripts, and use of control signals that temporally separate recombinant cell growth from recombinant enzyme production during fermentation. The activity of an expressed recombinant polypeptide of the present invention may be improved by fragmenting, modifying, or derivatizing polynucleotides encoding such a polypeptide.

Recombinant cells of the present invention can be used to produce one or more polypeptides of the present invention by culturing such cells under conditions effective to produce such a polypeptide, and recovering the polypeptide. Effective conditions to produce a polypeptide include, but are not limited to, appropriate media, bioreactor, temperature, pH and oxygen conditions that permit polypeptide production. An appropriate, or effective, medium refers to any medium in which a cell of the present invention, when cultured, is capable of producing an EG307 or EG1117 polypeptide of the present invention. Such a medium is typically an aqueous medium comprising assimilable carbon, nitrogen and phosphate sources, as well as appropriate salts, minerals, metals and other nutrients, such as vitamins. The medium may comprise complex nutrients or may be a defined minimal medium. Cells of the present invention can be cultured in conventional fermentation bioreactors, which include, but are not limited to, batch, fed-batch, cell recycle, and continuous fermentors. Culturing can also be conducted in shake flasks, test tubes, microtiter dishes, and petri plates. Culturing is carried out at a temperature, pH and oxygen content appropriate for the recombinant cell. Such culturing conditions are well within the expertise of one of ordinary skill in the art.

Depending on the vector and host system used for production, resultant polypeptides of the present invention may either remain within the recombinant cell; be secreted into the fermentation medium; be secreted into a space between two cellular membranes, such as the periplasmic space in E. coli; or be retained on the outer surface of a cell or viral membrane.

The phrase “recovering the polypeptide” refers simply to collecting the whole fermentation medium containing the polypeptide and need not imply additional steps of separation or purification. Polypeptides of the present invention can be purified using a variety of standard polypeptide purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. Polypeptides of the present invention are preferably retrieved in “substantially pure” form. As used herein, “substantially pure” refers to a purity that allows for the effective use of the polypeptide as a diagnostic or test compound, and means, with increasing preference, at least 50%, 60%, 70%, 80%, 90%, 95%, or 98% homogeneous.

F. Transfected Plant Cells and Transgenic Plants

With regard to EG307 and EG1117, particularly preferred recombinant cells are plant cells. By “plant cell” is meant any self-propagating cell bounded by a semi-permeable membrane and containing a plastid. Such a cell also requires a cell wall if further propagation is desired. Plant cell, as used herein includes, without limitation, algae, cyanobacteria, seeds, suspension cultures, embryos, meristematic regions, callus tissue, leaves, roots, shoots, gametophytes, sporophytes, pollen, and microspores.

In a particularly preferred embodiment, at least one (or both) of the EG307 or EG1117 polypeptides or an allele or mutant form thereof, of the invention is expressed in a higher organism, e.g., a plant. In this case, transgenic plants expressing effective amounts of the polypeptides exhibit improved economic productivity. A nucleotide sequence of the present invention is inserted into an expression cassette, which is then preferably stably integrated in the genome of said plant. In another preferred embodiment, the nucleotide sequence is included in a non-pathogenic self-replicating virus. Plants transformed in accordance with the present invention may be monocots or dicots and include, but are not limited to, maize, wheat, barley, rye, millet, chickpea, lentil, flax, olive, fig almond, pistachio, walnut, beet, parsnip, citrus fruits, including, but not limited to, orange, lemon, lime, grapefruit, tangerine, minneola, and tangelo, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugarbeet, sunflower, rapeseed, clover, tobacco, carrot, cotton, alfalfa, rice, potato, eggplant, cucumber, Arabidopsis, and woody plants such as coniferous and deciduous trees.

Once a desired nucleotide sequence has been transformed into a particular plant species, it may be propagated in that species or moved into other varieties of the same species, particularly including commercial varieties, using traditional breeding techniques.

Accordingly, the present invention provides a method for producing a transfected plant cell or transgenic plant comprising the steps of a) transfecting a plant cell to contain a heterologous DNA segment encoding a protein and derived from an EG307 and/or EG1117 polynucleotide not native to said cell (the polynucleotide indeed could be native but the expression pattern could be developmentally altered, still leading to the preferred effect); wherein said polynucleotide is operably linked to a promoter that can be used effectively for expression of transgenic proteins; b) optionally growing and maintaining said cell under conditions whereby a transgenic plant is regenerated therefrom; c) optionally growing said transgenic plant under conditions whereby said DNA is expressed, whereby the total amount of EG307 and/or EG1117 polypeptide in said plant is altered. In a preferred embodiment, the method further comprises the step of obtaining and growing additional generations of descendants of said transgenic plant which comprise said heterologous DNA segment wherein said heterologous DNA segment is expressed. As used herein, “heterologous DNA”, or in some cases, “transgene” refers to foreign genes or polynucleotides, or additional, or modified versions of native or endogenous genes or polynucleotides (perhaps driven by different promoters) in order to alter the traits of a plant in a specific manner.

The invention also provides plant cells which comprise heterologous DNA encoding an EG307 and/or EG1117 polypeptide. In a preferred embodiment, the transgenic plant cell is a propagation material of a transgenic plant. The present invention also provides a transfected host cell comprising a host cell transfected with a construct comprising a promoter, enhancer or intron polynucleotide from an evolutionarily significant EG307 and/or EG1117 polynucleotide, and a polynucleotide encoding a reporter protein.

The present invention also provides a method of providing improved economic productivity in a plant comprising: a) producing a transfected plant cell having a transgene encoding an EG307 and/or EG1117 polypeptide whereby EG307 and/or EG1117 expression in said plant cell is altered; and b) growing a transgenic plant from the transfected plant cell wherein the EG307 and/or EG1117 transgene is expressed in the transgenic plant. The expression of the transgene includes an increase in EG307 and/or EG1117 expression. In some embodiments, the expression of the transgene produces an RNA that may interfere with a native EG307 and/or EG1117 gene such that the expression of the native gene is either eliminated or reduced, resulting in a useful outcome.

The invention also provides a transgenic plant containing heterologous DNA which encodes an EG307 and/or EG1117 polypeptide that is expressed in plant tissue, including expression in a vector introduced into the plant.

The present invention also provides an isolated polynucleotide which includes a transcription control element operably linked to a polynucleotide that encodes the EG307 and/or EG1117 gene in plant tissue. In preferred embodiment, the transcription control element is the promoter native to an EG307 and/or EG1117 gene.

The present invention also provides a method of making a transfected cell comprising a) identifying an evolutionarily significant EG307 and/or EG1117 polynucleotide in a domesticated plant; b) using said EG307 and/or EG1117 polynucleotide to identify a non-polypeptide coding sequence that may be a transcription or translation regulatory element, enhancer, intron or other 5′ or 3′ flanking sequence; c) assembling a construct comprising said non-polypeptide coding sequence and a polynucleotide encoding a reporter protein; and d) transfecting said construct into a host cell. The present invention also provides a transfected cell produced according to this method. In one embodiment, the host cell is a plant cell, and the method further comprises the step of growing and maintaining the cell under conditions suitable for regenerating a transgenic plant. Also provided is a transgenic plant produced by the method.

A nucleotide sequence of this invention is preferably expressed in transgenic plants, thus causing the biosynthesis of the corresponding EG307 and/or EG1117 polypeptide in the transgenic plants. In this way, transgenic plants with characteristics related to improved economic productivity are generated. For their expression in transgenic plants, the nucleotide sequences of the invention may require modification and optimization. Although preferred gene sequences may be adequately expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17. 477-498 (1989)). All changes required to be made within the nucleotide sequences such as those described above are made using well known techniques of site directed mutagenesis, PCR, and synthetic gene construction using the methods described in the published patent applications EP 0 385 962 (to Monsanto), EP 0 359 472 (to Lubrizol), and WO 93/07278 (to Ciba-Geigy).

For efficient initiation of translation, sequences adjacent to the initiating methionine may require modification. For example, they can be modified by the inclusion of sequences known to be effective in plants. Joshi has suggested an appropriate consensus for plants (NAR 15: 6643-6653 (1987)) and Clontech suggests a further consensus translation initiator (1993/1994 catalog, page 210). These consensuses are suitable for use with the nucleotide sequences of this invention. The sequences are incorporated into constructions comprising the nucleotide sequences, up to and including the ATG (while leaving the second amino acid unmodified), or alternatively up to and including the GTC subsequent to the ATG (with the possibility of modifying the second amino acid of the transgene).

Expression of the nucleotide sequences in transgenic plants is driven by transcription control elements shown to be functional in plants. Transformation of plants with a polynucleotide under the control of these regulatory elements provides for controlled expression in the transformed plant. Such transcription control elements have been described above. In addition to the selection of a suitable initiator of transcription, constructions for expression of EG307 and/or EG1117 polypeptide in plants require an appropriate transcription terminator to be attached downstream of the heterologous nucleotide sequence. Several such terminators are available and known in the art (e.g. tm1 from CaMV, E9 from rbcS). Any available terminator known to function in plants can be used in the context of this invention.

Numerous other sequences can be incorporated into expression cassettes described in this invention. These include sequences which have been shown to enhance expression such as intron sequences (e.g. from Adh1 and bronze1) and viral leader sequences (e.g. from TMV, MCMV and AMV).

The present invention also provides a method of increasing yield in a plant comprising a) producing a transgenic plant cell having a transgene encoding an EG307 and/or EG1117 polypeptide and the transgene is under the control of regulatory sequences suitable for controlled expression of the gene(s); and b) growing a transgenic plant from the transgenic plant cell wherein the EG307 and/or EG1117 transgene is expressed in the transgenic plant.

The present invention also provides a method of increasing yield in a plant comprising a) producing a transfected plant cell having a transgene containing the EG307 and/or EG1117 gene under the control of a promoter providing constitutive expression of the EG307 and/or EG1117 gene; and b) growing a transgenic plant from the transgenic plant cell wherein the EG307 and/or EG1117 transgene is expressed constitutively in the transgenic plant.

The present invention also provides a method of providing controllable yield in a transgenic plant comprising: a) producing a transfected plant cell having a transgene containing the EG307 and/or EG1117 gene under the control of a promoter providing controllable expression of the EG307 and/or EG1117 gene; and b) growing a transgenic plant from the transgenic plant cell wherein the EG307 and/or EG1117 transgene is controllably expressed in the transgenic plant. In one embodiment, the EG307 and/or EG1117 gene is expressed using a tissue-specific or cell type-specific promoter, or by a promoter that is activated by the introduction of an external signal or agent, such as a chemical signal or agent.

It may be preferable to target expression of the nucleotide sequences of the present invention to different cellular localizations in the plant. In some cases, localization in the cytosol may be desirable, whereas in other cases, localization in some subcellular organelle may be preferred. Subcellular localization of heterologous DNA encoded polypeptides is undertaken using techniques well known in the art. Typically, the DNA encoding the target peptide from a known organelle-targeted gene product is manipulated and fused upstream of the nucleotide sequence. Many such target sequences are known for the chloroplast and their functioning in heterologous constructions has been shown. The expression of the nucleotide sequences of the present invention is also targeted to the endoplasmic reticulum or to the vacuoles of the host cells. Techniques to achieve this are well-known in the art.

Vectors suitable for plant transformation are described elsewhere in this specification. For Agrobacterium -mediated transformation, binary vectors or vectors carrying at least one T-DNA border sequence are suitable, whereas for direct gene transfer any vector is suitable and linear DNA containing only the construction of interest may be preferred. In the case of direct gene transfer, transformation with a single DNA species or co-transformation can be used (Schocher et al. Biotechnology 4: 1093-1096 (1986)). For both direct gene transfer and Agrobacterium-mediated transfer, transformation is usually (but not necessarily) undertaken with a selectable marker which may provide resistance to an antibiotic (kanamycin, hygromycin or methotrexate) or a herbicide (basta). The choice of selectable marker is not, however, critical to the invention.

In another preferred embodiment, a nucleotide sequence of the present invention is directly transformed into the plastid genome. A major advantage of plastid transformation is that plastids are capable of expressing multiple open reading frames under control of a single promoter. Plastid transformation technology is extensively described in U.S. Pat. Nos. 5,451,513, 5,545,817, and 5,545,818, in PCT application no. WO 95/16783, and in McBride et al. (1994) Proc. Natl. Acad. Sci. USA 91, 7301-7305. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Natl. Acad. Sci. USA 87, 8526-8530; Staub, J. M., and Maliga, P. (1992) Plant Cell 4, 39-45). This resulted in stable homoplasmic transformants at a frequency of approximately one per 100 bombardments of target leaves. The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-polypeptide antibiotic resistance genes with a dominant selectable marker, the bacterial aadA gene encoding the spectinomycin-detoxifying enzyme aminoglycoside-3′-adenyltransferase (Svab, Z., and Maliga, P. (1993) Proc. Natl. Acad. Sci. USA 90, 913-917). Previously, this marker had been used successfully for high-frequency transformation of the plastid genome of the green alga Chlamydomonas reinhardtii (Goldschmidt-Clermont, M. (1991) Nucl. Acids Res. 19: 4083-4089). Other selectable markers useful for plastid transformation are known in the art and encompassed within the scope of the invention. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant polypeptide. In a preferred embodiment, a nucleotide sequence of the present invention is inserted into a plastid targeting vector and transformed into the plastid genome of a desired plant host. Plants homoplastic for plastid genomes containing a nucleotide sequence of the present invention are obtained, and are preferentially capable of high expression of the nucleotide sequence.

The present invention also provides a method of identifying a plant yield-related gene comprising: a) providing a plant tissue sample; b) introducing into the plant tissue sample a candidate plant yield-related gene; c) expressing the candidate plant yield-related gene within the plant tissue sample; and d) determining whether the plant tissue sample exhibits change in yield response, whereby a change in response identifies a plant yield-related gene. The present invention also provides plant yield-related genes isolated according to the method.

Yield response, as used herein, is measured by techniques well known to those skilled in the art. In the cereals yield response is determined, for example, by one or more of the following metrics, grain weight, grain length, grain weight/1000 grains, size of panicle, number of panicles, and number of grains/panicle.

G. EG307 or EG1117 Antibodies

The present invention also includes isolated antibodies capable of selectively binding to an EG307 or EG1117 polypeptide of the present invention or to a mimetope thereof. Such antibodies are also referred to herein as anti-EG307 or anti-EG1117 antibodies. Particularly preferred antibodies of this embodiment include anti-O. sativa EG307 antibodies, anti-O. rufipogon EG307 antibodies, anti-Z. mays EG307 antibodies, anti-O. sativa EG1117 antibodies, anti-O. rufipogon EG1117 antibodies, anti-Z. mays EG117 antibodies.

Isolated antibodies are antibodies that have been removed from their natural milieu. The term “isolated” does not refer to the state of purity of such antibodies. As such, isolated antibodies can include anti-sera containing such antibodies, or antibodies that have been purified to varying degrees.

As used herein, the term “selectively binds to” refers to the ability of antibodies of the present invention to preferentially bind to specified polypeptides and mimetopes thereof of the present invention. Binding can be measured using a variety of methods known to those skilled in the art including immunoblot assays, immunoprecipitation assays, radioimmunoassays, enzyme immunoassays (e.g., ELISA), immunofluorescent antibody assays and immunoelectron microscopy; see, for example, Sambrook et al., ibid., and Harlow & Lane, 1990, ibid.

Antibodies of the present invention can be either polyclonal or monoclonal antibodies. Antibodies of the present invention include functional equivalents such as antibody fragments and genetically-engineered antibodies, including single chain antibodies, that are capable of selectively binding to at least one of the epitopes of the polypeptide or mimetope used to obtain the antibodies. Antibodies of the present invention also include chimeric antibodies that can bind to more than one epitope. Preferred antibodies are raised in response to polypeptides, or mimetopes thereof, that are encoded, at least in part, by a polynucleotide of the present invention.

A preferred method to produce antibodies of the present invention includes (a) administering to an animal an effective amount of a polypeptide or mimetope thereof of the present invention to produce the antibodies and (b) recovering the antibodies. In another method, antibodies of the present invention are produced recombinantly using techniques as heretofore disclosed to produce EG307 or EG1117 polypeptides of the present invention.

Antibodies of the present invention have a variety of potential uses that are within the scope of the present invention. For example, such antibodies can be used (a) as reagents in assays to detect expression of EG307 or EG1117 by plant and/or (b) as tools to screen expression libraries and/or to recover desired polypeptides of the present invention from a mixture of polypeptides and other contaminants. Furthermore, antibodies of the present invention can be used to target cytotoxic agents to plants in order to directly kill such plants. Targeting can be accomplished by conjugating (i.e., stably joining) such antibodies to the cytotoxic agents using techniques known to those skilled in the art. Suitable cytotoxic agents are known to those skilled in the art. Suitable cytotoxic agents include, but are not limited to: double-chain polypeptides (i.e., toxins having A and B chains), such as diphtheria toxin, ricin toxin, Pseudomonas exotoxin, modeccin toxin, abrin toxin, and shiga toxin; single-chain toxins, such as pokeweed antiviral polypeptide, α-amanitin, and ribosome inhibiting polypeptides; and chemical toxins, such as melphalan, methotrexate, nitrogen mustard, doxorubicin and daunomycin. Preferred double-chain toxins are modified to include the toxic domain and translocation domain of the toxin but lack the toxin's intrinsic cell binding domain.

H. Formulation of Growth-Enhancing Compositions

The invention also includes compositions comprising at least one or both of the EG307 or EG1117 polypeptides of the present invention. In order to effectively control growth such compositions preferably contain sufficient amounts of polypeptide. Such amounts vary depending on the target crop, and on the environmental conditions, such as humidity, temperature or type of soil. In a preferred embodiment, compositions comprising the EG307 and/or EG1117 polypeptide comprise host cells expressing the polypeptides without additional purification. In another preferred embodiment, the cells expressing the EG307 and/or EG1117 polypeptides are lyophilized prior to their use as a growth-enhancing agent. In another embodiment, the EG307 or EG1117 polypeptides are engineered to be secreted from the host cells. In cases where purification of the polypeptides from the host cells in which they are expressed is desired, various degrees of purification of the EG307 or EG1117 polypeptides are reached.

The present invention further embraces the preparation of compositions comprising at least one EG307 or EG1117 polypeptide of the present invention, which is homogeneously mixed with one or more compounds or groups of compounds described herein. The present invention also relates to methods of treating plants, which comprise application of the EG307 or EG1117 polypeptides or compositions containing the EG307 or EG1117 polypeptides, to plants. The EG307 or EG1117 polypeptides can be applied to the crop area in the form of compositions or plant to be treated, simultaneously or in succession, with further compounds. These compounds can be both fertilizers or micronutrient donors or other preparations that influence plant growth. They can also be selective herbicides, insecticides, fungicides, bactericides, nematicides, molluscicides or mixtures of several of these preparations, if desired together with further carriers, surfactants or application-promoting adjuvants customarily employed in the art of formulation. Suitable carriers and adjuvants can be solid or liquid and correspond to the substances ordinarily employed in formulation technology, e.g. natural or regenerated mineral substances, solvents, dispersants, wetting agents, tackifiers, binders or fertilizers.

A preferred method of applying EG307 or EG1117 polypeptides of the present invention is by spraying the soil, water, or foliage of plants. The number of applications and the rate of application depend on the type of plant and the desired increase in yield. The EG307 or EG1117 polypeptides can also penetrate the plant through the roots via the soil (systemic action) by impregnating the locus of the plant with a liquid composition, or by applying the compounds in solid form to the soil, e.g. in granular form (soil application). The EG307 or EG1117 polypeptides may also be applied to seeds (coating) by impregnating the seeds either with a liquid formulation containing EG307 or EG1117 polypeptides, or coating them with a solid formulation. In special cases, further types of application are also possible, for example, selective treatment of the plant stems or buds.

The EG307 or EG1117 polypeptides are used in unmodified form or, preferably, together with the adjuvants conventionally employed in the art of formulation, and are therefore formulated in known manner to emulsifiable concentrates, coatable pastes, directly sprayable or dilutable solutions, dilute emulsions, wettable powders, soluble powders, dusts, granulates, and also encapsulations, for example, in polymer substances. Like the nature of the compositions, the methods of application, such as spraying, atomizing, dusting, scattering or pouring, are chosen in accordance with the intended objectives and the prevailing circumstances.

The formulations, compositions or preparations containing the EG307 or EG1117 polypeptides and, where appropriate, a solid or liquid adjuvant, are prepared in a known manner, for example by homogeneously mixing and/or grinding the EG307 or EG1117 polypeptides with extenders, for example solvents, solid carriers and, where appropriate, surface-active compounds (surfactants).

Suitable solvents include aromatic hydrocarbons, preferably the fractions having 8 to 12 carbon atoms, for example, xylene mixtures or substituted naphthalenes, phthalates such as dibutyl phthalate or dioctyl phthalate, aliphatic hydrocarbons such as cyclohexane or paraffins, alcohols and glycols and their ethers and esters, such as ethanol, ethylene glycol monomethyl or monoethyl ether, ketones such as cyclohexanone, strongly polar solvents such as N-methyl-2-pyrrolidone, dimethyl sulfoxide or dimethyl formamide, as well as epoxidized vegetable oils such as epoxidized coconut oil or soybean oil or water.

The solid carriers used e.g. for dusts and dispersible powders, are normally natural mineral fillers such as calcite, talcum, kaolin, montmorillonite or attapulgite. In order to improve the physical properties it is also possible to add highly dispersed silicic acid or highly dispersed absorbent polymers. Suitable granulated adsorptive carriers are porous types, for example pumice, broken brick, sepiolite or bentonite; and suitable nonsorbent carriers are materials such as calcite or sand. In addition, a great number of pregranulated materials of inorganic or organic nature can be used, e.g. especially dolomite or pulverized plant residues.

Suitable surface-active compounds are nonionic, cationic and/or anionic surfactants having good emulsifying, dispersing and wetting properties. The term “surfactants” will also be understood as comprising mixtures of surfactants. Suitable anionic surfactants can be both water-soluble soaps and water-soluble synthetic surface-active compounds.

Suitable soaps are the alkali metal salts, alkaline earth metal salts or unsubstituted or substituted ammonium salts of higher fatty acids (chains of 10 to 22 carbon atoms), for example the sodium or potassium salts of oleic or stearic acid, or of natural fatty acid mixtures which can be obtained for example from coconut oil or tallow oil. The fatty acid methyltaurin salts may also be used.

More frequently, however, so-called synthetic surfactants are used, especially fatty sulfonates, fatty sulfates, sulfonated benzimidazole derivatives or alkylarylsulfonates.

The fatty sulfonates or sulfates are usually in the form of alkali metal salts, alkaline earth metal salts or unsubstituted or substituted ammonium salts and have a 8 to 22 carbon alkyl radical which also includes the alkyl moiety of alkyl radicals, for example, the sodium or calcium salt of lignonsulfonic acid, of dodecylsulfate or of a mixture of fatty alcohol sulfates obtained from natural fatty acids. These compounds also comprise the salts of sulfuric acid esters and sulfonic acids of fatty alcohol/ethylene oxide adducts. The sulfonated benzimidazole derivatives preferably contain 2 sulfonic acid groups and one fatty acid radical containing 8 to 22 carbon atoms. Examples of alkylarylsulfonates are the sodium, calcium or triethanolamine salts of dodecylbenzenesulfonic acid, dibutylnapthalenesulfonic acid, or of a naphthalenesulfonic acid/formaldehyde condensation product. Also suitable are corresponding phosphates, e.g. salts of the phosphoric acid ester of an adduct of p-nonylphenol with 4 to 14 moles of ethylene oxide.

Non-ionic surfactants are preferably polyglycol ether derivatives of aliphatic or cycloaliphatic alcohols, or saturated or unsaturated fatty acids and alkylphenols, said derivatives containing 3 to 30 glycol ether groups and 8 to 20 carbon atoms in the (aliphatic) hydrocarbon moiety and 6 to 18 carbon atoms in the alkyl moiety of the alkylphenols.

Further suitable non-ionic surfactants are the water-soluble adducts of polyethylene oxide with polypropylene glycol, ethylenediamine propylene glycol and alkylpolypropylene glycol containing 1 to 10 carbon atoms in the alkyl chain, which adducts contain 20 to 250 ethylene glycol ether groups and 10 to 100 propylene glycol ether groups. These compounds usually contain 1 to 5 ethylene glycol units per propylene glycol unit.

Representative examples of non-ionic surfactants are nonylphenolpolyethoxyethanols, castor oil polyglycol ethers, polypropylene/polyethylene oxide adducts, tributylphenoxypolyethoxyethanol, polyethylene glycol and octylphenoxyethoxyethanol. Fatty acid esters of polyoxyethylene sorbitan and polyoxyethylene sorbitan trioleate are also suitable non-ionic surfactants.

Cationic surfactants are preferably quaternary ammonium salts which have, as N-substituent, at least one C8-C22 alkyl radical and, as further substituents, lower unsubstituted or halogenated alkyl, benzyl or lower hydroxyalkyl radicals. The salts are preferably in the form of halides, methylsulfates or ethylsulfates, e.g. stearyltrimethylammonium chloride or benzyldi(2-chloroethyl)ethylammonium bromide. The surfactants customarily employed in the art of formulation are described, for example, in “McCutcheon's Detergents and Emulsifiers Annual,” MC Publishing Corp. Ringwood, N.J., 1979, and Sisely and Wood, “Encyclopedia of Surface Active Agents,” Chemical Publishing Co., Inc. New York, 1980.

### IV. IDENTIFICATION OF GENES EVOLVED UNDER NEUTRAL CONDITIONS

As described in detail herein, K_{A}/K_{S} analysis allows the identification of positively selected protein-coding genes; however, this type of analysis can also be used to identify another set of evolutionarily significant genes, those genes evolving under neutral conditions.

A K_{A}/K_{S} ratio>1 signifies the role of positive selection, while conversely, a K_{A}/K_{S} ratio<1 suggests that a protein-coding gene has been negatively selected (i.e., has been conserved). As noted elsewhere herein, most genes (in fact, the vast majority) are conserved. Only rare genes exhibit a K_{A}/K_{S} ratio>1, since very few genes are positively selected. As described herein, genes that were positively selected during domestication of the cereals (as well as other crops) have significant commercial value; however, another set of genes contained in the genomes of domesticated plants has been neither positively (to produce a desired, enhanced trait in the domesticated descendant) nor negatively selected (conserved). This subset of plant genes, as noted above, also has a significant commercial value, and this set of genes can be identified by using K_{A}/K_{S} analysis, to be described here.

These genes comprise those that render the plant resistant to drought, disease, pests (including, but not limited to, insects, animal herbivores, and microbes), high salt levels, and other stresses. Attacks by pests, and damage by drought or high salt levels, etc, are responsible for annual losses of billions of dollars to farmers, seed companies, and the large agricultural companies. The identification of genes that render wild plants resistant to these stresses is thus of great value, both socially (to a hungry world), and economically.

The method to detect these genes is as follows. After plants were first domesticated (and subsequently, as the descendents are further domesticated), they were “pampered”, in the sense, for example, that humans supply water in sufficient quantities to meet the plant's needs. Thus the plant is not required to deal with drought stress “on its own”. Similarly, humans remove insect pests (either physically, or through the use of pesticides), and segregate domesticated plants away from animal herbivores, such that the domesticated plant is not constantly confronted with the need to deal with these pests. In fact, it has been well documented that domesticated cereals, for example, are usually much more vulnerable to drought, high salt levels, pests, and other stresses than are their wild relatives/ancestors. This is because organisms generally do not maintain abilities that are not required to survive. As humans take over these roles, domesticated plants can save the high metabolic costs (“metabolic extravagance”) of maintaining genes that code for stress-related traits.

This loss of resistance must of course stem from genetic differences (i.e., changes) between the ancestor and its pampered domesticated descendent. These genetic changes that result in loss of function can occur through three different mechanisms. The genes that code for these traits may actually be lost from the genome of the descendent crop. Gene loss has been documented and is a well-known phenomenon. Similarly, the genes that code for “unneeded” traits in a descendent crop may still persist in the genome, but are no longer expressed, as a result of promoter changes, for example. Alternatively, the genes coding for these unneeded traits may still be part of the genome, and may still be expressed, but the genes may have accumulated nucleotide substitutions that render the protein product either nonfunctional or less fully functional than the ancestral homolog. These genes are thus evolving neutrally.

Neutral amino acid replacements accumulate in the protein product of a gene that is free of selective pressures (either positive or negative). For a domesticated plant that has been freed of the need to maintain a functional protein product for the gene of interest, a condition of molecular neutrality exits. This includes genes that code for traits like pest, disease, drought, salt, etc., resistance. Such fully unconstrained, neutrally evolving genes are perfect candidates for detection by K_{A}/K_{S} analysis, as a neutrally evolving gene will ideally exhibit a K_{A}/K_{S} ratio=1, when the homolog from the ancestral and descendant plants are compared.

Thus the method invented and described here involves high-throughput sequencing of a cDNA library for an ancestral plant, BLASTING the resulting ESTs against a database of ESTs from the modern descendent, and performing K_{A}/K_{S} analysis for homologous pairs. The details of this process are explained elsewhere in this patent, for the case of a positively selected gene. The genes with a K_{A}/K_{S} ratio=1 will be the set of genes that control important stress resistant traits, and that these genes can be effectively and swiftly identified by use of this ratio. This commercially valuable set of genes includes those coding for desirable traits such resistance to pests, disease, drought, high salt levels, etc. To best identify these genes, the EST sequencing from both the modem domesticated and the ancestral species should be performed very carefully, with a high standard of accuracy. While one can make use of cereal EST databases available in GenBank, one may also resequence ESTs from cDNA libraries prepared specifically for this purpose. The accuracy of sequencing is important, because this will give rise to a very narrow distribution of gene pair comparisons between ancestral and modern homologs that have a K_{A}/K_{S} ratio equal to one. This will reduce the number of false positives to a minimum, thus expediting the process.

When the accuracy of the screening process is not stringently controlled, or is unknown, it is possible that sequencing errors will obscure a K_{A}/K_{S} ratio of 1.0, and for this reason, K_{A}/K_{S} values of between about 0.75-1.25 are checked carefully for evidence of neutral evolution.

Polynucleotides that have evolved under neutral conditions can then be mapped onto one of the known quantitative trait loci, or QTL, whereby the specific stress-resistance trait controlled by that polynucleotide may be rapidly and conclusively identified.

### V. SCREENING METHODS FOR IDENTIFICATION OF AGENTS

The present invention also provides screening methods using the polynucleotides and polypeptides identified and characterized using the above-described methods. These screening methods are useful for identifying agents which may modulate the function(s) of the polynucleotides or polypeptides in a manner that would be useful for enhancing or diminishing a characteristic in a domesticated or ancestor organism. Generally, the methods entail contacting at least one agent to be tested with a domesticated organism, ancestor organism, or transgenic organism or cell that has been transfected with a polynucleotide sequence identified by the methods described above, or a preparation of the polypeptide encoded by such polynucleotide sequence, wherein an agent is identified by its ability to modulate function of either the polynucleotide sequence or the polypeptide. For example, an agent can be a compound that is applied or contacted with a domesticated plant or animal to induce expression of the identified gene at a desired time. Specifically in regard to plants, an agent could be used, for example, to induce flowering at an appropriate time.

As used herein, the term “agent” means a biological or chemical compound such as a simple or complex organic or inorganic molecule, a peptide, a protein or an oligonucleotide. A vast array of compounds can be synthesized, for example oligomers, such as oligopeptides and oligonucleotides, and synthetic organic and inorganic compounds based on various core structures, and these are also included in the term “agent”. In addition, various natural sources can provide compounds for screening, such as plant or animal extracts, and the like. Compounds can be tested singly or in combination with one another.

To “modulate function” of a polynucleotide or a polypeptide means that the function of the polynucleotide or polypeptide is altered when compared to not adding an agent. Modulation may occur on any level that affects function. A polynucleotide or polypeptide function may be direct or indirect, and measured directly or indirectly. A “function” of a polynucleotide includes, but is not limited to, replication, translation, and expression pattern(s). A polynucleotide function also includes functions associated with a polypeptide encoded within the polynucleotide. For example, an agent which acts on a polynucleotide and affects protein expression, conformation, folding (or other physical characteristics), binding to other moieties (such as ligands), activity (or other functional characteristics), regulation and/or other aspects of protein structure or function is considered to have modulated polynucleotide function. The ways that an effective agent can act to modulate the expression of a polynucleotide include, but are not limited to 1) modifying binding of a transcription factor to a transcription factor responsive element in the polynucleotide; 2) modifying the interaction between two transcription factors necessary for expression of the polynucleotide; 3) altering the ability of a transcription factor necessary for expression of the polynucleotide to enter the nucleus; 4) inhibiting the activation of a transcription factor involved in transcription of the polynucleotide; 5) modifying a cell-surface receptor which normally interacts with a ligand and whose binding of the ligand results in expression of the polynucleotide; 6) inhibiting the inactivation of a component of the signal transduction cascade that leads to expression of the polynucleotide; and 7) enhancing the activation of a transcription factor involved in transcription of the polynucleotide.

A “function” of a polypeptide includes, but is not limited to, conformation, folding (or other physical characteristics), binding to other moieties (such as ligands), activity (or other functional characteristics), and/or other aspects of protein structure or functions. For example, an agent that acts on a polypeptide and affects its conformation, folding (or other physical characteristics), binding to other moieties (such as ligands), activity (or other functional characteristics), and/or other aspects of protein structure or functions is considered to have modulated polypeptide function. The ways that an effective agent can act to modulate the function of a polypeptide include, but are not limited to 1) changing the conformation, folding or other physical characteristics; 2) changing the binding strength to its natural ligand or changing the specificity of binding to ligands; and 3) altering the activity of the polypeptide.

Generally, the choice of agents to be screened is governed by several parameters, such as the particular polynucleotide or polypeptide target, its perceived function, its three-dimensional structure (if known or surmised), and other aspects of rational compound design. Techniques of combinatorial chemistry can also be used to generate numerous permutations of candidates. Those of skill in the art can devise and/or obtain suitable agents for testing.

The in vivo screening assays described herein may have several advantages over conventional drug screening assays: 1) if an agent must enter a cell to achieve a desired therapeutic effect, an in vivo assay can give an indication as to whether the agent can enter a cell; 2) an in vivo screening assay can identify agents that, in the state in which they are added to the assay system are ineffective to elicit at least one characteristic which is associated with modulation of polynucleotide or polypeptide function, but that are modified by cellular components once inside a cell in such a way that they become effective agents; 3) most importantly, an in vivo assay system allows identification of agents affecting any component of a pathway that ultimately results in characteristics that are associated with polynucleotide or polypeptide function.

In general, screening can be performed by adding an agent to a sample of appropriate cells which have been transfected with a polynucleotide identified using the methods of the present invention, and monitoring the effect, i.e., modulation of a function of the polynucleotide or the polypeptide encoded within the polynucleotide. The experiment preferably includes a control sample which does not receive the candidate agent. The treated and untreated cells are then compared by any suitable phenotypic criteria, including but not limited to microscopic analysis, viability testing, ability to replicate, histological examination, the level of a particular RNA or polypeptide associated with the cells, the level of enzymatic activity expressed by the cells or cell lysates, the interactions of the cells when exposed to infectious agents, and the ability of the cells to interact with other cells or compounds. Differences between treated and untreated cells indicate effects attributable to the candidate agent. Optimally, the agent has a greater effect on experimental cells than on control cells. Appropriate host cells include, but are not limited to, eukaryotic cells, preferably plant or animal cells. The choice of cell will at least partially depend on the nature of the assay contemplated.

To test for agents that upregulate the expression of a polynucleotide, a suitable host cell transfected with a polynucleotide of interest, such that the polynucleotide is expressed (as used herein, expression includes transcription and/or translation) is contacted with an agent to be tested. An agent would be tested for its ability to result in increased expression of mRNA and/or polypeptide. Methods of making vectors and transfection are well known in the art. “Transfection” encompasses any method of introducing the exogenous sequence, including, for example, lipofection, transduction, infection or electroporation. The exogenous polynucleotide may be maintained as a non-integrated vector (such as a plasmid) or may be integrated into the host genome.

To identify agents that specifically activate transcription, transcription regulatory regions could be linked to a reporter gene and the construct added to an appropriate host cell. As used herein, the term “reporter gene” means a gene that encodes a gene product that can be identified (i.e., a reporter protein). Reporter genes include, but are not limited to, alkaline phosphatase, chloramphenicol acetyltransferase, β-galactosidase, luciferase and green fluorescence protein (GFP). Identification methods for the products of reporter genes include, but are not limited to, enzymatic assays and fluorimetric assays. Reporter genes and assays to detect their products are well known in the art and are described, for example in Ausubel et al. (1987) and periodic updates. Reporter genes, reporter gene assays, and reagent kits are also readily available from commercial sources. Examples of appropriate cells include, but are not limited to, plant, fungal, yeast, mammalian, and other eukaryotic cells. A practitioner of ordinary skill will be well acquainted with techniques for transfecting eukaryotic cells, including the preparation of a suitable vector, such as a viral vector; conveying the vector into the cell, such as by electroporation; and selecting cells that have been transformed, such as by using a reporter or drug sensitivity element. The effect of an agent on transcription from the regulatory region in these constructs would be assessed through the activity of the reporter gene product.

Besides the increase in expression under conditions in which it is normally repressed mentioned above, expression could be decreased when it would normally be expressed. An agent could accomplish this through a decrease in transcription rate and the reporter gene system described above would be a means to assay for this. The host cells to assess such agents would need to be permissive for expression.

Cells transcribing mRNA (from the polynucleotide of interest) could be used to identify agents that specifically modulate the half-life of mRNA and/or the translation of mRNA. Such cells would also be used to assess the effect of an agent on the processing and/or post-translational modification of the polypeptide. An agent could modulate the amount of polypeptide in a cell by modifying the turn-over (i.e., increase or decrease the half-life) of the polypeptide. The specificity of the agent with regard to the mRNA and polypeptide would be determined by examining the products in the absence of the agent and by examining the products of unrelated mRNAs and polypeptides. Methods to examine mRNA half-life, protein processing, and protein turn-over are well known to those skilled in the art.

In vivo screening methods could also be useful in the identification of agents that modulate polypeptide function through the interaction with the polypeptide directly. Such agents could block normal polypeptide-ligand interactions, if any, or could enhance or stabilize such interactions. Such agents could also alter a conformation of the polypeptide. The effect of the agent could be determined using immunoprecipitation reactions. Appropriate antibodies would be used to precipitate the polypeptide and any protein tightly associated with it. By comparing the polypeptides immunoprecipitated from treated cells and from untreated cells, an agent could be identified that would augment or inhibit polypeptide-ligand interactions, if any. Polypeptide-ligand interactions could also be assessed using cross-linking reagents that convert a close, but noncovalent interaction between polypeptides into a covalent interaction. Techniques to examine protein-protein interactions are well known to those skilled in the art. Techniques to assess protein conformation are also well known to those skilled in the art.

It is also understood that screening methods can involve in vitro methods, such as cell-free transcription or translation systems. In those systems, transcription or translation is allowed to occur, and an agent is tested for its ability to modulate function. For an assay that determines whether an agent modulates the translation of mRNA or a polynucleotide, an in vitro transcription/translation system may be used. These systems are available commercially and provide an in vitro means to produce mRNA corresponding to a polynucleotide sequence of interest. After mRNA is made, it can be translated in vitro and the translation products compared. Comparison of translation products between an in vitro expression system that does not contain any agent (negative control) with an in vitro expression system that does contain an agent indicates whether the agent is affecting translation. Comparison of translation products between control and test polynucleotides indicates whether the agent, if acting on this level, is selectively affecting translation (as opposed to affecting translation in a general, non-selective or non-specific fashion). The modulation of polypeptide function can be accomplished in many ways including, but not limited to, the in vivo and in vitro assays listed above as well as in in vitro assays using protein preparations. Polypeptides can be extracted and/or purified from natural or recombinant sources to create protein preparations. An agent can be added to a sample of a protein preparation and the effect monitored; that is whether and how the agent acts on a polypeptide and affects its conformation, folding (or other physical characteristics), binding to other moieties (such as ligands), activity (or other functional characteristics), and/or other aspects of protein structure or functions is considered to have modulated polypeptide function.

In an example for an assay for an agent that binds to a polypeptide encoded by a polynucleotide identified by the methods described herein, a polypeptide is first recombinantly expressed in a prokaryotic or eukaryotic expression system as a native or as a fusion protein in which a polypeptide (encoded by a polynucleotide identified as described above) is conjugated with a well-characterized epitope or protein. Recombinant polypeptide is then purified by, for instance, immunoprecipitation using appropriate antibodies or anti-epitope antibodies or by binding to immobilized ligand of the conjugate. An affinity column made of polypeptide or fusion protein is then used to screen a mixture of compounds which have been appropriately labeled. Suitable labels include, but are not limited to fluorochromes, radioisotopes, enzymes and chemiluminescent compounds. The unbound and bound compounds can be separated by washes using various conditions (e.g. high salt, detergent) that are routinely employed by those skilled in the art. Non-specific binding to the affinity column can be minimized by pre-clearing the compound mixture using an affinity column containing merely the conjugate or the epitope. Similar methods can be used for screening for an agent(s) that competes for binding to polypeptides. In addition to affinity chromatography, there are other techniques such as measuring the change of melting temperature or the fluorescence anisotropy of a protein which will change upon binding another molecule. For example, a BIAcore assay using a sensor chip (supplied by Pharmacia Biosensor, Stitt et al. (1995) Cell 80: 661-670) that is covalently coupled to polypeptide may be performed to determine the binding activity of different agents.

It is also understood that the in vitro screening methods of this invention include structural, or rational, drug design, in which the amino acid sequence, three-dimensional atomic structure or other property (or properties) of a polypeptide provides a basis for designing an agent which is expected to bind to a polypeptide. Generally, the design and/or choice of agents in this context is governed by several parameters, such as side-by-side comparison of the structures of a domesticated organism's and homologous ancestral polypeptides, the perceived function of the polypeptide target, its three-dimensional structure (if known or surmised), and other aspects of rational drug design. Techniques of combinatorial chemistry can also be used to generate numerous permutations of candidate agents.

Also contemplated in screening methods of the invention are transgenic animal and plant systems, which are known in the art.

The screening methods described above represent primary screens, designed to detect any agent that may exhibit activity that modulates the function of a polynucleotide or polypeptide. The skilled artisan will recognize that secondary tests will likely be necessary in order to evaluate an agent further. For example, a secondary screen may comprise testing the agent(s) in an assay using mice and other animal models (such as rat), which are known in the art or in the domesticated or ancestral plant or animal itself. In addition, a cytotoxicity assay would be performed as a further corroboration that an agent which tested positive in a primary screen would be suitable for use in living organisms. Any assay for cytotoxicity would be suitable for this purpose, including, for example the MTT assay (Promega).

The screening methods detailed earlier in this specification may be applied specifically to EG307 or EG1117. Accordingly, the invention provides a method of identifying an agent that modulates the function of the non-polypeptide coding regions of an EG307 or EG1117 polynucleotide, comprising contacting a host cell that has been transfected with a construct comprising the non-polypeptide coding region operably linked to a reporter gene coding region, with at least one candidate agent, wherein the agent is identified by its ability to modulate the transcription or translation of said reporter polynucleotide. The present invention also provides agents identified by the method.

The present invention also provides a method of identifying an agent that modulates the function of the non-polypeptide coding regions of an evolutionarily significant EG307 or EG1117 polynucleotide, comprising contacting a plant or transgenic plant containing an EG307 or EG1117 polynucleotide with at least one candidate agent, wherein the agent is identified by its ability to modulate the transcription or translation of said reporter polynucleotide. The present invention also provides agents identified by the method.

The present invention also provides a method of identifying an agent which may modulate yield, said method comprising contacting at least one candidate agent with a plant or cell comprising an EG307 or EG1117 gene, wherein the agent is identified by its ability to modulate yield. In one embodiment the plant or cell is transfected with a polynucleotide encoding and EG307 or EG1117 gene. The present invention also provides agents identified by the method. In one embodiment, the identified agent modulates yield by modulating a function of the polynucleotide encoding the polypeptide. In another embodiment, the identified agent modulates yield by modulating a function of the polypeptide.

The invention also includes agents identified by the screening methods described herein.

The following examples are provided to further assist those of ordinary skill in the art. Such examples are intended to be illustrative and therefore should not be regarded as limiting the invention. A number of exemplary modifications and variations are described in this application and others will become apparent to those of skill in this art. Such variations are considered to fall within the scope of the invention as described and claimed herein.

### EXAMPLES

### Example 1

### cDNA Library Construction

A domesticated plant or animal cDNA library is constructed using an appropriate tissue from the plant or animal. A person of ordinary skill in the art would know the appropriate tissue or tissues to analyze according to the trait of interest. Alternately, the whole organism may be used. For example, 1 day old plant seedlings are known to express most of the plant's genes.

Total RNA is extracted from the tissue (RNeasy kit, Quiagen; RNAse-free Rapid Total RNA kit, 5 Prime-3 Prime, Inc., or any similar and suitable product) and the integrity and purity of the RNA are determined according to conventional molecular cloning methods. Poly A+ RNA is isolated (Mini-Oligo(dT) Cellulose Spin Columns, 5 Prime-3 Prime, Inc., or any similar and suitable product) and used as template for the reverse-transcription of cDNA with oligo (dT) as a primer. The synthesized cDNA is treated and modified for cloning using commercially available kits. Recombinants are then packaged and propagated in a host cell line. Portions of the packaging mixes are amplified and the remainder retained prior to amplification. The library can be normalized and the numbers of independent recombinants in the library is determined.

### Example 2

### Sequence Comparison

Randomly selected ancestor cDNA clones from the cDNA library are sequenced using an automated sequencer, such as an ABI 377 or MegaBACE 1000 or any similar and suitable product. Commonly used primers on the cloning vector such as the M13 Universal and Reverse primers are used to carry out the sequencing. For inserts that are not completely sequenced by end sequencing, dye-labeled terminators or custom primers can be used to fill in remaining gaps.

The detected sequence differences are initially checked for accuracy, for example by finding the points where there are differences between the domesticated and ancestor sequences; checking the sequence fluorogram (chromatogram) to determine if the bases that appear unique to the domesticated organism correspond to strong, clear signals specific for the called base; checking the domesticated organism's hits to see if there is more than one sequence that corresponds to a sequence change; and other methods known in the art, as needed. Multiple domesticated organism sequence entries for the same gene that have the same nucleotide at a position where there is a different ancestor nucleotide provides independent support that the domesticated sequence is accurate, and that the domesticated/ancestor difference is real. Such changes are examined using public or commercial database information and the genetic code to determine whether these DNA sequence changes result in a change in the amino acid sequence of the encoded protein. The sequences can also be examined by direct sequencing of the encoded protein.

### Example 3

### Molecular Evolution Analysis

The domesticated plant or animal and wild ancestor sequences under comparison are subjected to K_{A}/K_{S} analysis. In this analysis, publicly or commercially available computer programs, such as Li 93 and INA, are used to determine the number of non-synonymous changes per site (K_{A}) divided by the number of synonymous changes per site (K_{S}) for each sequence under study as described above. Full-length coding regions or partial segments of a coding region can be used. The higher the K_{A}/K_{S} ratio, the more likely that a sequence has undergone adaptive evolution. Statistical significance of K_{A}/K_{S} values is determined using established statistic methods and available programs such as the t-test.

To further lend support to the significance of a high K_{A}/K_{S} ratio, the domesticated sequence under study can be compared to other evolutionarily proximate species. These comparisons allow further discrimination as to whether the adaptive evolutionary changes are unique to the domesticated plant or animal lineage compared to other closely related species. The sequences can also be examined by direct sequencing of the gene of interest from representatives of several diverse domesticated populations to assess to what degree the sequence is conserved in the domesticated plant or animal.

### Example 4

### cDNA Library Construction

A teosinte cDNA library is constructed using whole teosinte 1 day old seedlings, or other appropriate plant tissues. Total RNA is extracted from the seedling tissue and the integrity and purity of the RNA are determined according to conventional molecular cloning methods. Poly A+ RNA is selected and used as template for the reverse-transcription of cDNA with oligo (dT) as a primer. The synthesized cDNA is treated and modified for cloning using commercially available kits. Recombinants are then packaged and propagated in a host cell line. Portions of the packaging mixes are amplified and the remainder retained prior to amplification. Recombinant DNA is used to transfect E. coli host cells, using established methods. The library can be normalized and the numbers of independent recombinants in the library is determined.

### Example 5

### Sequence Comparison

Randomly selected teosinte seedling cDNA clones from the cDNA library are sequenced using an automated sequencer, such as the ABI 377. Commonly used primers on the cloning vector such as the M13 Universal and Reverse primers are used to carry out the sequencing. For inserts that are not completely sequenced by end sequencing, dye-labeled terminators are used to fill in remaining gaps.

The resulting teosinte sequences are compared to domesticated maize sequences via database searches. Genome databases are publicly or commercially available for a number of species, including maize. One example of a maize database can be found at the MaizeDB website at the University of Missouri. MaizeDB is a public Internet gateway to current knowledge about the maize genome and its expression. Other appropriate maize EST (expressed sequence tag) databases are privately owned and maintained. The high scoring “hits,” i.e., sequences that show a significant (e.g., >80%) similarity after homology analysis, are retrieved and analyzed. The two homologous sequences are then aligned using the alignment program CLUSTAL V developed by Higgins et al. Any sequence divergence, including nucleotide substitution, insertion and deletion, can be detected and recorded by the alignment.

The detected sequence differences are initially checked for accuracy by finding the points where there are differences between the teosinte and maize sequences; checking the sequence fluorogram (chromatogram) to determine if the bases that appear unique to maize correspond to strong, clear signals specific for the called base; checking the maize hits to see if there is more than one maize sequence that corresponds to a sequence change; and other methods known in the art as needed. Multiple maize sequence entries for the same gene that have the same nucleotide at a position where there is a different teosinte nucleotide provides independent support that the maize sequence is accurate, and that the teosinte/maize difference is real. Such changes are examined using public/commercial database information and the genetic code to determine whether these DNA sequence changes result in a change in the amino acid sequence of the encoded protein. The sequences can also be examined by direct sequencing of the encoded protein.

### Example 6

### Molecular Evolution Analysis

The teosinte and maize sequences under comparison are subjected to K_{A}/K_{S} analysis. In this analysis, publicly or commercially available computer programs, such as Li 93 and INA, are used to determine the number of non-synonymous changes per site (K_{A}) divided by the number of synonymous changes per site (K_{S}) for each sequence under study as described above. This ratio, K_{A}/K_{S}, has been shown to be a reflection of the degree to which adaptive evolution, i.e., positive selection, has been at work in the sequence under study. Typically, full-length coding regions have been used in these comparative analyses. However, partial segments of a coding region can also be used effectively. The higher the K_{A}/K_{S} ratio, the more likely that a sequence has undergone adaptive evolution. Statistical significance of K_{A}/K_{S} values is determined using established statistic methods and available programs such as the t-test. Those genes showing statistically high K_{A}/K_{S} ratios between teosinte and maize genes are very likely to have undergone adaptive evolution.

To further lend support to the significance of a high K_{A}/K_{S} ratio, the sequence under study can be compared in other ancestral maize species. These comparisons allow further discrimination as to whether the adaptive evolutionary changes are unique to the domesticated maize lineage compared to other ancestors. The sequences can also be examined by direct sequencing of the gene of interest from representatives of several diverse maize populations to assess to what degree the sequence is conserved in the maize species.

### Example 7

### Application of K_{A}/K_{S} Method to Maize and Teosinte Homologous Sequences Obtained from a Database

Comparison of domesticated maize and teosinte sequences available on Genbank (accessable through the Entrez Nucleotides database at the National Center for Biotechnology Information web site) revealed at least four homologous genes: waxy, A1*, A1 and globulin for which sequence was available from both maize and teosinte. All available sequences for these genes for both maize and teosinte were compared. The K_{A}/K_{S} ratios were determined using Li93 and/or INA:

| | Avr. No. Syn. | Avr. No. Non-Syn. | |
|---|---|---|---|
| Gene | Substitutions | Substitutions | K_{A}/K_{S} |
| Waxy | 4 | 1 | 0.068 |
| A1* | 10 | 3 | 0.011 |
| A1 | 3 | 2 | 0.44-0.89 |
| Globulin | 10 | 7 | 0.42 |

Although it was anticipated that the polymorphism (multiple allelic copies) and/or the polyploidy (more than 2 sets of chromosomes per cell) observed in maize might make a K_{A}/K_{S} analysis complex or difficult, it was found that this was not the case.

While the above K_{A}/K_{S} values indicate that these genes are not positively selected, this example illustrates that the K_{A}/K_{S} method can be applied to maize and its teosinte sequences obtained from a database.

### Example 8

### Study of Protein Function Using a Transgenic Plant

The functional roles of a positively selected maize gene obtained according to the methods of Examples 4-7 can be assessed by conducting assessments of each allele of the gene in a transgenic maize plant. A transgenic plant can be created using an adaptation of the method described in Peng et al. (1999) Nature 400:256-261. Physiological, morphological and/or biochemical examination of the transgenic plant or protein extracts thereof will permit association of each allele with a particular phenotype.

### Example 9

### Mapping of Positively Selected Genes to QTLs

QTL (quantitative trait locus) analysis has defined chromosomal regions that contain the genes that control several phenotypic traits of interest in maize, including plant height and oil content. By physically mapping each positively-selected gene identified by this method onto one of the known QTLs, the specific trait controlled by each positively-selected gene can be rapidly and conclusively identified.

### Example 10

### Discovery of New Gene EG307

A normalized cDNA library was constructed from pooled tissues (including leaves, panicles, and stems) of Oryza rufipogon, the species known to be ancestral to modern rice. A clone designated PBI0307H9 was first sequenced as part of a high-throughput sequencing project on a MegaBACE 1000 sequencer (AP Biotech). (SEQ ID NO:89) The sequence of this clone was used as a query sequence in a BLAST search of the GenBank database. Four anonymous rice ESTs (accession nos. AU093345, C29145, ISAJ0161, AU056792) were retrieved as hits. Further sequencing revealed that PBI307H9 was a partial cDNA clone. PBI307H9 had a high K_{A}/K_{S} ratio when compared to the domesticated rice (Oryza sativa) ESTs in GenBank. cDNA amplification and sequencing were accomplished as follows: Total RNA was isolated from O. rufipogon (strain NSGC5953) and O. sativa cv. Nipponbare (Qiagen RNeasy Plant Mini Kit: cat #74903). First strand cDNA was synthesized using a dT primer (AP Biotech Ready-to-Go T-Primed First-Strand Kit: cat #27-9263-01) and then used for PCR analysis (Qiagen HotStarTaq Master Mix Kit: cat#203445).

For ease in nomenclature, the gene contained in clone PBI0307H9 is named EG307, both here and throughout. Initially, before final sequence confirmation, the Ka/Ks ratio for EG307 derived from modern rice (O. sativa) and ancestral rice (O. rufipogon) EG307 was 1.7.

Once these partial sequences were confirmed in both O. rufipogon and O. sativa, 5′ RACE (Clontech SMART RACE cDNA Amplification Kit: cat #K1811-1) was performed with a gene specific primer to obtain the 5′ end of this gene. The complete gene, termed EG307, has a coding region 1344 bp long. Final confirmation of the complete EG307 CDS (1344 bp) in O. sativa and O. rufipogon allowed pairwise comparisons of a number of strains of O. rufipogon and O. sativa. Many of these comparisons yield K_{A}/K_{S} ratios greater than one, some with statistical significance. This is compelling evidence for the role of positive selection on the EG307 gene. As the selection pressure imposed upon ancestral rice was human imposed, this is compelling evidence that EG307 is a gene that was selected for during human domestication of rice. No homologs to EG307 were identified by BLAST search to the non-redundant section of GenBank, and, as noted above, only four rice genes were identified by BLAST in the EST section of GenBank (AU093345, AU056792, C29145, and ISA0161). All four ESTs were essentially uncharacterized.

### Example 11

### Further K_{A}/K_{S} Analysis of EG307

In order to ascertain the extent of genetic diversity present in O. sativa for the EG307 gene, genomic DNA was isolated from several different strains of O. sativa (acquired from the National Small Grains Collection, U.S.D.A., Aberdeen, Id.), using Qiagen's protocol (DNeasy Plant Mini Kit: cat #69103). EG307 was then sequenced in genomic DNA from six different O. sativa strains: Nipponbare, Lemont, IR64, Teqing, Azucena, and Kasalath. The K_{A}/K_{S} ratios for each of these strains varied when compared to O. rufipogon. Table 1 shows results for the entire 1344 bases of coding region.

TABLE 1
Full CDS Ka/Ks ratios for O. rufipogon (strain IRGC105491) vs.
all O. sativa strains examined.

| | | | | | Position | |
|---|---|---|---|---|---|---|
| | | | | | (bp) | |
| | Ka | Ks | Ka/Ks | size bp | in CDS | t |
| Azucena | 0.00668 | 0.00922 | 0.724 | 1341 | 1-1341 | 0.398 |
| Lemont | 0.00668 | 0.00922 | 0.724 | 1341 | 1-1341 | 0.398 |
| Nipponbare | 0.00668 | 0.00922 | 0.724 | 1341 | 1-1341 | 0.398 |
| Kasalath-1 | 0.00204 | 0.00483 | 0.422 | 1341 | 1-1341 | 0.552 |
| Kasalath-2 | 0.00293 | 0.00482 | 0.608 | 1341 | 1-1341 | 0.369 |
| Kasalath-3 | 0.00115 | 0.00483 | 0.238 | 1341 | 1-1341 | 0.740 |
| Kasalath-4 | 0.00204 | 0.00482 | 0.423 | 1341 | 1-1341 | 0.551 |
| IR64 | 0.00204 | 0.00700 | 0.291 | 1341 | 1-1341 | 0.902 |
| Teqing | 0.000 | 0.000 | DIV/0 | 1341 | 1-1341 | DIV/0 |

There were differences in the untranslated (UTR) regions between O. rufipogon and all these O. sativa strains. The wide range of K_{A}/K_{S} ratios was expected due to the differing degrees of cross breeding among the O. sativa strains. Some were more similar to O. rufipogon than others due to cross breeding between O. rufipogon with the domesticated strains. Sliding window analysis was performed for all pairwise comparisons between the protein coding region of O. rufipogon EG307 to the protein coding region of each of the O. sativa strains we sequenced. This allowed identification of the specific areas of the protein that have been selected during domestication. Such pinpointing will allow a targeted approach to characterization of the changes that are important between the ancestral protein and the protein of the domesticated descendent crop plant. This may permit development of agents that target these vital domains of the protein, with the goal of increasing yield.

The length of the “window” was in most cases 150 bp, with a 50 bp overlap with adjacent windows. (Thus, as an example, if reading from the 5′ end of a CDS, the first window was 150 bp in length, as was the adjacent second window to its 3′ side. The second window, also 150 in length, overlapped the first window by 50 bp at the 5′ end of the second window, and the third window, also 150 bp, overlapped the second window by 50 bp at the 5′ end of the third window. Thus, the second window overlapped both its adjacent neighbors, each by 50 bp.) In addition a second window analysis was completed in which the CDS was divided approximately into halves. This allows a greater sample size of nucleotides, so that an accurate statistical sampling can be undertaken. It should also be noted that Ka/Ks, although conventionally expressed as a ratio, is really a way of asking “Does the Ka value exceed the Ks value by a statistically significant amount?” Thus, when Ks=0, as often happens in ancestral rice-to-modern rice comparisons (because there are only some 7,000-8,000 years of domestication), a ratio cannot be computed, since the denominator of the fraction would equal zero. However, such comparisons may still detect the action of positive selection, if the (Ka−Ks) difference is statistically significant. Thus for several comparisons shown in the following tables, positive selection can be detected, as long as the comparison is statistically significant. Like those comparisons for which the Ka/Ks ratio is significant, these are shown in bold.

It should also be noted that as a result of the stochastic nature of the nucleotide substitution process, not all comparisons to modern rice strains are expected to reveal evidence of positive selection, particularly since some cross breeding between O. rufipogon and modern O. sativa is known to have occurred.

TABLE 2
Sliding Window Ka/Ks Ratios for O. rufipogon (strain NSGC 5948)
vs. O. sativa, strain “Nipponbare”.

| | | | | | Position | |
|---|---|---|---|---|---|---|
| | | | | | (bp) | |
| | Ka | Ks | Ka/Ks | size bp | in CDS | t |
| Window #1 | 0.000 | 0.0178 | 0.000 | 165 | 91-255 | 0.965 |
| Window #2 | 0.00790 | 0.000 | DIV/0 | 150 | 256-405 | 0.999 |
| Window #3 | 0.000 | 0.000 | DIV/0 | 150 | 355-504 | DIV/0 |
| Window #4 | 0.000 | 0.000 | DIV/0 | 150 | 454-603 | DIV/0 |
| Window #5 | 0.0203 | 0.000 | DIV/0 | 150 | 556-705 | 1.40 |
| Window #6 | 0.0106 | 0.000 | DIV/0 | 150 | 655-804 | 0.994 |
| Window #7 | 0.0083 | 0.000 | DIV/0 | 150 | 754-903 | 0.999 |
| Window #8 | 0.0183 | 0.000 | DIV/0 | 150 | 856-1005 | 1.40 |
| Window #9 | 0.000 | 0.000 | DIV/0 | 150 | 955-1104 | DIV/0 |
| Window #10 | 0.00990 | 0.02231 | 0.444 | 150 | 1054-1203 | 0.493 |
| Window #11 | 0.00847 | 0.03236 | 0.262 | 186 | 1156-1341 | 0.942 |
| 1st large | 0.00791 | 0.000 | DIV/0 | 543 | 256-798 | 1.72 |
| Window | | | | | | |
| 2^{nd} large | 0.00788 | 0.0108 | 0.728 | 543 | 799-1341 | 0.326 |
| Window | | | | | | |
| 80% CDS | 0.00789 | 0.00540 | 1.46 | 1086 | 256-1341 | 0.495 |
| Nearly full | 0.00684 | 0.00701 | 0.976 | 1251 | 91-1341 | 0.0343 |
| CDS | | | | | | |

Note that all statistically significant comparisons are shown in bold.

It is important to note here that there is statistical support for positive selection displayed in the comparison between O. rufipogon and Nipponbare, when the first large window is used. This is good evidence that positive selection has occurred (as a result of human domestication) between the ancestral O. rufipogon, and the domesticated O. sativa (strain Nipponbare) EG307 homologs. As noted above, as a result of the stochastic nature of the nucleotide substitution process, not all comparisons to modern rice strains are expected to reveal evidence of positive selection. In addition, as noted above, cross breeding has occurred between O. rufipogon and some domesticated strains, further obscuring the signal of selection. What this analysis makes clear, however, is that positive selection has occurred on the EG307 gene.

TABLE 3
Sliding Window Ka/Ks Ratios for O. rufipogon, strain NSGC 5948,
vs. O. sativa (strain “Lemont”).

| | | | | | Position | |
|---|---|---|---|---|---|---|
| | | | | | (bp) | |
| | Ka | Ks | Ka/Ks | size bp | in CDS | t |
| Window #1 | 0.000 | 0.0178 | 0.000 | 165 | 91-255 | 0.965 |
| Window #2 | 0.00790 | 0.000 | DIV/0 | 150 | 256-405 | 0.999 |
| Window #3 | 0.000 | 0.000 | DIV/0 | 150 | 355-504 | DIV/0 |
| Window #4 | 0.000 | 0.000 | DIV/0 | 150 | 454-603 | DIV/0 |
| Window #5 | 0.0203 | 0.000 | DIV/0 | 150 | 556-705 | 1.40 |
| Window #6 | 0.0106 | 0.000 | DIV/0 | 150 | 655-804 | 0.994 |
| Window #7 | 0.0083 | 0.000 | DIV/0 | 150 | 754-903 | 0.999 |
| Window #8 | 0.0183 | 0.000 | DIV/0 | 150 | 856-1005 | 1.40 |
| Window #9 | 0.000 | 0.000 | DIV/0 | 150 | 955-1104 | DIV/0 |
| Window #10 | 0.00990 | 0.02231 | 0.444 | 150 | 1054-1203 | 0.493 |
| Window #11 | 0.00847 | 0.03236 | 0.262 | 186 | 1156-1341 | 0.942 |
| 1st large | 0.00791 | 0.000 | DIV/0 | 543 | 256-798 | 1.72 |
| Window | | | | | | |
| 2^{nd} large | 0.00788 | 0.0108 | 0.728 | 543 | 799-1341 | 0.326 |
| Window | | | | | | |
| 80% CDS | 0.00789 | 0.00540 | 1.46 | 1086 | 256-1341 | 0.495 |
| Nearly full | 0.00684 | 0.00701 | 0.976 | 1251 | 91-1341 | 0.0343 |
| CDS | | | | | | |

Note that all statistically significant comparisons are shown in bold.

It is important to note here that there is statistical support for positive selection displayed in the comparison between O. rufipogon and Lemont, when the first large window is used. This is good evidence that positive selection has occurred (as a result of human domestication) between the ancestral O. rufipogon, and the domesticated O. sativa (strain Lemont) EG307 homologs. As noted above, as a result of the stochastic nature of the nucleotide substitution process, not all comparisons to modern rice strains are expected to reveal evidence of positive selection. In addition, as noted above, cross breeding has occurred between O. rufipogon and some domesticated strains, further obscuring the signal of selection. What this analysis makes clear, however, is that positive selection has occurred on the EG307 gene.

TABLE 4
Sliding Window Ka/Ks Ratios for O. rufipogon, strain NSGC 5948,
vs. O. sativa (strain “IR64”).

| | | | | | Position | |
|---|---|---|---|---|---|---|
| | | | | | (bp) | |
| | Ka | Ks | Ka/Ks | size bp | in CDS | t |
| Window #1 | 0.000 | 0.000 | DIV/0 | 165 | 91-255 | DIV/0 |
| Window #2 | 0.000 | 0.000 | DIV/0 | 150 | 256-405 | DIV/0 |
| Window #3 | 0.000 | 0.000 | DIV/0 | 150 | 355-504 | DIV/0 |
| Window #4 | 0.000 | 0.000 | DIV/0 | 150 | 454-603 | DIV/0 |
| Window #5 | 0.000 | 0.000 | DIV/0 | 150 | 556-705 | DIV/0 |
| Window #6 | 0.000 | 0.000 | DIV/0 | 150 | 655-804 | DIV/0 |
| Window #7 | 0.000 | 0.000 | DIV/0 | 150 | 754-903 | DIV/0 |
| Window #8 | 0.000 | 0.000 | DIV/0 | 150 | 856-1005 | DIV/0 |
| Window #9 | 0.000 | 0.000 | DIV/0 | 150 | 955-1104 | DIV/0 |
| Window #10 | 0.000 | 0.000 | DIV/0 | 150 | 1054-1203 | DIV/0 |
| Window #11 | 0.000 | 0.000 | DIV/0 | 186 | 1156-1341 | DIV/0 |
| 1st large Window | 0.000 | 0.000 | DIV/0 | 543 | 256-798 | DIV/0 |
| 2^{nd} large Window | 0.000 | 0.000 | DIV/0 | 543 | 799-1341 | DIV/0 |
| 80% CDS | 0.000 | 0.000 | DIV/0 | 1086 | 256-1341 | DIV/0 |
| Nearly full CDS | 0.000 | 0.000 | DIV/0 | 1251 | 91-1341 | DIV/0 |

Note that all statistically significant comparisons are shown in bold.

Note that the protein coding region sequences of EG307 from O. rufipogon and from the O. sativa strain IR64 are identical, thus, the Ka/Ks values are equal to zero. IR64 is a low yielding modern strain (personal communication, Shannon Pinson, Research Geneticist, USDA-ARS Rice Research Unit, Beaumont, Tex.), suspected of massive amounts of interbreeding with wild O. rufipogon.

TABLE 5
Sliding Window Ka/Ks Ratios for O. rufipogon, strain NSGC 5948,
vs. O. sativa (strain “Teqing”).

| | | | | | Position | |
|---|---|---|---|---|---|---|
| | | | | | (bp) | |
| | Ka | Ks | Ka/Ks | size bp | in CDS | t |
| Window #1 | 0.00985 | 0.000 | DIV/0 | 165 | 91-255 | 0.995 |
| Window #2 | 0.000 | 0.000 | DIV/0 | 150 | 256-405 | DIV/0 |
| Window #3 | 0.000 | 0.000 | DIV/0 | 150 | 355-504 | DIV/0 |
| Window #4 | 0.000 | 0.000 | DIV/0 | 150 | 454-603 | DIV/0 |
| Window #5 | 0.000 | 0.000 | DIV/0 | 150 | 556-705 | DIV/0 |
| Window #6 | 0.000 | 0.0343 | 0.000 | 150 | 655-804 | 0.987 |
| Window #7 | 0.00826 | 0.000 | DIV/0 | 150 | 754-903 | 0.999 |
| Window #8 | 0.00806 | 0.000 | DIV/0 | 150 | 856-1005 | 0.999 |
| Window #9 | 0.000 | 0.000 | DIV/0 | 150 | 955-1104 | DIV/0 |
| Window #10 | 0.000 | 0.000 | DIV/0 | 150 | 1054-1203 | DIV/0 |
| Window #11 | 0.000 | 0.0155 | 0.000 | 186 | 1156-1341 | 0.980 |
| 1st large | 0.000 | 0.0113 | 0.000 | 543 | 256-798 | 0.996 |
| Window | | | | | | |
| 2^{nd} large | 0.00218 | 0.00536 | 0.407 | 543 | 799-1341 | 0.547 |
| Window | | | | | | |
| 80% CDS | 0.0011 | 0.00854 | 0.129 | 1086 | 256-1341 | 1.14 |
| Nearly full | 0.00218 | 0.00767 | 0.284 | 1251 | 91-1341 | 0.909 |
| CDS | | | | | | |

Note that all statistically significant comparisons are shown in bold.

Note that no comparisons between the EG307 sequences from O. rufipogon and O. sativa strain Teqing exhibit Ka/Ks ratios greater than one. However, as noted above, as a result of the stochastic nature of the nucleotide substitution process, not all comparisons to modern rice strains are expected to reveal evidence of positive selection. In addition, as noted above, cross breeding has occurred between O. rufipogon and some domesticated strains, further obscuring the signal of selection.

TABLE 6
Sliding Window Ka/Ks Ratios for O. rufipogon, strain NSGC 5948,
vs. O. sativa (strain “Azucena”).

| | | | | | Position | |
|---|---|---|---|---|---|---|
| | | | | | (bp) | |
| | Ka | Ks | Ka/Ks | size bp | in CDS | t |
| Window #1 | 0.000 | 0.0178 | 0.000 | 165 | 91-255 | 0.965 |
| Window #2 | 0.00790 | 0.000 | DIV/0 | 150 | 256-405 | 0.999 |
| Window #3 | 0.000 | 0.000 | DIV/0 | 150 | 355-504 | DIV/0 |
| Window #4 | 0.000 | 0.000 | DIV/0 | 150 | 454-603 | DIV/0 |
| Window #5 | 0.0203 | 0.000 | DIV/0 | 150 | 556-705 | 1.40 |
| Window #6 | 0.0106 | 0.000 | DIV/0 | 150 | 655-804 | 0.994 |
| Window #7 | 0.0083 | 0.000 | DIV/0 | 150 | 754-903 | 0.999 |
| Window #8 | 0.0183 | 0.000 | DIV/0 | 150 | 856-1005 | 1.40 |
| Window #9 | 0.000 | 0.000 | DIV/0 | 150 | 955-1104 | DIV/0 |
| Window #10 | 0.00990 | 0.02231 | 0.444 | 150 | 1054-1203 | 0.493 |
| Window #11 | 0.00847 | 0.03236 | 0.262 | 186 | 1156-1341 | 0.942 |
| 1st large | 0.00791 | 0.000 | DIV/0 | 543 | 256-798 | 1.72 |
| Window | | | | | | |
| 2^{nd} large | 0.00788 | 0.0108 | 0.728 | 543 | 799-1341 | 0.326 |
| Window | | | | | | |
| 80% CDS | 0.00789 | 0.00540 | 1.46 | 1086 | 256-1341 | 0.495 |
| Nearly full | 0.00684 | 0.00701 | 0.976 | 1251 | 91-1341 | 0.0343 |
| CDS | | | | | | |

Note that all statistically significant comparisons are shown in bold.

It is important to note here that there is statistical support for positive selection displayed in the comparison between O. rufipogon and Azucena, when the first large window is used. This is again good evidence that positive selection has occurred (as a result of human domestication) between the ancestral O. rufipogon, and the domesticated O. sativa (strain Azucena) EG307 homologs. As noted above, as a result of the stochastic nature of the nucleotide substitution process, not all comparisons to modern rice strains are expected to reveal evidence of positive selection. In addition, as noted above, cross breeding has occurred between O. rufipogon and some domesticated strains, further obscuring the signal of selection. What this analysis once again makes clear, however, is that positive selection has occurred on the EG307 gene.

TABLE 7
Sliding Window Ka/Ks Ratios for O. rufipogon, strain NSGC 5948, vs. O. sativa
(strain “Kasalath 4”).

| | | | | | Position (bp) | |
|---|---|---|---|---|---|---|
| | Ka | Ks | Ka/Ks | size bp | in CDS | t |
| Window #1 | 0.000 | 0.000 | DIV/0 | 150 | 1-150 | DIV/0 |
| Window #2 | 0.000 | 0.000 | DIV/0 | 150 | 100-249 | DIV/0 |
| Window #3 | 0.000 | 0.000 | DIV/0 | 150 | 199-348 | DIV/0 |
| Window #4 | 0.000 | 0.000 | DIV/0 | 150 | 301-450 | DIV/0 |
| Window #5 | 0.000 | 0.000 | DIV/0 | 150 | 400-549 | DIV/0 |
| Window #6 | 0.00826 | 0.000 | DIV/0 | 150 | 499-648 | 0.999 |
| Window #7 | 0.0163 | 0.000 | DIV/0 | 150 | 601-750 | 1.41 |
| Window #8 | 0.00790 | 0.000 | DIV/0 | 150 | 700-849 | 0.999 |
| Window #9 | 0.000 | 0.000 | DIV/0 | 150 | 799-948 | DIV/0 |
| Window #10 | 0.000 | 0.0155 | 0.000 | 186 | 901-1086 | 0.980 |
| 1st Half Window | 0.000 | 0.000 | DIV/0 | 543 | 1-543 | DIV/0 |
| 2^{nd} Half Window | 0.00437 | 0.00534 | 0.818 | 543 | 544-1086 | 0.157 |
| Full CDS: Kasalath 1 | 0.000 | 0.00268 | 0.000 | 1086 | 1-1086 | 0.996 |
| Full CDS: Kasalath 2 | 0.00110 | 0.00268 | 0.410 | 1086 | 1-1086 | 0.544 |
| Full CDS: Kasalath 3 | 0.00110 | 0.00268 | 0.410 | 1086 | 1-1086 | 0.544 |
| Full CDS: Kasalath 4 | 0.00220 | 0.00268 | 0.821 | 1086 | 1-1086 | 0.154 |

Note that all statistically significant comparisons are shown in bold.

Note that sliding windows are shown only for Kasalath 4. There are 4 allelic differences (designated as Kasalath 1, 2, 3, and 4) in this sequence, and as they differ only by single nucleotides, we have chosen to show only one, for purposes of clarity. The Ka/Ks ratios for each of the full CDS sequences, is shown, however. Note that no comparisons between the EG307 sequences from O. rufipogon and O. sativa strain Kasalath exhibit Ka/Ks ratios greater than one. However, as noted above, as a result of the stochastic nature of the nucleotide substitution process, not all comparisons to modern rice strains are expected to reveal evidence of positive selection. In addition, as noted above, cross breeding has occurred between O. rufipogon and some domesticated strains, further obscuring the signal of selection.

Upon completion of sequencing of EG307 in the NSGC 5953 strain of O. rufipogon, the completed sequence was used to design amplification primers. These primers were then used in the Polymerase Chain Reaction (PCR) to amplify the EG307 gene from several other O. rufipogon strains, including NSGC 5948, NSGC 5949, and IRGC105491. The amplified EG307 gene was then sequenced for each of these strains.

### Example 12

### Mapping EG307

EG307 was then physically mapped in rice. Clemson University has developed a Rice Nipponbare bacterial artificial chromosome (BAC) Library; See Budiman, M. A. 1999, “Construction and characterization of deep coverage BAC libraries for two model crops: Tomato and rice, and initiation of a chromosome walk to jointless-2 in tomato”. Ph.D. thesis, Texas A & M University, College Station, Tex. Library clones are available from Clemson in the form of hybridization filters.

Two different rice BAC libraries used in screening were purchased from the Clemson University Genomics Institute (CUGI). The OSJNBa library was constructed at CUGI from genomic DNA of the japonica rice strain (Nipponbare variety), and has an average insert size of 130 kb, covering 11 genome equivalents. This is one of the most widely used libraries for the International Rice Genome Sequencing Project. It was constructed in the HindIII site of pBeloBAC11 and contains 36,864 clones. The OSJNBb library was also constructed at CUGI from genomic DNA of the japonica rice strain (Nipponbare variety), and has an average insert size of 120 kb, covering 15 genome equivalents. This is another of the most widely used libraries for the International Rice Genome Sequencing Project. It was constructed in the EcoR1 site of pIndigoBac536 and contains 55,296 clones.

The DIG protocol (BMB-Roche PCR DIG Probe Synthesis Kit cat #1636090) successfully labeled a unique EG307 494 bp PCR product (primers: 5′-GAGTTCACAGGACAGCAGCA-3′ (SEQ ID NO:87) and 5′-CAATTCTCTGAGATGCCTTGG-3′) (SEQ ID NO:88) to screen against rice BAC filters. The blots were detected easily using chemiluminescence as per the DIG protocol (BMB-Roche DIG Luminescent Detection Kit: cat #1636090). Two different O. sativa libraries, OSJNBa, and OSJNBb were screened for a total of 5 different filters, three covering the OSJNBb library, and two covering the OSJNBa library. Table 8 shows the individual BACs identified by all three screens:

TABLE 8
Individual BACs identified in all screens of BAC library with
EG307 494 bp PCR product.

| | | O. sativa |
|---|---|---|
| BAC | Contig | chromosome |
| b0008J24 | contig 80 | chromosome 3 |
| b0022E21 | contig 80 | chromosome 3 |
| b0025P07 | not mapped | — |
| b0029I04 | not mapped | — |
| b0047E13 | contig 80 | chromosome 3 |
| b0023J20 | contig 80 | chromosome 3 |
| b0033B08 | contig 80 | chromosome 3 |
| b0050N19 | contig 80 | chromosome 3 |
| b0054B15 | contig 80 | chromosome 3 |
| b0071C04 | contig 80 | chromosome 3 |
| b0053G15 | contig 80 | chromosome 3 |
| a0078K13 | contig 80 | chromosome 3 |
| a0087K16 | contig 80 | chromosome 3 |
| a0076M22 | contig 80 | chromosome 3 |
| a0095O02 | contig 80 | chromosome 3 |

The reference data that allows physical mapping of a gene to a particular contig or chromosome are known to those skilled in the art, and are available on a web page made known to purchasers of filter sets or libraries from CUGI. There were also several faint, not significant hybridizations to contig 113, which was also on chromosome 3.

Rice contig 80 was on chromosome 3 and contained 66 BACs and 7 markers. Judging by the overlap of all these BACs within contig 80, EG307 was approximately 200 kb upstream of marker CDO1387 on the short arm of chromosome 3.

Data formerly in RiceGenes, a publicly accessible genome database developed and curated by the USDA-ARS is now integrated in Gramene. Gramene was recently funded by the USDA IFAFS programme to create a curated, open-source, Web-accessible data resource for comparative genome analysis in the grasses. It provides a collection of rice genetic maps from Cornell University, the Japanese Rice Genome Research Program (JRGP), and the Korea Rice Genome Research Program (KRGRP), as well as comparisons with maps from other grasses (maize, oat, and wheat). The CDO1387 marker was mapped to several different rice maps using the RiceGenes website.

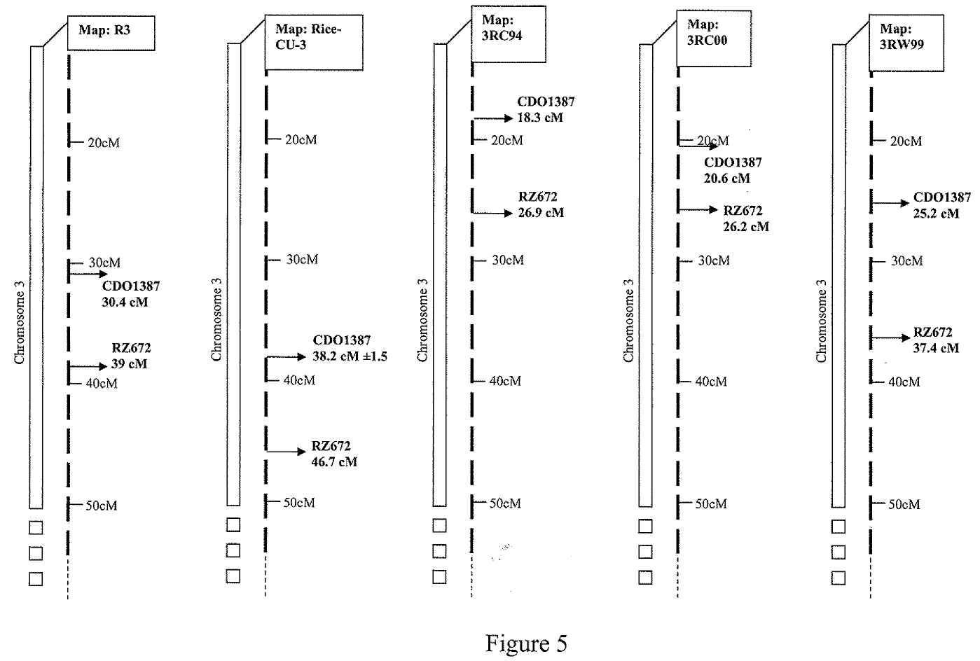

There were also several QTLs mapped to this region, but many of them had rather wide ranges that covered almost the entire chromosome. One well-documented QTL for 1000 grain weight was mapped to this region of chromosome 3 and was associated with marker RZ672 (S. R. McCouch, et al. Genetics 150:899-909 October 98). On one map (R3) CDO1387 mapped to 30.4 cM and RZ672 mapped to 39 cM, and both of these markers mapped to four other rice maps (Rice-CU-3, 3RC94, 3RC00, and 3RW99) in similar ranges (FIG. 5). Thus, EG307 was within ˜10 cM of this QTL marker. The R3 map also had a BAC, OSJNBa0091P11, mapped to 21.45 cM-21.95 cM. EG307 was negative for this BAC and any others in the same contig upon screening the rice BAC libraries. The grain weight QTL region of rice had also been involved in some synteny studies between rice and maize that indicated synteny between rice chromosome 3S and maize chromosomes 1S and 9L (W. A. Wilson, et al. Genetics 153(1): 453-473 September 99).

### Example 13

### Identification of EG307 in Maize and Teosinte

Searching the maize genome in GenBank by BLAST (using rice EG307 sequences) identified two maize ESTs, accession numbers BE511288 and BG320985, which appeared to be homologous. Primers were designed that allowed successful amplification of the maize (Zea mays) and teosinte (Zea mays parviglumis) EG307 homologs (SEQ ID NO:33 and SEQ ID NO:34, having a suggested open reading frame represented by SEQ ID NO:35, and SEQ ID NO:66, having a suggested open reading frame represented by SEQ ID NO:67). (Protein sequences for maize and teosinte were deduced; and are represented by SEQ ID NO:36 and SEQ ID NO:68.) Table 9 shows Ka/Ks estimates for a comparison between maize and teosinte.

TABLE 9
Ka/Ks Ratios for teosinte (Zea mays parviglumis) vs. modern maize
(Zea mays).

| | | | | | Position | |
|---|---|---|---|---|---|---|
| Maize | | | | | (bp) in | |
| (BS7) | Ka | Ks | Ka/Ks | size bp | CDS | t |
| Teosinte | 0.00970 | 0.0210 | 0.462 | 1347 | 1-1347 | 1.16 |
| (Benz | | | | | | |
| 967) | | | | | | |

Although these Ka/Ks values do not show ratios that are greater than one, there is still evidence for positive selection. All amino acid replacements between ancestral rice and its modern domesticated descendant were characterized, and the same analysis was performed for teosinte and its descendant, modern maize. In both (independent) cases of domestication, a consistent pattern is observed: nearly all amino acid replacements in the modern crop (whether maize or rice), as compared to the ancestral plant (teosinte or ancestral rice) result in increased charge/polarity, increased solubility, and decreased hydrophobicity. This pattern is most unlikely to have occurred by chance in these two independent domestication events. This suggests that these replacements were a similar response to human imposed domestication. This is powerful evidence that EG307 has been selected as a result of human domestication of these two cereals.

Upon completion of sequencing of EG307 in one strain of teosinte, the completed sequence was used to design amplification primers. These primers were then used in the Polymerase Chain Reaction (PCR) to amplify the EG307 gene from several other teosinte strains, as well as several strains of modern maize. The amplified EG307 gene was then sequenced for each of these strains.

Although the foregoing invention has been described in some detail by way of illustration and example for purposes of clarity and understanding, it will be apparent to those of ordinary skill in the art that certain changes and modifications can be practiced. Therefore, the description and examples should not be construed as limiting the scope of the invention, which is delineated by the appended claims.

### Example 14

### Discovery of New Gene EG1117 and K_{A} K_{S} Analysis

Clone IWF1117H5 (hereafter termed EG1117) was first sequenced during EG's high-throughput sequencing project, conducted on MegaBACE 1000 sequencers (AP Biotech). This clone was sequenced from a normalized cDNA library (Incyte Genomics) constructed from material from ancestral rice, Oryza rufipogon. GenBank BLAST results hit three anonymous rice ESTs (AU055884, AU055885, B1808367), two anonymous corn ESTs (AI783000, AWO00223), and two anonymous wheat ESTs (BE444456, BE443845). Further sequencing revealed that IWF1117H5 was a partial cDNA clone. It had a Ka/Ks ratio divisible by zero when compared to the domesticated rice, Oryza sativa, ESTs in GenBank.

Genomic DNA was isolated from several different cultivars of O. sativa following Qiagen's protocol (DNeasy Plant Mini Kit: cat #69103). Total RNA was isolated from O. rufipogon and O. sativa cv. Nipponbare (Qiagen RNeasy Plant Mini Kit: cat #74903). First strand cDNA was synthesized using a dT primer (AP Biotech Ready-To-Go T-Primed First-Strand Kit: cat #27-9263-01) and then used for PCR analysis (Qiagen HotStarTaq Master Mix Kit: cat #203445). These protocols were also performed on Zea mays (maize), Zea mays parviglumis (teosinte), and on Triticum aestivum (modern wheat).

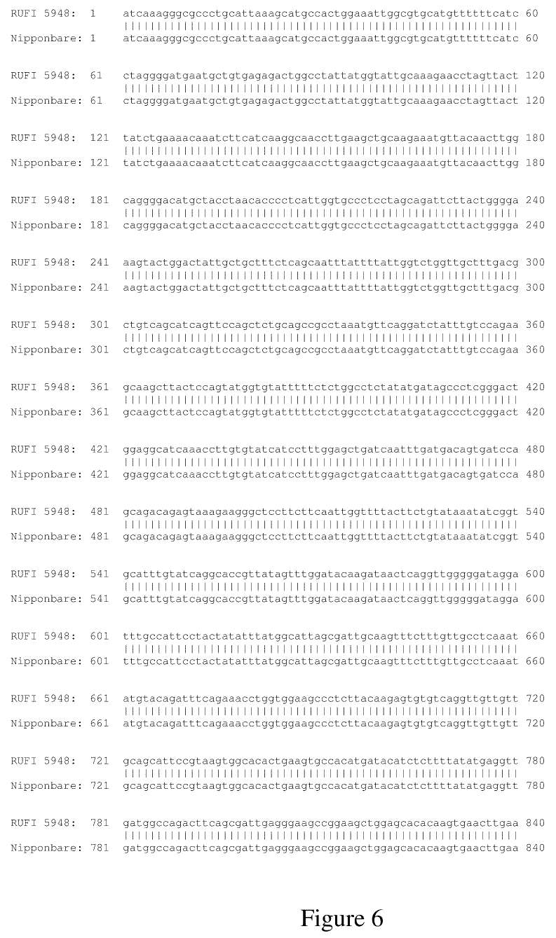

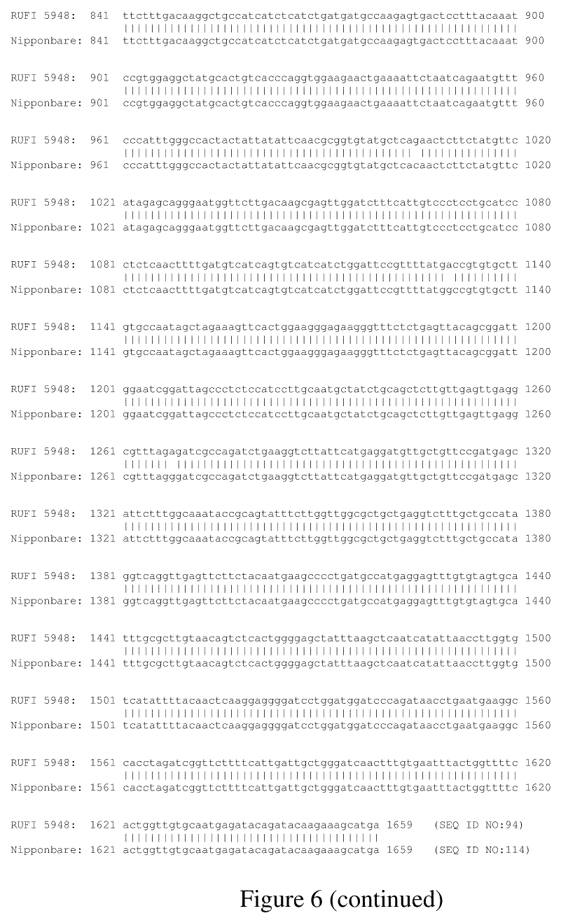

Once these partial sequences were confirmed in both O. rufipogon and O. sativa, inverse PCR was performed with gene specific primers to attempt to obtain the 5′ end of this gene. To date, 1659 bp of CDS in O. rufipogon and O. sativa (FIGS. 6 and 7) have been identified. This partial sequence includes the stop codon.

EG1117 was then partially sequenced in genomic DNA from six different O. sativa strains: Nipponbare, Lemont, IR64, Teqing, Azucena, and Kasalath. The Ka/Ks ratios for each of these strains varied when compared to O. rufipogon strain 5948. The Ka/Ks ratios for 1656 bases of coding region are as follows:

TABLE 10
Ka/Ks Ratios for O. rufipogon, strain NSGC 5948, vs. various
strains of O. sativa.

| Strain | Ka/Ks | t |
|---|---|---|
| Nipponbare: O. rufipogon | 1.5 | 0.37 |
| Lemont: O. rufipogon | 1.5 | 0.37 |
| Azucena: O. rufipogon | 1.5 | 0.37 |
| IR64: O. rufipogon | 0.0 | 1.0 |
| Teqing: O. rufipogon | 0.0 | 1.0 |
| Kasalath: O. rufipogon | 0.0 | 1.4 |

The wide range of Ka/Ks ratios is expected due to the amount of cross breeding among the O. sativa strains. Some resemble O. rufipogon because of cross breeding between O. rufipogon with the domesticated strains.

The deduced protein sequence of O. sativa strain Nipponbare was used to perform a BLAST search. A very strong protein BLAST hit to Arabidopsis thaliana PTR2-B (histidine transporting protein, NP_{—}178313) (SEQ ID NO: 170) suggests that only about 30 codons of CDS are missing from the rice sequence (FIG. 8).

Homology search results suggest that the EG1117 gene codes for a protein that is very similar to a family of peptide transport proteins that is found in a wide range of species including fungi, plants, insects and mammals. (See Koh, et al. (2002) Arabidopsis. Plant Physiol. 128:21-29; Hauser, et al. (2001) Mol. Membr. Biol. 18:105-12; Hauser, et al. (2000) J. Biol. Chem. 275:3037-42; Lubkowitz, et al. (1997) Microbiology 143-387-96; Steiner, et al. (1995) Mol Microbiol 16:825-34). EG1117 codes for a protein of 577 amino acids, that appears to have 12 putative transmembrane domain regions. K_{A}/K_{S} analysis of the EG1117 suggests that at least a portion of the EG1117 gene was strongly selected during the domestication of rice.

It is clear that this particular protein is unique even though it shows an apparent structural homology to a large number of well characterized peptide transport proteins (Steiner, et al. (1995)). The sequence appears to encode the predicted twelve transmembrane domains characteristic of this family of proteins. The EG1117 protein clearly has homology with not only peptide transport proteins, but also the oligopeptide transport proteins and nitrate transport proteins (Lubkowitz, et al. (1997); Lin, et al. (2000) Plant Physiol. 122:379-88; West, et al. (1998) Plant J. 15:221229). There is no homology to other types of transport proteins.

Peptide transport proteins are integral membrane proteins that typically contain twelve transmembrane domains in the case of the di/tripeptide transporters and may contain between twelve and fourteen transmembrane domains in the case of oligopeptide transporters. The peptide transport protein family (PTR family) has been extensively studied in yeast and plants. Typically, these proteins aid in the transport of di/tripeptides or oligopeptides across a cell membrane in a proton-dependent fashion. These carriers couple peptide movement across the membrane to movement of protons down an inwardly directed electrochemical proton gradient allowing the transport of peptides to occur against a substrate gradient (Nakazono, et al. (1996) Curr. Genet. 29:412-16; Matsukura, et al. (2000) Plant Physiol. 124:85-93; Toyofuku, et al., (2000) Plant Cell Physiol. 41:940-47; Hirose, et al. (1997) Plant Cell Physiol. 38:1389-1396; Horie, et al. (2001) Plant J. 27:129-38). Peptide transporters typically carry out sequence independent transport of all possible di- and tripeptides. All show stereoselectivity with peptides containing L-enantiomers of amino acids possessing a higher affinity for binding than peptides containing D-enantiomers. Currently, it is not possible to relate the structure of the various transporters to their substrate specificity or to their affinity.

Many different peptide transport proteins have been identified in a variety of species. Global alignments of these proteins allowed researchers to identify motifs in the primary amino acid sequence that are typically found in all members of this family. In PTR-2 members of the peptide transporters family, a “FYING” motif, named for the conserved F-Y-x-x-I-N-x-G-S-L residues in the fifth transmembrane domain (TMD5) and either a W-Q-I-P-Q-Y motif or a E-x-C-E-R-F-x-Y-Y-G motif in transmembrane domain 10 (TMD 10) have been identified (Becket, et al. (2001) in PEPTIDES: THE WAVE OF THE FUTURE, M. Lebl and R. A. Houghten, eds. American Peptide Society, 957-58). Interestingly, site directed mutagenesis of the FYING motif in S. cerevisiae results in attenuated growth on dipeptides, decreased sensitivity to toxic dipeptides and an elimination of radiolabeled dileucine. These data suggested that the FYING motif plays a crucial role in substrate recognition and/or translocation.

In the case of plants, there is evidence in the literature that peptide transporters are not only important in the nutritional uptake of peptides and nitrate, but also that these transporters affect the responses to auxins, pathogenic toxins and other developmental processes. In the case of an Arabidopsis peptide transporter, AtPRT2, it was demonstrated that root growth was affected by toxic ethionine-containing peptides thought to be transported by this particular transport protein (Steiner, et al. (1994) Plant Cell 6: 1289-99). In later studies, it was shown that either the over expression or inhibition of expression of the AtPTR2-B protein by recombinant expression of sense or antisense constructs of the AtPTR2-B gene resulted in delayed flowering and arrested seed development in transgenic Arabidopsis plants (Song, et al. (1997) Plant Physiol. 114:927-935). This suggests that peptide transporters can have a very profound effect on both the growth and development of plants.

Further analysis of the putative EG1117 peptide transporter demonstrates that a FYING motif is indeed present in TMD5 of EG1117 compared to other representative plant PTR-2 type proteins. In addition, the EG1117 has a WQVPQY motif in TMD10 identical to the other representative plant PTR-2 type proteins. The multiple sequence alignments created by the DIALIGN local alignment program (Morgenstern (1999) Bioinformatics 15:211-218, demonstrate that there is nearly 95% alignment of the diverse PTR-2 type plant protein sequences with the rice EG1117 protein with about 70% homology at the amino acid level. In the O. sativa and O. rufipogon proteins, there are only three non-synonymous amino acid replacements. These replacements are structurally significant replacements that may dramatically alter the function or specificity of the putative peptide transport protein. In one case, we have a change from a glutamine (polar uncharged) to a histidine (basic) amino acid. At the other two positions, we see a change from the acidic aspartic acid to an uncharged glycine and a change from a acidic glutamic acid to an uncharged glycine. In general, all three changes shift towards a more basic charge profile.

### Example 15

### Mapping EG1117

EG1117 was then physically mapped in rice. The DIG protocol (BMB-Roche PCR DIG Probe Synthesis Kit cat #1636090) successfully labeled a unique EG1117 657 bp PCR product (primers: 5′-TCCTGCATCCCTCTCAACTT-3′ and 5′-GCATTGGATTCGATGAATGT-3′) to screen against rice BAC filters from Clemson University. The blots were definitively detected using chemiluminescence as per the DIG protocol (BMB-Roche DIG Luminescent Detection Kit: cat #1636090). Two different O. sativa libraries (OSJNBa and OSJNBb) were screened for a total of 2 different filters. Below are the BACs identified by both screens:

TABLE 11
Individual BACs identified in all screens of BAC library
with EG1117 PCR product.

| | | O. sativa |
|---|---|---|
| BAC | Contig | chromosome |
| b0094D04 | contig 58 | chromosome 3 |
| b0067O19 | contig 58 | chromosome 3 |
| b0073E24 | contig 58 | chromosome 3 |
| b0053L18 | contig 58 | chromosome 3 |
| b0095H17 | contig 58 | chromosome 3 |
| a0004L21 | contig 58 | chromosome 3 |
| a0031E20 | contig 58 | chromosome 3 |
| a0035M21 | contig 58 | chromosome 3 |
| a0024M01 | contig 58 | chromosome 3 |

Rice contig 58 is on chromosome 3 and contains 181 BACs and 15 markers. EG1117 maps to the same BACs as markers CDO1387, C236, C875, R2778 and R2015. These all map to 35.8 cM on map 3RJ98. This marker is mapped to several different rice maps, as accessed through the RiceGenes or Gramene website. There are also several QTLs mapped to this region. One well-documented QTL for 1000-grain weight is in this region of chromosome 3 and is associated with marker RZ672 (McCouch, S. R. et al. Genetics 150:899-909). On one map CDO1387 maps to 30.4 cM and RZ672 maps to 39 cM, and both of these markers map to four other rice maps in similar ranges. This region of rice has also been involved in some studies between rice and maize that indicate synteny between rice chromosome 3S and maize chromosomes 1S and 9L (Wilson, W. A. et al. Genetics 153(1): 453-473).

### Example 16

### Relationship of EG307 and EG1117

EG1117 and the previously described gene, EG307 map to the same Clemson BAC contig, 58. EG1117 lies towards the end of the p-arm about 3 cM upstream of EG307. EG1117 maps to the same BACs as many of the markers on contig 58, and EG307 maps to the same contig, but has no markers directly mapped to its positive BACs.

A separate analysis was undertaken using data from a published YAC map for rice from the Rice Genome Project (RGP), a joint project of the National Institute of Agrobiological Sciences (NIAS) and the Institute of the Society for Techno-innovation of Agriculture, Forestry and Fisheries (STAFF) and a part of the Japanese Ministry of Agriculture, Forestry and Fisheries (MAFF) Genome Research Program.

The RGP database puts these two genes (EG1117 and EG307) 2 cM apart on chromosome 3. This YAC map has been accepted for publication in Plant Cell (Wu, J., et al., 2002 Plant Cell, prepublication copy). Upon a BLAST search, (see above), EG1117 hit AU055884 and AU055885. Both of these GenBank EST entries come from clone S20126 that maps to YACs Y2533 and Y5488. These YACs are anchored with S10968, which maps to Chromosome 3 at 33.5 cM.

The unexpected proximity of these two genes suggests a possible functional link. EG307 and EG1117 may work together to increase yield. We speculate that EG307 may be a transcription factor for EG1117, thus creating a plant operon. All indications are that both EG307 and EG1117 are logical candidates for genes that would have an impact on agriculturally important traits based upon: 1) the K_{A}/K_{S} analysis on rice domesticated and ancestral species, 2) linkage to a grain weight QTL, and 3) an evolutionary pattern of amino acid replacements between ancestral and domesticated species. EG1117 also shows evidence for a strong positive selection during domestication based upon the KA/KS analysis in rice. EG1117 codes for a protein homologous to a family of peptide transporters. Other members of this family have been shown in plants to influence growth, flowering and seed development. EG1117 is also linked to the QTL for grain weight. It is highly unlikely that this is a coincidence. These are ideal genes to use in the aims of this proposal to both validate the genes as agriculturally relevant.

### Example 17

### Validation of Yield Candidates: Association Analysis & Pedigree Analysis

The role of EG307 and EG1117 in controlling yield in the cereals can be validated by creation of transgenic plants, as described elsewhere in this patent; additional validation support comes from association analysis and pedigree analysis.

Association analysis involves sequencing each candidate gene in a large number of well-characterized rice strains to learn if the genes are associated with known traits. EG307 was sequenced in 13 well-characterized modern rice strains and it was determined that the derived, positively-selected allele is present in each of the 9 highest yielding strains, while the ancestral allele is present in the 4 lowest yielding strains. The pattern observed by examination of Table 12 is quite striking. This adds to the evidence that EG307 does influence yield, i.e., that it may be a so-called “yield” gene.

TABLE 12
Positively Selected EG307 Allele Partitions to High Yield Rice Strains

| | | 1000- | | |
|---|---|---|---|---|
| | Accession | Grain | Derived | Ancestral |
| Strain Name | Number | Weight | Allele | Allele |
| AC27 | PI 378579 | 45.97 | X | |
| Kokoku | PI 389321 | 40.55 | X | |
| Mochi | | | | |
| Razza 77 | PI 279988 | 38.64 | X | |
| Vary Voto | PI 400774 | 37.17 | X | |
| 277 | | | | |
| Azucena | PI 400077 | 32.08 | X | |
| Dalila | PI 388430 | 24.28 | X | |
| TOTO | PI 274213 | 23.97 | X | |
| Sathri Sufaid | PI 385876 | 23.95 | X | |
| Zenith | CIor 7787 | 23.93 | X | |
| Ngoat | 389239 | 9.57 | | X |
| BR52-8-1 | 408373 | 6.89 | | X |
| Jira Shahi | 392245 | 9.05 | | X |
| IR1545-339- | 408625 | 3.37 | | X |
| 2-2 | | | | |

Pedigree analysis takes advantage of two important sets of data. In addition to the available grain weight data, the derivation of many rice strains (i.e., in pedigrees) is well known. This allows a validation scheme in which yield-related candidate genes are plotted onto known rice strain pedigrees. For each strain, the known 1000-grain weight and the type of allele (i.e., the “derived”, adapted, modern allele) of EG307 and EG1117 are noted. The pattern of transmission of the adapted allele can be inferred from these data.

### Example 18

### Identification of EG1117 in Maize and Teosinte

Using methods well known to those skilled in the art described in Example 13, EG1117 was amplified from a number of maize strains (Zea mays mays) SEQ ID NOs 119, 122, 123, 124, 127, 128, 129, 132, 135, 136, 137, 140, 141, 144, 145, 146, 149, 150, 151, 154) and a number of teosinte strains (Zea mays parviglumis) (SEQ ID NOs 157, 160, 161, 162, 165, 166, 167).

### Example 19

### Determination of the Function of Gene Candidate EG307

To elucidate the function of the EG307 protein, the rice proteins it interacts with will be determined. This “guilt-by-association” approach is useful in situations where one wants to identify potential pathways or functions associated with the unknown protein (Editorial (2001) Nature 410). Two methods of determining interacting proteins include a global screening approach, such as the yeast two-hybrid approach, as well as a more direct approach using a recombinantly expressed form of the unknown protein to isolate interacting proteins based upon the affinity of their interaction. A brief outline of the experimental methods and design are presented for both methods.

A. Yeast two-hybrid (YTH) screen. The YTH screening method for interacting proteins relies upon the creation of recombinant fusions of the protein of interest with one half of a transcription activation factor protein binding domain (the bait) and the use of a cDNA library of potential protein coding regions fused with the other half of the transcription activation factor activation domain (target protein). If the bait interacts with a target protein, the two halves of the transcription factor (binding domain and activation domain) are brought together and one gets initiation of transcription of a reporter gene. There are two basic types of YTH systems typically used, a GAL4 based system for standard YTH (Fields, et al. (1989) Nature 340:2445-246) and a LexA based “Interaction-Trap” (IT) method (Golemis, et al. (1997) in CURRENT PROTOCOLS IN MOLECULAR BIOLOGY F. M. Asubel, et al., eds., John Wiley & Sons, NY; Golemis, et al (1997) in CELLS: A LABORATORY MANUAL D. L. SpSeptor, R. Goldman, and L. Leinwand, eds., Cold Spring Harbor Laboratory Press).

Two rice YTH cDNA libraries (L cv Mil-Yang) are available commercially from Eugentech, Inc. (Yusong Taejon, Korea). These libraries are created in the Stratagene HybriZAP® (GAL4 based system) from cDNA created using mRNA isolated from rice developing spikes that are either <2 cm or >2 cm in length. These libraries should encode proteins important in both early and late embryonic development. Importantly, we know from our RT-PCR analysis of EG307 expression, that the EG307 protein should be present in these tissues. Therefore, these libraries will likely express proteins that interact with the EG307 protein.

Experimental Details. Standard reagents, yeast strains, vectors and DNA isolation/sequencing specific for the HybriZAP YTH system will be obtained from Stratagene. The coding region of EG307 will be cloned using an RT-PCR amplification of O. sativa shoot mRNA. The PCR amplified insert will be cloned into a linearized pBD-GAL4 Cam phagemid vector and transformants carrying inserts will be selected on chloramphenicol plates to create the “bait” plasmid. The cloning junctions and coding region of EG307 will be sequenced using standard sequencing techniques at EG to ensure usage of the proper reading frame and that no mutations have been introduced during amplification of EG307.

Both commercial libraries from Eugentech are reported by the company to be single round amplified from the primary library and are supplied at 2×10⁸ pfu. Library I (<2 cm spike) has an initial complexity of 1×10⁶ pfu and an amplified library titer of 3.6×10⁸ pfu/ml. Insert sizes in library I range from 0.5 to 3.0 kb. Library II (>2 cm spike) has an initial complexity of 5×10⁵ pfu and an amplified library titer of 4×10⁶ pfu/ml. Insert sizes in library II range from 0.5 to 1.6 kb. Since these premade libraries are commercially available at a very reasonable cost, it is prudent to do an initial YTH hunt using this particular system.

Both the bait plasmid and target plasmid library will be co-transfected into yeast strain YRG-2 using Stratagene's YRG-2 Yeast competent cell library kit. Yeast carrying both plasmids will be selected for by the complementation of the YRG-2 auxotrophic mutations. In this case the bait and target plasmids should complement both the tryptophan and leucine auxotrophy of the YRG-2 strain. Colonies that grow up from this co-transfection will be used to create a yeast library that will next be screened for interacting target proteins by further selection on plates lacking histidine and containing X-gal. The YRG-2 strain carries two additional reporter plasmids. One carries a GAL4 binding sequence upstream of a HIS gene and is used to complement the YRG-2 histidine auxotrophic mutation. The other plasmid carries a GAL4 binding sequence upstream of a LacZ reporter gene. This enables blue/white screening for reporter gene expression when the yeast are plated on X-gal containing plates. During the interaction screening phase, only yeast containing an interacting bait:target combination will complement the histidine auxotrophy and also cause the expression of LacZ and conversion of the X-gal substrate to a blue product. This double screen for interactions helps to limit the number of false positive colonies identified. In addition, to some degree the intensity of the blue substrate production is some indication of the strength of the interaction between the bait and target proteins.

Interacting colonies will be picked, the DNA will be isolated, and sequence the target plasmid inserts from several hundred colonies will be sequenced. Sequences will be translated and searched against protein sequences, both full length coding regions and potential open reading frames from ESTs. When multiple identical sequences are identified as targets, it is likely that the protein has been preferentially selected and represents an interacting protein. If a sequence is only represented once or a few times, it is either a non-specific interacting protein, or a transcript represented a limited number of times in the cDNA library.

Multiple classes of interacting proteins should be identified this way. Ideally, proteins of known function will be highly represented and a logical function or pathway easily identified. If the interacting protein(s) are unknown, but homologous to known proteins, it may still be possible to design experiments to confirm the relevance of the interaction based on known information in the public domain.

Suspected protein-protein interactions will be validated by additional in vitro and in vivo studies. Simpler assays to confirm interactions will be performed during Phase I if time permits. Assays such as affinity pull-downs and far-westerns will be performed as additional reagents such as antibodies and recombinant proteins are made. Generation of recombinant proteins for both the bait protein (EG307) as well as putative interacting proteins will be done as necessary as epitope tagged fusion proteins (GST, myc, V5, biotin tags). Additional evidence for relevant in vivo interactions, such as fluorescence energy transfer between two appropriately constructed green fluorescent protein fusions, may be necessary to definitively prove an in vivo interaction and also measure factors that might influence that interaction. However, such experiments are clearly beyond the scope of Phase I.

It is possible that the YTH hunt will identify that the bait protein itself binds to the GAL4 transcription activation sequence and causes activation of the reporter systems. If this occurs, two bait constructs expressing the two halves of the EG307 protein independently will be constructed. These constructs would be tested for direct activation of GAL4 reporters in YRG-2. If negative for direct activation, the cDNA target library would be rescreened for bait:target interactions.

If no interacting proteins are identified from the commercial pre-made YTH libraries, it is possible that these libraries are of poor quality or that the GAL4 YTH system is not sensitive enough to identify the actual interacting proteins. In either case, having alternative libraries constructed in the interaction-trap system (LexA) can be considered. These libraries would then serve as a basis for further characterization of any other unknown candidate proteins.

### Example 20

### Direct Isolation and Identification of Interacting Proteins Using Physical Methods

To directly isolate interacting proteins from plant tissues, affinity isolation of proteins present in various solubilized plant tissues will be performed. Isolated interacting proteins will be subjected to limited proteolysis and the resulting peptide fragments will be analyzed by mass spectral analysis to identify whether peptide fragment patterns indicative of known or predicted proteins are produced.

The EG307 protein will be cloned into a bacterial expression system to generate a GST-EG307 fusion protein using Pharmacia's pGEX-5X-1 (Amersham Pharmacia). Bacterial lysates of the cultures where expression was induced by IPTG, will be used to purify the fusion protein on glutathione sepharose beads. The soluble protein will be eluted from the beads by competition for binding to the solid phase by passage of free glutathione over the column. The recombinant GST-EG307 can then be used as an affinity ligand when recoupled to fresh glutathione sepharose beads. Alternatively, free EG307 protein can also be obtained by removal of the GST domain by treatment of the fusion protein with Factor Xa. There are no Factor Xa protease sites in the predicted EG307 protein.

Plant cell lysates isolated from a large amount of O. sativa seedlings (200-300 grams) will be created by standard differential tissue disruption and clarification techniques. To isolate cytosolic proteins, the cells will be disrupted by mechanical shearing using a polytron tissue homogenizer and sonicator while keeping the tissue lysate cold in the presence of a protease inhibitor cocktail. The soluble cytosolic lysates will be clarified by differential centrifugation at low speed to remove debris, followed by high speed centrifugation to remove insoluble aggregated protein. To isolate proteins in the insoluble fraction, various detergents such as NP-40, Brij® 35, and deoxycholate will be used to solubilize membrane bound proteins isolated from these insoluble membrane fractions. Insoluble material will be removed by centrifugation.

The plant cell lysates will be individually passed over the glutathioine-sepharose:GST-EG307 beads. The beads will be washed with buffers of various ionic strengths to remove weakly bound protein. Bound proteins will then be eluted with either low pH, high salt or denaturing detergents. Pure proteins will be run on an SDS-PAGE gel and the bands will be stained with a mass spectroscopy compatible silver staining reagent or commassie blue. Bands of interest will be cut out of the gels and frozen for later analysis. These proteins will be sent to a Mass Spectroscopy facility for limited proteolysis followed by mass spectroscopy to determine the proteolytic peptide signature and identity of the interacting protein.

This technique should allow for the identification of the interacting proteins as long as the affinity of the interaction is specific and strong enough to ensure a tight binding between the EG307 protein and the potential interacting protein. These data would then allow for the identification of the interacting protein if that protein is homologous to other proteins. It is clearly possible that no proteins will be identified by this method because of a lack of affinity for the EG307 protein. Alternatively, it is possible that no interacting proteins are present in the lysates generated by the methods outlined above.

If too many proteins appear to bind to the glutathione-seph:GST-EG307 beads, it is possible that either those proteins are non-specifically binding to either the sepharose, glutathione, glutathioine synthestase or to an artificial epitope generated by creating the N-terminal GST fusion with EG307. To eliminate some of these non-specific interactions, lysates will be pre-cleared with sepharose beads alone, glutathione-sepharose as well as an irrelevant GST-fusion protein coupled to glutathione beads. If the nonspecific bands remain following the pre-clearance steps, more stringent washing and binding conditions such as higher salt, lower salt, increased or decreased pH, addition of non-ionic detergents such as Tween-20, will be employed to restrict the proteins that bind to this bait protein.

### Example 21

### Determine the Function of Gene Candidate EG1117 Coding for a Novel Protein with a Putative Peptide Transport Function

Since the EG 1117 protein likely encodes a form of peptide transport protein based upon the in silico homology data suggesting it is a member of the PTR-2 family of protein, experiments that directly assess this particular function will be carried out. Two complementary, but independent approaches will be used. First, a method of examining heterologous peptide transport protein functions in yeast will be used to examine the ability of both the domesticated and ancestral form of EG1117 to transport peptides across the cell membrane and complement auxotrophic amino acid requirements in PTR-2 deletion mutants of yeast. Second, the growth characteristics of both the domesticated and ancestral species of rice seedlings in the presence of toxic ethionine-containing peptides will be used to correlate the potential peptide transport protein mutations with a measurable phenotypic difference between the domesticated and ancestral species of rice.

Complementation analysis of heterologous peptide transport functions in auxotrophic yeast. For these studies, we will take advantage of the previously described methods used to identify novel Arabidopsis peptide transport proteins using specifically designed auxotrophic mutant yeast strains that also carried a mutation in their ability to transport di-/tripeptides (Steiner, et al., 1994). This heterologous system demonstrated that plant peptide transport proteins could be cloned into yeast cells and the recombinantly expressed protein function could be measured by the complementation of the auxotrophic amino acid requirements of the yeast strain. This is a simple, but powerful assay that will quickly yield information about the function of both forms of EG1117.

Parental yeast strain BY4742 [Matα, his3-, leu2-, lys2-, ura2-] and the YKR093W ORF deletion mutant from the “complete yeast deletion array collection” available from ATCC in which the PTR-2 gene is deleted, designated BY4742-ptr2. Both strains are available from ATCC. The domesticated and ancestral forms of EG1117 will be cloned into the pYES2.1-TOPO-TA vector (Invitrogen, Inc.) which allows for the recombinant expression of the putative PTR-2 proteins in the BY4742-ptr2 auxotrophic strain of yeast. Selection of transfectants will be performed on plates lacking uracil. Plasmid DNA will be reisolated from the transfectants and analyzed by EG 1117 specific primers to confirm that the strain carries the appropriate plasmid. Protein expression is controlled by the presence of galactose in the media. Transfectants will be grown in media containing galactose and EG 1117 protein expression will be monitored by western blot analysis of the C-terminal V5 epitope tag added by the vector. The following shows peptides used in complementation and root growth assays:

| Normal peptides | Toxic Peptides |
|---|---|
| Met-Leu | Eth-Leu |
| Met-Leu-Gly | Eth-Leu-Gly |
| Met-Leu-(Gly)₂ | Eth-Leu-(Gly)₂ |
| Met-Leu-(Gly)₃ | Eth-Leu-(Gly)₃ |

Eth = ethionine, is a toxic derivative of methionine. All selection for auxotrophic phenotype will be done on plates lacking leucine.

To test whether EG1117 codes for a peptide transport protein, the peptides listed above will be synthesized and purified commercially. Since each peptide carries a leucine, if the peptide is transported into the leucine auxotrophic strain BY4742-ptr2 transfected with a functional peptide transport protein and grown in the presence of galactose, that strain should be able to grow.

A second assay that will be performed is a inhibition assay. In this case, the BY4742-ptr2 EG307 transfectants as well as the BY4742 parental and BY4742-ptr2 deletion mutants as controls, will be plated as a lawn on YPG (yeast extract, peptone, galactose) plates and the toxic ethionine-peptide derivatives will be spotted onto membrane discs and placed on the yeast lawns (Steiner, et al., 1994). Zones of clearing around the disc would then indicate that the yeast expressed a functional transport protein the allowed the yeast to transport the toxic peptide into the cell, killing the cell.

If both the domesticated and ancestral forms of the EG1117 protein complement the amino acid auxotrophic mutations suggesting they both are functional transport proteins, the following experiments will be performed. To assess the pH or cation dependence, the pH or ionic strength of the plating media will be varied and the ability of the transfectants carrying the EG 1117 proteins to grow in the presence of complementary peptides or die in the presence of the toxic peptides will be determined. Likewise, to assess the potential differences in affinity for peptide, the dose response effects a specific peptide or toxic variant of that peptide on growth of the yeast transformants will be assessed.

It is very likely that we will find that the EG1117 protein indeed codes for a peptide transport protein. It is also likely that the two forms of the protein will display a measurable difference in this function, perhaps a change in specificity/selectivity, pH optimum or affinity will be evident. Although the non-synonymous changes in the amino acid sequence from acidic to more basic characteristics are present, it is possible that these alterations are in an unimportant region of the protein. Alternatively, it is possible that these changes do not alter the 3-dimensional structure of the protein sufficiently to alter its function.

It is unlikely that these proteins do not transport peptides, however it is possible that the EG1117 protein might be a transport protein for some other substrate. In this case, in the absence of any evidence that the EG1117 transports peptides, it may be possible to use the same system or at least the transfected yeast to sort out some of these other functions. For example, testing for monosaccharide or polysaccharide transport ability would be possible in the appropriate auxotrophic strains. Alternatively, other yeast deletion mutants for targeted transport functions could be mated with the existing transfectants. In this case, growth of the mated yeast on the selection plates would be indicative of complementation of that particular deletion mutation. Using this strategy, it would be possible to scan a large number of different yeast deletion mutants publicly available (Brachman, et al. (1998) Yeast 14:115-132.

### Example 22

### Differential Sensitivity of Ancestral and Domesticated Rice Seedlings to Ethionine-Containing Toxic Peptides

These studies represent an attempt to directly demonstrate in vivo that the EG1117 protein functions differently in the domesticated and ancestral strains of rice. The ability to transport toxic peptides results in death of the cells that take up the toxic peptides. A lack of function might be expressed as resistance to the effects of the toxic peptide on the continued growth of the seedling roots. A lack of a phenotypic difference would suggest either that EG 1117 has not been expressed, or that other transport proteins compensate for the altered function of the selected EG 1117 proteins.

A modification of the method used for Arabidopsis will be used in these studies (Steiner, et al., 1994). Rice seeds from O. sativa and O. rufipogon will be sprouted and then the seedlings are allowed to continue to grow on rice media in a dark, moist container. The seedlings will be exposed to discs impregnated with ethionine-containing toxic peptides or non-toxic peptides as a control. Initial experiments will focus on determining if dramatic differences in sensitivity to the toxic peptides exists. If no dramatic differences are observed, additional experiments using a dose range of toxic peptides will be used in a larger experiment to determine if a difference sensitivity to the toxic peptide dose exists. This would be suggestive of a difference in functional activity of the peptide transporters present in the two strains of rice.

The results from this set of studies will depend upon whether there are other peptide transport mechanisms that compensate for the hypothesized differences in the EG1117 encoded protein. As long as EG1117 is the primary peptide transport protein used by rice or has a unique function that is critical to the growth of the rice plant, any differences in EG1117's function between the domesticated and ancestral forms should be manifested by differences in susceptibility to the toxic peptides. Similar experiments in Arabidopsis successfully demonstrated that a single PTR-2 protein was part of a single peptide transport system (Steiner, et al., 1994). Therefore, mutants of the single peptide transporter yielded dramatic results on growth inhibition. In particular, these studies revealed that a deletion of the PTR-2 protein or a lack of PTR-2 protein expression due to a developmental block in PTR-2 expression in the early embryo, resulted in resistance of the plant to the presence of toxic peptides in the surrounding media. It is likely that rice seedling growth will be similar and easily reveal any differences in function of EG 1117 by alterations in rice seedling growth.

What is claimed is:

1. An isolated polynucleotide comprising a polynucleotide selected from the group consisting of:
a) a polynucleotide selected from the group consisting of SEQ ID NO:144 and SEQ ID NO:145; and
b) a polynucleotide having at least 85% homology to a polynucleotide of a), and which confers substantially the same yield as the polynucleotide of a).

2. An isolated polypeptide comprising a polynucleotide selected from the group consisting of:
a) a polypeptide encoded by a polynucleotide selected from the group consisting of SEQ ID NO:144, and SEQ ID NO:145; and
b) a polypeptide encoded by a polynucleotide having at least 85% homology to a polynucleotide of a), and which confers substantially the same yield as the polynucleotide of a).

3. A method for identifying a polynucleotide sequence comprising an EG1117 sequence in a plant, comprising the steps of:
a) comparing polypeptide-coding nucleotide sequences of said plant to polypeptide-coding nucleotide sequences comprising at least a portion of SEQ ID NO:44 or SEQ ID NO:45; and
b) selecting a corresponding polynucleotide sequence in the plant.

4. The method of claim 3, wherein the plant is corn.

5. A method for producing a transfected plant cell or transgenic plant comprising the steps of:
a) transfecting a plant cell to contain a heterologous DNA segment encoding a polypeptide and derived from an EG1117 polynucleotide comprising SEQ ID NO:44 or SEQ ID NO:45 not native to said cell; wherein said polynucleotide is operably linked to a promoter that can be used effectively for expression of transgenic proteins;
b) optionally growing and maintaining said cell under conditions whereby a transgenic plant is regenerated therefrom;
c) optionally growing said transgenic plant under conditions whereby said DNA is expressed, whereby the total amount of EG1117 polypeptide in said plant is increased.

6. Plant cells, comprising heterologous DNA encoding an EG1117 polypeptide derived from an EG1117 polynucleotide comprising SEQ ID NO:44 or SEQ ID NO:45.

7. A propagation material of a transgenic plant comprising the transgenic plant cell according to claim 6.

8. A transgenic plant containing heterologous DNA which encodes an EG1117 polypeptide derived from an EG1117 polynucleotide comprising SEQ ID NO:44 or SEQ ID NO:45 that is expressed in plant tissue.

9. An isolated polynucleotide which includes a promoter operably linked to a polynucleotide comprising SEQ ID NO:44 or SEQ ID NO:45 that encodes the EG1117 gene in plant tissue.

10. The method of claim 9, wherein the promoter is the promoter native to an EG1117 gene.

11. A method of identifying an agent that modulates the function of the non-polypeptide coding regions of an evolutionarily significant EG1117 polynucleotide, comprising contacting the transfected plant cell of claim 6 with at least one candidate agent, wherein the agent is identified by its ability to modulate the transcription or translation of said reporter polynucleotide.

12. A transfected host cell comprising a host cell transfected with a construct comprising a promoter, enhancer or intron polynucleotide from an evolutionarily significant EG1117 polynucleotide comprising SEQ ID NO:44 or SEQ ID NO:45 or any combination thereof, operably linked to a polynucleotide encoding a reporter protein.

13. A method of identifying an agent which may modulate yield, said method comprising contacting at least one candidate agent with a plant or cell comprising an EG1117 gene, wherein the agent is identified by its ability to modulate yield.

14. The method of claim 13, wherein the plant or cell is transfected with a polynucleotide encoding an EG1117 gene.

15. A method of providing increased yield in a plant comprising:
a) producing a transfected plant cell having heterologous DNA comprising SEQ ID NO:44 or SEQ ID NO:45 encoding an EG1117 polypeptide whereby EG1117 is expressed in said plant cell; and
b) growing a transgenic plant from the transfected plant cell wherein the EG1117 transgene is expressed in the transgenic plant.

16. A method of producing an EG1117 polypeptide comprising:
a) providing a cell transfected with a polynucleotide comprising SEQ ID NO:44 or SEQ ID NO:45 encoding an EG1117 polypeptide positioned for expression in the cell;
b) culturing the transfected cell under conditions for expressing the polynucleotide; and
c) isolating the EG1117 polypeptide.

17. The method of claim 16 wherein said heterologous DNA encoding the EG1117 gene is under the control of a promoter providing constitutive expression of the EG1117 gene.

18. The method of claim 16 wherein said heterologous DNA encoding the EG1117 gene is under the control of a promoter providing controllable expression of the EG1117 gene.

19. A method of isolating a yield-related gene or fragment thereof from a plant cell, comprising:
a) providing a sample of plant cell polynucleotides;
b) providing a pair of oligonucleotides having sequence homology to a conserved region of an EG1117 gene comprising SEQ ID NO:44 or SEQ ID NO:45;
c) combining the pair of oligonucleotides with the plant cell polynucleotides sample under conditions suitable for polymerase chain reaction-mediated polynucleotide amplification; and
d) isolating the amplified yield-related polynucleotide or fragment thereof.

20. A plant yield-related polynucleotide isolated according to the method of claim 19.

21. A method of isolating a yield-related polynucleotide comprising:
a) providing a preparation of polynucleotides selected from the group consisting of genomic plant cell DNA and recombinant plant cell library polynucleotides;
b) contacting the preparation with an EG1117 oligonucleotide comprising at least a portion of a polynucleotide comprising SEQ ID NO:44 or SEQ ID NO:45 under hybridization conditions providing detection of polynucleotides having 50% or greater sequence identity; and
c) isolating a yield-related polynucleotide by its association with the EG1117 oligonucleotide.

22. The method of claim 21, wherein the EG1117 oligonucleotide is detectably-labelled and the yield-related gene is isolated by its association with the detectable label.

23. The method of claim 21, wherein the EG1117 oligonucleotide is at least 12 nucleotides in length.

24. The method of claim 21, wherein the EG1117 oligonucleotide is at least 30 nucleotides in length.
